SEQUENCE LISTING (164 sequences)

SEQ ID NO: 1 - DNA, 1413 bp, Brachypodium distachyon
  source 1..1413: mol_type = unassigned DNA
ggagaagaactcgagagggaattgcagatcatgaggcagatggctatttttgtgtcacatatgcgcaaaaagagaggctatatttgtgtccctaggttcttcgttgtattgcagtttccatatcaatctgacttggtcgcatgagaaattgatggttaaataatttgaatctctcatgtagtatcaactattagatattattttcaccaaatatatttccatcggagaagaagaggctacagaggaagcagaagagaggggtgggagaatttttacacttttgtacacccacttaaacagcaaaatccgtatgaaaacaggcccaccaaaacaatgccacgataacaatccgtagaaacaaaagcttcatttaacagcggcgcaacaaagcacgcttatccatggtagttgtagtccgtatgcgatccaaagatcacgattcacgcgtgacggacggacgacgcgtgccacaccacaactaacggcatccatggtagttgtagtccgtatgcgatccaaagatcacgattcacgcgtgacggacggacgacgcgcgccacaccacaactaacagcgtgagccagcgtccaaactccggatggcaacggggacgaaacccgtcgggtagtcactgcccaaacccgtccccgcaaccttcatcccaaacccgtccccgtttccggtcgcgggtttcagttttctaccagacccgtccccatcgggtttttcatccccgtcgggaaatccgaacccgccagcatttcagcaccaagccaaagttgcagcagcaacatgaataaaaaacaacccgtttcaacaccaagataaaacaaaacattataatttagacaacatttcacacgtataacaataacatatagttctcacatataacaacaccatttcacacataaaacaacaccatttgggataaaaatatgggctatatcaggccatttttatgggccatattgagttttcgtgggtttcacaggtaccggatttgtagaatgctgaaccgggtttgaaccgtaaaatccgcgggtattgaatttgacccaatcccgtcgtcccctggtggggtaaaaacaccatcttgagtccaaacggccaccaaccaaactccgacggcaacaaacaaacggcgttgctttgctcctcggtatctccgtgaccgctcaatctcccggctgtttccccggaattgcgtggactctctcatccacacgcaaaccgcctctccctcctctctcgtcctatccgccccggtgccgtagcctcacgggactcttcttcctcccttgctataaaatccccgccccctcccgtctcctctccacacatccaaactctcaatcgcaccgagaaaaatctcctagcgatcgaagcgaagcctctcccgatcctctcaag

SEQ ID NO: 2 - DNA, 981 bp, Zea mays
  source 1..981: mol_type = unassigned DNA
tgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgatcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaag

SEQ ID NO: 3 - DNA, 1247 bp, Oryza sativa
  source 1..1247: mol_type = unassigned DNA
tagctagcatactcgaggtcattcatatgcttgagaagagagtcgggatagtccaaaataaaacaaaggtaagattacctggtcaaaagtgaaaacatcagttaaaaggtggtataagtaaaatatcggtaataaaaggtggcccaaagtgaaatttactcttttctactattataaaaattgaggatgttttgtcggtactttgatacgtcatttttgtatgaattggtttttaagtttattcgcgatttggaaatgcatatctgtatttgagtcggtttttaagttcgttgcttttgtaaatacagagggatttgtataagaaatatctttaaaaaacccatatgctaatttgacataatttttgagaaaaatatatattcaggcgaattccacaatgaacaataataagattaaaatagcttgcccccgttgcagcgatgggtattttttctagtaaaataaaagataaacttagactcaaaacatttacaaaaacaacccctaaagtcctaaagcccaaagtgctatgcacgatccatagcaagcccagcccaacccaacccaacccaacccaccccagtgcagccaactggcaaatagtctccacccccggcactatcaccgtgagttgtccgcaccaccgcacgactcgcagccaaaaaaaaaaaaagaaagaaaaaaaagaaaaagaaaaacagcagctgggtccgggtcgtgggggccggaaaagcgaggaggatcgcgagcagcgacgaggcccggccctccctccgcttccaaagaaacgccccccatcgccactatatacatacccccccctctcctcccatccccccaaccctaccaccaccaccaccaccacctcctcccccctcgctgccggacgacgagctcctcccccctccccctccgccgccgccggtaaccaccccgcccctctcctctttctttctccgttttttttttcgtcacggtctcgatctttggccttggtagtttgggtgggcgagagcggcttcgtcgcccagatcggtgcgcgggaggggcgggatctcgcggctggcgactccgggcgtgagtcggcccggatcctcgcggggaatggggctctcggatgtagatcttctttctttcttctttttgtggtagaatttgaatccctcagcattgttcatcggtagtttttcttttcatgatttgtgacaaatgcagcctcgtgcggagcttttttgtaggcctagaag

SEQ ID NO: 4 - DNA, 691 bp, synthetic construct
  misc_feature 1..691: note = Description of Artificial Sequence: Synthetic polynucleotide
  misc_feature 1..691: note = p35S promoter sequence
  source 1..691: mol_type = other DNA
gttcagaagaccagagggctattgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatacatggtggagcacgacactctcgtctactccaagaatatcaaagatacagtctcagaagaccagagggctattgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagg

SEQ ID NO: 5 - DNA, 519 bp, synthetic construct
  misc_feature 1..519: note = Description of Artificial Sequence: Synthetic polynucleotide
  source 1..519: mol_type = other DNA
gtacgccgctcgtcctcccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcag

SEQ ID NO: 6 - DNA, 1500 bp, synthetic construct
  misc_feature 1..1500: note = Description of Artificial Sequence: Synthetic polynucleotide
  misc_feature 1..1500: note = pZmUbi1+Ubi1 intron promoter sequence
  source 1..1500: mol_type = other DNA
tgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgatcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcag

SEQ ID NO: 7 - DNA, 2503 bp, Brachypodium distachyon
  source 1..2503: mol_type = unassigned DNA
ggagaagaactcgagagggaattgcagatcatgaggcagatggctatttttgtgtcacatatgcgcaaaaagagaggctatatttgtgtccctaggttcttcgttgtattgcagtttccatatcaatctgacttggtcgcatgagaaattgatggttaaataatttgaatctctcatgtagtatcaactattagatattattttcaccaaatatatttccatcggagaagaagaggctacagaggaagcagaagagaggggtgggagaatttttacacttttgtacacccacttaaacagcaaaatccgtatgaaaacaggcccaccaaaacaatgccacgataacaatccgtagaaacaaaagcttcatttaacagcggcgcaacaaagcacgcttatccatggtagttgtagtccgtatgcgatccaaagatcacgattcacgcgtgacggacggacgacgcgtgccacaccacaactaacggcatccatggtagttgtagtccgtatgcgatccaaagatcacgattcacgcgtgacggacggacgacgcgcgccacaccacaactaacagcgtgagccagcgtccaaactccggatggcaacggggacgaaacccgtcgggtagtcactgcccaaacccgtccccgcaaccttcatcccaaacccgtccccgtttccggtcgcgggtttcagttttctaccagacccgtccccatcgggtttttcatccccgtcgggaaatccgaacccgccagcatttcagcaccaagccaaagttgcagcagcaacatgaataaaaaacaacccgtttcaacaccaagataaaacaaaacattataatttagacaacatttcacacgtataacaataacatatagttctcacatataacaacaccatttcacacataaaacaacaccatttgggataaaaatatgggctatatcaggccatttttatgggccatattgagttttcgtgggtttcacaggtaccggatttgtagaatgctgaaccgggtttgaaccgtaaaatccgcgggtattgaatttgacccaatcccgtcgtcccctggtggggtaaaaacaccatcttgagtccaaacggccaccaaccaaactccgacggcaacaaacaaacggcgttgctttgctcctcggtatctccgtgaccgctcaatctcccggctgtttccccggaattgcgtggactctctcatccacacgcaaaccgcctctccctcctctctcgtcctatccgccccggtgccgtagcctcacgggactcttcttcctcccttgctataaaatccccgccccctcccgtctcctctccacacatccaaactctcaatcgcaccgagaaaaatctcctagcgatcgaagcgaagcctctcccgatcctctcaaggtacgcccgtttcccgtcgatcctcctccttccgttcgtgttctgtagccgatcgattcgattcccttacacccgttcgtgttctctcgtggatcgatcgattgtttgttgctagaaggaactcgtagatctggcgtttatgaactgtgattcgggttagtccagatcgattcaggtcggtcgtcgttgagcctctcggctatgtctggattatcgtgtagatctgctggttcagttgattatgttcttctaggagtaatttcgttgggtcagcgcgatttctgcttaatctatgctgcttattgcgcctgtacctatctactaagctatgtgcacctgtaattttgctagattattcgttcatcctcgtagttggtttgtcacagtaatccgtatgggttctgacgatgttattgttggtcatacctaggcttctccagattttattttgttaaaattggatagatctgctactgatagttgatgatggaatttggtgctgaatctatgctatttattgcgcctatacctgatctatcgggctatgtacggctgtagtttactggattattcgttcatcctcggtagttggttcatcgtttgggttctgacgataatattgttgattatgcgtaggcttctgcagattgttgttaaaattggatacatcggttactgatggttgatgatagatttgtgctgaacctatctgtttattgctcctatacctgatctatagggctatgtatgcctgtaatttaccagattattcgttcatcctcgtagttggttcatctctataattcgtatgggttcttatgatgttatcgttgattatgcctagtcttatacagattattgtgtcaagattgaatatacctgctactgatcggtgataatttggttagtagtttgcaatctgctaggaacacgttaccactgtaatctgtaaacatggtttgccagagtagtttgttctactactcttgatatggttgctgattttagtcgcctccttttggatcatgtattgatgtccttgcagatttccgtgtacttaccccggcttttgtgtacttcgtgttaacag

SEQ ID NO: 8 - DNA, 535 bp, Zea mays
  source 1..535: mol_type = unassigned DNA
gtccgccttgtttctcctctgtctcttgatctgactaatcttggtttatgattcgttgagtaattttggggaaagcttcgtccacagttttttttcgatgaacagtgccgcagtggcgctgatcttgtatgctatcctgcaatcgtggtgaacttatttcttttatatcctttactcccatgaaaaggctagtaatctttctcgatgtaacatcgtccagcactgctattaccgtgtggtccatccgacagtctggctgaacacatcatacgatctatggagcaaaaatctatcttccctgttctttaatgaaggacgtcattttcattagtatgatctaggaatgttgcaacttgcaaggaggcgtttctttctttgaatttaactaactcgttgagtggccctgtttctcggacgtaaggcctttgctgctccacacatgtccattcgaattttaccgtgtttagcaagggcgaaaagtttgcatcttgatgatttagcttgactatgcgattgctttcctggacccgtgcag

SEQ ID NO: 9 - DNA, 1090 bp, Brachypodium distachyon
  source 1..1090: mol_type = unassigned DNA
gtacgcccgtttcccgtcgatcctcctccttccgttcgtgttctgtagccgatcgattcgattcccttacacccgttcgtgttctctcgtggatcgatcgattgtttgttgctagaaggaactcgtagatctggcgtttatgaactgtgattcgggttagtccagatcgattcaggtcggtcgtcgttgagcctctcggctatgtctggattatcgtgtagatctgctggttcagttgattatgttcttctaggagtaatttcgttgggtcagcgcgatttctgcttaatctatgctgcttattgcgcctgtacctatctactaagctatgtgcacctgtaattttgctagattattcgttcatcctcgtagttggtttgtcacagtaatccgtatgggttctgacgatgttattgttggtcatacctaggcttctccagattttattttgttaaaattggatagatctgctactgatagttgatgatggaatttggtgctgaatctatgctatttattgcgcctatacctgatctatcgggctatgtacggctgtagtttactggattattcgttcatcctcggtagttggttcatcgtttgggttctgacgataatattgttgattatgcgtaggcttctgcagattgttgttaaaattggatacatcggttactgatggttgatgatagatttgtgctgaacctatctgtttattgctcctatacctgatctatagggctatgtatgcctgtaatttaccagattattcgttcatcctcgtagttggttcatctctataattcgtatgggttcttatgatgttatcgttgattatgcctagtcttatacagattattgtgtcaagattgaatatacctgctactgatcggtgataatttggttagtagtttgcaatctgctaggaacacgttaccactgtaatctgtaaacatggtttgccagagtagtttgttctactactcttgatatggttgctgattttagtcgcctccttttggatcatgtattgatgtccttgcagatttccgtgtacttaccccggcttttgtgtacttcgtgttaacag

SEQ ID NO: 10 - DNA, 517 bp, Zea mays
  source 1..517: mol_type = unassigned DNA
gtacgccgctcgtcctcccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcag

SEQ ID NO: 11 - DNA, 258 bp, synthetic construct
  misc_feature 1..258: note = Description of Artificial Sequence: Synthetic polynucleotide
  misc_feature 1..258: note = NosT terminator sequence
  source 1..258: mol_type = other DNA
cgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatc

SEQ ID NO: 12 - DNA, 204 bp, synthetic construct
  misc_feature 1..204: note = Description of Artificial Sequence: Synthetic polynucleotide
  misc_feature 1..204: note = 35S terminator sequence
  source 1..204: mol_type = other DNA
gtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaatta

SEQ ID NO: 13 - DNA, 3771 bp, synthetic construct
  misc_feature 1..3771: note = Description of Artificial Sequence: Synthetic polynucleotide
  misc_feature 1..3771: note = codon optimized polynulceotide
  source 1..3771: mol_type = other DNA
atggcatctagcatggcaccaaagaaaaaaaggaaagtttccaaacttgaaaaatttacaaactgctactccctttccaagacgcttaggtttaaagcgatccccgttggcaagacccaagagaatatcgataacaaaagacttctggtcgaagatgaaaaaagggccgaagactacaagggggtcaagaagttgctcgatcgctattatctttcctttatcaacgatgtgcttcattcaatcaaactgaagaacttgaataactacattagccttttcagaaagaaaacgaggactgaaaaggagaacaaggaacttgagaatcttgaaataaaccttcgcaaagaaattgcaaaagccttcaaggggaacgaaggatataaatctcttttcaaaaaagacattatagaaacaattttgcctgagtttcttgacgacaaggatgaaattgcgctcgtcaatagctttaacggatttacaactgccttcacagggttcttcgacaatagggagaatatgtttagcgaggaggcaaaaagcacatccatcgcattcagatgcatcaatgaaaatcttacccggtacatatcgaatatggacatatttgaaaaagtggatgcaatattcgataagcacgaagtccaggagataaaggaaaagatactgaatagcgactatgatgtcgaagattttttcgaaggtgagttcttcaactttgtcctgactcaagaaggcattgatgtctataatgcaataattggaggttttgtgactgagtctggcgagaagataaagggcttgaacgagtatatcaatctctacaaccagaagactaagcaaaagttgcctaaatttaaaccgctttacaagcaagttttgagcgaccgggaaagcctttccttttacggtgaaggatacacgagcgatgaagaagtcctcgaagtcttccgcaacacactcaacaagaactcagaaatcttttcctcaattaaaaaattggagaagcttttcaagaacttcgatgaatactcttcggcggggatttttgtgaagaacggcccggcaatttccacaatatctaaagacattttcggagaatggaacgtgataagagacaagtggaatgcggagtatgatgacatacacctgaagaagaaggcagttgtgactgaaaaatacgaagatgacaggagaaaaagctttaaaaagatcgggtccttttcactggaacagctgcaggagtatgccgacgccgatctttcggttgtcgaaaagctcaaagaaataattatccagaaggtcgatgaaatctacaaggtgtacggctcaagcgagaagctctttgatgctgacttcgtgttggagaagtctcttaaaaaaaacgacgcagtcgtcgcgataatgaaagatttgctggattcagtgaaatccttcgagaattatatcaaagccttcttcggcgaggggaaggagacaaacagggatgagtccttctatggagacttcgttctggcttacgacatccttcttaaggtcgaccacatctatgacgcaattcggaactatgtgacgcagaagccgtattcgaaagataagttcaagctctatttccaaaaccctcaatttatgggtgggtgggataaagacaaagagaccgattaccgggcaacaattttgcggtacgggtctaaatattacctcgctataatggataagaaatacgctaaatgtctccagaaaattgacaaagatgacgtcaacggcaattatgaaaaaatcaattataaactccttcctggcccaaataaaatgctcccgaaggtgtttttttccaaaaagtggatggcctattataatccatcagaggatattcagaaaatctataaaaatgggacctttaagaagggtgacatgtttaacctgaacgattgccacaagcttatagattttttcaaagactctattagccgctatcccaaatggtctaatgcttatgatttcaacttctctgaaactgaaaagtacaaagatattgcaggattctaccgcgaagttgaagaacaaggttataaggtttcctttgagtctgcgtccaagaaagaggtcgataagttggtcgaagaagggaaattgtatatgtttcaaatttacaataaagacttttccgacaagtcccatggtacacctaatctgcataccatgtacttcaaactgctgttcgatgagaataatcacggtcagattcgcctgagcggaggggcggaactcttcatgaggagagcatcgttgaaaaaagaggagctcgtcgtgcatccggctaacagccccattgctaacaagaatccggataatccaaagaagactactaccctctcctatgacgtctataaggataagagattctctgaggaccagtacgagttgcacatccctattgcgataaataaatgccctaagaacatctttaaaatcaatactgaggtcagagtcctgcttaagcacgacgacaacccgtatgtgatcgggattgataggggtgaaaggaacttgctttatattgtggttgtcgatggaaaaggtaatatagtggaacaatactctctgaatgaaattatcaacaacttcaatggcattaggatcaagaccgactatcattctctgttggacaagaaagagaaagagcgcttcgaggcacggcaaaactggacgtctattgagaacatcaaggagcttaaggctggttacatttctcaggttgtgcacaaaatttgcgaactggtcgagaaatatgatgccgttatcgcacttgaagatctcaacagcggatttaagaattctcgggtgaaagtcgaaaaacaggtgtatcaaaaattcgaaaagatgctgatcgacaagctcaattatatggttgataaaaagagcaacccatgcgccacggggggtgcgcttaagggctatcagattacgaacaaatttgaatccttcaagtcaatgtcgacgcaaaatgggtttatattctatataccggcgtggcttacatctaaaatagatcctagcactgggttcgtgaacctgctgaaaaccaagtacacttcaatcgcagattctaaaaaatttataagcagcttcgacagaatcatgtatgtgcccgaggaagacctcttcgagtttgcccttgattacaaaaatttctcaagaacggatgcagactacataaagaagtggaagctgtactcttatgggaaccggattcggatattcagaaatccgaaaaaaaacaatgtctttgattgggaggaagtttgtcttacctctgcttacaaagagctgttcaataaatatggcattaattaccagcaaggtgatatccgggcgctcctttgcgaacagtctgacaaagctttctattcttcatttatggcgctcatgtcattgatgctgcagatgaggaatagcattacggggaggactgatgttgactttctgatctcgcccgtgaaaaattctgatggaatcttctacgattccaggaattatgaggcccaggaaaatgctatccttcccaagaacgcagacgcaaatggcgcgtacaatatagctcgcaaggttttgtgggctataggccaattcaagaaagccgaagacgaaaagctggacaaagttaagattgctatatctaacaaagagtggcttgagtatgcgcaaacatctgttaaacacaaacgccccgcggctacaaagaaggctggccaggcaaagaagaagaagtga

SEQ ID NO: 14 - DNA, 3684 bp, synthetic construct
  misc_feature 1..3684: note = Description of Artificial Sequence: Synthetic polynucleotide
  misc_feature 1..3684: note = codon optimized polynucleotide
  source 1..3684: mol_type = other DNA
atgtccaaacttgaaaaatttacaaactgctactccctttccaagacgcttaggtttaaagcgatccccgttggcaagacccaagagaatatcgataacaaaagacttctggtcgaagatgaaaaaagggccgaagactacaagggggtcaagaagttgctcgatcgctattatctttcctttatcaacgatgtgcttcattcaatcaaactgaagaacttgaataactacattagccttttcagaaagaaaacgaggactgaaaaggagaacaaggaacttgagaatcttgaaataaaccttcgcaaagaaattgcaaaagccttcaaggggaacgaaggatataaatctcttttcaaaaaagacattatagaaacaattttgcctgagtttcttgacgacaaggatgaaattgcgctcgtcaatagctttaacggatttacaactgccttcacagggttcttcgacaatagggagaatatgtttagcgaggaggcaaaaagcacatccatcgcattcagatgcatcaatgaaaatcttacccggtacatatcgaatatggacatatttgaaaaagtggatgcaatattcgataagcacgaagtccaggagataaaggaaaagatactgaatagcgactatgatgtcgaagattttttcgaaggtgagttcttcaactttgtcctgactcaagaaggcattgatgtctataatgcaataattggaggttttgtgactgagtctggcgagaagataaagggcttgaacgagtatatcaatctctacaaccagaagactaagcaaaagttgcctaaatttaaaccgctttacaagcaagttttgagcgaccgggaaagcctttccttttacggtgaaggatacacgagcgatgaagaagtcctcgaagtcttccgcaacacactcaacaagaactcagaaatcttttcctcaattaaaaaattggagaagcttttcaagaacttcgatgaatactcttcggcggggatttttgtgaagaacggcccggcaatttccacaatatctaaagacattttcggagaatggaacgtgataagagacaagtggaatgcggagtatgatgacatacacctgaagaagaaggcagttgtgactgaaaaatacgaagatgacaggagaaaaagctttaaaaagatcgggtccttttcactggaacagctgcaggagtatgccgacgccgatctttcggttgtcgaaaagctcaaagaaataattatccagaaggtcgatgaaatctacaaggtgtacggctcaagcgagaagctctttgatgctgacttcgtgttggagaagtctcttaaaaaaaacgacgcagtcgtcgcgataatgaaagatttgctggattcagtgaaatccttcgagaattatatcaaagccttcttcggcgaggggaaggagacaaacagggatgagtccttctatggagacttcgttctggcttacgacatccttcttaaggtcgaccacatctatgacgcaattcggaactatgtgacgcagaagccgtattcgaaagataagttcaagctctatttccaaaaccctcaatttatgggtgggtgggataaagacaaagagaccgattaccgggcaacaattttgcggtacgggtctaaatattacctcgctataatggataagaaatacgctaaatgtctccagaaaattgacaaagatgacgtcaacggcaattatgaaaaaatcaattataaactccttcctggcccaaataaaatgctcccgaaggtgtttttttccaaaaagtggatggcctattataatccatcagaggatattcagaaaatctataaaaatgggacctttaagaagggtgacatgtttaacctgaacgattgccacaagcttatagattttttcaaagactctattagccgctatcccaaatggtctaatgcttatgatttcaacttctctgaaactgaaaagtacaaagatattgcaggattctaccgcgaagttgaagaacaaggttataaggtttcctttgagtctgcgtccaagaaagaggtcgataagttggtcgaagaagggaaattgtatatgtttcaaatttacaataaagacttttccgacaagtcccatggtacacctaatctgcataccatgtacttcaaactgctgttcgatgagaataatcacggtcagattcgcctgagcggaggggcggaactcttcatgaggagagcatcgttgaaaaaagaggagctcgtcgtgcatccggctaacagccccattgctaacaagaatccggataatccaaagaagactactaccctctcctatgacgtctataaggataagagattctctgaggaccagtacgagttgcacatccctattgcgataaataaatgccctaagaacatctttaaaatcaatactgaggtcagagtcctgcttaagcacgacgacaacccgtatgtgatcgggattgataggggtgaaaggaacttgctttatattgtggttgtcgatggaaaaggtaatatagtggaacaatactctctgaatgaaattatcaacaacttcaatggcattaggatcaagaccgactatcattctctgttggacaagaaagagaaagagcgcttcgaggcacggcaaaactggacgtctattgagaacatcaaggagcttaaggctggttacatttctcaggttgtgcacaaaatttgcgaactggtcgagaaatatgatgccgttatcgcacttgaagatctcaacagcggatttaagaattctcgggtgaaagtcgaaaaacaggtgtatcaaaaattcgaaaagatgctgatcgacaagctcaattatatggttgataaaaagagcaacccatgcgccacggggggtgcgcttaagggctatcagattacgaacaaatttgaatccttcaagtcaatgtcgacgcaaaatgggtttatattctatataccggcgtggcttacatctaaaatagatcctagcactgggttcgtgaacctgctgaaaaccaagtacacttcaatcgcagattctaaaaaatttataagcagcttcgacagaatcatgtatgtgcccgaggaagacctcttcgagtttgcccttgattacaaaaatttctcaagaacggatgcagactacataaagaagtggaagctgtactcttatgggaaccggattcggatattcagaaatccgaaaaaaaacaatgtctttgattgggaggaagtttgtcttacctctgcttacaaagagctgttcaataaatatggcattaattaccagcaaggtgatatccgggcgctcctttgcgaacagtctgacaaagctttctattcttcatttatggcgctcatgtcattgatgctgcagatgaggaatagcattacggggaggactgatgttgactttctgatctcgcccgtgaaaaattctgatggaatcttctacgattccaggaattatgaggcccaggaaaatgctatccttcccaagaacgcagacgcaaatggcgcgtacaatatagctcgcaaggttttgtgggctataggccaattcaagaaagccgaagacgaaaagctggacaaagttaagattgctatatctaacaaagagtggcttgagtatgcgcaaacatctgttaaacac

SEQ ID NO: 15 - protein, 1255 aa, synthetic construct
  REGION 1..1255: note = Description of Artificial Sequence: Synthetic polypeptide
  REGION 1..1255: note = codon optimized polypeptide
MASSMAPKKKRKVSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHKRPAATKKAGQAKKK

SEQ ID NO: 16 - protein, 1228 aa, unidentified
  REGION 1..1228: note = Description of Unknown: Lachnospiraceae bacterium sequence
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 17 - DNA, 37 bp, synthetic construct
  misc_feature 1..37: note = Description of Artificial Sequence: Synthetic oligonucleotide
  misc_feature 1..37: note = HH ribozyme encoding sequence
  source 1..37: mol_type = other DNA
ctgatgagtccgtgaggacgaaacgagtaagctcgtc

SEQ ID NO: 18 - RNA, 37 nt, synthetic construct
  misc_feature 1..37: note = Description of Artificial Sequence: Synthetic oligonucleotide
  misc_feature 1..37: note = HH ribozyme sequence
  source 1..37: mol_type = other RNA
ctgatgagtccgtgaggacgaaacgagtaagctcgtc

SEQ ID NO: 19 - DNA, 53 bp, Oryza sativa
  source 1..53: mol_type = unassigned DNA
ccgccaacactgccaatgccggtcccaagcccggataaaagtggagggggcgg

SEQ ID NO: 20 - RNA, 53 nt, Oryza sativa
  source 1..53: mol_type = unassigned RNA
ccgccaacactgccaatgccggtcccaagcccggataaaagtggagggggcgg

SEQ ID NO: 21 - DNA, 63 bp, Helianthus annuus
  source 1..63: mol_type = unassigned DNA
gcggggggcgtcagtcctactctgcacctcctcgtggtgtcgcctgggaaccctctttcgcaa

SEQ ID NO: 22 - RNA, 63 nt, Helianthus annuus
  source 1..63: mol_type = unassigned RNA
gcggggggcgtcagtcctactctgcacctcctcgtggtgtcgcctgggaaccctctttcgcaa

SEQ ID NO: 23 - DNA, 86 bp, Helianthus annuus
  source 1..86: mol_type = unassigned DNA
gcggggggcgtcagtcctactctgcacctcctcgtggtgtcgcctgggaaccctctttcgcaagaaagaggagccaagcagagagg

SEQ ID NO: 24 - RNA, 86 nt, Helianthus annuus
  source 1..86: mol_type = unassigned RNA
gcggggggcgtcagtcctactctgcacctcctcgtggtgtcgcctgggaaccctctttcgcaagaaagaggagccaagcagagagg

SEQ ID NO: 25 - DNA, 80 bp, Cynara scolymus
  source 1..80: mol_type = unassigned DNA
ggcgtcagtcctactctgcacctcctcgtggtgtcgcctgggaaccctctttcacaagaaagaggagccaagcagagagg

SEQ ID NO: 26 - RNA, 80 nt, Cynara scolymus
  source 1..80: mol_type = unassigned RNA
ggcgtcagtcctactctgcacctcctcgtggtgtcgcctgggaaccctctttcacaagaaagaggagccaagcagagagg

SEQ ID NO: 27 - DNA, 68 bp, Hepatitis Delta Virus
  source 1..68: mol_type = unassigned DNA
ggccggcatggtcccagcctcctcgctggcgccggctgggcaacatgcttcggcatggcgaatgggac

SEQ ID NO: 28 - RNA, 68 nt, Hepatitis Delta Virus
  source 1..68: mol_type = unassigned RNA
ggccggcatggtcccagcctcctcgctggcgccggctgggcaacatgcttcggcatggcgaatgggac

SEQ ID NO: 29 - DNA, 21 bp, synthetic construct
  misc_feature 1..21: note = Description of Artificial Sequence: Synthetic oligonucleotide
  misc_feature 1..21: note = scaffold RNA encoding sequnece
  source 1..21: mol_type = other DNA
taatttctactaagtgtagat

SEQ ID NO: 30 - RNA, 21 nt, synthetic construct
  misc_feature 1..21: note = Description of Artificial Sequence: Synthetic oligonucleotide
  misc_feature 1..21: note = scaffold RNA sequence
  source 1..21: mol_type = other RNA
taatttctactaagtgtagat

SEQ ID NO: 31 - DNA, 717 bp, synthetic construct
  misc_feature 1..717: note = Description of Artificial Sequence: Synthetic polynucleotide
  misc_feature 1..717: note = tdTomato polynucleotide
  source 1..717: mol_type = other DNA
atggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaagtctagaggtacctga

SEQ ID NO: 32 - DNA, 720 bp, synthetic construct
  misc_feature 1..720: note = Description of Artificial Sequence: Synthetic polynucleotide
  misc_feature 1..720: note = mNeoGreen polynucleotide
  source 1..720: mol_type = other DNA
atggcgtcctccgtgagtaaaggagaagaagataacatggcttcgcttccagccacacatgagcttcacatcttcggttccatcaacggcgttgacttcgatatggtcggacaaggcactgggaaccctaatgacggatacgaagagctgaacctcaagagcaccaaaggtgatcttcagttttctccatggattctggtgccacacattggctacggattccatcaataccttccataccctgacggaatgagtccattccaagcagccatggttgatggctccggataccaagtccacaggacaatgcagtttgaggacggtgcttcgctcaccgtcaactaccgttacacttacgaagggagccacatcaaaggagaagcccaagtgaaggggacaggctttcctgctgatggacctgtcatgaccaactccttaactgccgctgattggtgccggtccaagaaaacctaccctaacgacaagaccatcattagtaccttcaaatggtcttacaccacaggcaatggcaagagatatcgctctacagccaggactacctacacattcgctaaaccaatggccgctaactaccttaagaaccaacccatgtacgtgttccgtaagactgagttgaaacattccaagaccgaacttaacttcaaggagtggcagaaggcatttaccgacgtaatgggcatggatgaactatacaaataa

SEQ ID NO: 33 - DNA, 5693 bp, synthetic construct
  misc_feature 1..5693: note = Description of Artificial Sequence: Synthetic polynucleotide
  misc_feature 1..5693: note = pGEP424 tdT embedded gRNA delivery vector
  source 1..5693: mol_type = other DNA
ctgacgcgccctgtagcggcacgtcacctgcatcaggaattcaggagaagaactcgagagggaattgcagatcatgaggcagatggctatttttgtgtcacatatgcgcaaaaagagaggctatatttgtgtccctaggttcttcgttgtattgcagtttccatatcaatctgacttggtcgcatgagaaattgatggttaaataatttgaatctctcatgtagtatcaactattagatattattttcaccaaatatatttccatcggagaagaagaggctacagaggaagcagaagagaggggtgggagaatttttacacttttgtacacccacttaaacagcaaaatccgtatgaaaacaggcccaccaaaacaatgccacgataacaatccgtagaaacaaaagcttcatttaacagcggcgcaacaaagcacgcttatccatggtagttgtagtccgtatgcgatccaaagatcacgattcacgcgtgacggacggacgacgcgtgccacaccacaactaacggcatccatggtagttgtagtccgtatgcgatccaaagatcacgattcacgcgtgacggacggacgacgcgcgccacaccacaactaacagcgtgagccagcgtccaaactccggatggcaacggggacgaaacccgtcgggtagtcactgcccaaacccgtccccgcaaccttcatcccaaacccgtccccgtttccggtcgcgggtttcagttttctaccagacccgtccccatcgggtttttcatccccgtcgggaaatccgaacccgccagcatttcagcaccaagccaaagttgcagcagcaacatgaataaaaaacaacccgtttcaacaccaagataaaacaaaacattataatttagacaacatttcacacgtataacaataacatatagttctcacatataacaacaccatttcacacataaaacaacaccatttgggataaaaatatgggctatatcaggccatttttatgggccatattgagttttcgtgggtttcacaggtaccggatttgtagaatgctgaaccgggtttgaaccgtaaaatccgcgggtattgaatttgacccaatcccgtcgtcccctggtggggtaaaaacaccatcttgagtccaaacggccaccaaccaaactccgacggcaacaaacaaacggcgttgctttgctcctcggtatctccgtgaccgctcaatctcccggctgtttccccggaattgcgtggactctctcatccacacgcaaaccgcctctccctcctctctcgtcctatccgccccggtgccgtagcctcacgggactcttcttcctcccttgctataaaatccccgccccctcctgtctcctctccacacatccaaactctcaatcgcaccgagaaaaatctcctagcgatcgaagcgaagcctctcccgatcctctcaaggtacgcccgtttcccgtcgatcctcctccttccgttcgtgttctgtagccgatcgattcgattcccttacacccgttcgtgttctctcgtggatcgatcgattgtttgttgctagaaggaactcgtagatctggcgtttatgaactgtgattcgggttagtccagatcgattcaggtcggtcgtcgttgagcctctcggctatgtctggattatcgtgtagatctgctggttcagttgattatgttcttctaggagtaatttcgttgggtcagcgcgatttctgcttaatctatgctgcttattgcgcctgtacctatctactaagctatgtgcacctgtaattttgctagattattcgttcatcctcgtagttggtttgtcacagtaatccgtatgggttctgacgatgttattgttggtcatacctaggcttctccagattttattttgttaaaattggatagatctgctactgatagttgatgatggaatttggtgctgaatctatgctatttattgcgcctatacctgatctatcgggctatgtacggctgtagtttactggattattcgttcatcctcggtagttggttcatcgtttgggttctgacgataatattgttgattatgcgtaggcttctgcagattgttgttaaaattggatacatcggttactgatggttgatgatagatttgtgctgaacctatctgtttattgctcctatacctgatctatagggctatgtatgcctgtaatttaccagattattcgttcatcctcgtagttggttcatctctataattcgtatgggttcttatgatgttatcgttgattatgcctagtcttatacagattattgtgtcaagattgaatatacctgctactgatcggtgataatttggttagtagtttgcaatctgctaggaacacgttaccactgtaatctgtaaacatggtttgccagagtagtttgttctactactcttgatatggttgctgattttagtcgcctccttttggatcatgtattgatgtccttgcagatttccgtgtacttaccccggcttttgtgtacttcgtgttaacagctctagaggatcctctcaacacaacatatacaaaacaaacgaatctcaagcaatcaagcattctacttctattgcagcaatttaaatcatttcttttaaagcaaaagcaattttctgaaaattttcaccatttacgaacgatagggcgcgatcccgccaccatggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaagtctagaggtacctgataatttctactaagtgtagatgagacggagctcagtctgaccgcggcgtctcttaatttctactaagtgtagatcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgctcgacgcggccgccatggcctctagtggatcaggtgtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 34 - DNA, 4959 bp, synthetic construct
  misc_feature 1..4959: note = Description of Artificial Sequence: Synthetic polynucleotide
  misc_feature 1..4959: note = pGEP425 vector control guide RNA delivery without tdT
  source 1..4959: mol_type = other DNA
ctgacgcgccctgtagcggcacgtcacctgcatcaggaattcaggagaagaactcgagagggaattgcagatcatgaggcagatggctatttttgtgtcacatatgcgcaaaaagagaggctatatttgtgtccctaggttcttcgttgtattgcagtttccatatcaatctgacttggtcgcatgagaaattgatggttaaataatttgaatctctcatgtagtatcaactattagatattattttcaccaaatatatttccatcggagaagaagaggctacagaggaagcagaagagaggggtgggagaatttttacacttttgtacacccacttaaacagcaaaatccgtatgaaaacaggcccaccaaaacaatgccacgataacaatccgtagaaacaaaagcttcatttaacagcggcgcaacaaagcacgcttatccatggtagttgtagtccgtatgcgatccaaagatcacgattcacgcgtgacggacggacgacgcgtgccacaccacaactaacggcatccatggtagttgtagtccgtatgcgatccaaagatcacgattcacgcgtgacggacggacgacgcgcgccacaccacaactaacagcgtgagccagcgtccaaactccggatggcaacggggacgaaacccgtcgggtagtcactgcccaaacccgtccccgcaaccttcatcccaaacccgtccccgtttccggtcgcgggtttcagttttctaccagacccgtccccatcgggtttttcatccccgtcgggaaatccgaacccgccagcatttcagcaccaagccaaagttgcagcagcaacatgaataaaaaacaacccgtttcaacaccaagataaaacaaaacattataatttagacaacatttcacacgtataacaataacatatagttctcacatataacaacaccatttcacacataaaacaacaccatttgggataaaaatatgggctatatcaggccatttttatgggccatattgagttttcgtgggtttcacaggtaccggatttgtagaatgctgaaccgggtttgaaccgtaaaatccgcgggtattgaatttgacccaatcccgtcgtcccctggtggggtaaaaacaccatcttgagtccaaacggccaccaaccaaactccgacggcaacaaacaaacggcgttgctttgctcctcggtatctccgtgaccgctcaatctcccggctgtttccccggaattgcgtggactctctcatccacacgcaaaccgcctctccctcctctctcgtcctatccgccccggtgccgtagcctcacgggactcttcttcctcccttgctataaaatccccgccccctcctgtctcctctccacacatccaaactctcaatcgcaccgagaaaaatctcctagcgatcgaagcgaagcctctcccgatcctctcaaggtacgcccgtttcccgtcgatcctcctccttccgttcgtgttctgtagccgatcgattcgattcccttacacccgttcgtgttctctcgtggatcgatcgattgtttgttgctagaaggaactcgtagatctggcgtttatgaactgtgattcgggttagtccagatcgattcaggtcggtcgtcgttgagcctctcggctatgtctggattatcgtgtagatctgctggttcagttgattatgttcttctaggagtaatttcgttgggtcagcgcgatttctgcttaatctatgctgcttattgcgcctgtacctatctactaagctatgtgcacctgtaattttgctagattattcgttcatcctcgtagttggtttgtcacagtaatccgtatgggttctgacgatgttattgttggtcatacctaggcttctccagattttattttgttaaaattggatagatctgctactgatagttgatgatggaatttggtgctgaatctatgctatttattgcgcctatacctgatctatcgggctatgtacggctgtagtttactggattattcgttcatcctcggtagttggttcatcgtttgggttctgacgataatattgttgattatgcgtaggcttctgcagattgttgttaaaattggatacatcggttactgatggttgatgatagatttgtgctgaacctatctgtttattgctcctatacctgatctatagggctatgtatgcctgtaatttaccagattattcgttcatcctcgtagttggttcatctctataattcgtatgggttcttatgatgttatcgttgattatgcctagtcttatacagattattgtgtcaagattgaatatacctgctactgatcggtgataatttggttagtagtttgcaatctgctaggaacacgttaccactgtaatctgtaaacatggtttgccagagtagtttgttctactactcttgatatggttgctgattttagtcgcctccttttggatcatgtattgatgtccttgcagatttccgtgtacttaccccggcttttgtgtacttcgtgttaacagctctagaggatcctctcaacacaacatatacaaaacaaacgaatctcaagcaatcaagcattctacttctattgcagcaatttaaatcatttcttttaaagcaaaagcaattttctgaaaattttcaccatttacgaacgatagtaatttctactaagtgtagatgagacggagctcagtctgaccgcggcgtctcttaatttctactaagtgtagatcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgctcgacgcggccgccatggcctctagtggatcaggtgtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 35 - DNA, 10434 bp, synthetic construct
  misc_feature 1..10434: note = Description of Artificial Sequence: Synthetic polynucleotide
  misc_feature 1..10434: note = pGEP372 LbCpf1 RR construct
  source 1..10434: mol_type = other DNA
actgctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgatcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgaagcttgaagcaaacatggcatctagcatggcaccaaagaaaaaaaggaaagtttccaaacttgaaaaatttacaaactgctactccctttccaagacgcttaggtttaaagcgatccccgttggcaagacccaagagaatatcgataacaaaagacttctggtcgaagatgaaaaaagggccgaagactacaagggggtcaagaagttgctcgatcgctattatctttcctttatcaacgatgtgcttcattcaatcaaactgaagaacttgaataactacattagccttttcagaaagaaaacgaggactgaaaaggagaacaaggaacttgagaatcttgaaataaaccttcgcaaagaaattgcaaaagccttcaaggggaacgaaggatataaatctcttttcaaaaaagacattatagaaacaattttgcctgagtttcttgacgacaaggatgaaattgcgctcgtcaatagctttaacggatttacaactgccttcacagggttcttcgacaatagggagaatatgtttagcgaggaggcaaaaagcacatccatcgcattcagatgcatcaatgaaaatcttacccggtacatatcgaatatggacatatttgaaaaagtggatgcaatattcgataagcacgaagtccaggagataaaggaaaagatactgaatagcgactatgatgtcgaagattttttcgaaggtgagttcttcaactttgtcctgactcaagaaggcattgatgtctataatgcaataattggaggttttgtgactgagtctggcgagaagataaagggcttgaacgagtatatcaatctctacaaccagaagactaagcaaaagttgcctaaatttaaaccgctttacaagcaagttttgagcgaccgggaaagcctttccttttacggtgaaggatacacgagcgatgaagaagtcctcgaagtcttccgcaacacactcaacaagaactcagaaatcttttcctcaattaaaaaattggagaagcttttcaagaacttcgatgaatactcttcggcggggatttttgtgaagaacggcccggcaatttccacaatatctaaagacattttcggagaatggaacgtgataagagacaagtggaatgcggagtatgatgacatacacctgaagaagaaggcagttgtgactgaaaaatacgaagatgacaggagaaaaagctttaaaaagatcgggtccttttcactggaacagctgcaggagtatgccgacgccgatctttcggttgtcgaaaagctcaaagaaataattatccagaaggtcgatgaaatctacaaggtgtacggctcaagcgagaagctctttgatgctgacttcgtgttggagaagtctcttaaaaaaaacgacgcagtcgtcgcgataatgaaagatttgctggattcagtgaaatccttcgagaattatatcaaagccttcttcggcgaggggaaggagacaaacagggatgagtccttctatggagacttcgttctggcttacgacatccttcttaaggtcgaccacatctatgacgcaattcggaactatgtgacgcagaagccgtattcgaaagataagttcaagctctatttccaaaaccctcaatttatgcgtgggtgggataaagacaaagagaccgattaccgggcaacaattttgcggtacgggtctaaatattacctcgctataatggataagaaatacgctaaatgtctccagaaaattgacaaagatgacgtcaacggcaattatgaaaaaatcaattataaactccttcctggcccaaataaaatgctcccgagggtgtttttttccaaaaagtggatggcctattataatccatcagaggatattcagaaaatctataaaaatgggacctttaagaagggtgacatgtttaacctgaacgattgccacaagcttatagattttttcaaagactctattagccgctatcccaaatggtctaatgcttatgatttcaacttctctgaaactgaaaagtacaaagatattgcaggattctaccgcgaagttgaagaacaaggttataaggtttcctttgagtctgcgtccaagaaagaggtcgataagttggtcgaagaagggaaattgtatatgtttcaaatttacaataaagacttttccgacaagtcccatggtacacctaatctgcataccatgtacttcaaactgctgttcgatgagaataatcacggtcagattcgcctgagcggaggggcggaactcttcatgaggagagcatcgttgaaaaaagaggagctcgtcgtgcatccggctaacagccccattgctaacaagaatccggataatccaaagaagactactaccctctcctatgacgtctataaggataagagattctctgaggaccagtacgagttgcacatccctattgcgataaataaatgccctaagaacatctttaaaatcaatactgaggtcagagtcctgcttaagcacgacgacaacccgtatgtgatcgggattgataggggtgaaaggaacttgctttatattgtggttgtcgatggaaaaggtaatatagtggaacaatactctctgaatgaaattatcaacaacttcaatggcattaggatcaagaccgactatcattctctgttggacaagaaagagaaagagcgcttcgaggcacggcaaaactggacgtctattgagaacatcaaggagcttaaggctggttacatttctcaggttgtgcacaaaatttgcgaactggtcgagaaatatgatgccgttatcgcacttgaagatctcaacagcggatttaagaattctcgggtgaaagtcgaaaaacaggtgtatcaaaaattcgaaaagatgctgatcgacaagctcaattatatggttgataaaaagagcaacccatgcgccacggggggtgcgcttaagggctatcagattacgaacaaatttgaatccttcaagtcaatgtcgacgcaaaatgggtttatattctatataccggcgtggcttacatctaaaatagatcctagcactgggttcgtgaacctgctgaaaaccaagtacacttcaatcgcagattctaaaaaatttataagcagcttcgacagaatcatgtatgtgcccgaggaagacctcttcgagtttgcccttgattacaaaaatttctcaagaacggatgcagactacataaagaagtggaagctgtactcttatgggaaccggattcggatattcagaaatccgaaaaaaaacaatgtctttgattgggaggaagtttgtcttacctctgcttacaaagagctgttcaataaatatggcattaattaccagcaaggtgatatccgggcgctcctttgcgaacagtctgacaaagctttctattcttcatttatggcgctcatgtcattgatgctgcagatgaggaatagcattacggggaggactgatgttgactttctgatctcgcccgtgaaaaattctgatggaatcttctacgattccaggaattatgaggcccaggaaaatgctatccttcccaagaacgcagacgcaaatggcgcgtacaatatagctcgcaaggttttgtgggctataggccaattcaagaaagccgaagacgaaaagctggacaaagttaagattgctatatctaacaaagagtggcttgagtatgcgcaaacatctgttaaacacaaacgccccgcggctacaaagaaggctggccaggcaaagaagaagaagtgagtcgaccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatcccgggatatcgcggccgcgtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcacgtctaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgacatgatcagggagctctagattatttgtatagttcatccatgcccattacgtcggtaaatgccttctgccactccttgaagttaagttcggtcttggaatgtttcaactcagtcttacggaacacgtacatgggttggttcttaaggtagttagcggccattggtttagcgaatgtgtaggtagtcctggctgtagagcgatatctcttgccattgcctgtggtgtaagaccatttgaaggtactaatgatggtcttgtcgttagggtaggttttcttggaccggcaccaatcagcggcagttaaggagttggtcatgacaggtccatcagcaggaaagcctgtccccttcacttgggcttctcctttgatgtggctcccttcgtaagtgtaacggtagttgacggtgagcgaagcaccgtcctcaaactgcattgtcctgtggacttggtatccggagccatcaaccatggctgcttggaatggactcattccgtcagggtatggaaggtattgatggaatccgtagccaatgtgtggcaccagaatccatggagaaaactgaagatcacctttggtgctcttgaggttcagctcttcgtatccgtcattagggttcccagtgccttgtccgaccatatcgaagtcaacgccgttgatggaaccgaagatgtgaagctcatgtgtggctggaagcgaagccatgttatcttcttctcctttactcacggaggacgccatggtggcgggatcgcgccctatcgttcgtaaatggtgaaaattttcagaaaattgcttttgctttaaaagaaatgatttaaattgctgcaatagaagtagaatgcttgattgcttgagattcgtttgttttgtatatgttgtgttgagaggatcctcaagcttcgacctgcagaagtaacaccaaacaacagggtgagcatcgacaaaagaaacagtaccaagcaaataaatagcgtatgaaggcagggctaaaaaaatccacatatagctgctgcatatgccatcatccaagtatatcaagatcaaaataattataaaacatacttgtttattataatagataggtactcaaggttagagcatatgaatagatgctgcatatgccatcatgtatatgcatcagtaaaacccacatcaacatgtatacctatcctagatcgatatttccatccatcttaaactcgtaactatgaagatgtatgacacacacatacagttccaaaattaataaatacaccaggtagtttgaaacagtattctactccgatctagaacgaatgaacgaccgcccaaccacaccacatcatcacaaccaagcgaacaaaagcatctctgtatatgcatcagtaaaacccgcatcaacatgtatacctatcctagatcgatatttccatccatcatcttcaattcgtaactatgaatatgtatggcacacacatacagatccaaaattaataaatccaccaggtagtttgaaacagaattctactccgatctagaacgaccgcccaaccagaccacatcatcacaaccaagacaaaaaaaagcatgaaaagatgacccgacaaacaagtgcacggcatatattgaaataaaggaaaagggcaaaccaaaccctatgcaacgaaacaaaaaaaatcatgaaatcgatcccgtctgcggaacggctagagccatcccaggattccccaaagagaaacactggcaagttagcaatcagaacgtgtctgacgtacaggtcgcatccgtgtacgaacgctagcagcacggatctaacacaaacacggatctaacacaaacatgaacagaagtagaactaccgggccctaaccatggaccggaacgccgatctagagaaggtagagagggggggggaggacgagcggcgtaccttgaagcggaggtgccgacgggtggatttgggggagatccactagttctagagcggccgccaccgcggtggaattctcgaggtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatcaagtgaccgatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaattcacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatcaagtgacagatagctgggcaatggaatccgaggaggtttcccgatattaccctttgttgaaaagtctcaatagccctttggtcttctgagacttgcaggcaagcaagcatgaatgcctgggcgcgccgatatc

SEQ ID NO: 36 - DNA, 10434 bp, synthetic construct
  misc_feature 1..10434: note = Description of Artificial Sequence: Synthetic polynucleotide
  misc_feature 1..10434: note = pGEP373 LbCpf1 RVR construct
  source 1..10434: mol_type = other DNA
actgctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgatcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgaagcttgaagcaaacatggcatctagcatggcaccaaagaaaaaaaggaaagtttccaaacttgaaaaatttacaaactgctactccctttccaagacgcttaggtttaaagcgatccccgttggcaagacccaagagaatatcgataacaaaagacttctggtcgaagatgaaaaaagggccgaagactacaagggggtcaagaagttgctcgatcgctattatctttcctttatcaacgatgtgcttcattcaatcaaactgaagaacttgaataactacattagccttttcagaaagaaaacgaggactgaaaaggagaacaaggaacttgagaatcttgaaataaaccttcgcaaagaaattgcaaaagccttcaaggggaacgaaggatataaatctcttttcaaaaaagacattatagaaacaattttgcctgagtttcttgacgacaaggatgaaattgcgctcgtcaatagctttaacggatttacaactgccttcacagggttcttcgacaatagggagaatatgtttagcgaggaggcaaaaagcacatccatcgcattcagatgcatcaatgaaaatcttacccggtacatatcgaatatggacatatttgaaaaagtggatgcaatattcgataagcacgaagtccaggagataaaggaaaagatactgaatagcgactatgatgtcgaagattttttcgaaggtgagttcttcaactttgtcctgactcaagaaggcattgatgtctataatgcaataattggaggttttgtgactgagtctggcgagaagataaagggcttgaacgagtatatcaatctctacaaccagaagactaagcaaaagttgcctaaatttaaaccgctttacaagcaagttttgagcgaccgggaaagcctttccttttacggtgaaggatacacgagcgatgaagaagtcctcgaagtcttccgcaacacactcaacaagaactcagaaatcttttcctcaattaaaaaattggagaagcttttcaagaacttcgatgaatactcttcggcggggatttttgtgaagaacggcccggcaatttccacaatatctaaagacattttcggagaatggaacgtgataagagacaagtggaatgcggagtatgatgacatacacctgaagaagaaggcagttgtgactgaaaaatacgaagatgacaggagaaaaagctttaaaaagatcgggtccttttcactggaacagctgcaggagtatgccgacgccgatctttcggttgtcgaaaagctcaaagaaataattatccagaaggtcgatgaaatctacaaggtgtacggctcaagcgagaagctctttgatgctgacttcgtgttggagaagtctcttaaaaaaaacgacgcagtcgtcgcgataatgaaagatttgctggattcagtgaaatccttcgagaattatatcaaagccttcttcggcgaggggaaggagacaaacagggatgagtccttctatggagacttcgttctggcttacgacatccttcttaaggtcgaccacatctatgacgcaattcggaactatgtgacgcagaagccgtattcgaaagataagttcaagctctatttccaaaaccctcaatttatgcgtgggtgggataaagacgtagagaccgatcgccgggcaacaattttgcggtacgggtctaaatattacctcgctataatggataagaaatacgctaaatgtctccagaaaattgacaaagatgacgtcaacggcaattatgaaaaaatcaattataaactccttcctggcccaaataaaatgctcccgaaggtgtttttttccaaaaagtggatggcctattataatccatcagaggatattcagaaaatctataaaaatgggacctttaagaagggtgacatgtttaacctgaacgattgccacaagcttatagattttttcaaagactctattagccgctatcccaaatggtctaatgcttatgatttcaacttctctgaaactgaaaagtacaaagatattgcaggattctaccgcgaagttgaagaacaaggttataaggtttcctttgagtctgcgtccaagaaagaggtcgataagttggtcgaagaagggaaattgtatatgtttcaaatttacaataaagacttttccgacaagtcccatggtacacctaatctgcataccatgtacttcaaactgctgttcgatgagaataatcacggtcagattcgcctgagcggaggggcggaactcttcatgaggagagcatcgttgaaaaaagaggagctcgtcgtgcatccggctaacagccccattgctaacaagaatccggataatccaaagaagactactaccctctcctatgacgtctataaggataagagattctctgaggaccagtacgagttgcacatccctattgcgataaataaatgccctaagaacatctttaaaatcaatactgaggtcagagtcctgcttaagcacgacgacaacccgtatgtgatcgggattgataggggtgaaaggaacttgctttatattgtggttgtcgatggaaaaggtaatatagtggaacaatactctctgaatgaaattatcaacaacttcaatggcattaggatcaagaccgactatcattctctgttggacaagaaagagaaagagcgcttcgaggcacggcaaaactggacgtctattgagaacatcaaggagcttaaggctggttacatttctcaggttgtgcacaaaatttgcgaactggtcgagaaatatgatgccgttatcgcacttgaagatctcaacagcggatttaagaattctcgggtgaaagtcgaaaaacaggtgtatcaaaaattcgaaaagatgctgatcgacaagctcaattatatggttgataaaaagagcaacccatgcgccacggggggtgcgcttaagggctatcagattacgaacaaatttgaatccttcaagtcaatgtcgacgcaaaatgggtttatattctatataccggcgtggcttacatctaaaatagatcctagcactgggttcgtgaacctgctgaaaaccaagtacacttcaatcgcagattctaaaaaatttataagcagcttcgacagaatcatgtatgtgcccgaggaagacctcttcgagtttgcccttgattacaaaaatttctcaagaacggatgcagactacataaagaagtggaagctgtactcttatgggaaccggattcggatattcagaaatccgaaaaaaaacaatgtctttgattgggaggaagtttgtcttacctctgcttacaaagagctgttcaataaatatggcattaattaccagcaaggtgatatccgggcgctcctttgcgaacagtctgacaaagctttctattcttcatttatggcgctcatgtcattgatgctgcagatgaggaatagcattacggggaggactgatgttgactttctgatctcgcccgtgaaaaattctgatggaatcttctacgattccaggaattatgaggcccaggaaaatgctatccttcccaagaacgcagacgcaaatggcgcgtacaatatagctcgcaaggttttgtgggctataggccaattcaagaaagccgaagacgaaaagctggacaaagttaagattgctatatctaacaaagagtggcttgagtatgcgcaaacatctgttaaacacaaacgccccgcggctacaaagaaggctggccaggcaaagaagaagaagtgagtcgaccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatcccgggatatcgcggccgcgtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcacgtctaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgacatgatcagggagctctagattatttgtatagttcatccatgcccattacgtcggtaaatgccttctgccactccttgaagttaagttcggtcttggaatgtttcaactcagtcttacggaacacgtacatgggttggttcttaaggtagttagcggccattggtttagcgaatgtgtaggtagtcctggctgtagagcgatatctcttgccattgcctgtggtgtaagaccatttgaaggtactaatgatggtcttgtcgttagggtaggttttcttggaccggcaccaatcagcggcagttaaggagttggtcatgacaggtccatcagcaggaaagcctgtccccttcacttgggcttctcctttgatgtggctcccttcgtaagtgtaacggtagttgacggtgagcgaagcaccgtcctcaaactgcattgtcctgtggacttggtatccggagccatcaaccatggctgcttggaatggactcattccgtcagggtatggaaggtattgatggaatccgtagccaatgtgtggcaccagaatccatggagaaaactgaagatcacctttggtgctcttgaggttcagctcttcgtatccgtcattagggttcccagtgccttgtccgaccatatcgaagtcaacgccgttgatggaaccgaagatgtgaagctcatgtgtggctggaagcgaagccatgttatcttcttctcctttactcacggaggacgccatggtggcgggatcgcgccctatcgttcgtaaatggtgaaaattttcagaaaattgcttttgctttaaaagaaatgatttaaattgctgcaatagaagtagaatgcttgattgcttgagattcgtttgttttgtatatgttgtgttgagaggatcctcaagcttcgacctgcagaagtaacaccaaacaacagggtgagcatcgacaaaagaaacagtaccaagcaaataaatagcgtatgaaggcagggctaaaaaaatccacatatagctgctgcatatgccatcatccaagtatatcaagatcaaaataattataaaacatacttgtttattataatagataggtactcaaggttagagcatatgaatagatgctgcatatgccatcatgtatatgcatcagtaaaacccacatcaacatgtatacctatcctagatcgatatttccatccatcttaaactcgtaactatgaagatgtatgacacacacatacagttccaaaattaataaatacaccaggtagtttgaaacagtattctactccgatctagaacgaatgaacgaccgcccaaccacaccacatcatcacaaccaagcgaacaaaagcatctctgtatatgcatcagtaaaacccgcatcaacatgtatacctatcctagatcgatatttccatccatcatcttcaattcgtaactatgaatatgtatggcacacacatacagatccaaaattaataaatccaccaggtagtttgaaacagaattctactccgatctagaacgaccgcccaaccagaccacatcatcacaaccaagacaaaaaaaagcatgaaaagatgacccgacaaacaagtgcacggcatatattgaaataaaggaaaagggcaaaccaaaccctatgcaacgaaacaaaaaaaatcatgaaatcgatcccgtctgcggaacggctagagccatcccaggattccccaaagagaaacactggcaagttagcaatcagaacgtgtctgacgtacaggtcgcatccgtgtacgaacgctagcagcacggatctaacacaaacacggatctaacacaaacatgaacagaagtagaactaccgggccctaaccatggaccggaacgccgatctagagaaggtagagagggggggggaggacgagcggcgtaccttgaagcggaggtgccgacgggtggatttgggggagatccactagttctagagcggccgccaccgcggtggaattctcgaggtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatcaagtgaccgatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaattcacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatcaagtgacagatagctgggcaatggaatccgaggaggtttcccgatattaccctttgttgaaaagtctcaatagccctttggtcttctgagacttgcaggcaagcaagcatgaatgcctgggcgcgccgatatc

SEQ ID NO: 37 - DNA, 10432 bp, synthetic construct
  misc_feature 1..10432: note = Description of Artificial Sequence: Synthetic polynucleotide
  misc_feature 1..10432: note = pGEP315 Corn strategy LbCpf1 vector
  source 1..10432: mol_type = other DNA
actgctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgatcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgaagcttgaagcaaacatggcatctagcatggcaccaaagaaaaaaaggaaagtttccaaacttgaaaaatttacaaactgctactccctttccaagacgcttaggtttaaagcgatccccgttggcaagacccaagagaatatcgataacaaaagacttctggtcgaagatgaaaaaagggccgaagactacaagggggtcaagaagttgctcgatcgctattatctttcctttatcaacgatgtgcttcattcaatcaaactgaagaacttgaataactacattagccttttcagaaagaaaacgaggactgaaaaggagaacaaggaacttgagaatcttgaaataaaccttcgcaaagaaattgcaaaagccttcaaggggaacgaaggatataaatctcttttcaaaaaagacattatagaaacaattttgcctgagtttcttgacgacaaggatgaaattgcgctcgtcaatagctttaacggatttacaactgccttcacagggttcttcgacaatagggagaatatgtttagcgaggaggcaaaaagcacatccatcgcattcagatgcatcaatgaaaatcttacccggtacatatcgaatatggacatatttgaaaaagtggatgcaatattcgataagcacgaagtccaggagataaaggaaaagatactgaatagcgactatgatgtcgaagattttttcgaaggtgagttcttcaactttgtcctgactcaagaaggcattgatgtctataatgcaataattggaggttttgtgactgagtctggcgagaagataaagggcttgaacgagtatatcaatctctacaaccagaagactaagcaaaagttgcctaaatttaaaccgctttacaagcaagttttgagcgaccgggaaagcctttccttttacggtgaaggatacacgagcgatgaagaagtcctcgaagtcttccgcaacacactcaacaagaactcagaaatcttttcctcaattaaaaaattggagaagcttttcaagaacttcgatgaatactcttcggcggggatttttgtgaagaacggcccggcaatttccacaatatctaaagacattttcggagaatggaacgtgataagagacaagtggaatgcggagtatgatgacatacacctgaagaagaaggcagttgtgactgaaaaatacgaagatgacaggagaaaaagctttaaaaagatcgggtccttttcactggaacagctgcaggagtatgccgacgccgatctttcggttgtcgaaaagctcaaagaaataattatccagaaggtcgatgaaatctacaaggtgtacggctcaagcgagaagctctttgatgctgacttcgtgttggagaagtctcttaaaaaaaacgacgcagtcgtcgcgataatgaaagatttgctggattcagtgaaatccttcgagaattatatcaaagccttcttcggcgaggggaaggagacaaacagggatgagtccttctatggagacttcgttctggcttacgacatccttcttaaggtcgaccacatctatgacgcaattcggaactatgtgacgcagaagccgtattcgaaagataagttcaagctctatttccaaaaccctcaatttatgggtgggtgggataaagacaaagagaccgattaccgggcaacaattttgcggtacgggtctaaatattacctcgctataatggataagaaatacgctaaatgtctccagaaaattgacaaagatgacgtcaacggcaattatgaaaaaatcaattataaactccttcctggcccaaataaaatgctcccgaaggtgtttttttccaaaaagtggatggcctattataatccatcagaggatattcagaaaatctataaaaatgggacctttaagaagggtgacatgtttaacctgaacgattgccacaagcttatagattttttcaaagactctattagccgctatcccaaatggtctaatgcttatgatttcaacttctctgaaactgaaaagtacaaagatattgcaggattctaccgcgaagttgaagaacaaggttataaggtttcctttgagtctgcgtccaagaaagaggtcgataagttggtcgaagaagggaaattgtatatgtttcaaatttacaataaagacttttccgacaagtcccatggtacacctaatctgcataccatgtacttcaaactgctgttcgatgagaataatcacggtcagattcgcctgagcggaggggcggaactcttcatgaggagagcatcgttgaaaaaagaggagctcgtcgtgcatccggctaacagccccattgctaacaagaatccggataatccaaagaagactactaccctctcctatgacgtctataaggataagagattctctgaggaccagtacgagttgcacatccctattgcgataaataaatgccctaagaacatctttaaaatcaatactgaggtcagagtcctgcttaagcacgacgacaacccgtatgtgatcgggattgataggggtgaaaggaacttgctttatattgtggttgtcgatggaaaaggtaatatagtggaacaatactctctgaatgaaattatcaacaacttcaatggcattaggatcaagaccgactatcattctctgttggacaagaaagagaaagagcgcttcgaggcacggcaaaactggacgtctattgagaacatcaaggagcttaaggctggttacatttctcaggttgtgcacaaaatttgcgaactggtcgagaaatatgatgccgttatcgcacttgaagatctcaacagcggatttaagaattctcgggtgaaagtcgaaaaacaggtgtatcaaaaattcgaaaagatgctgatcgacaagctcaattatatggttgataaaaagagcaacccatgcgccacggggggtgcgcttaagggctatcagattacgaacaaatttgaatccttcaagtcaatgtcgacgcaaaatgggtttatattctatataccggcgtggcttacatctaaaatagatcctagcactgggttcgtgaacctgctgaaaaccaagtacacttcaatcgcagattctaaaaaatttataagcagcttcgacagaatcatgtatgtgcccgaggaagacctcttcgagtttgcccttgattacaaaaatttctcaagaacggatgcagactacataaagaagtggaagctgtactcttatgggaaccggattcggatattcagaaatccgaaaaaaaacaatgtctttgattgggaggaagtttgtcttacctctgcttacaaagagctgttcaataaatatggcattaattaccagcaaggtgatatccgggcgctcctttgcgaacagtctgacaaagctttctattcttcatttatggcgctcatgtcattgatgctgcagatgaggaatagcattacggggaggactgatgttgactttctgatctcgcccgtgaaaaattctgatggaatcttctacgattccaggaattatgaggcccaggaaaatgctatccttcccaagaacgcagacgcaaatggcgcgtacaatatagctcgcaaggttttgtgggctataggccaattcaagaaagccgaagacgaaaagctggacaaagttaagattgctatatctaacaaagagtggcttgagtatgcgcaaacatctgttaaacacaaacgccccgcggctacaaagaaggctggccaggcaaagaagaagaagtgagtcgaccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatcccgggatatcgcggccggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtatcggcacgtctaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgacatgatcagggagccctagattatttgtatagttcatccatgcccattacgtcggtaaatgccttctgccactccttgaagttaagttcggtcttggaatgtttcaactcagtcttacggaacacgtacatgggttggttcttaaggtagttagcggccattggtttagcgaatgtgtaggtagtcctggctgtagagcgatatctcttgccattgcctgtggtgtaagaccatttgaaggtactaatgatggtcttgtcgttagggtaggttttcttggaccggcaccaatcagcggcagttaaggagttggtcatgacaggtccatcagcaggaaagcctgtccccttcacttgggcttctcctttgatgtggctcccttcgtaagtgtaacggtagttgacggtgagcgaagcaccgtcctcaaactgcattgtcctgtggacttggtatccggagccatcaaccatggctgcttggaatggactcattccgtcagggtatggaaggtattgatggaatccgtagccaatgtgtggcaccagaatccatggagaaaactgaagatcacctttggtgctcttgaggttcagctcttcgtatccgtcattagggttcccagtgccttgtccgaccatatcgaagtcaacgccgttgatggaaccgaagatgtgaagctcatgtgtggctggaagcgaagccatgttatcttcttctcctttactcacggaggacgccatggtggcgggatcgcgccctatcgttcgtaaatggtgaaaattttcagaaaattgcttttgctttaaaagaaatgatttaaattgctgcaatagaagtagaatgcttgattgcttgagattcgtttgttttgtatatgttgtgttgagaggatcctcaagcttcgacctgcagaagtaacaccaaacaacagggtgagcatcgacaaaagaaacagtaccaagcaaataaatagcgtatgaaggcagggctaaaaaaatccacatatagctgctgcatatgccatcatccaagtatatcaagatcaaaataattataaaacatacttgtttattataatagataggtactcaaggttagagcatatgaatagatgctgcatatgccatcatgtatatgcatcagtaaaacccacatcaacatgtatacctatcctagatcgatatttccatccatcttaaactcgtaactatgaagatgtatgacacacacatacagttccaaaattaataaatacaccaggtagtttgaaacagtattctactccgatctagaacgaatgaacgaccgcccaaccacaccacatcatcacaaccaagcgaacaaaaagcatctctgtatatgcatcagtaaaacccgcatcaacatgtatacctatcctagatcgatatttccatccatcatcttcaattcgtaactatgaatatgtatggcacacacatacagatccaaaattaataaatccaccaggtagtttgaaacagaattctactccgatctagaacgaccgcccaaccagaccacatcatcacaaccaagacaaaaaaaagcatgaaaagatgacccgacaaacaagtgcacggcatatattgaaataaaggaaaagggcaaaccaaaccctatgcaacgaaacaaaaaaaatcatgaaatcgatcccgtctgcggaacggctagagccatcccaggattccccaaagagaaacactggcaagttagcaatcagaacgtgtctgacgtacaggtcgcatccgtgtacgaacgctagcagcacggatctaacacaaacacggatctaacacaaacatgaacagaagtagaactaccgggccctaaccatggaccggaacgccgatctagagaaggtagagaggggggggggggaggacgagcggcgtaccttgaagcggaggtgccgacgggtggatttgggggagatccactagttctagagcggccgccaccgcggtggaattctcgaggtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgttcctcgtgggtgggggtccatctttgggaccactgtcggtagaggcatcttgaacgatagcctttcctttatcgcaatgatggcatttgtagaagccatcttccttttctactgtcctttcgatgaagtgacagatagctgggcaatggaatccgaggaggtttcccgatattaccctttgttgaaaagtctcaatagccctctggtcttctgagactgtatctttgatattcttggagtagacgagagtgtcgtgctccaccatgtatcacatcaatccacttgctttgaagacgtggttggaacgtcttctttttccacgatgttcctcgtgggtgggggtccatctttgggaccactgtcggtagaggcatcttgaacgatagcctttcctttatcgcaatgatggcatttgtagaagccatcttccttttctactgtcctttcgatgaagtgacagatagctgggcaatggaatccgaggaggtttcccgatattaccctttgttgaaaagtctcaatagccctctggtcttctgaacctgcaggcaagcaagcatgaatgcctgggcgcgccgatatc

SEQ ID NO: 38 - DNA, 3684 bp, synthetic construct
  misc_feature 1..3684: note = Description of Artificial Sequence: Synthetic polynucleotide
  misc_feature 1..3684: note = LbCpf1 RR coding sequence codon optimized
  source 1..3684: mol_type = other DNA
atgtccaaacttgaaaaatttacaaactgctactccctttccaagacgcttaggtttaaagcgatccccgttggcaagacccaagagaatatcgataacaaaagacttctggtcgaagatgaaaaaagggccgaagactacaagggggtcaagaagttgctcgatcgctattatctttcctttatcaacgatgtgcttcattcaatcaaactgaagaacttgaataactacattagccttttcagaaagaaaacgaggactgaaaaggagaacaaggaacttgagaatcttgaaataaaccttcgcaaagaaattgcaaaagccttcaaggggaacgaaggatataaatctcttttcaaaaaagacattatagaaacaattttgcctgagtttcttgacgacaaggatgaaattgcgctcgtcaatagctttaacggatttacaactgccttcacagggttcttcgacaatagggagaatatgtttagcgaggaggcaaaaagcacatccatcgcattcagatgcatcaatgaaaatcttacccggtacatatcgaatatggacatatttgaaaaagtggatgcaatattcgataagcacgaagtccaggagataaaggaaaagatactgaatagcgactatgatgtcgaagattttttcgaaggtgagttcttcaactttgtcctgactcaagaaggcattgatgtctataatgcaataattggaggttttgtgactgagtctggcgagaagataaagggcttgaacgagtatatcaatctctacaaccagaagactaagcaaaagttgcctaaatttaaaccgctttacaagcaagttttgagcgaccgggaaagcctttccttttacggtgaaggatacacgagcgatgaagaagtcctcgaagtcttccgcaacacactcaacaagaactcagaaatcttttcctcaattaaaaaattggagaagcttttcaagaacttcgatgaatactcttcggcggggatttttgtgaagaacggcccggcaatttccacaatatctaaagacattttcggagaatggaacgtgataagagacaagtggaatgcggagtatgatgacatacacctgaagaagaaggcagttgtgactgaaaaatacgaagatgacaggagaaaaagctttaaaaagatcgggtccttttcactggaacagctgcaggagtatgccgacgccgatctttcggttgtcgaaaagctcaaagaaataattatccagaaggtcgatgaaatctacaaggtgtacggctcaagcgagaagctctttgatgctgacttcgtgttggagaagtctcttaaaaaaaacgacgcagtcgtcgcgataatgaaagatttgctggattcagtgaaatccttcgagaattatatcaaagccttcttcggcgaggggaaggagacaaacagggatgagtccttctatggagacttcgttctggcttacgacatccttcttaaggtcgaccacatctatgacgcaattcggaactatgtgacgcagaagccgtattcgaaagataagttcaagctctatttccaaaaccctcaatttatgcgtgggtgggataaagacaaagagaccgattaccgggcaacaattttgcggtacgggtctaaatattacctcgctataatggataagaaatacgctaaatgtctccagaaaattgacaaagatgacgtcaacggcaattatgaaaaaatcaattataaactccttcctggcccaaataaaatgctcccgagggtgtttttttccaaaaagtggatggcctattataatccatcagaggatattcagaaaatctataaaaatgggacctttaagaagggtgacatgtttaacctgaacgattgccacaagcttatagattttttcaaagactctattagccgctatcccaaatggtctaatgcttatgatttcaacttctctgaaactgaaaagtacaaagatattgcaggattctaccgcgaagttgaagaacaaggttataaggtttcctttgagtctgcgtccaagaaagaggtcgataagttggtcgaagaagggaaattgtatatgtttcaaatttacaataaagacttttccgacaagtcccatggtacacctaatctgcataccatgtacttcaaactgctgttcgatgagaataatcacggtcagattcgcctgagcggaggggcggaactcttcatgaggagagcatcgttgaaaaaagaggagctcgtcgtgcatccggctaacagccccattgctaacaagaatccggataatccaaagaagactactaccctctcctatgacgtctataaggataagagattctctgaggaccagtacgagttgcacatccctattgcgataaataaatgccctaagaacatctttaaaatcaatactgaggtcagagtcctgcttaagcacgacgacaacccgtatgtgatcgggattgataggggtgaaaggaacttgctttatattgtggttgtcgatggaaaaggtaatatagtggaacaatactctctgaatgaaattatcaacaacttcaatggcattaggatcaagaccgactatcattctctgttggacaagaaagagaaagagcgcttcgaggcacggcaaaactggacgtctattgagaacatcaaggagcttaaggctggttacatttctcaggttgtgcacaaaatttgcgaactggtcgagaaatatgatgccgttatcgcacttgaagatctcaacagcggatttaagaattctcgggtgaaagtcgaaaaacaggtgtatcaaaaattcgaaaagatgctgatcgacaagctcaattatatggttgataaaaagagcaacccatgcgccacggggggtgcgcttaagggctatcagattacgaacaaatttgaatccttcaagtcaatgtcgacgcaaaatgggtttatattctatataccggcgtggcttacatctaaaatagatcctagcactgggttcgtgaacctgctgaaaaccaagtacacttcaatcgcagattctaaaaaatttataagcagcttcgacagaatcatgtatgtgcccgaggaagacctcttcgagtttgcccttgattacaaaaatttctcaagaacggatgcagactacataaagaagtggaagctgtactcttatgggaaccggattcggatattcagaaatccgaaaaaaaacaatgtctttgattgggaggaagtttgtcttacctctgcttacaaagagctgttcaataaatatggcattaattaccagcaaggtgatatccgggcgctcctttgcgaacagtctgacaaagctttctattcttcatttatggcgctcatgtcattgatgctgcagatgaggaatagcattacggggaggactgatgttgactttctgatctcgcccgtgaaaaattctgatggaatcttctacgattccaggaattatgaggcccaggaaaatgctatccttcccaagaacgcagacgcaaatggcgcgtacaatatagctcgcaaggttttgtgggctataggccaattcaagaaagccgaagacgaaaagctggacaaagttaagattgctatatctaacaaagagtggcttgagtatgcgcaaacatctgttaaacac

SEQ ID NO: 39 - DNA, 3684 bp, synthetic construct
  misc_feature 1..3684: note = Description of Artificial Sequence: Synthetic polynucleotide
  misc_feature 1..3684: note = LbCpf1 RVR coding sequence codon optimized
  source 1..3684: mol_type = other DNA
atgtccaaacttgaaaaatttacaaactgctactccctttccaagacgcttaggtttaaagcgatccccgttggcaagacccaagagaatatcgataacaaaagacttctggtcgaagatgaaaaaagggccgaagactacaagggggtcaagaagttgctcgatcgctattatctttcctttatcaacgatgtgcttcattcaatcaaactgaagaacttgaataactacattagccttttcagaaagaaaacgaggactgaaaaggagaacaaggaacttgagaatcttgaaataaaccttcgcaaagaaattgcaaaagccttcaaggggaacgaaggatataaatctcttttcaaaaaagacattatagaaacaattttgcctgagtttcttgacgacaaggatgaaattgcgctcgtcaatagctttaacggatttacaactgccttcacagggttcttcgacaatagggagaatatgtttagcgaggaggcaaaaagcacatccatcgcattcagatgcatcaatgaaaatcttacccggtacatatcgaatatggacatatttgaaaaagtggatgcaatattcgataagcacgaagtccaggagataaaggaaaagatactgaatagcgactatgatgtcgaagattttttcgaaggtgagttcttcaactttgtcctgactcaagaaggcattgatgtctataatgcaataattggaggttttgtgactgagtctggcgagaagataaagggcttgaacgagtatatcaatctctacaaccagaagactaagcaaaagttgcctaaatttaaaccgctttacaagcaagttttgagcgaccgggaaagcctttccttttacggtgaaggatacacgagcgatgaagaagtcctcgaagtcttccgcaacacactcaacaagaactcagaaatcttttcctcaattaaaaaattggagaagcttttcaagaacttcgatgaatactcttcggcggggatttttgtgaagaacggcccggcaatttccacaatatctaaagacattttcggagaatggaacgtgataagagacaagtggaatgcggagtatgatgacatacacctgaagaagaaggcagttgtgactgaaaaatacgaagatgacaggagaaaaagctttaaaaagatcgggtccttttcactggaacagctgcaggagtatgccgacgccgatctttcggttgtcgaaaagctcaaagaaataattatccagaaggtcgatgaaatctacaaggtgtacggctcaagcgagaagctctttgatgctgacttcgtgttggagaagtctcttaaaaaaaacgacgcagtcgtcgcgataatgaaagatttgctggattcagtgaaatccttcgagaattatatcaaagccttcttcggcgaggggaaggagacaaacagggatgagtccttctatggagacttcgttctggcttacgacatccttcttaaggtcgaccacatctatgacgcaattcggaactatgtgacgcagaagccgtattcgaaagataagttcaagctctatttccaaaaccctcaatttatgcgtgggtgggataaagacgtagagaccgatcgccgggcaacaattttgcggtacgggtctaaatattacctcgctataatggataagaaatacgctaaatgtctccagaaaattgacaaagatgacgtcaacggcaattatgaaaaaatcaattataaactccttcctggcccaaataaaatgctcccgaaggtgtttttttccaaaaagtggatggcctattataatccatcagaggatattcagaaaatctataaaaatgggacctttaagaagggtgacatgtttaacctgaacgattgccacaagcttatagattttttcaaagactctattagccgctatcccaaatggtctaatgcttatgatttcaacttctctgaaactgaaaagtacaaagatattgcaggattctaccgcgaagttgaagaacaaggttataaggtttcctttgagtctgcgtccaagaaagaggtcgataagttggtcgaagaagggaaattgtatatgtttcaaatttacaataaagacttttccgacaagtcccatggtacacctaatctgcataccatgtacttcaaactgctgttcgatgagaataatcacggtcagattcgcctgagcggaggggcggaactcttcatgaggagagcatcgttgaaaaaagaggagctcgtcgtgcatccggctaacagccccattgctaacaagaatccggataatccaaagaagactactaccctctcctatgacgtctataaggataagagattctctgaggaccagtacgagttgcacatccctattgcgataaataaatgccctaagaacatctttaaaatcaatactgaggtcagagtcctgcttaagcacgacgacaacccgtatgtgatcgggattgataggggtgaaaggaacttgctttatattgtggttgtcgatggaaaaggtaatatagtggaacaatactctctgaatgaaattatcaacaacttcaatggcattaggatcaagaccgactatcattctctgttggacaagaaagagaaagagcgcttcgaggcacggcaaaactggacgtctattgagaacatcaaggagcttaaggctggttacatttctcaggttgtgcacaaaatttgcgaactggtcgagaaatatgatgccgttatcgcacttgaagatctcaacagcggatttaagaattctcgggtgaaagtcgaaaaacaggtgtatcaaaaattcgaaaagatgctgatcgacaagctcaattatatggttgataaaaagagcaacccatgcgccacggggggtgcgcttaagggctatcagattacgaacaaatttgaatccttcaagtcaatgtcgacgcaaaatgggtttatattctatataccggcgtggcttacatctaaaatagatcctagcactgggttcgtgaacctgctgaaaaccaagtacacttcaatcgcagattctaaaaaatttataagcagcttcgacagaatcatgtatgtgcccgaggaagacctcttcgagtttgcccttgattacaaaaatttctcaagaacggatgcagactacataaagaagtggaagctgtactcttatgggaaccggattcggatattcagaaatccgaaaaaaaacaatgtctttgattgggaggaagtttgtcttacctctgcttacaaagagctgttcaataaatatggcattaattaccagcaaggtgatatccgggcgctcctttgcgaacagtctgacaaagctttctattcttcatttatggcgctcatgtcattgatgctgcagatgaggaatagcattacggggaggactgatgttgactttctgatctcgcccgtgaaaaattctgatggaatcttctacgattccaggaattatgaggcccaggaaaatgctatccttcccaagaacgcagacgcaaatggcgcgtacaatatagctcgcaaggttttgtgggctataggccaattcaagaaagccgaagacgaaaagctggacaaagttaagattgctatatctaacaaagagtggcttgagtatgcgcaaacatctgttaaacac

SEQ ID NO: 40 - protein, 1228 aa, synthetic construct
  REGION 1..1228: note = Description of Artificial Sequence: Synthetic polypeptide
  REGION 1..1228: note = LbCpf1 RR polypeptide
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMRGWDKDKETDYRATILRYGSKYYLAIMDKKYAKCLQKIDKDDVNGNYEKINYKLLPGPNKMLPRVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 41 - protein, 1228 aa, synthetic construct
  REGION 1..1228: note = Description of Artificial Sequence: Synthetic polypeptide
  REGION 1..1228: note = LbCpf1 RVR polypeptide
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMRGWDKDVETDRRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 42 - DNA, 27 bp, synthetic construct
  misc_feature 1..27: note = Description of Artificial Sequence: Synthetic oligonucleotide
  misc_feature 1..27: note = SV40 NLS
  source 1..27: mol_type = other DNA
atggcaccaaagaaaaaaaggaaagtt

SEQ ID NO: 43 - DNA, 48 bp, synthetic construct
  misc_feature 1..48: note = Description of Artificial Sequence: Synthetic oligonucleotide
  misc_feature 1..48: note = Nucleoplasmin NLS
  source 1..48: mol_type = other DNA
aaacgccccgcggctacaaagaaggctggccaggcaaagaagaagaag

SEQ ID NO: 44 - DNA, 3849 bp, synthetic construct
  misc_feature 1..3849: note = Description of Artificial Sequence: Synthetic polynucleotide
  misc_feature 1..3849: note = pGEP296 Ribozyme strategy vector
  source 1..3849: mol_type = other DNA
ctgacgcgccctgtagcggcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgatcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccaaattactgatgagtccgtgaggacgaaacgagtaagctcgtctaatttctactaagtgtagatgagacggagctcagtctgaccgcggcgtctctggccggcatggtcccagcctcctcgctggcgccggctgggcaacatgcttcggcatggcgaatgggaccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatcgtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 45 - DNA, 3834 bp, synthetic construct
  misc_feature 1..3834: note = Description of Artificial Sequence: Synthetic polynucleotide
  misc_feature 1..3834: note = pGEP374 vector with rice HDV-like ribozyme sequence
  source 1..3834: mol_type = other DNA
ctgacgcgccctgtagcggcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgatcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccaaattactgatgagtccgtgaggacgaaacgagtaagctcgtctaatttctactaagtgtagatgagacggagctcagtctgaccgcggcgtctctccgccaacactgccaatgccggtcccaagcccggataaaagtggagggggcggcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatcgtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 46 - DNA, 3844 bp, synthetic construct
  misc_feature 1..3844: note = Description of Artificial Sequence: Synthetic polynucleotide
  misc_feature 1..3844: note = pGEP375 vector with sunflower HDV-like sequence
  source 1..3844: mol_type = other DNA
ctgacgcgccctgtagcggcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgatcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccaaattactgatgagtccgtgaggacgaaacgagtaagctcgtctaatttctactaagtgtagatgagacggagctcagtctgaccgcggcgtctctgcggggggcgtcagtcctactctgcacctcctcgtggtgtcgcctgggaaccctctttcgcaacgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatcgtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 47 - DNA, 3867 bp, synthetic construct
  misc_feature 1..3867: note = Description of Artificial Sequence: Synthetic polynucleotide
  misc_feature 1..3867: note = pGEP375 vector with sunflower HDV-like sequence_long
  source 1..3867: mol_type = other DNA
ctgacgcgccctgtagcggcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgatcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccaaattactgatgagtccgtgaggacgaaacgagtaagctcgtctaatttctactaagtgtagatgagacggagctcagtctgaccgcggcgtctctgcggggggcgtcagtcctactctgcacctcctcgtggtgtcgcctgggaaccctctttcgcaagaaagaggagccaagcagagaggcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatcgtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 48 - DNA, 3861 bp, synthetic construct
  misc_feature 1..3861: note = Description of Artificial Sequence: Synthetic polynucleotide
  misc_feature 1..3861: note = pGEP376 vector with artichoke HDV-like sequence
  source 1..3861: mol_type = other DNA
ctgacgcgccctgtagcggcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgatcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccaaattactgatgagtccgtgaggacgaaacgagtaagctcgtctaatttctactaagtgtagatgagacggagctcagtctgaccgcggcgtctctggcgtcagtcctactctgcacctcctcgtggtgtcgcctgggaaccctctttcacaagaaagaggagccaagcagagaggcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatcgtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 49 - DNA, 24 bp, Zea mays
  source 1..24: mol_type = unassigned DNA
ctcgtcacgattcccctctcctgg

SEQ ID NO: 50 - DNA, 24 bp, Zea mays
  source 1..24: mol_type = unassigned DNA
cccacctgaaaagttcgaccagga

SEQ ID NO: 51 - DNA, 24 bp, Zea mays
  source 1..24: mol_type = unassigned DNA
tgtgtggtcacacttgccagccag

SEQ ID NO: 52 - DNA, 24 bp, Zea mays
  source 1..24: mol_type = unassigned DNA
gtggtcggatttctggcatcgctg

SEQ ID NO: 53 - DNA, 23 bp, Zea mays
  source 1..23: mol_type = unassigned DNA
gtctatgtcgatgaccagcagat

SEQ ID NO: 54 - DNA, 24 bp, Zea mays
  source 1..24: mol_type = unassigned DNA
cctctcctggtcgaacttttcagg

SEQ ID NO: 55 - DNA, 24 bp, Zea mays
  source 1..24: mol_type = unassigned DNA
accaggagaggggaatcgtgacga

SEQ ID NO: 56 - DNA, 24 bp, Zea mays
  source 1..24: mol_type = unassigned DNA
ttatagcacgacaaaagtaaaaat

SEQ ID NO: 57 - DNA, 24 bp, Zea mays
  source 1..24: mol_type = unassigned DNA
attgtcgtcatcatcggctaacat

SEQ ID NO: 58 - DNA, 24 bp, Zea mays
  source 1..24: mol_type = unassigned DNA
tactttgacttttcccttaatgac

SEQ ID NO: 59 - DNA, 24 bp, Zea mays
  source 1..24: mol_type = unassigned DNA
gggccggtcataaagcagctctca

SEQ ID NO: 60 - DNA, 24 bp, Zea mays
  source 1..24: mol_type = unassigned DNA
acggatagcgctcctcgttggcgc

SEQ ID NO: 61 - DNA, 24 bp, Zea mays
  source 1..24: mol_type = unassigned DNA
acaatgttagccgatgatgacgac

SEQ ID NO: 62 - DNA, 24 bp, Zea mays
  source 1..24: mol_type = unassigned DNA
ggtaaccgtcctccgtacgtcgtc

SEQ ID NO: 63 - DNA, 24 bp, Zea mays
  source 1..24: mol_type = unassigned DNA
cctctctacgacgacgtacggagg

SEQ ID NO: 64 - DNA, 24 bp, Zea mays
  source 1..24: mol_type = unassigned DNA
gttacgggcagtgcagttgagcaa

SEQ ID NO: 65 - DNA, 24 bp, Zea mays
  source 1..24: mol_type = unassigned DNA
ctgactgtccagtggccacctaga

SEQ ID NO: 66 - skipped

SEQ ID NO: 67 - DNA, 1226 bp, synthetic construct
  misc_feature 1..1226: note = Description of Artificial Sequence: Synthetic polynucleotide
  misc_feature 1..1226: note = p35S+Adh1 intron promoter sequence
  source 1..1226: mol_type = other DNA
gttcagaagaccagagggctattgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatacatggtggagcacgacactctcgtctactccaagaatatcaaagatacagtctcagaagaccagagggctattgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagggtccgccttgtttctcctctgtctcttgatctgactaatcttggtttatgattcgttgagtaattttggggaaagcttcgtccacagttttttttcgatgaacagtgccgcagtggcgctgatcttgtatgctatcctgcaatcgtggtgaacttatttcttttatatcctttactcccatgaaaaggctagtaatctttctcgatgtaacatcgtccagcactgctattaccgtgtggtccatccgacagtctggctgaacacatcatacgatctatggagcaaaaatctatcttccctgttctttaatgaaggacgtcattttcattagtatgatctaggaatgttgcaacttgcaaggaggcgtttctttctttgaatttaactaactcgttgagtggccctgtttctcggacgtaaggcctttgctgctccacacatgtccattcgaattttaccgtgtttagcaagggcgaaaagtttgcatcttgatgatttagcttgactatgcgattgctttcctggacccgtgcag

SEQ ID NO: 68 - DNA, 5770 bp, synthetic construct
  misc_feature 1..5770: note = Description of Artificial Sequence: Synthetic polynucleotide
  misc_feature 1..5770: note = pGEP471 mRNA array vector construct
  source 1..5770: mol_type = other DNA
ctgacgcgccctgtagcggcacgtcacctgcatcaggaattcaggagaagaactcgagagggaattgcagatcatgaggcagatggctatttttgtgtcacatatgcgcaaaaagagaggctatatttgtgtccctaggttcttcgttgtattgcagtttccatatcaatctgacttggtcgcatgagaaattgatggttaaataatttgaatctctcatgtagtatcaactattagatattattttcaccaaatatatttccatcggagaagaagaggctacagaggaagcagaagagaggggtgggagaatttttacacttttgtacacccacttaaacagcaaaatccgtatgaaaacaggcccaccaaaacaatgccacgataacaatccgtagaaacaaaagcttcatttaacagcggcgcaacaaagcacgcttatccatggtagttgtagtccgtatgcgatccaaagatcacgattcacgcgtgacggacggacgacgcgtgccacaccacaactaacggcatccatggtagttgtagtccgtatgcgatccaaagatcacgattcacgcgtgacggacggacgacgcgcgccacaccacaactaacagcgtgagccagcgtccaaactccggatggcaacggggacgaaacccgtcgggtagtcactgcccaaacccgtccccgcaaccttcatcccaaacccgtccccgtttccggtcgcgggtttcagttttctaccagacccgtccccatcgggtttttcatccccgtcgggaaatccgaacccgccagcatttcagcaccaagccaaagttgcagcagcaacatgaataaaaaacaacccgtttcaacaccaagataaaacaaaacattataatttagacaacatttcacacgtataacaataacatatagttctcacatataacaacaccatttcacacataaaacaacaccatttgggataaaaatatgggctatatcaggccatttttatgggccatattgagttttcgtgggtttcacaggtaccggatttgtagaatgctgaaccgggtttgaaccgtaaaatccgcgggtattgaatttgacccaatcccgtcgtcccctggtggggtaaaaacaccatcttgagtccaaacggccaccaaccaaactccgacggcaacaaacaaacggcgttgctttgctcctcggtatctccgtgaccgctcaatctcccggctgtttccccggaattgcgtggactctctcatccacacgcaaaccgcctctccctcctctctcgtcctatccgccccggtgccgtagcctcacgggactcttcttcctcccttgctataaaatccccgccccctcctgtctcctctccacacatccaaactctcaatcgcaccgagaaaaatctcctagcgatcgaagcgaagcctctcccgatcctctcaaggtacgcccgtttcccgtcgatcctcctccttccgttcgtgttctgtagccgatcgattcgattcccttacacccgttcgtgttctctcgtggatcgatcgattgtttgttgctagaaggaactcgtagatctggcgtttatgaactgtgattcgggttagtccagatcgattcaggtcggtcgtcgttgagcctctcggctatgtctggattatcgtgtagatctgctggttcagttgattatgttcttctaggagtaatttcgttgggtcagcgcgatttctgcttaatctatgctgcttattgcgcctgtacctatctactaagctatgtgcacctgtaattttgctagattattcgttcatcctcgtagttggtttgtcacagtaatccgtatgggttctgacgatgttattgttggtcatacctaggcttctccagattttattttgttaaaattggatagatctgctactgatagttgatgatggaatttggtgctgaatctatgctatttattgcgcctatacctgatctatcgggctatgtacggctgtagtttactggattattcgttcatcctcggtagttggttcatcgtttgggttctgacgataatattgttgattatgcgtaggcttctgcagattgttgttaaaattggatacatcggttactgatggttgatgatagatttgtgctgaacctatctgtttattgctcctatacctgatctatagggctatgtatgcctgtaatttaccagattattcgttcatcctcgtagttggttcatctctataattcgtatgggttcttatgatgttatcgttgattatgcctagtcttatacagattattgtgtcaagattgaatatacctgctactgatcggtgataatttggttagtagtttgcaatctgctaggaacacgttaccactgtaatctgtaaacatggtttgccagagtagtttgttctactactcttgatatggttgctgattttagtcgcctccttttggatcatgtattgatgtccttgcagatttccgtgtacttaccccggcttttgtgtacttcgtgttaacagctctagaggatcctctcaacacaacatatacaaaacaaacgaatctcaagcaatcaagcattctacttctattgcagcaatttaaatcatttcttttaaagcaaaagcaattttctgaaaattttcaccatttacgaacgatagggcgcgatcccgccaccatggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaagtctagaggtacctgataatttctactaagtgtagatctcgtcacgattcccctctcctggaatttctactcttgtagattgtgtggtcacacttgccagccagaatttctactcttgtagatgtctatgtcgatgaccagcagattaatttctactaagtgtagatcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgctcgacgcggccgccatggcctctagtggatcaggtgtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 69 - DNA, 5770 bp, synthetic construct
  misc_feature 1..5770: note = Description of Artificial Sequence: Synthetic polynucleotide
  misc_feature 1..5770: note = pGEP472 mRNA array vector construct
  source 1..5770: mol_type = other DNA
ctgacgcgccctgtagcggcacgtcacctgcatcaggaattcaggagaagaactcgagagggaattgcagatcatgaggcagatggctatttttgtgtcacatatgcgcaaaaagagaggctatatttgtgtccctaggttcttcgttgtattgcagtttccatatcaatctgacttggtcgcatgagaaattgatggttaaataatttgaatctctcatgtagtatcaactattagatattattttcaccaaatatatttccatcggagaagaagaggctacagaggaagcagaagagaggggtgggagaatttttacacttttgtacacccacttaaacagcaaaatccgtatgaaaacaggcccaccaaaacaatgccacgataacaatccgtagaaacaaaagcttcatttaacagcggcgcaacaaagcacgcttatccatggtagttgtagtccgtatgcgatccaaagatcacgattcacgcgtgacggacggacgacgcgtgccacaccacaactaacggcatccatggtagttgtagtccgtatgcgatccaaagatcacgattcacgcgtgacggacggacgacgcgcgccacaccacaactaacagcgtgagccagcgtccaaactccggatggcaacggggacgaaacccgtcgggtagtcactgcccaaacccgtccccgcaaccttcatcccaaacccgtccccgtttccggtcgcgggtttcagttttctaccagacccgtccccatcgggtttttcatccccgtcgggaaatccgaacccgccagcatttcagcaccaagccaaagttgcagcagcaacatgaataaaaaacaacccgtttcaacaccaagataaaacaaaacattataatttagacaacatttcacacgtataacaataacatatagttctcacatataacaacaccatttcacacataaaacaacaccatttgggataaaaatatgggctatatcaggccatttttatgggccatattgagttttcgtgggtttcacaggtaccggatttgtagaatgctgaaccgggtttgaaccgtaaaatccgcgggtattgaatttgacccaatcccgtcgtcccctggtggggtaaaaacaccatcttgagtccaaacggccaccaaccaaactccgacggcaacaaacaaacggcgttgctttgctcctcggtatctccgtgaccgctcaatctcccggctgtttccccggaattgcgtggactctctcatccacacgcaaaccgcctctccctcctctctcgtcctatccgccccggtgccgtagcctcacgggactcttcttcctcccttgctataaaatccccgccccctcctgtctcctctccacacatccaaactctcaatcgcaccgagaaaaatctcctagcgatcgaagcgaagcctctcccgatcctctcaaggtacgcccgtttcccgtcgatcctcctccttccgttcgtgttctgtagccgatcgattcgattcccttacacccgttcgtgttctctcgtggatcgatcgattgtttgttgctagaaggaactcgtagatctggcgtttatgaactgtgattcgggttagtccagatcgattcaggtcggtcgtcgttgagcctctcggctatgtctggattatcgtgtagatctgctggttcagttgattatgttcttctaggagtaatttcgttgggtcagcgcgatttctgcttaatctatgctgcttattgcgcctgtacctatctactaagctatgtgcacctgtaattttgctagattattcgttcatcctcgtagttggtttgtcacagtaatccgtatgggttctgacgatgttattgttggtcatacctaggcttctccagattttattttgttaaaattggatagatctgctactgatagttgatgatggaatttggtgctgaatctatgctatttattgcgcctatacctgatctatcgggctatgtacggctgtagtttactggattattcgttcatcctcggtagttggttcatcgtttgggttctgacgataatattgttgattatgcgtaggcttctgcagattgttgttaaaattggatacatcggttactgatggttgatgatagatttgtgctgaacctatctgtttattgctcctatacctgatctatagggctatgtatgcctgtaatttaccagattattcgttcatcctcgtagttggttcatctctataattcgtatgggttcttatgatgttatcgttgattatgcctagtcttatacagattattgtgtcaagattgaatatacctgctactgatcggtgataatttggttagtagtttgcaatctgctaggaacacgttaccactgtaatctgtaaacatggtttgccagagtagtttgttctactactcttgatatggttgctgattttagtcgcctccttttggatcatgtattgatgtccttgcagatttccgtgtacttaccccggcttttgtgtacttcgtgttaacagctctagaggatcctctcaacacaacatatacaaaacaaacgaatctcaagcaatcaagcattctacttctattgcagcaatttaaatcatttcttttaaagcaaaagcaattttctgaaaattttcaccatttacgaacgatagggcgcgatcccgccaccatggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaagtctagaggtacctgataatttctactaagtgtagatgtctatgtcgatgaccagcagataatttctactcttgtagatctcgtcacgattcccctctcctggaatttctactcttgtagattgtgtggtcacacttgccagccagtaatttctactaagtgtagatcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgctcgacgcggccgccatggcctctagtggatcaggtgtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 70 - DNA, 3938 bp, synthetic construct
  misc_feature 1..3938: note = Description of Artificial Sequence: Synthetic polynucleotide
  misc_feature 1..3938: note = pGEP473 ribozyme array vector construct
  source 1..3938: mol_type = other DNA
ctgacgcgccctgtagcggcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgatcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccaaattactgatgagtccgtgaggacgaaacgagtaagctcgtctaatttctactaagtgtagatctcgtcacgattcccctctcctggaatttctactcttgtagattgtgtggtcacacttgccagccagaatttctactcttgtagatgtctatgtcgatgaccagcagatggcgtcagtcctactctgcacctcctcgtggtgtcgcctgggaaccctctttcacaagaaagaggagccaagcagagaggcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatcgtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 71 - DNA, 3938 bp, synthetic construct
  misc_feature 1..3938: note = Description of Artificial Sequence: Synthetic polynucleotide
  misc_feature 1..3938: note = pGEP474 ribozyme array vector construct
  source 1..3938: mol_type = other DNA
ctgacgcgccctgtagcggcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgatcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccaaattactgatgagtccgtgaggacgaaacgagtaagctcgtctaatttctactaagtgtagatgtctatgtcgatgaccagcagataatttctactcttgtagatctcgtcacgattcccctctcctggaatttctactcttgtagattgtgtggtcacacttgccagccagggcgtcagtcctactctgcacctcctcgtggtgtcgcctgggaaccctctttcacaagaaagaggagccaagcagagaggcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatcgtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 72 - DNA, 3759 bp, synthetic construct
  misc_feature 1..3759: note = Description of Artificial Sequence: Synthetic polynucleotide
  misc_feature 1..3759: note = codon optimized LbCpf1
  source 1..3759: mol_type = other DNA
atggcgccgaagaagaagcgcaaggtgtccaagctcgagaagttcacgaactgctactccctctccaagaccctccgcttcaaggccatccccgtgggcaagacccaggagaacatcgacaacaagcgcctcctggtcgaggacgagaagagggcggaggactacaagggcgtgaagaagctcctggaccgctactacctctccttcatcaacgacgtcctgcacagcatcaagctcaagaacctgaacaactacatctccctgttccgcaagaagacgaggaccgagaaggagaacaaggagctcgagaacctggagatcaacctccgcaaggagatcgccaaggcgttcaagggcaacgagggctacaagagcctgttcaagaaggacatcatcgagacgatcctcccggagttcctggacgacaaggacgagatcgccctcgtgaactccttcaacggcttcaccacggcgttcaccggcttcttcgacaaccgcgagaacatgttcagcgaggaggccaagtccacgagcatcgcgttccgctgcatcaacgagaacctgaccaggtacatctccaacatggacatcttcgagaaggtcgacgccatcttcgacaagcacgaggtgcaggagatcaaggagaagatcctcaacagcgactacgacgtcgaggacttcttcgagggcgagttcttcaacttcgtcctgacgcaggagggcatcgacgtgtacaacgccatcatcggtggcttcgtgaccgagtccggcgagaagatcaagggcctcaacgagtacatcaacctgtacaaccagaagaccaagcagaagctcccgaagttcaagcccctctacaagcaggtcctgtccgaccgcgagtccctgagcttctacggcgagggctacacgagcgacgaggaggtcctcgaggtgttcaggaacaccctgaacaagaacagcgagatcttctccagcatcaagaagctcgagaagctgttcaagaacttcgacgagtactccagcgccggcatcttcgtcaagaacggcccggcgatctccacgatcagcaaggatatcttcggcgagtggaacgtgatcagggacaagtggaacgccgagtacgacgacatccacctcaagaagaaggcggtggtcaccgagaagtacgaggacgaccgcaggaagtccttcaagaagatcggctccttcagcctcgagcagctgcaggagtacgccgacgcggacctctccgtggtcgagaagctgaaggagatcatcatccagaaggtcgacgagatctacaaggtgtacggctccagcgagaagctgttcgacgccgacttcgtcctcgagaagtccctgaagaagaacgacgccgtggtcgcgatcatgaaggacctcctggactccgtgaagagcttcgagaactacatcaaggcgttcttcggcgagggcaaggagacgaaccgcgacgagtccttctacggcgacttcgtcctcgcctacgacatcctcctgaaggtggaccacatctacgacgcgatcaggaactacgtgacccagaagccgtacagcaaggacaagttcaagctgtacttccagaacccccagttcatgggcggctgggacaaggacaaggagacggactaccgcgccaccatcctccgctacggcagcaagtactacctggccatcatggacaagaagtacgcgaagtgcctccagaagatcgacaaggacgacgtcaacggcaactacgagaagatcaactacaagctcctgccgggccccaacaagatgctgccgaaggtgttcttctccaagaagtggatggcctactacaaccccagcgaggacatccagaagatctacaagaacggcacgttcaagaagggcgacatgttcaacctcaacgactgccacaagctgatcgacttcttcaaggactccatcagccgctacccgaagtggtccaacgcctacgacttcaacttcagcgagacagagaagtacaaggacatcgcgggcttctacagggaggtcgaggagcagggctacaaggtgtccttcgagtccgccagcaagaaggaggtcgacaagctcgtggaggagggcaagctgtacatgttccagatctacaacaaggacttctccgacaagagccacggcacgcccaacctccacaccatgtacttcaagctcctgttcgacgagaacaaccacggccagatccgcctctccggcggcgccgagctgttcatgaggagggcgagcctcaagaaggaggagctggtggtccaccccgctaacagcccaatcgcgaacaagaacccggacaaccccaagaagaccacgaccctctcctacgacgtgtacaaggacaagcgcttcagcgaggaccagtacgagctgcacatcccgatcgccatcaacaagtgccccaagaacatcttcaagatcaacaccgaggtcagggtgctcctgaagcacgacgacaacccctacgtgatcggcatcgaccgcggcgagaggaacctcctgtacatcgtggtcgtggacggcaagggcaacatcgtggagcagtactccctgaacgagatcatcaacaacttcaacggcatccgcatcaagacggactaccacagcctcctggacaagaaggagaaggagcgcttcgaggccaggcagaactggacctccatcgagaacatcaaggagctcaaggcgggctacatcagccaggtcgtgcacaagatctgcgagctggtcgagaagtacgacgccgtgatcgcgctcgaggacctgaactccggcttcaagaacagcagggtcaaggtggagaagcaggtctaccagaagttcgagaagatgctcatcgacaagctgaactacatggtggacaagaagtccaacccgtgcgctacgggcggcgcgctcaagggctaccagatcaccaacaagttcgagagcttcaagtccatgagcacccagaacggcttcatcttctacatcccggcctggctgacgtccaagatcgaccccagcaccggcttcgtcaacctcctgaagacgaagtacacctccatcgcggacagcaagaagttcatctccagcttcgaccgcatcatgtatgtgccggaggaggacctcttcgagttcgccctggactacaagaacttctccaggacggacgcggattacatcaagaagtggaagctctacagctacggcaaccgcatcaggatcttccgcaaccccaagaagaacaacgtcttcgactgggaggaggtgtgcctcacctccgcctacaaggagctgttcaacaagtacggcatcaactaccagcagggcgacatcagggcgctcctgtgcgagcagagcgacaaggccttctactccagcttcatggcgctcatgtccctcatgctgcagatgcgcaacagcatcacgggcaggaccgacgtcgacttcctgatctccccggtgaagaacagcgacggcatcttctacgacagccgcaactacgaggcccaggagaacgcgatcctgccaaagaacgcggacgccaacggcgcctacaacatcgcgaggaaggtgctgtgggccatcggccagttcaagaaggcggaggacgagaagctcgacaaggtcaagatcgccatctccaacaaggagtggctggagtacgcgcagacctcggtgaagcacaagaggcccgctgccaccaagaaggcgggccaggccaagaagaagaagtga

SEQ ID NO: 73 - DNA, 3759 bp, synthetic construct
  misc_feature 1..3759: note = Description of Artificial Sequence: Synthetic polynucleotide
  misc_feature 1..3759: note = codon optimimized LbCpf1
  source 1..3759: mol_type = other DNA
atggcccccaagaagaagcgcaaggtgagcaagctggagaagtttacaaactgctactccctgtctaagactctgcgcttcaaggccatccctgtgggcaagacccaggagaacatcgacaataagcggctgctggtggaggacgagaagagagccgaggattataagggcgtgaagaagctgctggatcgctactatctgtcttttatcaacgacgtgctgcacagcatcaagctgaagaatctgaacaattacatcagcctgttccggaagaaaaccagaaccgagaaggagaataaggagctggagaacctggagatcaatctgcggaaggagatcgccaaggccttcaagggcaacgagggctacaagtccctgtttaagaaggatatcatcgagacaatcctgccagagttcctggacgataaggacgagatcgccctggtgaacagcttcaatggctttaccacagccttcaccggcttctttgataacagagagaatatgttttccgaggaggccaagagcacatccatcgccttcaggtgtatcaacgagaatctgacccgctacatctctaatatggacatcttcgagaaggtggacgccatctttgataagcacgaggtgcaggagatcaaggagaagatcctgaacagcgactatgatgtggaggatttctttgagggcgagttctttaactttgtgctgacacaggagggcatcgacgtgtataacgccatcatcggcggcttcgtgaccgagagcggcgagaagatcaagggcctgaacgagtacatcaacctgtataatcagaaaaccaagcagaagctgcctaagtttaagccactgtataagcaggtgctgagcgatcgggagtctctgagcttctacggcgagggctatacatccgatgaggaggtgctggaggtgtttagaaacaccctgaacaagaacagcgagatcttcagctccatcaagaagctggagaagctgttcaagaattttgacgagtactctagcgccggcatctttgtgaagaacggccccgccatcagcacaatctccaaggatatcttcggcgagtggaacgtgatccgggacaagtggaatgccgagtatgacgatatccacctgaagaagaaggccgtggtgaccgagaagtacgaggacgatcggagaaagtccttcaagaagatcggctccttttctctggagcagctgcaggagtacgccgacgccgatctgtctgtggtggagaagctgaaggagatcatcatccagaaggtggatgagatctacaaggtgtatggctcctctgagaagctgttcgacgccgattttgtgctggagaagagcctgaagaagaacgacgccgtggtggccatcatgaaggacctgctggattctgtgaagagcttcgagaattacatcaaggccttctttggcgagggcaaggagacaaacagggacgagtccttctatggcgattttgtgctggcctacgacatcctgctgaaggtggaccacatctacgatgccatccgcaattatgtgacccagaagccctactctaaggataagttcaagctgtattttcagaaccctcagttcatgggcggctgggacaaggataaggagacagactatcgggccaccatcctgagatacggctccaagtactatctggccatcatggataagaagtacgccaagtgcctgcagaagatcgacaaggacgatgtgaacggcaattacgagaagatcaactataagctgctgcccggccctaataagatgctgccaaaggtgttcttttctaagaagtggatggcctactataaccccagcgaggacatccagaagatctacaagaatggcacattcaagaagggcgatatgtttaacctgaatgactgtcacaagctgatcgacttctttaaggatagcatctcccggtatccaaagtggtccaatgcctacgatttcaacttttctgagacagagaagtataaggacatcgccggcttttacagagaggtggaggagcagggctataaggtgagcttcgagtctgccagcaagaaggaggtggataagctggtggaggagggcaagctgtatatgttccagatctataacaaggacttttccgataagtctcacggcacacccaatctgcacaccatgtacttcaagctgctgtttgacgagaacaatcacggacagatcaggctgagcggaggagcagagctgttcatgaggcgcgcctccctgaagaaggaggagctggtggtgcacccagccaactcccctatcgccaacaagaatccagataatcccaagaaaaccacaaccctgtcctacgacgtgtataaggataagaggttttctgaggaccagtacgagctgcacatcccaatcgccatcaataagtgccccaagaacatcttcaagatcaatacagaggtgcgcgtgctgctgaagcacgacgataacccctatgtgatcggcatcgataggggcgagcgcaatctgctgtatatcgtggtggtggacggcaagggcaacatcgtggagcagtattccctgaacgagatcatcaacaacttcaacggcatcaggatcaagacagattaccactctctgctggacaagaaggagaaggagaggttcgaggcccgccagaactggacctccatcgagaatatcaaggagctgaaggccggctatatctctcaggtggtgcacaagatctgcgagctggtggagaagtacgatgccgtgatcgccctggaggacctgaactctggctttaagaatagccgcgtgaaggtggagaagcaggtgtatcagaagttcgagaagatgctgatcgataagctgaactacatggtggacaagaagtctaatccttgtgcaacaggcggcgccctgaagggctatcagatcaccaataagttcgagagctttaagtccatgtctacccagaacggcttcatcttttacatccctgcctggctgacatccaagatcgatccatctaccggctttgtgaacctgctgaaaaccaagtataccagcatcgccgattccaagaagttcatcagctcctttgacaggatcatgtatgtgcccgaggaggatctgttcgagtttgccctggactataagaacttctctcgcacagacgccgattacatcaagaagtggaagctgtactcctacggcaaccggatcagaatcttccggaatcctaagaagaacaacgtgttcgactgggaggaggtgtgcctgaccagcgcctataaggagctgttcaacaagtacggcatcaattatcagcagggcgatatcagagccctgctgtgcgagcagtccgacaaggccttctactctagctttatggccctgatgagcctgatgctgcagatgcggaacagcatcacaggccgcaccgacgtggattttctgatcagccctgtgaagaactccgacggcatcttctacgatagccggaactatgaggcccaggagaatgccatcctgccaaagaacgccgacgccaatggcgcctataacatcgccagaaaggtgctgtgggccatcggccagttcaagaaggccgaggacgagaagctggataaggtgaagatcgccatctctaacaaggagtggctggagtacgcccagaccagcgtgaagcacaaaaggccggcggccacgaaaaaggccggccaggcaaaaaagaaaaagtga

SEQ ID NO: 74 - DNA, 3783 bp, synthetic construct
  misc_feature 1..3783: note = Description of Artificial Sequence: Synthetic polynucleotide
  misc_feature 1..3783: note = codon optimized LbCpf1
  source 1..3783: mol_type = other DNA
atggctcctaagaagaagcggaaggttggtattcacggggtgcctgcggcttcaaagctcgagaaattcaccaactgttattcgttgagcaaaacactgcggtttaaagcgattccagtcggcaagactcaagagaatatagacaataagcggctgttggtggaagatgaaaagcgcgcggaagactacaaaggggtgaagaagttgttggacagatactacctctcttttatcaatgatgtcttgcactcaatcaaattgaagaatctgaacaactacatctccctcttcagaaagaaaacaaggacagaaaaggagaataaggaacttgaaaatttggagatcaatctgaggaaagagatcgcgaaagcctttaaaggcaacgaaggatacaaaagtctgttcaagaaggatataattgagacaattttgccagagttcctcgatgacaaggacgagattgcgctggtcaattcgttcaacggattcacaacagcattcacaggcttctttgataatcgggaaaatatgttctctgaggaggcaaagtccacttctattgcgttcaggtgtatcaatgagaatctcactaggtacatttccaacatggatatctttgagaaggttgacgcaatttttgacaagcacgaagttcaggagattaaggagaagatcctcaattccgattatgacgttgaggacttcttcgaaggtgagttttttaatttcgtgctcactcaagagggtatcgacgtgtataatgcgatcatcggtgggttcgtgactgagtccggtgaaaagattaagggattgaacgagtatatcaacctttacaaccaaaagacgaaacagaagctgccaaagttcaagcctctttacaaacaggttctttcagaccgcgagtcactctcgttctatggggagggctacacttcggatgaggaagtcctggaggtgttcaggaatactctcaataagaattcggagattttctcttctataaaaaaactggaaaagttgtttaagaattttgacgaatactctagcgccggcatatttgtgaaaaacggcccggccatatcaacgataagtaaagatatcttcggcgaatggaacgtgatcagagacaaatggaacgcggagtatgacgatattcacctgaagaagaaggctgtcgtaacggagaagtacgaggatgatcgcaggaaaagcttcaaaaagatcggaagtttcagcctggaacagttgcaggagtatgctgacgccgatcttagcgtcgtcgagaagttgaaggagataatcatccaaaaggtcgacgagatatataaagtctatggatcaagtgaaaaactgttcgacgccgacttcgttttggagaagtccctgaagaagaacgacgctgttgttgccattatgaaggatctgctcgacagcgtgaagagtttcgagaactatattaaggcttttttcggggaggggaaggagactaacagagatgagtccttctacggagacttcgtcctcgcgtacgatatactccttaaggtagaccacatctacgacgcaatcagaaattacgtgacacaaaagccgtacagcaaggacaagttcaaactctacttccagaacccccagttcatgggcggctgggacaaggacaaggaaacggattacagggctacgatcttgaggtatggttcaaaatactacttggcgattatggacaagaagtacgccaagtgtctccagaagattgacaaagacgatgtcaatggcaattatgagaagatcaactacaagctgcttccgggtccgaacaagatgctcccaaaggttttcttcagcaagaaatggatggcctactataacccaagcgaggacatccagaagatttataagaacggtacgttcaagaagggcgacatgttcaatcttaacgactgtcacaagctgatcgacttcttcaaagactcaattagccggtacccaaagtggtctaacgcctatgacttcaacttttcggaaaccgagaagtacaaggatatagccggattttatagagaggtggaagagcagggctacaaggtgtcattcgagtccgccagcaagaaggaagtggacaagctcgtggaagagggtaagctctacatgttccagatttataataaagactttagcgataagagccacgggacacctaatctccacacaatgtatttcaagctgctcttcgacgagaataaccacggccaaatcaggttgtcaggaggggctgaactcttcatgcggcgcgctagccttaagaaggaggagcttgtagtccaccctgcgaatagtccaattgcgaataagaacccggacaatcctaaaaagactacaacattgagctacgacgtgtacaaggataagaggttttccgaggatcagtacgagctccacatcccgattgcgatcaacaagtgcccaaagaatattttcaagataaacacagaggtgcgtgtactcctgaagcatgacgacaatccttacgtcattgggattgatcggggcgagaggaacctcctctatattgtggtggtggacgggaaggggaacatagtcgaacagtactcccttaacgaaataattaacaatttcaacggcatccgtatcaagaccgactaccattcgttgctggacaagaaggagaaggagagatttgaggcgcggcaaaattggacaagtatcgagaacatcaaggaactcaaagcaggttatatctctcaagttgtgcataagatatgcgagctggttgagaagtatgacgcagtgatcgctcttgaggacctcaactcgggctttaagaattctagagttaaagtggagaagcaggtctatcaaaagttcgagaagatgcttatagataagctcaactacatggtcgataagaaatcgaacccatgtgccaccggcggcgcactcaaaggttaccaaataacaaacaaattcgagtccttcaaatcgatgagtactcagaatgggttcatattttatataccggcgtggcttacgtctaagatcgacccgtcaactggttttgtcaacctgttgaagacgaaatacacgtccattgccgattcgaaaaagttcatatctagttttgatcgtattatgtacgtcccagaggaagatcttttcgagtttgctctcgactacaaaaacttttcgcggaccgatgcggattacattaaaaaatggaaactctattcgtacggcaacagaatcaggatttttcgcaaccctaagaagaataacgtctttgattgggaggaagtttgcttgactagcgcgtacaaggagctctttaataagtatggcattaactaccaacagggtgatatcagagcactgctttgcgaacaatctgacaaggctttctactcatccttcatggctttgatgagcctgatgctccagatgagaaattcaattacaggcagaaccgacgtggatttcttgatctccccggttaaaaattctgatggcatcttttacgatagcaggaactatgaagcgcaagagaatgcgattctgccaaaaaatgcagacgccaacggtgcctataacatcgccaggaaagtcctgtgggcgatcggccagttcaaaaaggccgaagacgaaaaattggacaaggtcaaaatcgctatcagcaacaaagagtggctggagtatgctcagacatccgtaaagcataagcgtcctgctgccaccaaaaaggccggacaggctaagaaaaagaagtga

SEQ ID NO: 75 - DNA, 3759 bp, synthetic construct
  misc_feature 1..3759: note = Description of Artificial Sequence: Synthetic polynucleotide
  misc_feature 1..3759: note = codon optimized LbCpf1
  source 1..3759: mol_type = other DNA
atggccccgaagaagaagaggaaggtcagcaagctcgagaagttcaccaactgctacagcctgagcaagaccctgaggttcaaggctatcccggtgggcaagacccaagagaacatcgacaacaagaggctgctggtcgaggacgagaagcgcgctgaggattacaagggcgtgaagaagctgctggacaggtactacctgagcttcatcaacgacgtgctgcacagcatcaagctgaagaacctgaacaactacatcagcctgttccgcaagaaaaccaggaccgagaaagagaacaaagagcttgagaacctcgagatcaacctgaggaaagagatcgccaaggccttcaagggcaacgagggctacaagagcctgttcaagaaggacatcatcgagactatcctgccagagttcctggacgacaaggacgagatcgccctggtgaacagcttcaacggcttcacgaccgccttcaccggtttcttcgacaaccgcgagaatatgttcagcgaggaagccaagagcacctctatcgccttccgctgcatcaacgagaacctgacgcgctacatctccaacatggatatcttcgagaaggtggacgccatcttcgataagcacgaggtgcaagagatcaaagaaaagatcctgaacagcgactacgacgtcgaggacttcttcgagggcgagttcttcaacttcgtgctcacccaagagggcatcgatgtgtacaacgccatcatcggcggcttcgtgactgagagcggcgagaagatcaagggcctgaacgagtacatcaacctctacaatcaaaagaccaagcagaagctgccgaagttcaagccgctgtacaagcaggttctgagcgaccgcgagagcctgtctttctacggcgagggttacaccagcgacgaagaggtgttggaggttttccgcaacaccctgaacaagaacagcgagatcttcagctccatcaagaagctggaaaagctgtttaagaacttcgacgagtacagcagcgccggcatcttcgtgaagaacggcccagctatcagcaccatcagcaaggacatcttcggcgagtggaacgtgatcagggacaagtggaacgccgagtacgacgacatccacctgaagaaaaaggccgtggtgaccgagaagtacgaggacgacaggcgcaagagcttcaagaagatcggctccttcagcctcgagcagctgcaagagtacgctgacgctgacctgagcgtggtcgagaagctcaaagagatcatcatccagaaggtcgacgagatctacaaggtgtacggcagcagcgagaagcttttcgacgccgacttcgtccttgagaagtccctcaagaaaaacgacgccgtggtggccatcatgaaggacctgctggactccgtgaagtccttcgagaactacattaaggctttcttcggtgagggcaaagagactaacagggacgagagcttctacggggatttcgtgctggcctacgacatcctgctcaaggtggaccacatctacgacgccatccgcaactacgtgacccagaagccgtactccaaggacaagtttaagctgtacttccagaatccgcagttcatgggcggctgggacaaagacaaagaaaccgactacagggccaccatcctgaggtacggctccaagtactacctcgccatcatggacaagaaatacgccaagtgcctgcagaagatcgataaggacgacgtgaacggcaactacgagaagattaactacaagctgctgccagggccgaacaagatgctcccgaaggtgttctttagcaagaaatggatggcctactacaacccgagcgaggatatccagaaaatctacaagaacggcaccttcaagaaaggcgacatgttcaacctgaacgactgccacaagctgatcgatttcttcaaggacagcatctctcgctacccgaagtggtccaacgcctacgatttcaacttcagcgagactgaaaagtacaaggatatcgccggcttctaccgcgaggtcgaggaacagggttacaaggtgagcttcgagagcgccagcaagaaagaggtggacaagctggtcgaagagggcaagctgtacatgttccagatctataacaaggacttctccgacaagagccacggcaccccaaacctgcacaccatgtacttcaagttgctgttcgacgagaacaaccacggccagatcaggctttctggcggcgctgagcttttcatgagaagggccagcctgaaaaaagaggaactggtcgttcacccggcgaacagcccaatcgccaacaagaacccggacaacccgaaaaagaccaccacgctgagctacgacgtgtacaaggacaaaaggttctccgaggaccagtacgagctgcacatcccgatcgccatcaacaagtgcccgaagaacatcttcaagatcaacaccgaggtgagggtgctgctgaagcacgacgacaacccatacgtgatcggcatcgataggggcgagcgcaacctgctctacatcgtggtggttgacggcaagggcaatatcgtcgagcagtacagccttaacgagatcattaacaacttcaatggcatcaggatcaagaccgactaccacagcctgctcgacaagaaagaaaaagagcgcttcgaggccaggcagaactggaccagcatcgagaatatcaaagagctgaaggccggctacattagccaggtggtgcacaagatctgcgagctggtggaaaagtacgacgcggtgatcgctctcgaggacctgaactccgggttcaagaactcccgcgtgaaggttgagaagcaggtctaccaaaagttcgagaagatgctgatcgacaagctcaactacatggtggacaaaaagagcaacccctgcgccacaggcggcgctcttaagggctaccagatcacgaacaagttcgagtccttcaagagcatgagcacccagaatggcttcatcttctacatcccggcctggctgaccagcaagatcgatccatctaccggcttcgtcaacctcctcaagaccaagtacaccagcattgccgacagcaagaagttcatctccagcttcgacaggatcatgtacgtgccggaagaggacctgttcgagttcgcgctcgattacaagaacttcagcaggaccgacgcggactatattaagaagtggaagctctacagctacggcaacaggatccgcatcttcagaaacccgaagaaaaacaacgtgttcgactgggaagaagtgtgcctgaccagcgcctacaaagaactgttcaacaagtacggcatcaactaccagcagggcgacatcagggctctgctgtgcgagcagtctgacaaggcgttctacagctccttcatggccctgatgagcctgatgctgcagatgaggaacagcatcaccggcaggacggacgtcgacttcctgatcagcccagtgaagaattccgacggcattttctacgactctaggaactacgaggctcaagagaacgccatcctgccgaagaacgccgatgctaacggcgcgtacaacattgcccgcaaggtgctgtgggctatcggccagtttaagaaggccgaggacgaaaaactggacaaggtgaagatcgccattagcaacaaagagtggctcgagtacgcccagaccagcgtgaagcacaaaaggccagccgccactaagaaggctggccaggccaaaaagaagaagtga

SEQ ID NO: 76 - DNA, 3759 bp, synthetic construct
  misc_feature 1..3759: note = Description of Artificial Sequence: Synthetic polynucleotide
  misc_feature 1..3759: note = codon optimized LbCpf1
  source 1..3759: mol_type = other DNA
atggcacccaagaagaagcgcaaagtgtcaaagctcgagaagttcacaaactgttactccttatctaaaaccctgcgctttaaagcaattcctgtcggtaaaacccaagagaacatcgacaacaagagactgctcgttgaagatgaaaagagagccgaggattacaagggcgtgaagaagctcctcgatcgctattacctgtccttcattaacgatgtgctccatagcatcaagctcaagaaccttaacaactatatctcattattccgcaagaaaactagaacggagaaggaaaacaaagagctagagaaccttgaaatcaacctcagaaaggaaatagccaaggcgtttaaggggaatgaaggctacaagagtttgtttaagaaagacatcattgagacaatattacctgaattccttgatgacaaggacgagatcgctctagtgaacagctttaatggtttcaccactgcgttcacgggcttcttcgataacagggagaatatgttttctgaggaagccaagtcaacttccatcgcgtttcgctgcatcaacgagaatctgacccgttacataagtaacatggacatattcgagaaagttgacgctatcttcgacaagcatgaggtgcaagaaatcaaggaaaagatccttaactctgactacgacgtcgaggacttcttcgagggagaattcttcaatttcgttctgacccaggagggcatcgacgtgtacaatgctattatcggtggattcgtgacagagtccggagaaaagattaagggccttaacgagtatatcaacctttataaccaaaagacgaagcaaaaactccccaagtttaaacctctttacaaacaagttctatcggatagagaaagcctttcgttttacggggaaggatacacctctgatgaagaggttctcgaggtgttccgtaacaccctgaacaagaactccgagatattctcgtcgattaagaaactcgaaaagttgttcaaaaacttcgacgaatactcatctgccggaatttttgtgaagaacggcccggctatttcgaccatttccaaggatatcttcggagagtggaacgttatacgagataagtggaacgcagagtatgatgatatccaccttaagaagaaggcggtcgtgacggaaaaatacgaggacgatcgtaggaagtctttcaagaagattggtagcttcagcctcgagcaactgcaggaatacgcggatgctgatctgagcgtggtcgagaagcttaaggagataatcatccaaaaggttgacgagatatacaaggtttatggttcgtcagagaagttgttcgacgccgacttcgtccttgagaagtccctgaagaagaatgacgcggtcgtggcaatcatgaaagacctcctcgactccgtcaaatcctttgagaattatattaaggcgttcttcggcgaagggaaggagacaaatagggatgagagtttttacggcgattttgtgctagcttacgacattctgctgaaagttgaccacatatacgacgctatccgaaactatgtcacccaaaagccttactcaaaagacaagttcaagctgtactttcaaaacccgcagttcatgggaggatgggataaggacaaggaaactgactacagagccacgattctccgctacgggtccaaatactacctcgccattatggataagaagtacgcgaagtgcctgcagaagatcgacaaagacgacgtgaacggaaactacgagaagatcaactataagctgctgccagggcccaacaagatgctgccgaaggttttctttagcaagaagtggatggcctactacaacccgtccgaggacatacagaaaatctacaaaaatggaactttcaaaaagggcgacatgttcaacttgaacgattgccacaagttaattgacttcttcaaggacagcatttcgcgatatccgaagtggtctaatgcctatgacttcaatttttctgaaaccgagaagtataaggatatcgcgggtttttatagggaagttgaggagcaaggttacaaagtatcatttgaatctgcctccaaaaaggaggtcgacaaactggttgaagagggcaaactatacatgtttcagatctacaacaaagatttctcggataagtcgcacgggacgccgaacttacacaccatgtacttcaaactgctgtttgatgagaacaatcacggccagatccgtctaagcggcggtgccgagcttttcatgcgccgcgcgagcttgaaaaaggaggagttggtggtccaccctgctaattcaccgattgctaacaagaaccccgataaccccaaaaagaccaccactcttagctacgatgtctataaggataagcgcttcagcgaagatcagtatgagttgcatattccgattgccatcaacaaatgccccaaaaatattttcaagatcaacactgaggtccgcgtgctgctcaaacatgacgataacccgtacgtaatcggcatcgatagaggcgaacggaacttactatacatcgtggtagtagacgggaagggaaatatcgtcgaacagtacagtctgaatgaaattattaacaatttcaacgggatccggatcaagacagactaccactccttgctcgacaagaaggaaaaggagcggttcgaggcccgacaaaactggacttcgattgagaacattaaggagctcaaagcggggtacatctcccaagtggtgcataaaatctgtgaactggttgagaaatatgatgcagtgatagctctcgaggacctcaattctggcttcaagaactccagggttaaggtagaaaaacaggtctaccaaaaatttgaaaagatgcttattgataagttaaattacatggtcgacaaaaagtcgaatccgtgtgccacaggaggcgccctcaagggataccagattacgaataagtttgaatccttcaagtctatgagcacacagaacggcttcattttctatatccccgcgtggctcacttctaaaatcgacccatccaccggcttcgtgaatttgcttaagacaaagtacactagcatcgcggactcgaagaaattcatttcgtcgtttgaccggattatgtatgtgccagaagaagatctattcgagtttgctctcgactataaaaacttctcccgcaccgacgccgactacataaagaagtggaagttgtatagctacgggaaccgcatcaggatattccggaatccgaagaagaacaatgtgtttgattgggaggaggtctgcctcacgtcagcttacaaggagctgttcaacaaatacggtataaactatcagcagggcgacatccgggcccttctgtgtgaacagagcgacaaagcattttactcttcttttatggctctgatgtccttgatgctgcaaatgcgcaattcaatcacggggagaaccgatgtagactttctgattagtccggtcaagaatagcgacggcatattctacgattcaaggaattacgaagcccaggagaacgcgatcctgccaaaaaatgcagatgcgaatggtgcatacaatattgcaaggaaagtgttatgggccatcggccagttcaagaaagctgaggacgagaagcttgacaaggtcaagatcgcaatctcaaacaaggaatggcttgaatatgcgcagactagtgtgaaacataagcgcccagctgccaccaagaaggccggccaggccaagaaaaagaagtga

SEQ ID NO: 77 - DNA, 24 bp, Triticum aestivum
  source 1..24: mol_type = unassigned DNA
cagcatggcatggagggtgacgat

SEQ ID NO: 78 - DNA, 24 bp, Triticum aestivum
  source 1..24: mol_type = unassigned DNA
agcatggcatggagggtgacgatg

SEQ ID NO: 79 - DNA, 24 bp, Triticum aestivum
  source 1..24: mol_type = unassigned DNA
cgcaggaggaggaggagctcatcg

SEQ ID NO: 80 - DNA, 24 bp, Triticum aestivum
  source 1..24: mol_type = unassigned DNA
cgcaccgcttcagccctgcagcac

SEQ ID NO: 81 - DNA, 24 bp, Triticum aestivum
  source 1..24: mol_type = unassigned DNA
gcaccgcttcagccctgcagcacg

SEQ ID NO: 82 - DNA, 21 bp, Beta vulgaris
  source 1..21: mol_type = unassigned DNA
gctgctaaacaatcaacattt

SEQ ID NO: 83 - DNA, 21 bp, Beta vulgaris
  source 1..21: mol_type = unassigned DNA
taaacaatcaacatttaggta

SEQ ID NO: 84 - DNA, 20 bp, Beta vulgaris
  source 1..20: mol_type = unassigned DNA
atttaggtatggttgtccaa

SEQ ID NO: 85 - DNA, 20 bp, Beta vulgaris
  source 1..20: mol_type = unassigned DNA
tttagcagcattatcttaac

SEQ ID NO: 86 - DNA, 20 bp, Beta vulgaris
  source 1..20: mol_type = unassigned DNA
tatagaacctatcttcccat

SEQ ID NO: 87 - RNA, 86 nt, Helianthus annuus; identical to SEQ ID NO: 24
  source 1..86: mol_type = unassigned RNA

SEQ ID NO: 88 - DNA, 24 bp, synthetic construct
  misc_feature 1..24: note = Description of Artificial Sequence: Synthetic oligonucleotide
  misc_feature 1..24: note = crALS1 protospacer
  source 1..24: mol_type = other DNA
caccctaattgtagccaactcttg

SEQ ID NO: 89 - DNA, 24 bp, synthetic construct
  misc_feature 1..24: note = Description of Artificial Sequence: Synthetic oligonucleotide
  misc_feature 1..24: note = crALS2 protospacer
  source 1..24: mol_type = other DNA
gcagcattatcttaactgggagat

SEQ ID NO: 90 - DNA, 24 bp, synthetic construct
  misc_feature 1..24: note = Description of Artificial Sequence: Synthetic oligonucleotide
  misc_feature 1..24: note = crALS3 protospacer
  source 1..24: mol_type = other DNA
ggtatggttgtccaatgggaagat

SEQ ID NO: 91 - DNA, 24 bp, synthetic construct
  misc_feature 1..24: note = Description of Artificial Sequence: Synthetic oligonucleotide
  misc_feature 1..24: note = crALS4 protospacer
  source 1..24: mol_type = other DNA
caaggtatgtatgtgcccggttag

SEQ ID NO: 92 - DNA, 24 bp, synthetic construct
  misc_feature 1..24: note = Description of Artificial Sequence: Synthetic oligonucleotide
  misc_feature 1..24: note = crALS5 protospacer
  source 1..24: mol_type = other DNA
gaagggtttccaaggtatgtatgt

SEQ ID NO: 93 - DNA, 8828 bp, synthetic construct
  misc_feature 1..8828: note = Description of Artificial Sequence: Synthetic polynucleotide
  misc_feature 1..8828: note = expression plasmid
  source 1..8828: mol_type = other DNA
gattgaaagaaatatagtttaaatatttattgataaaataacaagtcaggtattatagtccaagcaaaaacataaatttattgatgcaagtttaaattcagaaatatttcaataactgattatatcagctggtacattgccgtagatgaaagactgagtgcgatattatgtgtaatacataaattgatgatatagctagcttagctcatcgggtcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgagtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgaagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatccaagctcccattttctgtgattttggatgtgtttttgtatggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcattccggactgtggggccatatcccagaactggttgagtcggtccaacacctgggtgccaatcatgtcgatggtggggtatggccaattttttttcaattcaaaaatgtagatgtccgcagcgttattataaaatgaaagtacattttgataaaacgacaaattacgatccgtcgtatttataggcgaaagcaataaacaaattattctaattcggaaatctttatttcgacgtgtctacattcacgtccaaatgggggcttagatgagaaacttcacgatcggctctagaggccatggcggccgctcgagcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggaaattcgatctacacttagtagaaattacaagagttggctacaattagggtgatctacacttagtagaaattatcaggtacctctagacttgtacagctcgtccatgccgtacaggaacaggtggtggcggccctcggagcgctcgtactgttccacgatggtgtagtcctcgttgtgggaggtgatgtccagcttggtgtccacgtagtagtagccgggcagttgcacgggcttcttggccatgtagatggtcttgaactccaccaggtagtggccgccgtccttcagcttcagggcctggtggatctcgcccttcagcacgccgtcgcgggggtacaggcgctcggtggaggcctcccagcccatggtcttcttctgcattacggggccgtcgggggggaagttggtgccgcgcatcttcaccttgtagatcagcgtgccgtcctgcagggaggagtcctgggtcacggtcaccagaccgccgtcctcgaagttcatcacgcgctcccacttgaagccctcggggaaggacagcttcttgtaatcggggatgtcggcggggtgcttcacgtacgccttggagccgtacatgaactggggggacaggatgtcccaggcgaagggcagggggccgcccttggtcaccttcagcttggcggtctgggtgccctcgtaggggcggccctcgccctcgccctcgatctcgaactcgtggccgttcatggagccctccatgcgcaccttgaagcgcatgaactctttgatgacctcctcgcccttgctcaccatggatcccctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgttcctcgtgggtgggggtccatctttgggaccactgtcggtagaggcatcttgaacgatagcctttcctttatcgcaatgatggcatttgtagaagccatcttccttttctactgtcctttcgatgaagtgacagatagctgggcaatggaatccgaggaggtttcccgatattaccctttgttgaaaagtctcaatagccctctggtcttctgagactgtatctttgatattcttggagtagacgagagtgtcgtgctccaccatgtatcacatcaatccacttgctttgaagacgtggttggaacgtcttctttttccacgatgttcctcgtgggtgggggtccatctttgggaccactgtcggtagaggcatcttgaacgatagcctttcctttatcgcaatgatggcatttgtagaagccatcttccttttctactgtcctttcgatgaagtgacagatagctgggcaatggaatccgaggaggtttcccgatattaccctttgttgaaaagtctcaatagccctctggtcttctgaacactagtaaggccttaagggccagatcccccgggctgcaggaattcgatctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaattcaaaaattacggatatgaatataggcatatccgtatccgaattatccgtttgacagctagcaacgattgtacaattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctcacaattcatcatctttctttctctacccccaattttaagaaatcctctcttctcctcttcattttcaaggtaaatctctctctctctctctctctctgttattccttgttttaattaggtatgtattattgctagtttgttaatctgcttatcttatgtatgccttatgtgaatatctttatcttgttcatctcatccgtttagaagctataaatttgttgatttgactgtgtatctacacgtggttatgtttatatctaatcagatatgaatttcttcatattgttgcgtttgtgtgtaccaatccgaaatcgttgatttttttcatttaatcgtgtagctaattgtacgtatacatatggatctacgtatcaattgttcatctgtttgtgtttgtatgtatacagatctgaaaacatcacttctctcatctgattgtgttgttacatacatagatatagatctgttatatcattttttttattaattgtgtatatatatatgtgcatagatctggattacatgattgtgattatttacatgattttgttatttacgtatgtatatatgtagatctggactttttggagttgttgacttgattgtatttgtgtgtgtatatgtgtgttctgatcttgatatgttatgtatgtgcagcgaattcggcgcgccaaacaatggcttcctccatggctcctaagaagaagaggaaggttagcaagctcgagaagtttaccaactgctacagcctctctaagaccctcaggttcaaggctatccctgtgggaaagacccaagagaatatcgacaacaagaggctcctcgtcgaggatgagaagagagctgaagattacaagggcgtgaagaagctcctcgacaggtactacctcagcttcatcaacgatgtgctccacagcatcaagctcaagaacctcaacaactacatcagcctcttccgtaagaaaaccaggaccgagaaagagaacaaagagcttgagaacctcgagatcaacctccgtaaagagatcgccaaggctttcaagggaaacgagggatacaagagcctcttcaagaaggatattatcgagacaatcctgcctgagttcctggacgataaggatgagatcgctctcgtgaacagcttcaacggattcactactgccttcaccggattcttcgacaacagggaaaacatgttcagcgaagaggccaagagcacctctatcgctttcagatgcatcaacgagaacctcacgcgttacatcagcaacatggacatcttcgagaaggtggacgccatcttcgataagcacgaggtgcaagaaatcaaagagaagatcctcaacagcgactacgacgtcgaggacttttttgaaggggagttcttcaacttcgttctcacccaagagggcatcgacgtgtacaacgctattatcggaggattcgtgaccgagtctggggagaagattaagggactcaacgagtacatcaacctgtacaaccagaaaacgaagcagaagctcccgaagttcaagccgctctacaagcaggttctctctgatcgtgagagcctctcattttacggtgagggttacacctctgacgaggaagtgcttgaggttttccgtaacaccctcaacaagaacagcgagatcttctcgtccatcaagaagttggagaaacttttcaagaacttcgacgagtacagcagcgctgggatcttcgttaagaacggacctgctatcagcaccatcagcaaggatattttcggcgagtggaacgtgatcagggacaagtggaatgctgagtacgatgacatccacctcaagaagaaggctgtcgtcactgagaagtacgaggatgacaggcgtaagtcgttcaagaagatcggctctttcagcctcgagcagcttcaagaatacgctgatgctgatctcagcgtggtcgagaagctcaaagagatcatcatccagaaggtcgacgagatctacaaggtgtacgggtcctctgagaagttgttcgatgctgatttcgtcctcgagaagagtctgaagaagaacgacgctgtcgtcgcgatcatgaaggatttgctcgacagcgtgaagtccttcgagaactatatcaaggccttcttcggagagggcaaagagactaatagggacgagtctttctacggggatttcgtgctcgcttacgatatcctcctcaaggtggaccatatctacgacgccatcagaaactacgtgacccagaagccttacagcaaggacaagttcaagttgtactttcagaacccgcagttcatgggcggatgggacaaagacaaagagacagattacagggccaccatcctcaggtacgggtctaagtactacctggccatcatggacaagaaatacgccaagtgcctccaaaagatcgacaaggatgacgtgaacgggaactatgagaagatcaactacaagctccttccgggaccgaacaagatgcttcctaaggtgttcttcagcaagaaatggatggcctactacaacccgtctgaggacatccagaaaatctacaagaacgggaccttcaagaaaggcgacatgttcaacctcaacgactgccacaagctcatcgatttcttcaaggacagcatctcgcgttacccgaagtggtctaacgcttacgactttaacttcagcgagacagaaaagtacaaggatatcgccgggttctaccgtgaggttgaggaacagggttacaaggttagcttcgagagcgcctccaagaaagaggttgacaagttggtcgaagagggcaagctctacatgttccagatctataacaaggacttctccgacaagagccacggaactcctaacctccatacgatgtacttcaagctgcttttcgacgagaacaaccacgggcagatcagactttctggtggtgctgaactcttcatgcgtagggcctcactcaagaaagaagagttggttgttcacccggccaactctccaatcgctaacaagaatcctgacaacccgaaaaagaccaccacgctgtcttacgacgtctacaaggacaaaaggttcagcgaggaccagtacgagcttcatatcccgatcgctatcaacaagtgcccgaagaacatcttcaagatcaataccgaggtgagggtgctgctcaagcacgatgataacccttacgtgatcggaatcgatcgtggtgagagaaacctcctctacatcgttgtggtggacggaaagggaaacatcgtcgagcagtacagcctgaacgagattatcaacaatttcaacggcatcaggatcaagaccgactaccactcactcctcgataagaaagaaaaagagcgtttcgaggccaggcagaactggacttctatcgaaaacatcaaagagttgaaggccggctacatctctcaggtggtgcataagatctgcgagctggtggaaaagtacgatgctgtgatcgctcttgaggacctcaactctgggttcaagaacagtagagtgaaggttgagaagcaggtctaccaaaagttcgagaagatgctcatcgacaagctcaactacatggtggacaaaaagagcaacccttgcgctaccggtggtgctcttaagggataccagatcacgaacaagttcgagtccttcaagagcatgagcacccagaacggcttcatcttctatatccctgcttggctcaccagcaagatcgatccttctactggtttcgtgaacctgctcaagaccaagtacacctcgatcgccgacagcaagaagttcatctcgtctttcgacaggatcatgtacgtgccggaagaggatcttttcgagttcgctctcgactataagaacttcagcaggaccgacgccgactacattaagaagtggaagctctactcctacgggaaccgtatcaggatcttccgaaatccgaagaaaaacaacgtgttcgactgggaagaagtgtgcctcacctctgcctacaaagaactgttcaacaagtacggcatcaactaccagcagggtgatatcagggctcttttgtgcgagcagagcgacaaggcattctacagctcattcatggccctcatgtctctcatgctccagatgaggaactctatcaccggaaggaccgatgtggacttccttatctctccggtcaagaactctgacgggatcttctacgacagccgtaactatgaggctcaagagaacgctatcctgccgaagaatgctgatgcaaacggggcttacaacattgcgagaaaggttctctgggctatcgggcagtttaagaaagcggaagatgagaagctcgacaaggtgaagatcgccatctccaacaaagagtggcttgagtacgctcagacctccgttaagcacaagaggcctgctgctactaagaaagctggccaggccaaaaagaagaagtgaggcgcgccgagctccaggcctcccagctttcgtccgtatcatcggtttcgacaacgttcgtcaagttcaatgcatcagtttcattgcccacacaccagaatcctactaagtttgagtattatggcattggaaaagctgttttcttctatcatttgttctgcttgtaatttactgtgttctttcagtttttgttttcggacatcaaaatgcaaatggatggataagagttaataaatgatatggtccttttgttcattctcaaattattattatctgttgtttttactttaatgggttgaatttaagtaagaaaggaactaacagtgtgatattaaggtgcaatgttagacatataaaacagtctttcacctctctttggttatgtcttgaattggtttgtttcttcacttatctgtgtaatcaagtttactatgagtctatgatcaagtaattatgcaatcaagttaagtacagtataggctt

SEQ ID NO: 94 - DNA, 8201 bp, synthetic construct
  misc_feature 1..8201: note = Description of Artificial Sequence: Synthetic polynucleotide
  misc_feature 1..8201: note = expression plasmid
  source 1..8201: mol_type = other DNA
ttgaaagaaatatagtttaaatatttattgataaaataacaagtcaggtattatagtccaagcaaaaacataaatttattgatgcaagtttaaattcagaaatatttcaataactgattatatcagctggtacattgccgtagatgaaagactgagtgcgatattatgtgtaatacataaattgatgatatagctagcttagctcatcgggtcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgagtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgaagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatccaagctcccattttctgtgattttggatgtgtttttgtatggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcattccggactgtggggccatatcccagaactggttgagtcggtccaacacctgggtgccaatcatgtcgatggtggggtatggccaattttttttcaattcaaaaatgtagatgtccgcagcgttattataaaatgaaagtacattttgataaaacgacaaattacgatccgtcgtatttataggcgaaagcaataaacaaattattctaattcggaaatctttatttcgacgtgtctacattcacgtccaaatgggggcttagatgagaaacttcacgatcggctctagaggccatggcggccgctcgagcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcgcctctctgcttggctcctctttcttgtgaaagagggttcccaggcgacaccacgaggaggtgcagagtaggactgacgcccaagagttggctacaattagggtgatctacacttagtagaaattagacgagcttactcgtttcgtcctcacggactcatcagtaatttggatcccctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgttcctcgtgggtgggggtccatctttgggaccactgtcggtagaggcatcttgaacgatagcctttcctttatcgcaatgatggcatttgtagaagccatcttccttttctactgtcctttcgatgaagtgacagatagctgggcaatggaatccgaggaggtttcccgatattaccctttgttgaaaagtctcaatagccctctggtcttctgagactgtatctttgatattcttggagtagacgagagtgtcgtgctccaccatgtatcacatcaatccacttgctttgaagacgtggttggaacgtcttctttttccacgatgttcctcgtgggtgggggtccatctttgggaccactgtcggtagaggcatcttgaacgatagcctttcctttatcgcaatgatggcatttgtagaagccatcttccttttctactgtcctttcgatgaagtgacagatagctgggcaatggaatccgaggaggtttcccgatattaccctttgttgaaaagtctcaatagccctctggtcttctgaacactagtaaggccttaagggccagatcccccgggctgcaggaattcgatctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaattcaaaaattacggatatgaatataggcatatccgtatccgaattatccgtttgacagctagcaacgattgtacaattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctcacaattcatcatctttctttctctacccccaattttaagaaatcctctcttctcctcttcattttcaaggtaaatctctctctctctctctctctctgttattccttgttttaattaggtatgtattattgctagtttgttaatctgcttatcttatgtatgccttatgtgaatatctttatcttgttcatctcatccgtttagaagctataaatttgttgatttgactgtgtatctacacgtggttatgtttatatctaatcagatatgaatttcttcatattgttgcgtttgtgtgtaccaatccgaaatcgttgatttttttcatttaatcgtgtagctaattgtacgtatacatatggatctacgtatcaattgttcatctgtttgtgtttgtatgtatacagatctgaaaacatcacttctctcatctgattgtgttgttacatacatagatatagatctgttatatcattttttttattaattgtgtatatatatatgtgcatagatctggattacatgattgtgattatttacatgattttgttatttacgtatgtatatatgtagatctggactttttggagttgttgacttgattgtatttgtgtgtgtatatgtgtgttctgatcttgatatgttatgtatgtgcagcgaattcggcgcgccaaacaatggcttcctccatggctcctaagaagaagaggaaggttagcaagctcgagaagtttaccaactgctacagcctctctaagaccctcaggttcaaggctatccctgtgggaaagacccaagagaatatcgacaacaagaggctcctcgtcgaggatgagaagagagctgaagattacaagggcgtgaagaagctcctcgacaggtactacctcagcttcatcaacgatgtgctccacagcatcaagctcaagaacctcaacaactacatcagcctcttccgtaagaaaaccaggaccgagaaagagaacaaagagcttgagaacctcgagatcaacctccgtaaagagatcgccaaggctttcaagggaaacgagggatacaagagcctcttcaagaaggatattatcgagacaatcctgcctgagttcctggacgataaggatgagatcgctctcgtgaacagcttcaacggattcactactgccttcaccggattcttcgacaacagggaaaacatgttcagcgaagaggccaagagcacctctatcgctttcagatgcatcaacgagaacctcacgcgttacatcagcaacatggacatcttcgagaaggtggacgccatcttcgataagcacgaggtgcaagaaatcaaagagaagatcctcaacagcgactacgacgtcgaggacttttttgaaggggagttcttcaacttcgttctcacccaagagggcatcgacgtgtacaacgctattatcggaggattcgtgaccgagtctggggagaagattaagggactcaacgagtacatcaacctgtacaaccagaaaacgaagcagaagctcccgaagttcaagccgctctacaagcaggttctctctgatcgtgagagcctctcattttacggtgagggttacacctctgacgaggaagtgcttgaggttttccgtaacaccctcaacaagaacagcgagatcttctcgtccatcaagaagttggagaaacttttcaagaacttcgacgagtacagcagcgctgggatcttcgttaagaacggacctgctatcagcaccatcagcaaggatattttcggcgagtggaacgtgatcagggacaagtggaatgctgagtacgatgacatccacctcaagaagaaggctgtcgtcactgagaagtacgaggatgacaggcgtaagtcgttcaagaagatcggctctttcagcctcgagcagcttcaagaatacgctgatgctgatctcagcgtggtcgagaagctcaaagagatcatcatccagaaggtcgacgagatctacaaggtgtacgggtcctctgagaagttgttcgatgctgatttcgtcctcgagaagagtctgaagaagaacgacgctgtcgtcgcgatcatgaaggatttgctcgacagcgtgaagtccttcgagaactatatcaaggccttcttcggagagggcaaagagactaatagggacgagtctttctacggggatttcgtgctcgcttacgatatcctcctcaaggtggaccatatctacgacgccatcagaaactacgtgacccagaagccttacagcaaggacaagttcaagttgtactttcagaacccgcagttcatgggcggatgggacaaagacaaagagacagattacagggccaccatcctcaggtacgggtctaagtactacctggccatcatggacaagaaatacgccaagtgcctccaaaagatcgacaaggatgacgtgaacgggaactatgagaagatcaactacaagctccttccgggaccgaacaagatgcttcctaaggtgttcttcagcaagaaatggatggcctactacaacccgtctgaggacatccagaaaatctacaagaacgggaccttcaagaaaggcgacatgttcaacctcaacgactgccacaagctcatcgatttcttcaaggacagcatctcgcgttacccgaagtggtctaacgcttacgactttaacttcagcgagacagaaaagtacaaggatatcgccgggttctaccgtgaggttgaggaacagggttacaaggttagcttcgagagcgcctccaagaaagaggttgacaagttggtcgaagagggcaagctctacatgttccagatctataacaaggacttctccgacaagagccacggaactcctaacctccatacgatgtacttcaagctgcttttcgacgagaacaaccacgggcagatcagactttctggtggtgctgaactcttcatgcgtagggcctcactcaagaaagaagagttggttgttcacccggccaactctccaatcgctaacaagaatcctgacaacccgaaaaagaccaccacgctgtcttacgacgtctacaaggacaaaaggttcagcgaggaccagtacgagcttcatatcccgatcgctatcaacaagtgcccgaagaacatcttcaagatcaataccgaggtgagggtgctgctcaagcacgatgataacccttacgtgatcggaatcgatcgtggtgagagaaacctcctctacatcgttgtggtggacggaaagggaaacatcgtcgagcagtacagcctgaacgagattatcaacaatttcaacggcatcaggatcaagaccgactaccactcactcctcgataagaaagaaaaagagcgtttcgaggccaggcagaactggacttctatcgaaaacatcaaagagttgaaggccggctacatctctcaggtggtgcataagatctgcgagctggtggaaaagtacgatgctgtgatcgctcttgaggacctcaactctgggttcaagaacagtagagtgaaggttgagaagcaggtctaccaaaagttcgagaagatgctcatcgacaagctcaactacatggtggacaaaaagagcaacccttgcgctaccggtggtgctcttaagggataccagatcacgaacaagttcgagtccttcaagagcatgagcacccagaacggcttcatcttctatatccctgcttggctcaccagcaagatcgatccttctactggtttcgtgaacctgctcaagaccaagtacacctcgatcgccgacagcaagaagttcatctcgtctttcgacaggatcatgtacgtgccggaagaggatcttttcgagttcgctctcgactataagaacttcagcaggaccgacgccgactacattaagaagtggaagctctactcctacgggaaccgtatcaggatcttccgaaatccgaagaaaaacaacgtgttcgactgggaagaagtgtgcctcacctctgcctacaaagaactgttcaacaagtacggcatcaactaccagcagggtgatatcagggctcttttgtgcgagcagagcgacaaggcattctacagctcattcatggccctcatgtctctcatgctccagatgaggaactctatcaccggaaggaccgatgtggacttccttatctctccggtcaagaactctgacgggatcttctacgacagccgtaactatgaggctcaagagaacgctatcctgccgaagaatgctgatgcaaacggggcttacaacattgcgagaaaggttctctgggctatcgggcagtttaagaaagcggaagatgagaagctcgacaaggtgaagatcgccatctccaacaaagagtggcttgagtacgctcagacctccgttaagcacaagaggcctgctgctactaagaaagctggccaggccaaaaagaagaagtgaggcgcgccgagctccaggcctcccagctttcgtccgtatcatcggtttcgacaacgttcgtcaagttcaatgcatcagtttcattgcccacacaccagaatcctactaagtttgagtattatggcattggaaaagctgttttcttctatcatttgttctgcttgtaatttactgtgttctttcagtttttgttttcggacatcaaaatgcaaatggatggataagagttaataaatgatatggtccttttgttcattctcaaattattattatctgttgtttttactttaatgggttgaatttaagtaagaaaggaactaacagtgtgatattaaggtgcaatgttagacatataaaacagtctttcacctctctttggttatgtcttgaattggtttgtttcttcacttatctgtgtaatcaagtttactatgagtctatgatcaagtaattatgcaatcaagttaagtacagtataggctt

SEQ ID NO: 95 - DNA, 87 bp, synthetic construct
  misc_feature 1..87: note = Description of Artificial Sequence: Synthetic oligonucleotide
  misc_feature 1..87: note = expression casssette
  source 1..87: mol_type = other DNA
cggggaaattcgatctacacttagtagaaattacaagagttggctacaattagggtgatctacacttagtagaaattatcaggtacc

SEQ ID NO: 96 - DNA, 141 bp, Beta vulgaris
  source 1..141: mol_type = unassigned DNA
ggacaaccatacctaaatgttgattgtttagcagcattatcttaactgggagattttccaccctaattgtagccaactcttgaacattcataataaaactgccatccccatcaatatcgacaaccactgcatctggtcgag

SEQ ID NO: 97 - DNA, 135 bp, synthetic construct
  misc_feature 1..135: note = Description of Artificial Sequence: Synthetic polynucleotide
  misc_feature 1..135: note = InDel1
  source 1..135: mol_type = other DNA
ggacaaccatacctaaatgttgattgtttagcagcattatcttaactttttccaccctaattgtagccaactcttgaacattcataataaaactgccatccccatcaatatcgacaaccactgcatctggtcgag

SEQ ID NO: 98 - DNA, 134 bp, synthetic construct
  misc_feature 1..134: note = Description of Artificial Sequence: Synthetic polynucleotide
  misc_feature 1..134: note = InDel2
  source 1..134: mol_type = other DNA
ggacaaccatacctaaatgttgattgtttagcagcattatcttagattttccaccctaattgtagccaactcttgaacattcataataaaactgccatccccatcaatatcgacaaccactgcatctggtcgag

SEQ ID NO: 99 - DNA, 126 bp, synthetic construct
  misc_feature 1..126: note = Description of Artificial Sequence: Synthetic polynucleotide
  misc_feature 1..126: note = InDel3
  source 1..126: mol_type = other DNA
ggacaaccatacctaaatgttgattgtttagcagcattatttccaccctaattgtagccaactcttgaacattcataataaaactgccatccccatcaatatcgacaaccactgcatctggtcgag

SEQ ID NO: 100 - DNA, 103 bp, synthetic construct
  misc_feature 1..103: note = Description of Artificial Sequence: Synthetic polynucleotide
  misc_feature 1..103: note = InDel4
  source 1..103: mol_type = other DNA
ggacaaccatacctaaatgttgattgtttagcagcattatcttgaacattcataataaaactgccatccccatcaatatcgacaaccactgcatctggtcgag

SEQ ID NO: 101 - DNA, 83 bp, synthetic construct
  misc_feature 1..83: note = Description of Artificial Sequence: Synthetic oligonucleotide
  misc_feature 1..83: note = InDel5
  source 1..83: mol_type = other DNA
ggacaaccatacctaaatgttgattgtttagcagcattatctgccatccccatcaatatcgacaaccactgcatctggtcgag

SEQ ID NO: 102 - DNA, 76 bp, synthetic construct
  misc_feature 1..76: note = Description of Artificial Sequence: Synthetic oligonucleotide
  misc_feature 1..76: note = InDel6
  source 1..76: mol_type = other DNA
ggacaaccatacctaaatgttgattgtttagcagcattatccccatcaatatcgacaaccactgcatctggtcgag

SEQ ID NO: 103 - DNA, 64 bp, synthetic construct
  misc_feature 1..64: note = Description of Artificial Sequence: Synthetic oligonucleotide
  misc_feature 1..64: note = InDel7
  source 1..64: mol_type = other DNA
ggacaaccatacctaaatgttgattgtttagcagcattatcgacaaccactgcatctggtcgag

SEQ ID NO: 104 - DNA, 59 bp, Zea mays
  source 1..59: mol_type = unassigned DNA
tttaatttactgtcacgattcccctctcctggtcgaacttttcaggtggggaaagctgc

SEQ ID NO: 105 - DNA, 53 bp, synthetic construct
  misc_feature 1..53: note = Description of Artificial Sequence: Synthetic oligonucleotide
  misc_feature 1..53: note = InDel at crGEP5 target locus
  source 1..53: mol_type = other DNA
tttgatttactgtcacgattccctggtcgaacttttcaggtggggaaagctgc

SEQ ID NO: 106 - DNA, 58 bp, synthetic construct
  misc_feature 1..58: note = Description of Artificial Sequence: Synthetic oligonucleotide
  misc_feature 1..58: note = InDel at crGEP5 target locus
  source 1..58: mol_type = other DNA
tttgatttactgtcacgattcccctctcctgtcgaacttttcaggtggggaaagctgc

SEQ ID NO: 107-108 - skipped (2 entries)

SEQ ID NO: 109 - DNA, 39 bp, synthetic construct
  misc_feature 1..39: note = Description of Artificial Sequence: Synthetic oligonucleotide
  misc_feature 1..39: note = InDel at crGEP5 target locus
  source 1..39: mol_type = other DNA
tttgatttactgtctaacttttcaggtggggaaagctgc

SEQ ID NO: 110 - DNA, 58 bp, synthetic construct
  misc_feature 1..58: note = Description of Artificial Sequence: Synthetic oligonucleotide
  misc_feature 1..58: note = InDel at crGEP5 target locus
  source 1..58: mol_type = other DNA
tttgatttactgtcatgattcccctccctggtcgaacttttcaggtggggaaagctgc

SEQ ID NO: 111 - DNA, 60 bp, Zea mays
  source 1..60: mol_type = unassigned DNA
ctgtggcactaccaagctcctgctcaccattccaagaatccttgagcttgctgaagagct

SEQ ID NO: 112 - DNA, 54 bp, synthetic construct
  misc_feature 1..54: note = Description of Artificial Sequence: Synthetic oligonucleotide
  misc_feature 1..54: note = InDel at crGEP75 target locus
  source 1..54: mol_type = other DNA
ctgtggcactaccaagctcctgctcaccagaatccttgagcttgctgaagagct

SEQ ID NO: 113 - DNA, 56 bp, synthetic construct
  misc_feature 1..56: note = Description of Artificial Sequence: Synthetic oligonucleotide
  misc_feature 1..56: note = InDel at crGEP75 target locus
  source 1..56: mol_type = other DNA
ctgtggcactaccaagctcctgctcaccttggaatccttgagcttgctgaagagct

SEQ ID NO: 114 - DNA, 51 bp, synthetic construct
  misc_feature 1..51: note = Description of Artificial Sequence: Synthetic oligonucleotide
  misc_feature 1..51: note = InDel at crGEP75 target locus
  source 1..51: mol_type = other DNA
ctgtggcactaccaagctcctgctcagaatccttgagcttgctgaagagct

SEQ ID NO: 115 - DNA, 36 bp, synthetic construct
  misc_feature 1..36: note = Description of Artificial Sequence: Synthetic oligonucleotide
  misc_feature 1..36: note = InDel at crGEP75 target locus
  source 1..36: mol_type = other DNA
ctgtggcactaccaagctcctgcttgctgaagagct

SEQ ID NO: 116 - DNA, 53 bp, synthetic construct
  misc_feature 1..53: note = Description of Artificial Sequence: Synthetic oligonucleotide
  misc_feature 1..53: note = InDel at crGEP75 target locus
  source 1..53: mol_type = other DNA
ctgtggcactaccaagctcctgctcaatgaatccttgagcttgctgaagagct

SEQ ID NO: 117 - DNA, 57 bp, Zea mays
  source 1..57: mol_type = unassigned DNA
gcaggagctgcctggaggcgggctcctcgtgtaccagagcttctgtgctgaagacgc

SEQ ID NO: 118 - DNA, 49 bp, synthetic construct
  misc_feature 1..49: note = Description of Artificial Sequence: Synthetic oligonucleotide
  misc_feature 1..49: note = InDel at crGEP77 target locus
  source 1..49: mol_type = other DNA
gcaggagctgcctggaggcgggctcctcgagcttctgtgctgaagacgc

SEQ ID NO: 119 - DNA, 39 bp, synthetic construct
  misc_feature 1..39: note = Description of Artificial Sequence: Synthetic oligonucleotide
  misc_feature 1..39: note = InDel at crGEP77 target locus
  source 1..39: mol_type = other DNA
gcaggagctgcctggaggcgggctctgtgctgaagacgc

SEQ ID NO: 120 - DNA, 51 bp, synthetic construct
  misc_feature 1..51: note = Description of Artificial Sequence: Synthetic oligonucleotide
  source 1..51: mol_type = other DNA
gcaggagctgcctggaggcgggctcctcgtgagcttctgtgctgaagacgc

SEQ ID NO: 121 - DNA, 48 bp, synthetic construct
  misc_feature 1..48: note = Description of Artificial Sequence: Synthetic oligonucleotide
  misc_feature 1..48: note = InDel at crGEP77 target locus
  source 1..48: mol_type = other DNA
gcaggagctgcctggaggcgggctcctgagcttctgtgctgaagacgc

SEQ ID NO: 122 - DNA, 51 bp, synthetic construct
  misc_feature 1..51: note = Description of Artificial Sequence: Synthetic oligonucleotide
  misc_feature 1..51: note = InDel at crGEP77 target locus
  source 1..51: mol_type = other DNA
gcaggagctgcctggaggcgggctcctcgggagcttctgtgctgaagacgc

SEQ ID NO: 123 - DNA, 48 bp, synthetic construct
  misc_feature 1..48: note = Description of Artificial Sequence: Synthetic oligonucleotide
  misc_feature 1..48: note = InDel at crGEP77 target locus
  source 1..48: mol_type = other DNA
gcaggagctgcctggaggcgggctcctcagcttctgtgctgaagacgc

SEQ ID NO: 124 - DNA, 50 bp, synthetic construct
  misc_feature 1..50: note = Description of Artificial Sequence: Synthetic oligonucleotide
  misc_feature 1..50: note = InDel at crGEP77 target locus
  source 1..50: mol_type = other DNA
gcaggagctgcctggaggcgggctcctcgtagcttctgtgctgaagacgc

SEQ ID NO: 125 - DNA, 60 bp, Triticum aestivum
  source 1..60: mol_type = unassigned DNA
gctcttcccgcaccgcttcagccctgcagcacgcacccatccatgacgcaacacacatca

SEQ ID NO: 126 - DNA, 57 bp, synthetic construct
  misc_feature 1..57: note = Description of Artificial Sequence: Synthetic oligonucleotide
  misc_feature 1..57: note = InDel in A genome
  source 1..57: mol_type = other DNA
gctcttcccgcaccgcttcagccctgcacgcacccatccatgacgcaacacacatca

SEQ ID NO: 127 - DNA, 53 bp, synthetic construct
  misc_feature 1..53: note = Description of Artificial Sequence: Synthetic oligonucleotide
  misc_feature 1..53: note = InDel in A genome
  source 1..53: mol_type = other DNA
gctcttcccgcaccgcttcagccccgcacccatccatgacgcaacacacatca

SEQ ID NO: 128 - DNA, 55 bp, synthetic construct
  misc_feature 1..55: note = Description of Artificial Sequence: Synthetic oligonucleotide
  misc_feature 1..55: note = InDel in A genome
  source 1..55: mol_type = other DNA
gctcttcccgcaccgcttcagccctacgcacccatccatgacgcaacacacatca

SEQ ID NO: 129 - DNA, 56 bp, synthetic construct
  misc_feature 1..56: note = Description of Artificial Sequence: Synthetic oligonucleotide
  misc_feature 1..56: note = InDel in A genome
  source 1..56: mol_type = other DNA
gctcttcccgcaccgcttcagcctgcacgcacccatccatgacgcaacacacatca

SEQ ID NO: 130 - DNA, 54 bp, synthetic construct
  misc_feature 1..54: note = Description of Artificial Sequence: Synthetic oligonucleotide
  misc_feature 1..54: note = InDel in A genome
  source 1..54: mol_type = other DNA
gctcttcccgcaccgcttcagccctggcacccatccatgacgcaacacacatca

SEQ ID NO: 131 - DNA, 46 bp, synthetic construct
  misc_feature 1..46: note = Description of Artificial Sequence: Synthetic oligonucleotide
  misc_feature 1..46: note = InDel in A genome
  source 1..46: mol_type = other DNA
gctcttcccgcaccgcttcagcccatccatgacgcaacacacatca

SEQ ID NO: 132 - DNA, 48 bp, synthetic construct
  misc_feature 1..48: note = Description of Artificial Sequence: Synthetic oligonucleotide
  misc_feature 1..48: note = InDel in A genome
  source 1..48: mol_type = other DNA
gctcttcccgcaccgcttcagcccccatccatgacgcaacacacatca

SEQ ID NO: 133 - DNA, 48 bp, synthetic construct
  misc_feature 1..48: note = Description of Artificial Sequence: Synthetic oligonucleotide
  misc_feature 1..48: note = InDel in A genome
  source 1..48: mol_type = other DNA
gctcttcccgcaccgcttcagcacccatccatgacgcaacacacatca

SEQ ID NO: 134 - DNA, 60 bp, Triticum aestivum; identical to SEQ ID NO: 125
  source 1..60: mol_type = unassigned DNA

SEQ ID NO: 135 - DNA, 50 bp, synthetic construct
  misc_feature 1..50: note = Description of Artificial Sequence: Synthetic oligonucleotide
  misc_feature 1..50: note = InDel in B genome
  source 1..50: mol_type = other DNA
gctcttcccgcaccgcttcagcccacccatccatgacgcaacacacatca

SEQ ID NO: 136 - DNA, 54 bp, synthetic construct
  misc_feature 1..54: note = Description of Artificial Sequence: Synthetic oligonucleotide
  misc_feature 1..54: note = InDel in B genome
  source 1..54: mol_type = other DNA
gctcttcccgcaccgcttcagcccacgcacccatccatgacgcaacacacatca

SEQ ID NO: 137 - DNA, 53 bp, synthetic construct
  misc_feature 1..53: note = Description of Artificial Sequence: Synthetic oligonucleotide
  misc_feature 1..53: note = InDel in B genome
  source 1..53: mol_type = other DNA
gctcttcccgcaccgcttcagccacgcacccatccatgacgcaacacacatca

SEQ ID NO: 138 - DNA, 51 bp, synthetic construct
  misc_feature 1..51: note = Description of Artificial Sequence: Synthetic oligonucleotide
  misc_feature 1..51: note = InDel in B genome
  source 1..51: mol_type = other DNA
gctcttcccgcaccgcttcagccgcacccatccatgacgcaacacacatca

SEQ ID NO: 139 - DNA, 50 bp, synthetic construct
  misc_feature 1..50: note = Description of Artificial Sequence: Synthetic oligonucleotide
  misc_feature 1..50: note = InDel in B genome
  source 1..50: mol_type = other DNA
gctcttcccgcaccgcttcagcgcacccatccatgacgcaacacacatca

SEQ ID NO: 140 - DNA, 60 bp, synthetic construct
  misc_feature 1..60: note = Description of Artificial Sequence: Synthetic oligonucleotide
  misc_feature 1..60: note = InDel in B genome
  source 1..60: mol_type = other DNA
catccatgacgcaccacacatcagcccagcacgcacccatccatgacgcaacacacatca

SEQ ID NO: 141 - DNA, 60 bp, Triticum aestivum; identical to SEQ ID NO: 125
  source 1..60: mol_type = unassigned DNA

SEQ ID NO: 142 - DNA, 53 bp, synthetic construct; identical to SEQ ID NO: 127
  misc_feature 1..53: note = Description of Artificial Sequence: Synthetic oligonucleotide
  misc_feature 1..53: note = InDel in D genome
  source 1..53: mol_type = other DNA

SEQ ID NO: 143 - DNA, 47 bp, synthetic construct
  misc_feature 1..47: note = Description of Artificial Sequence: Synthetic oligonucleotide
  misc_feature 1..47: note = InDel in D genome
  source 1..47: mol_type = other DNA
gctcttcccgcaccgcttcagccccatccatgacgcaacacacatca

SEQ ID NO: 144 - DNA, 51 bp, synthetic construct
  misc_feature 1..51: note = Description of Artificial Sequence: Synthetic oligonucleotide
  misc_feature 1..51: note = InDel in D genome
  source 1..51: mol_type = other DNA
gctcttcccgcaccgcttcagccctacccatccatgacgcaacacacatca

SEQ ID NO: 145 - DNA, 96 bp, synthetic construct
  misc_feature 1..96: note = Description of Artificial Sequence: Synthetic oligonucleotide
  misc_feature 1..96: note = expression cassette
  source 1..96: mol_type = other DNA
aattcgatctacacttagtagaaattaatctcccagtagaaattaatctcccagttaagataatgctgcatctacacttagtagaaattatcaggt

SEQ ID NO: 146 - DNA, 24 bp, synthetic construct
  misc_feature 1..24: note = Description of Artificial Sequence: Synthetic oligonucleotide
  misc_feature 1..24: note = protospacer
  source 1..24: mol_type = other DNA
caccctaattgtagccaactcttg

SEQ ID NO: 147 - DNA, 24 bp, synthetic construct
  misc_feature 1..24: note = Description of Artificial Sequence: Synthetic oligonucleotide
  misc_feature 1..24: note = protospacer
  source 1..24: mol_type = other DNA
gcagcattatcttaactgggagat

SEQ ID NO: 148 - DNA, 24 bp, synthetic construct
  misc_feature 1..24: note = Description of Artificial Sequence: Synthetic oligonucleotide
  misc_feature 1..24: note = protospacer
  source 1..24: mol_type = other DNA
ggtatggttgtccaatgggaagat

SEQ ID NO: 149 - DNA, 24 bp, synthetic construct
  misc_feature 1..24: note = Description of Artificial Sequence: Synthetic oligonucleotide
  misc_feature 1..24: note = protospacer
  source 1..24: mol_type = other DNA
caaggtatgtatgtgcccggttag

SEQ ID NO: 150 - DNA, 24 bp, synthetic construct
  misc_feature 1..24: note = Description of Artificial Sequence: Synthetic oligonucleotide
  misc_feature 1..24: note = protospacer
  source 1..24: mol_type = other DNA
gaagggtttccaaggtatgtatgt

SEQ ID NO: 151 - DNA, 187 bp, synthetic construct
  misc_feature 1..187: note = Description of Artificial Sequence: Synthetic polynucleotide
  misc_feature 1..187: note = expression cassettte
  source 1..187: mol_type = other DNA
cgatcgcctctctgcttggctcctctttcttgtgaaagagggttcccaggcgacaccacgaggaggtgcagagtaggactgacgcccaagagttggctacaattagggtgatctacacttagtagaaattagacgagcttactcgtttcgtcctcacggactcatcagtaatttggatcccctctcc

SEQ ID NO: 152 - protein, 1252 aa, unidentified
  REGION 1..1252: note = Description of Unknown: Lachnospiraceae bacterium sequence
MAPKKKRKVSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHKRPAATKKAGQAKKKK

SEQ ID NO: 153 - protein, 1252 aa, unidentified; identical to SEQ ID NO: 152
  REGION 1..1252: note = Description of Unknown: Lachnospiraceae bacterium sequence

SEQ ID NO: 154 - protein, 1260 aa, unidentified
  REGION 1..1260: note = Description of Unknown: Lachnospiraceae bacterium sequence
MAPKKKRKVGIHGVPAASKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHKRPAATKKAGQAKKKK

SEQ ID NO: 155 - protein, 1252 aa, unidentified; identical to SEQ ID NO: 152
  REGION 1..1252: note = Description of Unknown: Lachnospiraceae bacterium sequence

SEQ ID NO: 156 - protein, 1252 aa, unidentified; identical to SEQ ID NO: 152
  REGION 1..1252: note = Description of Unknown: Lachnospiraceae bacterium sequence

SEQ ID NO: 157 - DNA, 3948 bp, synthetic construct
  misc_feature 1..3948: note = Description of Artificial Sequence: Synthetic polynucleotide
  misc_feature 1..3948: note = variant I of Cpf1 (SEQ ID NO: 72) including an intronic sequence
  misc_feature 862..1050: note = Intron
  source 1..3948: mol_type = other DNA
atggcgccgaagaagaagcgcaaggtgtccaagctcgagaagttcacgaactgctactccctctccaagaccctccgcttcaaggccatccccgtgggcaagacccaggagaacatcgacaacaagcgcctcctggtcgaggacgagaagagggcggaggactacaagggcgtgaagaagctcctggaccgctactacctctccttcatcaacgacgtcctgcacagcatcaagctcaagaacctgaacaactacatctccctgttccgcaagaagacgaggaccgagaaggagaacaaggagctcgagaacctggagatcaacctccgcaaggagatcgccaaggcgttcaagggcaacgagggctacaagagcctgttcaagaaggacatcatcgagacgatcctcccggagttcctggacgacaaggacgagatcgccctcgtgaactccttcaacggcttcaccacggcgttcaccggcttcttcgacaaccgcgagaacatgttcagcgaggaggccaagtccacgagcatcgcgttccgctgcatcaacgagaacctgaccaggtacatctccaacatggacatcttcgagaaggtcgacgccatcttcgacaagcacgaggtgcaggagatcaaggagaagatcctcaacagcgactacgacgtcgaggacttcttcgagggcgagttcttcaacttcgtcctgacgcaggagggcatcgacgtgtacaacgccatcatcggtggcttcgtgaccgagtccggcgagaagatcaagggcctcaacgagtacatcaacctgtacaaccagaagaccaagcagaagctcccgaagttcaagcccctctacaagcaggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgtgcaggtcctgtccgaccgcgagtccctgagcttctacggcgagggctacacgagcgacgaggaggtcctcgaggtgttcaggaacaccctgaacaagaacagcgagatcttctccagcatcaagaagctcgagaagctgttcaagaacttcgacgagtactccagcgccggcatcttcgtcaagaacggcccggcgatctccacgatcagcaaggatatcttcggcgagtggaacgtgatcagggacaagtggaacgccgagtacgacgacatccacctcaagaagaaggcggtggtcaccgagaagtacgaggacgaccgcaggaagtccttcaagaagatcggctccttcagcctcgagcagctgcaggagtacgccgacgcggacctctccgtggtcgagaagctgaaggagatcatcatccagaaggtcgacgagatctacaaggtgtacggctccagcgagaagctgttcgacgccgacttcgtcctcgagaagtccctgaagaagaacgacgccgtggtcgcgatcatgaaggacctcctggactccgtgaagagcttcgagaactacatcaaggcgttcttcggcgagggcaaggagacgaaccgcgacgagtccttctacggcgacttcgtcctcgcctacgacatcctcctgaaggtggaccacatctacgacgcgatcaggaactacgtgacccagaagccgtacagcaaggacaagttcaagctgtacttccagaacccccagttcatgggcggctgggacaaggacaaggagacggactaccgcgccaccatcctccgctacggcagcaagtactacctggccatcatggacaagaagtacgcgaagtgcctccagaagatcgacaaggacgacgtcaacggcaactacgagaagatcaactacaagctcctgccgggccccaacaagatgctgccgaaggtgttcttctccaagaagtggatggcctactacaaccccagcgaggacatccagaagatctacaagaacggcacgttcaagaagggcgacatgttcaacctcaacgactgccacaagctgatcgacttcttcaaggactccatcagccgctacccgaagtggtccaacgcctacgacttcaacttcagcgagacagagaagtacaaggacatcgcgggcttctacagggaggtcgaggagcagggctacaaggtgtccttcgagtccgccagcaagaaggaggtcgacaagctcgtggaggagggcaagctgtacatgttccagatctacaacaaggacttctccgacaagagccacggcacgcccaacctccacaccatgtacttcaagctcctgttcgacgagaacaaccacggccagatccgcctctccggcggcgccgagctgttcatgaggagggcgagcctcaagaaggaggagctggtggtccaccccgctaacagcccaatcgcgaacaagaacccggacaaccccaagaagaccacgaccctctcctacgacgtgtacaaggacaagcgcttcagcgaggaccagtacgagctgcacatcccgatcgccatcaacaagtgccccaagaacatcttcaagatcaacaccgaggtcagggtgctcctgaagcacgacgacaacccctacgtgatcggcatcgaccgcggcgagaggaacctcctgtacatcgtggtcgtggacggcaagggcaacatcgtggagcagtactccctgaacgagatcatcaacaacttcaacggcatccgcatcaagacggactaccacagcctcctggacaagaaggagaaggagcgcttcgaggccaggcagaactggacctccatcgagaacatcaaggagctcaaggcgggctacatcagccaggtcgtgcacaagatctgcgagctggtcgagaagtacgacgccgtgatcgcgctcgaggacctgaactccggcttcaagaacagcagggtcaaggtggagaagcaggtctaccagaagttcgagaagatgctcatcgacaagctgaactacatggtggacaagaagtccaacccgtgcgctacgggcggcgcgctcaagggctaccagatcaccaacaagttcgagagcttcaagtccatgagcacccagaacggcttcatcttctacatcccggcctggctgacgtccaagatcgaccccagcaccggcttcgtcaacctcctgaagacgaagtacacctccatcgcggacagcaagaagttcatctccagcttcgaccgcatcatgtatgtgccggaggaggacctcttcgagttcgccctggactacaagaacttctccaggacggacgcggattacatcaagaagtggaagctctacagctacggcaaccgcatcaggatcttccgcaaccccaagaagaacaacgtcttcgactgggaggaggtgtgcctcacctccgcctacaaggagctgttcaacaagtacggcatcaactaccagcagggcgacatcagggcgctcctgtgcgagcagagcgacaaggccttctactccagcttcatggcgctcatgtccctcatgctgcagatgcgcaacagcatcacgggcaggaccgacgtcgacttcctgatctccccggtgaagaacagcgacggcatcttctacgacagccgcaactacgaggcccaggagaacgcgatcctgccaaagaacgcggacgccaacggcgcctacaacatcgcgaggaaggtgctgtgggccatcggccagttcaagaaggcggaggacgagaagctcgacaaggtcaagatcgccatctccaacaaggagtggctggagtacgcgcagacctcggtgaagcacaagaggcccgctgccaccaagaaggcgggccaggccaagaagaagaagtga

SEQ ID NO: 158 - DNA, 3948 bp, synthetic construct
  misc_feature 1..3948: note = Description of Artificial Sequence: Synthetic polynucleotide
  misc_feature 1..3948: note = variant III of Cpf1 (SEQ ID NO: 75) including an intronic
  misc_feature 628..816: note = Intron
  source 1..3948: mol_type = other DNA
atggccccgaagaagaagaggaaggtcagcaagctcgagaagttcaccaactgctacagcctgagcaagaccctgaggttcaaggctatcccggtgggcaagacccaagagaacatcgacaacaagaggctgctggtcgaggacgagaagcgcgctgaggattacaagggcgtgaagaagctgctggacaggtactacctgagcttcatcaacgacgtgctgcacagcatcaagctgaagaacctgaacaactacatcagcctgttccgcaagaaaaccaggaccgagaaagagaacaaagagcttgagaacctcgagatcaacctgaggaaagagatcgccaaggccttcaagggcaacgagggctacaagagcctgttcaagaaggacatcatcgagactatcctgccagagttcctggacgacaaggacgagatcgccctggtgaacagcttcaacggcttcacgaccgccttcaccggtttcttcgacaaccgcgagaatatgttcagcgaggaagccaagagcacctctatcgccttccgctgcatcaacgagaacctgacgcgctacatctccaacatggatatcttcgagaaggtggacgccatcttcgataagcacgaggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgtgcaggtgcaagagatcaaagaaaagatcctgaacagcgactacgacgtcgaggacttcttcgagggcgagttcttcaacttcgtgctcacccaagagggcatcgatgtgtacaacgccatcatcggcggcttcgtgactgagagcggcgagaagatcaagggcctgaacgagtacatcaacctctacaatcaaaagaccaagcagaagctgccgaagttcaagccgctgtacaagcaggttctgagcgaccgcgagagcctgtctttctacggcgagggttacaccagcgacgaagaggtgttggaggttttccgcaacaccctgaacaagaacagcgagatcttcagctccatcaagaagctggaaaagctgtttaagaacttcgacgagtacagcagcgccggcatcttcgtgaagaacggcccagctatcagcaccatcagcaaggacatcttcggcgagtggaacgtgatcagggacaagtggaacgccgagtacgacgacatccacctgaagaaaaaggccgtggtgaccgagaagtacgaggacgacaggcgcaagagcttcaagaagatcggctccttcagcctcgagcagctgcaagagtacgctgacgctgacctgagcgtggtcgagaagctcaaagagatcatcatccagaaggtcgacgagatctacaaggtgtacggcagcagcgagaagcttttcgacgccgacttcgtccttgagaagtccctcaagaaaaacgacgccgtggtggccatcatgaaggacctgctggactccgtgaagtccttcgagaactacattaaggctttcttcggtgagggcaaagagactaacagggacgagagcttctacggggatttcgtgctggcctacgacatcctgctcaaggtggaccacatctacgacgccatccgcaactacgtgacccagaagccgtactccaaggacaagtttaagctgtacttccagaatccgcagttcatgggcggctgggacaaagacaaagaaaccgactacagggccaccatcctgaggtacggctccaagtactacctcgccatcatggacaagaaatacgccaagtgcctgcagaagatcgataaggacgacgtgaacggcaactacgagaagattaactacaagctgctgccagggccgaacaagatgctcccgaaggtgttctttagcaagaaatggatggcctactacaacccgagcgaggatatccagaaaatctacaagaacggcaccttcaagaaaggcgacatgttcaacctgaacgactgccacaagctgatcgatttcttcaaggacagcatctctcgctacccgaagtggtccaacgcctacgatttcaacttcagcgagactgaaaagtacaaggatatcgccggcttctaccgcgaggtcgaggaacagggttacaaggtgagcttcgagagcgccagcaagaaagaggtggacaagctggtcgaagagggcaagctgtacatgttccagatctataacaaggacttctccgacaagagccacggcaccccaaacctgcacaccatgtacttcaagttgctgttcgacgagaacaaccacggccagatcaggctttctggcggcgctgagcttttcatgagaagggccagcctgaaaaaagaggaactggtcgttcacccggcgaacagcccaatcgccaacaagaacccggacaacccgaaaaagaccaccacgctgagctacgacgtgtacaaggacaaaaggttctccgaggaccagtacgagctgcacatcccgatcgccatcaacaagtgcccgaagaacatcttcaagatcaacaccgaggtgagggtgctgctgaagcacgacgacaacccatacgtgatcggcatcgataggggcgagcgcaacctgctctacatcgtggtggttgacggcaagggcaatatcgtcgagcagtacagccttaacgagatcattaacaacttcaatggcatcaggatcaagaccgactaccacagcctgctcgacaagaaagaaaaagagcgcttcgaggccaggcagaactggaccagcatcgagaatatcaaagagctgaaggccggctacattagccaggtggtgcacaagatctgcgagctggtggaaaagtacgacgcggtgatcgctctcgaggacctgaactccgggttcaagaactcccgcgtgaaggttgagaagcaggtctaccaaaagttcgagaagatgctgatcgacaagctcaactacatggtggacaaaaagagcaacccctgcgccacaggcggcgctcttaagggctaccagatcacgaacaagttcgagtccttcaagagcatgagcacccagaatggcttcatcttctacatcccggcctggctgaccagcaagatcgatccatctaccggcttcgtcaacctcctcaagaccaagtacaccagcattgccgacagcaagaagttcatctccagcttcgacaggatcatgtacgtgccggaagaggacctgttcgagttcgcgctcgattacaagaacttcagcaggaccgacgcggactatattaagaagtggaagctctacagctacggcaacaggatccgcatcttcagaaacccgaagaaaaacaacgtgttcgactgggaagaagtgtgcctgaccagcgcctacaaagaactgttcaacaagtacggcatcaactaccagcagggcgacatcagggctctgctgtgcgagcagtctgacaaggcgttctacagctccttcatggccctgatgagcctgatgctgcagatgaggaacagcatcaccggcaggacggacgtcgacttcctgatcagcccagtgaagaattccgacggcattttctacgactctaggaactacgaggctcaagagaacgccatcctgccgaagaacgccgatgctaacggcgcgtacaacattgcccgcaaggtgctgtgggctatcggccagtttaagaaggccgaggacgaaaaactggacaaggtgaagatcgccattagcaacaaagagtggctcgagtacgcccagaccagcgtgaagcacaaaaggccagccgccactaagaaggctggccaggccaaaaagaagaagtga

SEQ ID NO: 159 - skipped

SEQ ID NO: 160 - DNA, 37 bp, synthetic construct
  misc_feature 1..37: note = Description of Artificial Sequence: Synthetic oligonucleotide
  source 1..37: mol_type = other DNA
tttgatttactcgtcacgattccctggtcgaactttt

SEQ ID NO: 161 - DNA, 37 bp, synthetic construct
  misc_feature 1..37: note = Description of Artificial Sequence: Synthetic oligonucleotide
  source 1..37: mol_type = other DNA
tttaatttactcgtcacgattccctggtcgaactttt

SEQ ID NO: 162 - DNA, 43 bp, Zea mays
  source 1..43: mol_type = unassigned DNA
tttgatttactcgtcacgattcccctctcctggtcgaactttt

SEQ ID NO: 163 - DNA, 78 bp, synthetic construct
  misc_feature 1..78: note = Description of Artificial Sequence: Synthetic oligonucleotide
  source 1..78: mol_type = other DNA
aattcgatctacacttagtagaaattaatctcccagttaagataatgctgcatctacacttagtagaaattatcaggt

SEQ ID NO: 164 - protein, 4 aa, unidentified
DEAD

(19) United States
(12) Patent Application Publication
(10) Pub. No.: US 2024/0409949 A1
(43) Pub. Date: Dec. 12, 2024

(54) OPTIMIZED PLANT CRISPR/CPF1 SYSTEMS

(71) Applicant: KWS SAAT SE & Co. KGaA, Einbeck (DE)

(72) Inventors: Aaron HUMMEL, Hillsborough, NC (US); Zarir VAGHCHHIPAWALA, Ballwin, MO (US)

(73) Assignee: KWS SAAT SE & Co. KGaA, Einbeck (DE)

(21) Appl. No.: 18/813,194

(22) Filed: Aug. 23, 2024

Related U.S. Application Data
(63) Continuation of application No. 16/961,303, filed on Jul. 10, 2020, now Pat. No. 12,098,374.
(60) Provisional application No. 62/616,136, filed on Jan. 11, 2018.

(51) Int. Cl. C12N 15/82; C12N 9/22

(52) U.S. Cl. CPC C12N 15/82; C12N 9/22; C12N 2310/20

(57) ABSTRACT

The present invention relates to optimized systems for the genome editing of eukaryotic cells, preferably plant cells. Also provided is a plant delivery system comprising at least one Cpf1 enzyme, or a plant optimized construct encoding the same, to be synergistically combined with a Cpf1 guide RNA system. Further provided is a Cpf1 guide RNA system being flanked by a Hammerhead ribozyme sequence at the 5′ end and by a plant-derived Hepatitis Delta Virus (HDV)-like ribozyme sequence at the 3′ end, or being embedded within a coding or non-coding region, of a sequence encoding a frame sequence. Novel plant-derived HDV-like ribozyme sequences are also provided. Further provided are methods for improved genome editing, and the use of the various systems provided herein to obtain transformed plants, plant cells, tissues, organs, or a progeny thereof, or a plant material, modified in a targeted way even at difficult to access target sites.

### CROSS-REFERENCE TO RELATED APPLICATIONS

This application is a continuation of U.S. application Ser. No. 16/961,303, filed Jul. 10, 2020, which is a U.S. National Phase of International Patent Application No. PCT/EP2019/050655, filed on Jan. 11, 2019, which claims priority to U.S. Provisional Application No. 62/616,136, filed Jan. 11, 2018. The entire contents of these applications are incorporated herein by reference in their entirety.

### SEQUENCE LISTING

The instant application contains a Sequence Listing which has been submitted electronically in XML format and is hereby incorporated by reference in its entirety. Said XML copy, created on Feb. 29, 2024, is named 245761_000235_SL.xml and is 392,192 bytes in size.

### TECHNICAL FIELD

The present invention relates to optimized systems for the genome editing of eukaryotic cells, preferably plant cells. Also provided is a plant delivery system comprising at least one Cpf1 enzyme, or a plant optimized construct encoding the same, to be synergistically combined with a Cpf1 guide RNA system. Further provided is a Cpf1 guide RNA system being flanked by a Hammerhead ribozyme sequence at the 5′ end and by a plant-derived Hepatitis Delta Virus (HDV)-like ribozyme sequence at the 3′ end, or being embedded within a coding or non-coding region, of a sequence encoding a frame sequence. Novel plant-derived HDV-like ribozyme sequences are also provided. Further provided are methods for improved genome editing, and the use of the various systems provided herein to obtain transformed plants, plant cells, tissues, organs, or a progeny thereof, or a plant material, modified in a targeted way even at difficult to access target sites.

### BACKGROUND OF THE INVENTION

Site-directed modification of a given genome of interest has been the leading edge in plant biological researches for the recent years. Since 1996, researchers have reported the directed DNA cleaving activities of zinc finger nucleases (ZFNs) (Kim et al., 1996), transcription activator-like effector nucleases (TALENs) (Christian et al., 2010), or the clustered regularly interspaced short palindromic repeat (CRISPR), mainly the CRISPR/Cas9 technology (Jinek et al., 2012), which have been widely applied in animal and plant genome site-directed modifications, and significantly promoted the basic and applied researches on the genetic engineering of eukaryotes. The CRISPR/Cas9 system discovered recently is a genomic immune system in the (ancient) bacteria against the invasion of exogenous nucleic acid molecules which is different from the “restriction-modification” system: it is clear through researches that, the homologous sequences in the DNA double helix can be recognized, after pairing the homologous regions of a sequence specific crRNA (CRISPR RNA) and another tracrRNA molecule (trans-activating crRNA), so as to guide Cas9 protein to recognize and cleave the target DNA double helix, and create site-directed DSB (Jinek et al., 2012). In artificial CRISPR systems, a synthetic non-coding RNA and a CRISPR nuclease and/or optionally a modified CRISPR nuclease, modified to act as nickase or lacking any nuclease function, can be used in combination with at least one synthetic or artificial guide RNA or(s) gRNA combining the function of a crRNA and/or a tracrRNA (Makarova et al., Nature Rev. Microbiol., 2015).

Since the cell endogenous DNA site-directed modification activity of the CRISPR/Cas9 system has been proven in 2013, researchers have adopted CRISPR/Cas9 system to realize the RNA-guided site-directed modification of the genomes in animal and plant systems, such as the macaque Macaca fascicularis, zebrafish, mice and human cell line, Arabidopsis thaliana and rice. Although CRISPR is much simpler than the previous gene editing methods, and also considered as a revolutionary technology in the genetic research field, researchers still urgently seek for improvements and developments of the CRISPR technology, in hope to realize an even simpler and more precise genome site-directed modification, in particular in complex eukaryotic genomes, like plant genome, where poor accessibility of certain target sequences and the problem of off-target effects and transformation/transfection problems are still a dominating issue hampering the broad applicability of site-directed gene or genome editing (GE) in eukaryotic cells, including plant and animal cells.

In 2015 (Zetsche et al. (2015) Cell, 163 (3):759-771.), the researchers discovered a novel CRISPR system (CRISPR/Cpf1), which can cleave DNA substrates in human cells. When comparing with CRISPR/Cas9 system, CRISPR/Cpf1 has the following four advantages: 1) Cpf1 protein is smaller than the standard Cas9 protein, and only one crRNA molecule is required for CRISPR/Cpf1 to cleave DNA, while CRISPR/Cas9 requires two molecules tracrRNA and crRNA with a longer sequence to jointly recognize and cleave the DNA substrate; 2) CRISPR/Cpf1 is a sticky-end cleaving, while CRISPR/Cas9 is blunt-end cleaving, whereas it has been shown that DNA insertion is more controllable with sticky-end cleaving which is also more beneficial for the editing and repair after the DNA cleaving; 3) CRISPR/Cpf1 and CRISPR/Cas9 recognize different PAM (protospacer adjacent motif) sites on the DNA substrate (CRISPR/Cpf1 recognizes inter alia the PAM site of 5′-TTTN-3, while CRISPR/Cas9 recognizes inter alia the PAM site of 5′-NGG-3′), which has broadened the options for the design of CRISPR editing loci; 4) the CRISPR/Cpf1 cleaving locus is located at the 3′-end of the PAM site, while the CRISPR/Cas9 cleaving locus is located at the 5′-end of the PAM site, which can introduce more flexibility to design GE experiments.

Existing evidence shows that CRISPR/Cpf1 can be applied in human cells and mice to carry out genetic editing, which indicates it has an application prospect. Recent research also showed that CRISPR/Cpf1 can be applied in plants to efficiently carry out genome editing. Two research groups have published initial reports on CRISPR/Cpf1 (Endo A et al. (2006) Scientific Reports, 6:38169 and Xu et al. (2016) Plant Biotechnol. Journal, doi:10.1111/pbi.12669.), however, the results indicated that CRISPR/Cpf1 has a low cleaving and editing efficiency and poor genetic stability in plant cells and might thus be a far less promising tool for plant biotechnological applications. This has cast the doubt as to whether CRISPR/Cpf1 can be similarly used as CRISPR/Cas9 as an effective site-directed GE tool in plants, which presently strongly limits the research and application of CRISPR/Cpf1 in the site-directed genome modification of plants.

In view of the outstanding challenges associated with the implementation of an effective CRISPR/Cpf1 platform in plants, there remains an ongoing need to provide improved CRISPR/Cpf1 systems that allow for efficient GE in plant cells, in particular for relevant crop plants like wheat and corn (Zea mays).

Another problem frequently associated with any CRISPR-based GE approach is the fact that the CRISPR systems are RNA-guided systems. Therefore, availability of the gRNA and its stability play a crucial role for any CRISPR-based GE assay. Production of gRNAs directly in a cell, in particular in plant cells, is still strongly limited due to the lack of suitable promoters and expression constructs. For example, commonly used promoters, like the small nuclear RNA U6 and U3 promoter, do not provide sufficient specificity and in vitro transcription rates to guarantee sufficient gRNA availability. For example, the RNA polymerase III-dependent U6 promoter or the T7 promoter require a G or GG, respectively, at the 5′ end of the RNA to be transcribed. As a result, standard full-length or truncated gRNAs expressed from these promoters are limited to targeting sites that match the forms GN₁₆₋₁₉NGG or GGN₁₅₋₁₈NGG, sites that each occur every 1 in 32 bps or 1 in 128 bps, respectively, in random DNA strongly limiting the targeting range when applied in a CRISPR-based GE approach (Sander and Young, 2014, Nat. Biotechnol., 32 (4):347-355).

The design of suitable gRNAs depends on a variety of factors, i.e., inter alia the kind of edit planned, the target genome and its complexity, including potential off-target sites, the availability of PAM sequences, the optimum interaction of a design gRNA and the cognate CRISPR effector (the complex formation between gRNA/CRISPR effector) and the stability of the gRNA provided pre-synthesized, or on a suitable expression construct. PAM specificity and in turn target range restrictions are a common problem in any CRISPR-based GE approach, as the CRISPR effector and the cognate gRNA must be able to interact with each other for proper PAM recognition. Therefore, many adjusting screws have to be individually adapted to provide a CRISPR-based system including all components needed for functionality in a target cell of interest—to ultimately edit a genomic target site of interest successfully. Despite recent progress in this area, the predictability of a successful GE experiment planned in silico is thus still rather low, in particular for plant genomes, as many of the CRISPR work is performed in animal cells, whereas the complexity and specific features of plant genomes and plant metabolism still require fundamental research effort to establish suitable CRISPR systems effective in a variety of different plants, including economically relevant crop plants.

The objective underlying the present invention therefore was to provide improved CRISPR systems, mainly CRISPR/Cpf1 systems recently described to be less efficient in plant cells, which systems can be used for efficient plant GE approaches. It was another aim of the present invention to overcome poor guide RNA availability by identifying new plant-compatible systems for providing gRNAs in a stable manner and thus to dramatically increase successful GE rates suitable for in vitro and in vivo applications.

Furthermore, it was an aim to identify ribozyme-based gRNA delivery systems, in particular systems not relying on common systems like the hepatitis-delta virus (HDV) ribozyme derived from a human pathogenic virus, to provide safe and plant-optimized gRNA delivery tools having superior performance, in particular in the plant system, and not necessitating complex deregulation processes in product development.

To this end, it was an aim to provide GE tools relying on optimized CRISPR effector nucleases as well as optimized guide RNA delivery techniques, including embedding and ribozyme activation of guide RNAs, to provide active GE tools having superior qualities in a synergistic manner, in particular for targeted GE in plants and plant cells.

It was a final aim of the present invention to combine the optimized tools to achieve reliable and predictable GE in a variety of different plant cells, wherein the methods and uses should be practicable for multiplexing to generate targeted GE events for different loci of interest with significantly reduced costs and time expenses.

### SUMMARY OF THE INVENTION

The present invention thus relates to several aspects to establish and improve the efficiency of CRISPR/Cpf1 systems in plants cells; in particular in corn (e.g. Zea mays), by providing individual components fine-tuned to each other to obtain optimum genome editing efficiencies.

The above objectives have been achieved by providing, in a first aspect, a plant delivery system, the delivery system comprising (a) at least one Cpf1 enzyme or an active fragment thereof, or a nucleic acid sequence encoding the same; and (b) at least one Cpf1 guide RNA system, or a nucleic acid sequence encoding the same, the at least one Cpf1 guide RNA system comprising at least one Cpf1 guide RNA specific for a genomic target sequence of interest in a plant or part of a plant; wherein the at least one Cpf1 guide RNA, or the nucleic acid sequence encoding the same, is (i) flanked by a Hammerhead ribozyme sequence at the 5′-end and by a plant-derived Hepatitis Delta Virus (HDV)-like ribozyme sequence at the 3′-end; and/or is (ii) embedded within a non-coding region, preferably a 3′ untranslated region (UTR), of a sequence encoding a frame sequence.

In one embodiment there is provided a plant delivery system, wherein the plant delivery system comprises a first nucleotide molecule comprising a nucleic acid sequence encoding the at least one Cpf1 enzyme or an active fragment thereof, and a second nucleotide molecule comprising a nucleic acid sequence encoding the at least one Cpf1 guide RNA system, preferably wherein the first and the second molecule are provided on separate constructs, or wherein the first and the second molecule are provided on a single transcript construct.

In another embodiment there is provided a plant delivery system, wherein the first construct comprising the first nucleotide molecule and the second construct comprising the second nucleotide molecule each comprise at least one promoter functional in a plant or part of a plant, preferably wherein the at least one promoter driving expression of the first and the second nucleotide molecule is different in the first and the second construct, respectively.

In yet another embodiment there is provided a plant delivery system, wherein the at least one promoter is independently selected from a (p)BdUbi10 promoter (SEQ ID NO: 1, a (p)ZmUbi1 promoter (SEQ ID NO: 2), a (p)OsActin promoter (SEQ ID NO: 3), and a single or double 35S promoter (SEQ ID NO: 4), optionally including an ZmUbi1 intron, an BdUbi10 intron and/or an Adh1 intron, (SEQ ID NOs: 5 to 10 or 67), or any combination thereof, or a sequence having at least 90%, 91%, 92%, 93%, 94%, 95%, 96%, 97%, 98%, or 99% identity when compared over the whole length of the respective sequence of any one of SEQ ID NOs: 1 to 10, or 67. Bd means originating from Brachypodium distachyon, Zm means originating from Zea mays, Adh1 means originating from alcohol dehydrogenase-1, and Os means originating from Oryza sativa.

In yet a further embodiment there is provided a plant delivery system, wherein the first construct comprising the first nucleotide molecule and the second construct comprising the second nucleotide molecule comprise at least one terminator functional in a plant or part of a plant.

In one embodiment there is provided a plant delivery system, wherein the at least one terminator is a NosT terminator (SEQ ID NO: 11) or a 35S terminator (SEQ ID NO: 12), or any combination thereof, or a sequence having at least 90%, 91%, 92%, 93%, 94%, 95%, 96%, 97%, 98%, or 99% identity when compared over the whole length of the respective sequence of any one of SEQ ID NOs: 11 or 12.

In a further embodiment there is provided a plant delivery system, wherein the nucleic acid sequence encoding the Cpf1 enzyme or an active fragment thereof is codon-optimized for expression in a plant or part of a plant. In a further embodiment there is provided a plant delivery system, wherein the nucleic acid sequence encoding the Cpf1 enzyme or an active fragment thereof contains at least one intron. In another further embodiment there is provided a plant delivery system, wherein the nucleic acid sequence encoding the Cpf1 enzyme or an active fragment thereof is codon-optimized for expression in a plant or part of a plant and contains at least one intron.

In yet a further embodiment there is provided a plant delivery system, wherein the nucleic acid sequence encoding the Cpf1 enzyme or an active fragment thereof is selected from SEQ ID NOs: 13, 14, 38, 39, 72, 73, 74, 75, 76, 157, or 158, or a sequence having at least 85%, 86%, 87%, 88%, 89%, 90%, 91%, 92%, 93%, 94%, 95%, 96%, 97%, 98%, or 99% identity over the whole length of the respective sequence of SEQ ID NOs: 13, 14, 38, 39, 72, 73, 74, 75, 76, 157, or 158. Preferably the nucleic acid sequence encoding the Cpf1 enzyme or an active fragment thereof is SEQ ID NO: 72 or 75, or a sequence having at least 85%, 86%, 87%, 88%, 89%, 90%, 91%, 92%, 93%, 94%, 95%, 96%, 97%, 98%, or 99% identity over the whole length of the respective sequence of SEQ ID NO: 72 or 75.

In one embodiment there is provided a plant delivery system, wherein the Hammerhead ribozyme sequence, or a sequence encoding the same, is selected from SEQ ID NO: 17 or 18 (HH Ribozyme sequence), and/or wherein the plant-derived Hepatitis Delta Virus (HDV)-like ribozyme sequence, or a sequence encoding the same, is selected from any one of SEQ ID NOs: 19 to 26, or a sequence having at least 95%, 96%, 97%, 98%, or 99% identity over the whole length of any one of SEQ ID NOs: 17 to 26.

In another embodiment there is provided a plant delivery system, wherein the at least one Cpf1 guide RNA, or the nucleotide sequence encoding the same, which is (i) flanked by the Hammerhead ribozyme sequence at the 5′ and the plant-derived Hepatitis Delta Virus (HDV)-like ribozyme sequence at the 3′-end, further comprises a scaffold RNA sequence, or a sequence encoding the same, at the 5′-end; and/or which is (ii) embedded within the non-coding region, preferably the 3′ untranslated region (UTR), of the sequence encoding a frame sequence, further comprises a scaffold RNA sequence, or a sequence encoding the same, at the 5′ and 3′-end.

In yet another embodiment there is provided a plant delivery system, wherein the scaffold RNA sequence, or a sequence encoding the same, is selected from SEQ ID NO: 29 or 30, or a sequence having at least 95%, 96%, 97%, 98%, or 99% identity over the whole length of SEQ ID NO: 29 or 30.

In one embodiment there is provided a plant delivery system, wherein the at least one Cpf1 enzyme or an active fragment thereof, or a nucleic acid sequence encoding the same, is selected from any one of SEQ ID NOs: 13 to 16 or 38 to 41 or 72 to 76 or 152 to 156 or 157 to 158, or a sequence having at least 60%, 65%, 70%, 75%, 80%, 85%, 90%, 91%, 92%, 93%, 94%, 95%, 96%, 97%, 98%, 99% or more sequence identity over the whole length of the respective nucleic acid sequence of SEQ ID NOs: 13, 14, 38, 39, 72, 73, 74, 75, 76, 157, or 158 or a sequence having at least 90%, 91%, 92%, 93%, 94%, 95%, 96%, 97%, 98%, or 99% identity over the whole length of the respective amino acid sequence of SEQ ID NOs: 15, 16, 40, 41, 152, 153, 154, 155, or 156, respectively.

In another embodiment there is provided a plant delivery system, wherein the at least one Cpf1 enzyme, or an active fragment thereof, or a nucleic acid sequence encoding the same, comprises at least one mutation in comparison to a wild-type sequence resulting in an altered PAM recognition, preferably wherein the at least mutation is selected from G532R/K595R, or G532R/K538V/Y542R in comparison to the sequence of SEQ ID NO: 16.

In still another embodiment there is provided a plant delivery system, wherein the at least one Cpf1 enzyme, or an active fragment thereof, or a nucleic acid sequence encoding the same, comprises at least one mutation in comparison to a wild-type sequence (SEQ ID NO: 16) resulting in an altered PAM recognition, wherein the altered PAM recognition is a recognition of a TYCV, or of a TATV PAM sequence.

In one embodiment there is provided a plant delivery system, wherein the at least one Cpf1 enzyme or an active fragment thereof, or a nucleic acid sequence encoding the same, and/or the at least one Cpf1 guide RNA system, or a nucleic acid sequence encoding the same, are provided as at least one vector construct, or are provided as at least one linear construct.

In a further embodiment there is provided a plant delivery system, wherein the at least one Cpf1 guide RNA system comprises at least two guide RNAs, wherein the at least two guide RNAs are separated by a nucleotide sequence comprising direct repeats.

In yet a further embodiment there is provided a plant delivery system, wherein the sequence encoding a frame sequence is selected from a translatable or non-translatable sequence being selected from a marker gene, including an antibiotic marker or a fluorescent marker, a gene encoding a structural protein, a gene encoding an RNA species, an internal ribosomal entry site (IRES) encoding sequence.

In another embodiment there is provided a plant delivery system, wherein the sequence encoding a frame sequence is selected from any one of SEQ ID NOs: 31, 32, 13, 14, 38, 39, 72, 73, 74, 75, 76, 157, or 158, or a sequence having at least 85%, 86%, 87%, 88%, 89%, 90%, 91%, 92%, 93%, 94%, 95%, 96%, 97%, 98%, or 99% identity over the whole length of the respective sequence of SEQ ID NOs: 31, 32, 13, 14, 38, 39, 72, 73, 74, 75, 76, 157, or 158.

In still another embodiment there is provided a plant delivery system, wherein the part of a plant is selected from the group consisting of a plant cell, a plant tissue and a plant organ. leaves, stems, roots, emerged radicles, flowers, flower parts, petals, fruits, pollen, pollen tubes, anther filaments, ovules, embryo sacs, egg cells, ovaries, zygotes, embryos, zygotic embryos, somatic embryos, apical meristems, vascular bundles, pericycles, seeds, roots, gametophytes, spores and cuttings.

In one embodiment there is provided a plant delivery system, wherein the plant or part of a plant originates from a genus selected from the group consisting of Hordeum, Sorghum, Saccharum, Zea, Setaria, Oryza, Triticum, Secale, Triticale, Malus, Brachypodium, Aegilops, Daucus, Beta, Eucalyptus, Nicotiana, Solanum, Coffea, Vitis, Erythrante, Genlisea, Cucumis, Marus, Arabidopsis, Crucihimalaya, Cardamine, Lepidium, Capsella, Olmarabidopsis, Arabis, Brassica, Eruca, Raphanus, Citrus, Jatropha, Populus, Medicago, Cicer, Cajanus, Phaseolus, Glycine, Gossypium, Astragalus, Lotus, Torenia, Allium, or Helianthus. More preferably, the plant or part of a plant originates from a species selected from the group consisting of Hordeum vulgare, Hordeum bulbusom, Sorghum bicolor, Saccharum officinarium, Zea spp., including Zea mays, Setaria italica, Oryza minuta, Oryza sativa, Oryza australiensis, Oryza alta, Triticum aestivum, Triticum durum, Secale cereale, Triticale, Malus domestica, Brachypodium distachyon, Hordeum marinum, Aegilops tauschii, Daucus glochidiatus, Beta spp., including Beta vulgaris, Daucus pusillus, Daucus muricatus, Daucus carota, Eucalyptus grandis, Nicotiana sylvestris, Nicotiana tomentosiformis, Nicotiana tabacum, Nicotiana benthamiana, Solanum lycopersicum, Solanum tuberosum, Coffea canephora, Vitis vinifera, Erythrante guttata, Genlisea aurea, Cucumis sativus, Marus notabilis, Arabidopsis arenosa, Arabidopsis lyrata, Arabidopsis thaliana, Crucihimalaya himalaica, Crucihimalaya wallichii, Cardamine nexuosa, Lepidium virginicum, Capsella bursa pastoris, Olmarabidopsis pumila, Arabis hirsute, Brassica napus, Brassica oleracea, Brassica rapa, Raphanus sativus, Brassica juncacea, Brassica nigra, Eruca vesicaria subsp. sativa, Citrus sinensis, Jatropha curcas, Populus trichocarpa, Medicago truncatula, Cicer yamashitae, Cicer bijugum, Cicer arietinum, Cicer reticulatum, Cicer judaicum, Cajanus cajanifolius, Cajanus scarabaeoides, Phaseolus vulgaris, Glycine max, Gossypium sp., Astragalus sinicus, Lotus japonicas, Torenia fournieri, Allium cepa, Allium fistulosum, Allium sativum, Helianthus annuus, Helianthus tuberosus and/or Allium tuberosum. Particularly preferred are Beta vulgaris, Zea mays, Triticum aestivum, Hordeum vulgare, Secale cereale, Helianthus annuus, Solanum tuberosum, Sorghum bicolor, Brassica rapa, Brassica napus, Brassica juncacea, Brassica oleracea, Raphanus sativus, Oryza sativa, Glycine max, and/or Gossypium sp.

In another aspect there is provided a host cell comprising a plant delivery system of the various aspects and embodiments disclosed herein.

In a further aspect there is provided a plant, or a plant cell, tissue, organ or material, or a derivative or progeny thereof, comprising a plant delivery system of the various aspects and embodiments disclosed herein.

In one embodiment, there is provided a plant delivery system, wherein the genomic target sequence of interest is a difficult to access target site.

In yet a further aspect there is provided a method for modifying a genomic target sequence of interest in a plant or part of a plant, wherein the method comprises the steps of: (a) providing at least one Cpf1 enzyme or an active fragment thereof, or a nucleic acid sequence encoding the same; preferably, wherein the at least one nucleic acid sequence encoding the Cpf1 enzyme or an active fragment thereof is codon-optimized for the expression in the plant or part of the plant; and (b) providing at least one Cpf1 guide RNA system, or a nucleic acid sequence encoding the same, the at least one Cpf1 guide RNA system comprising at least one Cpf1 guide RNA specific for a genomic target sequence of interest in the plant or part of the plant; wherein the at least one Cpf1 guide RNA, or the nucleic acid sequence encoding the same, is (i) flanked by a Hammerhead ribozyme sequence at the 5′ and a plant-derived Hepatitis Delta Virus (HDV)-like ribozyme sequence at the 3′-end; and/or is (ii) embedded within a non-coding region, preferably a 3′ untranslated region (UTR), of a sequence encoding a frame sequence (c) optionally: providing at least one repair template nucleic acid sequence, wherein the at least one repair template nucleic acid sequence is preferably flanked by one or more homology sequence(s) complementary to one or both adjacent region(s) of the genomic sequence of interest in the plant or part of the plant; (d) introducing the at least one Cpf1 enzyme or an active fragment thereof, or a nucleic acid sequence encoding the same from step (a); and introducing the at least one Cpf1 guide RNA system, or a nucleic acid sequence encoding the same from step (b) and optionally: introducing the at least one repair template nucleic acid sequence from step (c) into the plant or part of the plant; and (e) obtaining a plant or part of a plant, or a progeny thereof, comprising a modification in the genomic target sequence of interest.

In one embodiment there is provided a method, wherein the at least one Cpf1 enzyme or an active fragment thereof, or a nucleic acid sequence encoding the same, and the at least one Cpf1 guide RNA system, or a nucleic acid sequence encoding the same, and optionally wherein the at least one repair template nucleic acid sequence, are provided on separate constructs, wherein the at least two separate constructs are introduced simultaneously, or subsequently.

In another embodiment there is provided a method, wherein the at least one Cpf1 enzyme or an active fragment thereof, or a nucleic acid sequence encoding the same, and the at least one Cpf1 guide RNA system, or a nucleic acid sequence encoding the same, are provided on a single transcript construct.

In still another embodiment there is provided a method, wherein the at least one Cpf1 enzyme or an active fragment thereof, or a nucleic acid sequence encoding the same, and the at least one Cpf1 guide RNA system, or a nucleic acid sequence encoding the same, are provided on a multiplexing construct.

In a further embodiment there is provided a method, wherein the molecules of step (a), (b) and optionally of step (c) are provided as plant delivery system as defined according to the first aspect of the present invention, wherein the plant delivery system comprises a first nucleotide molecule comprising a nucleic acid sequence encoding the at least one Cpf1 enzyme or an active fragment thereof, and a second nucleotide molecule comprising a nucleic acid sequence encoding the at least one Cpf1 guide RNA system, wherein the first and the second molecule are provided on separate constructs, or wherein the first and the second molecule are provided on a single transcript construct.

In one embodiment there is provided a method, wherein (i) the at least one nucleic acid sequence encoding the Cpf1 enzyme or an active fragment thereof, and the nucleic acid sequence encoding at least one Cpf1 guide RNA system; or (ii) wherein the first construct comprising the first nucleotide molecule and the second construct comprising the second nucleotide molecule, each comprise at least one promoter functional in a plant or part of a plant.

In another embodiment there is provided a method, wherein the at least one promoter is independently selected from a (p)BdUbi10 promoter (SEQ ID NO: 1), a (p)ZmUbi1 promoter (SEQ ID NO: 2), a (p)OsActin promoter (SEQ ID NO: 3), and a single or double 35S promoter (SEQ ID NO: 4), optionally including an ZmUbi1 intron, an BdUbi10 intron and/or an Adh1 intron, (SEQ ID NOs: 5 to 10, or 67), or any combination thereof, or a sequence having at least 90%, 91%, 92%, 93%, 94%, 95%, 96%, 97%, 98%, or 99% identity when compared over the whole length of the respective sequence of any one of SEQ ID NOs: 1 to 10, or 67.

In yet another embodiment there is provided a method, wherein (i) the at least one nucleic acid sequence encoding the Cpf1 enzyme or an active fragment thereof, and the nucleic acid sequence encoding at least one Cpf1 guide RNA system; or (ii) wherein the first construct comprising the first nucleotide molecule and the second construct comprising the second nucleotide molecule; comprise at least one terminator functional in a plant or part of a plant.

In still another embodiment there is provided a method, wherein the at least one terminator is independently selected from a NosT terminator (SEQ ID NO: 11), or a 35S terminator (SEQ ID NO: 12), or any combination thereof or a sequence having at least 90%, 91%, 92%, 93%, 94%, 95%, 96%, 97%, 98%, or 99% identity when compared over the whole length of the respective sequence of any one of SEQ ID NOs: 11 or 12.

In one embodiment there is provided a method, wherein the nucleic acid sequence encoding the Cpf1 enzyme or an active fragment thereof is codon-optimized for expression in a plant or part of a plant.

In another embodiment there is provided a method, wherein the nucleic acid sequence encoding the Cpf1 enzyme or an active fragment thereof is selected from SEQ ID NOs: 13, 14, 38, 39, 72, 73, 74, 75, 76, 157, or 158, or a sequence having at least 85%, 86%, 87%, 88%, 89%, 90%, 91%, 92%, 93%, 94%, 95%, 96%, 97%, 98%, or 99% identity over the whole length of the respective sequence of SEQ ID NOs: 13, 14, 38, 39, 72, 73, 74, 75, 76, 157, or 158.

In still another embodiment there is provided a method, wherein the Hammerhead ribozyme sequence, or a sequence encoding the same, is selected from SEQ ID NO: 17 or 18, and/or wherein the plant-derived Hepatitis Delta Virus (HDV)-like ribozyme sequence, or a sequence encoding the same, is selected from any one of SEQ ID NOs: 19 to 26, or a sequence having at least 95%, 96%, 97%, 98%, or 99% identity over the whole length of any one of SEQ ID NOs: 17 to 26.

In one embodiment there is provided a method, wherein the at least one Cpf1 guide RNA, or the nucleotide sequence encoding the same, which is (i) flanked by the Hammerhead ribozyme sequence at the 5′ and the plant-derived Hepatitis Delta Virus (HDV)-like ribozyme sequence at the 3′ end, further comprises a scaffold RNA sequence at the 5′-end; and/or which is (ii) embedded within the non-coding region, preferably the 3′ untranslated region (UTR), of the sequence encoding a frame sequence, further comprises a scaffold RNA sequence at the 5′ and 3′-end.

In another embodiment there is provided a method, wherein the scaffold RNA sequence, or a sequence encoding the same, is selected from SEQ ID NO: 29 or 30, or a sequence having at least 95%, 96%, 97%, 98%, or 99% identity over the whole length of SEQ ID NO: 29 or 30.

In yet another embodiment there is provided a method, wherein the at least one Cpf1 enzyme or an active fragment thereof, or a nucleic acid sequence encoding the same, is selected from any one of SEQ ID NOs: 13 to 16 or 38 to 41 or 72 to 76 or 152 to 156 or 157 to 158, or a sequence having at least 60%, 65%, 70%, 75%, 80%, 85%, 90%, 91%, 92%, 93%, 94%, 95%, 96%, 97%, 98%, 99% or more sequence identity over the whole length of the respective nucleic acid sequence of SEQ ID NOs: 13, 14, 38, 39, 72, 73, 74, 75, 76, 157, or 158, or a sequence having at least 90%, 91%, 92%, 93%, 94%, 95%, 96%, 97%, 98%, or 99% identity over the whole length of the respective amino acid sequence of SEQ ID NOs: 15, 16, 40, 41, 152, 153, 154, 155, or 156, respectively.

In another embodiment there is provided a method, wherein the at least one Cpf1 enzyme, or an active fragment thereof, or a nucleic acid sequence encoding the same, comprises at least one mutation in comparison to a wild-type sequence (SEQ ID NO: 16) resulting in an altered PAM recognition, preferably wherein the at least one mutation is selected from G532R/K595R, or G532R/K538V/Y542R in comparison to the sequence of SEQ ID NO: 16.

In one embodiment there is provided a method, wherein the at least one Cpf1 enzyme, or an active fragment thereof, or a nucleic acid sequence encoding the same, comprises at least one mutation in comparison to a wild-type sequence (SEQ ID NO: 16) resulting in an altered PAM recognition, wherein the altered PAM recognition is a recognition of a TYCV, or of a TATV PAM sequence.

In another embodiment there is provided a method, wherein the at least one Cpf1 enzyme or an active fragment thereof, or a nucleic acid sequence encoding the same, and/or the at least one Cpf1 guide RNA system, or a nucleic acid sequence encoding the same, are provided as at least one vector construct, or are provided as at least one linear construct.

In yet another embodiment there is provided a method, wherein the at least one Cpf1 guide RNA system comprises at least two guide RNAs, wherein the at least two guide RNAs are separated by a nucleotide sequence comprising direct repeats and wherein the at least two guide RNAs, or nucleotide sequences encoding the same, may each further comprise a scaffold RNA sequence at their 5′-ends.

In one embodiment there is provided a method, wherein the sequence encoding a frame sequence is selected from a translatable or non-translatable sequence being selected from a marker gene, including an antibiotic marker or a fluorescent marker, a gene encoding a structural protein, a gene encoding an RNA species, an IRES encoding sequence.

In a further embodiment there is provided a method, wherein the sequence encoding a frame sequence is selected from any one of SEQ ID NOs: 31, 32, 13, 14, 38, 39, 72, 73, 74, 75, 76, 157, or 158 or a sequence having at least 85%, 86%, 87%, 88%, 89%, 90%, 91%, 92%, 93%, 94%, 95%, 96%, 97%, 98%, or 99% identity over the whole length of the respective sequence of SEQ ID NOs: 31, 32, 13, 14, 38, 39, 152, 153, 154, 155, or 156.

In yet a further embodiment there is provided a method, wherein the part of a plant is selected from the group consisting of a plant cell, a plant tissue and a plant organ.

In one embodiment there is provided a method, wherein the plant or a part of a plant originates from a genus selected from the group consisting of Hordeum, Sorghum, Saccharum, Zea, Setaria, Oryza, Triticum, Secale, Triticale, Malus, Brachypodium, Aegilops, Daucus, Beta, Eucalyptus, Nicotiana, Solanum, Coffea, Vitis, Erythrante, Genlisea, Cucumis, Marus, Arabidopsis, Crucihimalaya, Cardamine, Lepidium, Capsella, Olmarabidopsis, Arabis, Brassica, Eruca, Raphanus, Citrus, Jatropha, Populus, Medicago, Cicer, Cajanus, Phaseolus, Glycine, Gossypium, Astragalus, Lotus, Torenia, Allium, or Helianthus. More preferably, the plant or part of a plant originates from a species selected from the group consisting of Hordeum vulgare, Hordeum bulbusom, Sorghum bicolor, Saccharum officinarium, Zea spp., including Zea mays, Setaria italica, Oryza minuta, Oryza sativa, Oryza australiensis, Oryza alta, Triticum aestivum, Triticum durum, Secale cereale, Triticale, Malus domestica, Brachypodium distachyon, Hordeum marinum, Aegilops tauschii, Daucus glochidiatus, Beta spp., including Beta vulgaris, Daucus pusillus, Daucus muricatus, Daucus carota, Eucalyptus grandis, Nicotiana sylvestris, Nicotiana tomentosiformis, Nicotiana tabacum, Nicotiana benthamiana, Solanum lycopersicum, Solanum tuberosum, Coffea canephora, Vitis vinifera, Erythrante guttata, Genlisea aurea, Cucumis sativus, Marus notabilis, Arabidopsis arenosa, Arabidopsis lyrata, Arabidopsis thaliana, Crucihimalaya himalaica, Crucihimalaya wallichii, Cardamine nexuosa, Lepidium virginicum, Capsella bursa pastoris, Olmarabidopsis pumila, Arabis hirsute, Brassica napus, Brassica oleracea, Brassica rapa, Raphanus sativus, Brassica juncacea, Brassica nigra, Eruca vesicaria subsp. sativa, Citrus sinensis, Jatropha curcas, Populus trichocarpa, Medicago truncatula, Cicer yamashitae, Cicer bijugum, Cicer arietinum, Cicer reticulatum, Cicer judaicum, Cajanus cajanifolius, Cajanus scarabaeoides, Phaseolus vulgaris, Glycine max, Gossypium sp., Astragalus sinicus, Lotus japonicas, Torenia fournieri, Allium cepa, Allium fistulosum, Allium sativum, Helianthus annuus, Helianthus tuberosus and/or Allium tuberosum. Particularly preferred are Beta vulgaris, Zea mays, Triticum aestivum, Hordeum vulgare, Secale cereale, Helianthus annuus, Solanum tuberosum, Sorghum bicolor, Brassica rapa, Brassica napus, Brassica juncacea, Brassica oleracea, Raphanus sativus, Oryza sativa, Glycine max, and/or Gossypium sp.

In a further embodiment there is provided a method, wherein the plant or part of the plant is a monocotyledonous plant, preferably Zea mays.

In another aspect, there is provided a plant or a part of a plant, or a progeny thereof, obtainable by a method as detailed above.

Further provided is a use of a plant delivery system as defined according to the first aspect of the present invention; or use of a Cpf1 enzyme or an active fragment thereof, or of a nucleic acid sequence encoding the same as defined according to the first aspect of the present invention; and/or a use of at least one guide RNA system, or the nucleic acid sequence encoding the same as defined according to the first aspect of the present invention; in a method of modifying a genomic target sequence of interest in a plant or part of a plant.

In one embodiment of the above use, there is provided a use, wherein the genomic target sequence of interest is a difficult to access target site.

Due to the fact that the present invention provides a variety of constructs to be combined with each other in a modular manner, the various embodiments as disclosed herein can be independently combined in the various aspects provided herein based on the disclosure provided herein.

Further aspects and embodiments of the present invention can be derived from the subsequent detailed description, the drawings, the sequence listing as well as the attached set of claims.

### BRIEF DESCRIPTION OF THE DRAWINGS

FIG. 1 (FIG. 1A to 1E) shows (FIG. 1A) an example of an embedded gRNA construct of the present invention. In this setting (on the top) the construct comprises a BdUbi10 promoter, a tdTomato (tdT) sequence as frame sequence and two scaffold regions (SC) for a cognate Cpf1 enzyme. The gRNA/crRNA is located between the two scaffold sequences. The construct further comprises a NosT terminator. The sequence on the bottom is a control construct not comprising the tdT sequence as frame sequence. Corresponding sequences are provided with SEQ ID NO: 33 and SEQ ID NO: 34, respectively. FIG. 1B shows a generic embedded RNA construct, wherein the frame sequence (frame) may be selected from any naturally occurring or artificial sequence that is transcribable when introduced into a target cell or cellular system of interest, which is long enough to guarantee that it can be properly transcribed by RNA polymerase II and/or recognized by a Cpf1 enzyme of interest. The construct further comprise a promoter and terminator (Term.) which are suitable for optimized expression in a target cell or cellular system of interest. The gRNA/crRNA is located between the scaffold RNA sequences (sR) which are required for proper recognition and processing of the gRNA/crRNA by a Cpf1 enzyme. FIG. 1C shows the functional mechanism underlying the process underlying the excision of gRNA/crRNA excision from an mRNA construct by a Cpf1 enzyme. An mRNA encoding a suitable frame sequence (Frame) with two scaffold RNA sequences (sR) separated by a gRNA/crRNA in its 3′ UTR (frame-sR-gR-sR) can be cleaved by a Cpf1 enzyme. If both sRs are cleaved, a functional gRNA/crRNA can be produced and loaded into a Cpf1 enzyme to perform genome editing. FIG. 1D shows the advantages of the embedded constructs of the present invention in comparison to a ribozyme control system when targeting two different loci of interest (cf. Example 4 below). The ribozyme control used in these experiments is a conventional ribozyme two component system (Cpf1 and crRNA provided on different expression constructs) having the HH and the HDV ribozyme sequences published by Tang et al., 2017 (“A CRISPR-Cpf1 system for efficient genome editing and transcriptional repression in plants.” Nat Plants 3: 17018.). This experiment was done by co-delivering an LbCpf1 plasmid and the mRNA/non-mRNA embedded guide RNA into protoplasts of corn leaf cells and after 24 hrs cells are counted in a flow cytometer using reporter gene fluorescence. After genomic DNA isolation from these cells, the target sites are amplified and the PCR products get sequenced through NGS. The indel frequency seen from the samples are then normalized against the protoplast transformation efficiency seen from the flow cytometer as shown in FIG. 1E showing a graph of the protoplast transformation efficiency for each sample, in triplicate. The y-axis shown the transformation efficiency in %.

FIG. 2 (FIG. 2A to C) shows (FIG. 2A) an exemplary expression construct for a ribozyme-based RNA delivery system of the present invention. “HD” represent the HDV-like ribozyme portion, wherein this portion may be a plant-derived sequence as disclosed herein, e.g., being derived from rice, sunflower or artichoke. Notably, the LbCpf1 component and the ribozyme-based guide RNA (delivery) system can be used as single transcript system, or as two component system, wherein the LbCpf1 and the crRNA are delivered on two individual plasmids/constructs. FIG. 2B and FIG. 2C show activity data obtain for the different ribozyme-based guide RNA system (cf. Example 5 below).

FIG. 3 (FIGS. 3A and B) shows (FIG. 3A) exemplary LbCpf1 construct. On top, a schematic LbCpf1 construct and a ribozyme-flanked crRNA expression cassette, both under the control of an individual pZmUbi1 promoter and a NosT terminator is depicted. This construct can be provided as two separate expression constructs, as further disclosed below. In the middle, A LbCpf1 RR mutant variant is shown, i.e., the CRISPR nuclease expression construct (SEQ ID NO: 35). On the bottom, FIG. 3A shows another LbCpf1 variant, RVR, based expression construct (SEQ ID NO: 36). FIG. 3B shows the results of an experiment to test different PAM variants, i.e., a RR and RVR as detailed in Example 6 below.

FIG. 4 (FIGS. 4A and B) shows promoter swaps on Cpf1 and crRNA modules to identify optimum expression strategies. Each bar represents the average of three replicates. Each graph represents the results of two experiments. In these optimization series (FIG. 4A) the promoter driving the LbCpf1 nuclease expression pZmUbi1, was replaced with pBdUbi10 or pOsActin1 and their activities were compared at two targets crGEP5 and crGEP7 (cf. Table 1). Clearly pBdUbi10 outperformed the control pZmUbi1 at target crGEP5 while there was no difference in activity at target crGEP7. In the bottom graph (FIG. 4B), the promoter, pZmUbi1 driving the guide RNA expression from the ribozyme construct was swapped for pBdUbi10 and pOsActin1 and their activities were compared at two targets crGEP5 and crGEP7. Clearly pZmUbi1 outperformed the other two at target crGEP5 while there was no difference in activity at target crGEP7. For details, see Example 7 below.

FIG. 5 (FIGS. 5A and B) shows: FIG. 5A an exemplary expression construct unit according to the present invention comprising an LbCpf1 unit and a ribozyme flanked LbCpf1 crRNA. Notably, the arrangement is an exemplary one. Promoters and/or terminators may be exchanged, the use of a single transcript unit or the use of two individual expression constructs, or the use of a specific plant-derived HDV-like sequence may be preferred according to the disclosure provided herein. FIG. 5B shows the results on an experiment, wherein the ability of the ribozyme system to disrupt more than one target simultaneously was tested by expressing three guides, crGEP5, crGEP7 and crGEP43 (cf. Table 1 below) from an array format. A second array with the three guides in the order 43, 5 and 7 was also tested. Based on these results, the indel activity at each guide in decreasing order remained the same irrespective of its position in the array. Further details can be found in Example 9 below. FIG. 5C shows, as a control, the transformation efficiency of the various arrays presented in FIG. 5B.

FIG. 6 (FIG. 6) shows an experiment for the testing of promoter strength to drive expression of RR or RVR version of LbCpf1. pZmUbi1 versus pBdUbi10 were tested in a targeted manner for driving nuclease expression and testing activity at same targets (cf. Table 1) for both constructs. For details, see Example 9 below.

FIG. 7 (FIG. 7) shows the targeted comparison of two arrays with three targets in different order across the two systems of the present invention, the mRNA embedded delivery system and the Artichoke Ribozyme based array, respectively. For further details, see Table 1 and Example 10 below.

FIG. 8 (FIGS. 8A and B) shows the design of the binary vectors used for in planta proof of LbCpf1 mediated genome editing. The LbCpf1_tDTcrRNA_ALS #1 construct harbors expression cassettes for nptII, LbCpf1 and crRNA_ALS #1, which is directed to the target BvALS #1. The crRNA is embedded in a Pol II-driven RNA transcript as described before in Zhong et al., 2017 (FIG. 8A). The binary vector in FIG. 8B shows expression cassettes for nptII, LbCpf1 and crRNA_ALS1, to the BvALS1 sequence (flanked by the HH and HDV ribozymes). This system of expression of crRNA is known from Tang et al. (2017).

FIG. 9 (FIG. 9) shows the results of Cpf1-mediated InDel frequencies in five different target regions (ALS #1-5) of BvALS. For each target region two different crRNA versions (tDT_crRNA [mRNA strategy] or Ribo_crRNA [ribozyme strategy]) were tested. Bars represent mean value of two biological replicates. Error bars represent the standard deviation error.

FIG. 10 (FIG. 10): Comparison of activity of Sunflower-HDV like sequence to the original HDV sequence in the ribozyme-mediated delivery of guide RNA to two targets in a protoplast assay. Using constructs expressing LbCpf1 and guide RNAs flanked by either HH and HDV or HH and Sunflower HDV sequence, the normalized INDELs [%] seen at either target 43 and 7 are equivalent, suggesting that these two sequences are equally active in their function.

FIG. 11 (FIGS. 11A, B and C): Sequencing of target loci through NGS shows LbCpf1 activity inducing InDels in the expected sequence context downstream from the PAM sequence (in bold). Sequences in (A) are targets of WT LbCpf1 which recognizes a TTTV PAM, while sequences in (B) and (C) are targets of the RR variant of LbCpf1 which recognizes a TYCV PAM sequence. FIG. 12 (FIGS. 12A, B, C and D): LbCpf1 activity demonstration in wheat protoplasts at the TaTDF target in the wheat genome. (A) Normalized INDELs percentage resulting from LbCpf1 activity across the three wheat genomes and 5 guides (crGEP52-56). (B-C-D) Sequence alignment resulting from NGS showing LbCpf1 activity at a single target site across the A genome (FIG. 12B), B genome (FIG. 12C) and D genome (FIG. 12D). Sequences shown in bold box are insertions at the target site.

FIG. 13 (FIG. 13): Demonstration of Cpf1 editing and transmission of the edit in whole plants. T0 plants were screened by Cappillary Electrophoresis (CE) for editing leading to identification of three clonal plants with same 6 bp edit at the V3 leaf stage. New leaf growth to v5 leaf stage was checked by CE and showed presence of same amplicon. NGS sequencing of pooled leaves showed presence of the same edits with half traces of wildtype sequence and half traces showing edits conforming a heterozygous plant. T1 progeny was also sequenced shown to contain the edit segregating in a mendelian fashion. FIG. 13 discloses SEQ ID NOS 160-162, respectively, in order of appearance.

FIG. 14 (FIG. 14) shows the design of the binary vector construct LbCpf1-tDTcrRNA_ALS2, which was used for stable transformation of sugar beet. The LbCpf1_tDTcrRNA_ALS2 construct harbors expression cassettes for nptII, LbCpf1, and crRNA_ALS2, which is directed against the target region BvALS2. The crRNA is embedded in a Pol II-driven RNA transcript as described before in Zhong et al., 2017. Terminators: pAG7, nosT, Tpea, PolII promoters: Pnos, PcUbi4. nptII=neomycin phosphotransferase. FIG. 14 discloses SEQ ID NO: 163.

FIG. 15 (FIG. 15) shows a sequence alignment of the BvALS target regions in different edited shoots, which were regenerated after stable callus transformation. In the wildtype sequence of the BvALS gene PAM and protospacer are indicated. The triangle marks the proposed cutting site.

FIG. 16 (FIG. 16): Demonstration of increased activity through a multiplexed guide RNA module. Constructs containing a repeat of a module containing the LbCpf1 scaffold (S) and guide RNA sequence (in this case crGEP7 (7)) were tested for increased INDEL activity in protoplast via delivery through HDV Riobzyme system (HDV) or Artichoke Ribozyme system (Art) or mRNA delivery system (mRNA). As demonstrated here, for target 7 increased delivery through a 3×7-HDV module showed approximately three times higher INDEL activity over a 1×7-HDV module. With the Atichoke ribozyme system (Art) and mRNA delivery system (mRNA) the 3×7 module showed only 50% activity compared to the 1×7 module. NTE=normalized to transformation efficiency.

FIG. 17 (FIGS. 17A, B and C) Modifying the relative dosage of the Cpf1 protein and crRNA modulates the editing efficiency in plant cells (A, B). Adjusting the molar ratios (from 1:0.5 [1_0.5]-1:8 [1_8]) of the nuclease and crRNA constructs shows a demonstrable increase in INDEL activity at targets crGEP5 and crGEP7 in a protoplast assay. (C) Continuing the nuclease: crRNA ratio comparison for target cr7 in B which showed an upward trend from 1:1 [1_1] to a 1:32 [1_32] ratio. NTE=normalized to transformation efficiency.

FIG. 18 (FIG. 18) shows the performance of Cpf1 variants compared to original Cpf1 version in protoplast assays. Four designed versions ordered at commercial sequence providers (variants I (SEQ ID NO: 72), II (SEQ ID NO: 73), III (SEQ ID NO: 75) and IV (SEQ ID NO: 76)) and one published version from Yiping Qi's group (Tang et al., 2017 Nature Plants 3:17018) (variant YQ (SEQ ID NO: 74)). Each block of bars represents a separate experiment wherein the variants were compared to the original version (*). Black bars=Target crGEP5; striped bars=Target crGEP51 and white bars=Target crGEP7. Target INDEL quantification done by ddPCR to get Raw INDELs and the normalized INDEL score was obtained by multiplying raw score with transformation efficiency %. In particular variants I and III outperform the original Cpf1 version.

FIG. 19 (FIG. 19) shows the performance of Cpf1 variants compared to original Cpf1 version in corn Immature Embryos bombardment assay. Four designed versions ordered at commercial sequence providers (variants I (SEQ ID NO: 72), II (SEQ ID NO: 73), III (SEQ ID NO: 75) and IV (SEQ ID NO: 76)) and one published version from Yiping Qi's group (Tang et al., 2017 Nature Plants 3:17018) (variant YQ (SEQ ID NO: 74)). Each block of bars represents a separate experiment wherein the variants were compared to the original version (*). Black bars=Target crGEP5; striped bars=Target crGEP51 and white bars=Target crGEP7. Target INDEL quantification done by NGS to get INDELs %.

FIG. 20 (FIG. 20) shows INDELs activity of Cpf1 variants containing introns in the CDS vs. without introns in protoplast assay system at target crGEP5. The black bars represent raw indels from ddPCR based quantification and the white bars represent normalized scores to the transformation efficiency.

### DEFINITIONS

The terms “amino acid (sequence)”, “polypeptide” and “protein” are used interchangeably herein for specifying an amino acid based structure linked by peptidic bonds. Usually, polypeptides comprising at least 100 amino acids are referred to as proteins, whereas smaller condensation products of amino acids, comprising two to around 100 amino acid building blocks, are denoted as “peptides”.

The terms “associated with” or “in association with” according to the present disclosure are to be construed broadly and, therefore, according to present invention imply that a molecule (DNA, RNA, amino acid, comprising naturally occurring and/or synthetic building blocks) is provided in physical association with another molecule, the association being either of covalent or non-covalent nature. For example, a repair template can be associated with a gRNA of a CRISPR nuclease, wherein the association can be of non covalent nature (complementary base pairing), or the molecules can be physically attached to each other by a covalent bond.

The term “(catalytically) active fragment” as used herein referring to amino acid sequences denotes the core sequence derived from a given template amino acid sequence, or a nucleic acid sequence encoding the same, comprising all or part of the active site of the template sequence with the proviso that the resulting catalytically active fragment still possesses the activity characterizing the template sequence, for which the active site of the native enzyme or a variant thereof is responsible. Said modifications are suitable to generate less bulky amino acid sequences still having the same activity as a template sequence making the catalytically active fragment a more versatile or more stable tool being sterically less demanding.

A “covalent attachment” or “covalent bond” is an attachment that involves the sharing of electron pairs between atoms of the molecules or sequences covalently attached to each other. A “non-covalent” interaction differs from a covalent bond in that it does not involve the sharing of electrons, but rather involves more dispersed variations of electromagnetic interactions between molecules/sequences or within a molecule/sequence. Non-covalent interactions or attachments thus comprise electrostatic interactions, van der Waals forces, π-effects and hydrophobic effects. Of special importance in the context of nucleic acid molecules are hydrogen bonds as electrostatic interaction. A hydrogen bond (H-bond) is a specific type of dipole-dipole interaction that involves the interaction between a partially positive hydrogen atom and a highly electronegative, partially negative oxygen, nitrogen, sulfur, or fluorine atom not covalently bound to said hydrogen atom. Any “association” or “physical association” as used herein thus implies a covalent or non-covalent interaction or attachment. In the case of molecular complexes, e.g. a complex formed by a CRISPR nuclease, a gRNA and a RT, more covalent and non-covalent interactions can be present for linking and thus associating the different components of a molecular complex of interest.

The terms “CRISPR polypeptide”, “CRISPR endonuclease”, “CRISPR nuclease”, “CRISPR protein”, “CRISPR effector” or “CRISPR enzyme” are used interchangeably herein and refer to any naturally occurring or artificial amino acid sequence, or the nucleic acid sequence encoding the same, acting as site-specific DNA nuclease or nickase, wherein the “CRISPR polypeptide” is derived from a CRISPR system of any organism, which can be cloned and used for targeted genome engineering. The terms “CRISPR nuclease” or “CRISPR polypeptide” also comprise mutants or catalytically active fragments or fusions of a naturally occurring CRISPR effector sequences, or the respective sequences encoding the same. A “CRISPR nuclease” or “CRISPR polypeptide” may thus, for example, also refer to a CRISPR nickase or even a nuclease-deficient variant of a CRISPR polypeptide having endonucleolytic function in its natural environment. Preferably, the disclosure of the present invention relies on nuclease-deficient CRISPR nucleases, still possessing their inherent DNA recognition and binding properties assisted by a cognate CRISPR RNA.

Nucleic acid sequences disclosed herein can be “codon-optimized”. “Codon optimization” implies that a DNA or RNA synthetically produced or isolated from a donor organism is adapted to the codon usage of different acceptor organism to improve transcription rates, mRNA processing and/or stability, and/or translation rates, and/or subsequent protein folding of said recombinant nucleic acid in the cell or organism of interest. The skilled person is well aware of the fact that a target nucleic acid can be modified at one position due to the codon degeneracy, whereas this modification will still lead to the same amino acid sequence at that position after translation, which is achieved by codon optimization to take into consideration the species-specific codon usage of a target cell or organism. In turn, nucleic acid sequences as defined herein may have a certain degree of identity to a different sequence, encoding the same protein, but having been codon optimized.

“Complementary” or “complementarity” as used herein describes the relationship between two (c) DNA, two RNA, or between an RNA and a (c) DNA nucleic acid region. Defined by the nucleobases of the DNA or RNA, two nucleic acid regions can hybridize to each other in accordance with the lock-and-key model. To this end the principles of Watson-Crick base pairing have the basis adenine and thymine/uracil as well as guanine and cytosine, respectively, as complementary bases apply. Furthermore, also non-Watson-Crick pairing, like reverse-Watson-Crick, Hoogsteen, reverse-Hoogsteen and Wobble pairing are comprised by the term “complementary” as used herein as long as the respective base pairs can build hydrogen bonding to each other, i.e. two different nucleic acid strands can hybridize to each other based on said complementarity.

The term “derivative” or “descendant” or “progeny” as used herein in the context of a eukaryotic cell, preferably a plant or plant cell or plant material according to the present disclosure relates to the descendants of such a cell or material which result from natural reproductive propagation including sexual and asexual propagation. It is well known to the person having skill in the art that said propagation can lead to the introduction of mutations into the genome of an organism resulting from natural phenomena which results in a descendant or progeny, which is genomically different to the parental organism or cell, however, still belongs to the same genus/species and possesses mostly the same characteristics as the parental recombinant host cell. Such derivatives or descendants or progeny resulting from natural phenomena during reproduction or regeneration are thus comprised by the term of the present disclosure and can be readily identified by the skilled person when comparing the “derivative” or “descendant” or “progeny” to the respective parent or ancestor.

Furthermore, the term “derivative”, in the context of a chemical substance or nucleic acid or amino acid molecule and not referring to a replicating cell or organism, can imply a substance or molecule derived from or originating from the original substance or molecule by chemical and/or biotechnological means, or natural phenomena, like naturally occurring mutations. The resulting derivative will have characteristics allowing the skilled person to clearly define the original or parent molecule the derivative stems from. Furthermore, the derivative might have additional or varying biological functionalities, still a derivative or an “active fragment” of an original molecule will still share at least one biological function of the parent molecule, even though the derivative or active fragment might be shorter/longer than the parent sequence and might comprise certain mutations, deletions or insertions in comparison to the respective parent sequence. A “derivative” in the chemical sense will thus imply a compound that is derived from a similar compound by a chemical reaction. For biomolecules, the term implies that the derivative is not an arbitrary combination of any nucleic acid or amino acid sequence, but the derivative shows a significant degree of identity to the parent sequence when comparing a contiguous stretch of the derivative to a contiguous stretch of the parent sequence.

A “eukaryotic cell” as used herein refers to a cell having a true nucleus, a nuclear membrane and organelles belonging to any one of the kingdoms of Protista, Plantae, Fungi, or Animalia. Eukaryotic organisms can comprise monocellular and multicellular organisms. Preferred eukaryotic cells and organisms according to the present invention are plant cells (see below).

As used herein, “fusion” can refer to a protein and/or nucleic acid sequence, or a domain and/or part thereof, comprising one or more non-native sequences (e.g., moieties) covalently or non-covalently associated with each other to artificially create a fusion molecule. A fusion molecule can comprise different building blocks including nucleic acid sequences (DNA and/or RNA), amino acid sequences and/or non-naturally occurring sequences.

“Functional” in the context of a construct or sequence as disclosed herein implies that a construct comprises at least one coding sequence encoding a RNA or protein sequence as well as further sequences, including, for example regulatory sequences, including promoters and terminators, optimized for a cell or cellular system of interest, or including sequences encoding localization sequences for proper targeting of at least one coding sequence to a subcellular compartment of interest, wherein the thus assembled construct covalently and operably liked together results in the transcription and/or translation of the at least one coding sequence in a cell or cellular system of interest.

Any nucleic acid sequence or amino acid sequence according to the present invention can thus be provided in the form of a fusion molecule by, for example, artificially combining moieties, per se occurring or not occurring in nature, to form a new molecule of at least two molecular building blocks. A fusion can be attached to the N-terminal or C-terminal end of the modified nucleic acid sequence or protein, respectively, or both, or within the molecule as separate domain. For nucleic acid molecules, the fusion molecule can be attached at the 5′ or 3′-end, or at any suitable position in between. A fusion can be a transcriptional and/or translational fusion. A fusion can comprise one or more of the same non-native sequences. A fusion can comprise one 10 or more of different non-native sequences. A fusion can be a chimera. A fusion can comprise a nucleic acid affinity tag. A fusion can comprise a barcode. A fusion can comprise a peptide affinity tag. A fusion can provide for subcellular localization of the at least one synthetic transcription factor as disclosed herein (e.g., a nuclear localization signal (NLS) for targeting (e.g., a site-specific nuclease) to the nucleus, a mitochondrial localization signal for targeting to the mitochondria, a chloroplast localization signal for targeting to a chloroplast, an endoplasmic reticulum (ER) retention signal, and the like).

Further, a “fusion” can result in a “promoter swap” and/or a “terminator swap”, i.e., the exchange of at least one promoter/terminator against another promoter/terminator to identify the best regulatory sequence of a fusion construct of interest for being functional in a cell or organism of interest.

A fusion can provide a non-native sequence (e.g., affinity tag) that can be used to track or purify. A fusion can be a small molecule such as biotin or a dye such as alexa fluor dyes, Cyanine3 dye, Cyanine5 dye. The fusion can provide for increased or decreased stability. In some embodiments, a fusion can comprise a detectable label, including a moiety that can provide a detectable signal. Suitable detectable labels and/or moieties that can provide a detectable signal can include, but are not limited to, an enzyme, a radioisotope, a member of a specific binding pair; a fluorophore; a fluorescent reporter or fluorescent protein; a quantum dot; and the like. A fusion can comprise a member of a FRET pair, or a fluorophore/quantum dot donor/acceptor pair. A fusion can comprise an enzyme. Suitable enzymes can include, but are not limited to, horse radish peroxidase, luciferase, beta-25 galactosidase, and the like. A fusion can comprise a fluorescent protein. Suitable fluorescent proteins can include, but are not limited to, a green fluorescent protein (GFP), (e.g., a GFP from Aequoria victoria, fluorescent proteins from Anguilla japonica, or a mutant or derivative thereof), a red fluorescent protein, a yellow fluorescent protein, a yellow-green fluorescent protein (e.g., mNeonGreen derived from a tetrameric fluorescent protein from the cephalochordate Branchiostoma lanceolatum) any of a variety of fluorescent and colored proteins. A fusion can comprise a nanoparticle. Suitable nanoparticles can include fluorescent or luminescent nanoparticles, and magnetic nanoparticles, or nanodiamonds, optionally linked to a nanoparticle. Any optical or magnetic property or characteristic of the nanoparticle(s) can be detected. A fusion can comprise a helicase, a nuclease (e.g., Fokl), an endonuclease, an exonuclease (e.g., a 5′ exonuclease and/or 3′ exonuclease), a ligase, a nickase, a nuclease-helicase (e.g., Cas3), a DNA methyltransferase (e.g., Dam), or DNA demethylase, a histone methyltransferase, a histone demethylase, an acetylase (including for example and not limitation, a histone acetylase), a deacetylase (including for example and not limitation, a histone deacetylase), a phosphatase, a kinase, a transcription (co-) activator, a transcription (co-) factor, an RNA polymerase subunit, a transcription repressor, a DNA binding protein, a DNA structuring protein, a long non-coding RNA, a DNA repair protein (e.g., a protein involved in repair of either single-and/or double-stranded breaks, e.g., proteins involved in base excision repair, nucleotide excision repair, mismatch repair, NHEJ, HR, microhomology-mediated end joining (MMEJ), and/or alternative non-homologous end-joining (ANHEJ), such as for example and not limitation, HR regulators and HR complex assembly signals), a marker protein, a reporter protein, a fluorescent protein, a ligand binding protein (e.g., mCherry or a heavy metal binding protein), a signal peptide (e.g., Tat-signal sequence), a targeting protein or peptide, a subcellular localization sequence (e.g., nuclear localization sequence, a chloroplast localization sequence), and/or an antibody epitope, or any combination thereof.

A “gene” as used herein refers to a DNA region encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

The term “gene expression” or “expression” as used herein refers to the conversion of the information, contained in a gene, into a “gene product”. A “gene product” can be the direct transcriptional product of a gene (e.g., mRNA, tRNA, rRNA, antisense RNA, ribozyme, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

The term “gene activation” or “augmentation/augmenting/activating/upregulating (of) gene expression” refer to any process which results in an increase in production of a gene product. A gene product can be either RNA (including, but not limited to, mRNA, rRNA, tRNA, and structural RNA) or a protein. Accordingly, gene activation includes those processes which increase transcription of a gene and/or translation of an mRNA. Examples of gene activation processes which increase transcription include, but are not limited to, those which facilitate formation of a transcription initiation complex, those which increase transcription initiation rate, those which increase transcription elongation rate, those which increase processivity of transcription and those which relieve transcriptional repression (by, for example, blocking the binding of a transcriptional repressor). Gene activation can constitute, for example, inhibition of repression as well as stimulation of expression above an existing level. Examples of gene activation processes which increase translation include those which increase translational initiation, those which increase translational elongation and those which increase mRNA stability. In general, gene activation comprises any detectable increase in the production of a gene product, preferably an increase in production of a gene product by about 2-fold, more preferably from about 2- to about 5-fold or any integral value therebetween, more preferably between about 5- and about 10-fold or any integral value therebetween, more preferably between about 10- and about 20-fold or any integral value therebetween, still more preferably between about 20- and about 50-fold or any integral value therebetween, more preferably between about 50- and about 100-fold or any integral value therebetween, more preferably 100-fold or more.

In contrast, the terms “gene repression” or “inhibition/inhibiting/repressing/downregulating (of) gene expression” refer to any process which results in a decrease in production of a gene product. A gene product can be either RNA (including, but not limited to, mRNA, rRNA, tRNA, and structural RNA) or protein. Accordingly, gene repression includes those processes which decrease transcription of a gene and/or translation of a mRNA. Examples of gene repression processes which decrease transcription include, but are not limited to, those which inhibit formation of a transcription initiation complex, those which decrease transcription initiation rate, those which decrease transcription elongation rate, those which decrease processivity of transcription and those which antagonize transcriptional activation (by, for example, blocking the binding of a transcriptional activator). Gene repression can constitute, for example, prevention of activation as well as inhibition of expression below an existing level. Examples of gene repression processes which decrease translation include those which decrease translational initiation, those which decrease translational elongation and those which decrease mRNA stability. Transcriptional repression includes both reversible and irreversible inactivation of gene transcription. In general, gene repression comprises any detectable decrease in the production of a gene product, preferably a decrease in production of a gene product by about 2-fold, more preferably from about 2- to about 5-fold or any integral value therebetween, more preferably between about 5- and about 10-fold or any integral value therebetween, more preferably between about 10- and about 20-fold or any integral value therebetween, still more preferably between about 20- and about 50-fold or any integral value therebetween, more preferably between about 50- and about 100 fold or any integral value therebetween, more preferably 100-fold or more. Most preferably, gene repression results in complete inhibition of gene expression, such that no gene product is detectable.

The terms “genetic construct” or “recombinant construct”, “vector”, or “plasmid (vector)” (e.g., in the context of at least one nucleic acid sequence to be introduced into a cellular system) are used herein to refer to a construct comprising, inter alia, plasmids or (plasmid) vectors, cosmids, artificial yeast-or bacterial artificial chromosomes (YACs and BACs), phagemides, bacterial phage based vectors, an expression cassette, isolated single-stranded or double-stranded nucleic acid sequences, comprising DNA and RNA sequences in linear or circular form, or amino acid sequences, viral vectors, including modified viruses, and a combination or a mixture thereof, for introduction or transformation, transfection or transduction into any prokaryotic or eukaryotic target cell, including a plant, plant cell, tissue, organ or material according to the present disclosure. The construct or vector can thus have a circular, or a linear architecture.

“Recombinant” in the context of a biological material, e.g., a cell or vector, thus implies an artificially produced material comprising at least one human intervention in vitro. A recombinant construct according to the present disclosure can comprise an effector domain, either in the form of a nucleic acid or an amino acid sequence, wherein an effector domain represents a molecule, which can exert an effect in a target cell and includes a transgene, an single-stranded or double-stranded RNA molecule, including a guide RNA ((s)gRNA), a miRNA or an siRNA, or an amino acid sequences, including, inter alia, an enzyme or a catalytically active fragment thereof, a binding protein, an antibody, a transcription factor, a nuclease, preferably a site specific nuclease, and the like. Furthermore, the recombinant construct can comprise regulatory sequences and/or localization sequences. The recombinant construct can be integrated into a vector, including a plasmid vector, and/or it can be present isolated from a vector structure, for example, in the form of a polypeptide sequence or as a non-vector connected single-stranded or double-stranded nucleic acid. After its introduction, e.g. by transformation or transfection by biological or physical means, the genetic construct can either persist extrachromosomally, i.e. non integrated into the genome of the target cell, for example in the form of a double-stranded or single-stranded DNA, a double-stranded or single-stranded RNA or as an amino acid sequence. Alternatively, the genetic construct, or parts thereof, according to the present disclosure can be stably integrated into the genome of a target cell, including the nuclear genome or further genetic elements of a target cell, including the genome of plastids like mitochondria or chloroplasts. The term plasmid vector as used in this connection refers to a genetic construct originally obtained from a plasmid. A plasmid usually refers to a circular autonomously replicating extrachromosomal element in the form of a double-stranded nucleic acid sequence. In the field of genetic engineering these plasmids are routinely subjected to targeted modifications by inserting, for example, genes encoding a resistance against an antibiotic or an herbicide, a gene encoding a target nucleic acid sequence, a localization sequence, a regulatory sequence, a tag sequence, a marker gene, including an antibiotic marker or a fluorescent marker, a sequence, optionally encoding, a readily identifiable and the like. The structural components of the original plasmid, like the origin of replication, are maintained. According to certain embodiments of the present invention, the localization sequence can comprise a nuclear localization sequence (NLS), a plastid localization sequence, preferably a mitochondrion localization sequence or a chloroplast localization sequence. Said localization sequences are available to the skilled person in the field of plant biotechnology. A variety of plasmid vectors for use in different target cells of interest is commercially available and the modification thereof is known to the skilled person in the respective field.

A “genome” as used herein includes both the genes (the coding regions), the non-coding DNA and, if present, the genetic material of the mitochondria and/or chloroplasts, or the genomic material encoding a virus, or part of a virus. The “genome” or “genetic material” of an organism usually consists of DNA, wherein the genome of a virus may consist of RNA (single-stranded or double-stranded).

The terms “genome editing”, “gene editing”, “GE” and “genome engineering” are used interchangeably herein and refer to strategies and techniques for the targeted, specific modification of any genetic information or genome of a living organism at at least one position. As such, the terms comprise gene editing, but also the editing of regions other than gene encoding regions of a genome. It further comprises the editing or engineering of the nuclear (if present) as well as other genetic information of a cell. Furthermore, the terms “genome editing” and “genome engineering” also comprise an epigenetic editing or engineering, i.e. the targeted modification of, e.g. methylation, histone modification or of non-coding RNAs possibly causing heritable changes in gene expression.

The terms “guide RNA”, “gRNA”, “CRISPR nucleic acid sequence”, “single guide RNA”, or “sgRNA” are used interchangeably herein and either refer to a synthetic fusion of a CRISPR RNA (crRNA) and a trans-activating crRNA (tracrRNA), or the term refers to a single RNA molecule consisting only of a crRNA and/or a tracrRNA, or the term refers to a gRNA individually comprising a crRNA or a tracrRNA moiety. A tracr and a crRNA moiety, if present as required by the respective CRISPR polypeptide, thus do not necessarily have to be present on one covalently attached RNA molecule, yet they can also be comprised by two individual RNA molecules, which can associate or can be associated by non-covalent or covalent interaction to provide a gRNA according to the present disclosure. In the case of single RNA-guided endonucleases like Cpf1 (see Zetsche et al., 2015), for example, a crRNA as single guide nucleic acid sequence might be sufficient for mediating DNA targeting.

The term “hybridization” as used herein refers to the pairing of complementary nucleic acids, i.e., DNA and/or RNA, using any process by which a strand of nucleic acid joins with a complementary strand through base pairing to form a hybridized complex. Hybridization and the strength of hybridization (i.e., the strength of the association between the nucleic acids) is impacted by such factors as the degree and length of complementarity between the nucleic acids, stringency of the conditions involved, the Tm of the formed hybrid, and the G:C ratio within the nucleic acids. The term hybridized complex refers to a complex formed between two nucleic acid sequences by virtue of the formation of hydrogen bounds between complementary G and C bases and between complementary A and T/U bases. A hybridized complex or a corresponding hybrid construct can be formed between two DNA nucleic acid molecules, between two RNA nucleic acid molecules or between a DNA and an RNA nucleic acid molecule. For all constellations, the nucleic acid molecules can be naturally occurring nucleic acid molecules generated in vitro or in vivo and/or artificial or synthetic nucleic acid molecules. Hybridization as detailed above, e.g., Watson-Crick base pairs, which can form between DNA, RNA and DNA/RNA sequences, are dictated by a specific hydrogen bonding pattern, which thus represents a non-covalent attachment form according to the present invention. In the context of hybridization, the term “stringent hybridization conditions” should be understood to mean those conditions under which a hybridization takes place primarily only between homologous nucleic acid molecules. The term “hybridization conditions” in this respect refers not only to the actual conditions prevailing during actual agglomeration of the nucleic acids, but also to the conditions prevailing during the subsequent washing steps. Examples of stringent hybridization conditions are conditions under which primarily only those nucleic acid molecules that have at least 70%, preferably at least 75%, at least 80%, at least 85%, at least 90%, at least 91%, at least 92%, at least 93%, at least 94%, at least 95%, at least 96%, at least 97%, at least 98%, at least 99%, or at least 99.50% sequence identity undergo hybridization. Stringent hybridization conditions are, for example: 4×SSC at 65° C. and subsequent multiple washes in 0.1×SSC at 65° C. for approximately 1 hour. The term “stringent hybridization conditions” as used herein may also mean: hybridization at 68° C. in 0.25 M sodium phosphate, pH 7.2, 7% SDS, 1 mM EDTA and 1% BSA for 16 hours and subsequently washing twice with 2×SSC and 0.1% SDS at 68° C. Preferably, hybridization takes place under stringent conditions.

The term “indel” or “INDEL” as used herein means and insertion and/or deletion in the genome of an organism, or in the genomic material of a cell or cellular system of interest.

The terms “nucleotide” and “nucleic acid” with reference to a sequence or a molecule are used interchangeably herein and refer to a single- or double-stranded DNA or RNA of natural or synthetic origin. The term nucleotide sequence is thus used for any DNA or RNA sequence independent of its length, so that the term comprises any nucleotide sequence comprising at least one nucleotide, but also any kind of larger oligonucleotide or polynucleotide. The term(s) thus refer to natural and/or synthetic deoxyribonucleic acids (DNA) and/or ribonucleic acid (RNA) sequences, which can optionally comprise synthetic nucleic acid analoga. A nucleic acid according to the present disclosure can optionally be codon optimized. Codon optimization implies that the codon usage of a DNA or RNA is adapted to that of a cell or organism of interest to improve the transcription rate of said recombinant nucleic acid in the cell or organism of interest. The skilled person is well aware of the fact that a target nucleic acid can be modified at one position due to the codon degeneracy, whereas this modification will still lead to the same amino acid sequence at that position after translation, which is achieved by codon optimization to take into consideration the species-specific codon usage of a target cell or organism. Nucleic acid sequences according to the present application can carry specific codon optimization for the following non limiting list of organisms: Hordeum vulgare, Hordeum bulbusom, Sorghum bicolor, Saccharum officinarium, Zea spp., including Zea mays, Setaria italica, Oryza minuta, Oryza sativa, Oryza australiensis, Oryza alta, Triticum aestivum, Triticum durum, Secale cereale, Triticale, Malus domestica, Brachypodium distachyon, Hordeum marinum, Aegilops tauschii, Daucus glochidiatus, Beta spp., including Beta vulgaris, Daucus pusillus, Daucus muricatus, Daucus carota, Eucalyptus grandis, Nicotiana sylvestris, Nicotiana tomentosiformis, Nicotiana tabacum, Nicotiana benthamiana, Solanum lycopersicum, Solanum tuberosum, Coffea canephora, Vitis vinifera, Erythrante guttata, Genlisea aurea, Cucumis sativus, Marus notabilis, Arabidopsis arenosa, Arabidopsis lyrata, Arabidopsis thaliana, Crucihimalaya himalaica, Crucihimalaya wallichii, Cardamine nexuosa, Lepidium virginicum, Capsella bursa pastoris, Olmarabidopsis pumila, Arabis hirsute, Brassica napus, Brassica oleracea, Brassica rapa, Raphanus sativus, Brassica juncacea, Brassica nigra, Eruca vesicaria subsp. sativa, Citrus sinensis, Jatropha curcas, Populus trichocarpa, Medicago truncatula, Cicer yamashitae, Cicer bijugum, Cicer arietinum, Cicer reticulatum, Cicer judaicum, Cajanus cajanifolius, Cajanus scarabaeoides, Phaseolus vulgaris, Glycine max, Gossypium sp., Astragalus sinicus, Lotus japonicas, Torenia fournieri, Allium cepa, Allium fistulosum, Allium sativum, Helianthus annuus, Helianthus tuberosus and/or Allium tuberosum. Particularly preferred are Beta vulgaris, Zea mays, Triticum aestivum, Hordeum vulgare, Secale cereale, Helianthus annuus, Solanum tuberosum, Sorghum bicolor, Brassica rapa, Brassica napus, Brassica juncacea, Brassica oleracea, Raphanus sativus, Oryza sativa, Glycine max, and/or Gossypium sp.

As used herein, “non-native”, or “non-naturally occurring”, or “artificial”, or “synthetic” can refer to a nucleic acid or polypeptide sequence, or any other biomolecule like biotin or fluorescein that is not found in a native nucleic acid or protein. Non-native can refer to affinity tags. Non-native can refer to fusions. Non-native can refer to a naturally occurring nucleic acid or polypeptide sequence that comprises mutations, insertions and/or deletions. A non-native sequence may exhibit and/or encode for an activity (e.g., enzymatic activity, methyltransferase activity, acetyltransferase activity, kinase activity, ubiquitinating activity, etc.) that can also be exhibited by the nucleic acid and/or polypeptide sequence to which the non-native sequence is fused. A non-native nucleic acid or polypeptide sequence may be linked to a naturally-occurring nucleic acid or polypeptide sequence (or a variant thereof) by genetic engineering to generate a chimeric nucleic acid and/or polypeptide sequence encoding a chimeric nucleic acid and/or polypeptide. A non-native sequence can refer to a 3′ hybridizing extension sequence, or a nucleas localization signal attached to a molecule. A “synthetic transcription factor” as used herein thus refers to a molecule comprising at least two domains, a recognition domain and an activation domain not naturally occurring in nature.

An “organism” as used herein refers to an individual eukaryotic life form, including a plant, part of a plant, plant organ, plant tissue or plant cell.

The term “particle bombardment” as used herein, also named “biolistic transfection” or “biolistic bombardment” or “microparticle-mediated gene transfer”, refers to a physical delivery method for transferring a coated microparticle or nanoparticle comprising a nucleic acid or a genetic construct of interest into a target cell or tissue. The micro- or nanoparticle functions as projectile and is fired on the target structure of interest under high pressure using a suitable device, often called “gene-gun”. The transformation via particle bombardment uses a microprojectile of metal covered with the gene of interest, which is then shot onto the target cells using an equipment known as “gene-gun” (Sandford et al. 1987) at high velocity fast enough to penetrate the cell wall of a target tissue, but not harsh enough to cause cell death. For protoplasts, which have their cell wall entirely removed, the conditions are different logically. The precipitated nucleic acid or the genetic construct on the at least one microprojectile is released into the cell after bombardment, and integrated into the genome or expressed transiently according to the definition given above. The acceleration of microprojectiles is accomplished by a high voltage electrical discharge or compressed gas (helium). Concerning the metal particles used it is mandatory that they are non-toxic, non-reactive, and that they have a smaller diameter than the target cell. The most commonly used are gold or tungsten. There is plenty of information publicly available from the manufacturers and providers of gene-guns and associated system concerning their general use.

The terms “plant” or “plant cell” as used herein refer to a plant organism, a plant organ, differentiated and undifferentiated plant tissues, plant cells, seeds, and derivatives and progeny thereof. Plant cells include without limitation, for example, cells from seeds, from mature and immature cells or organs, including embryos, meristematic tissues, seedlings, callus tissues in different differentiation states, leaves, flowers, roots, shoots, male or female gametophytes, sporophytes, pollen, pollen tubes and microspores, protoplasts, macroalgae and microalgae. The different eukaryotic cells, for example plant cells, can have any degree of ploidity, i.e. they may either be haploid, diploid, tetraploid, hexaploid or polyploid.

A “promoter” refers to a DNA sequence capable of controlling expression of a coding sequence, i.e., a gene or part thereof, or of a functional RNA, i.e. a RNA which is active without being translated, for example, a miRNA, a siRNA, an inverted repeat RNA or a hairpin forming RNA. A promoter is usually located at the 5′ part of a gene. Promoter structures occur in all kingdoms of life, i.e., in bacteria, archaea, and eucaryots, where they have different architectures. The promoter sequence usually consists of proximal and distal elements in relation to the regulated sequence, the latter being often referred to as enhancers. Promoters can have a broad spectrum of activity, but they can also have tissue or developmental stage specific activity. For example, they can be active in cells of roots, seeds and meristematic cells, etc. A promoter can be active in a constitutive way, or it can be inducible. The induction can be stimulated by a variety of environmental conditions and stimuli. There exist strong promoters which can enable a high transcription of the regulated sequence, and weak promoters. Often promoters are highly regulated. A promoter of the present disclosure may include an endogenous promoter natively present in a cell, or an artificial or transgenic promoter, either from another species, or an artificial or chimeric promoter, i.e. a promoter that does not naturally occur in nature in this composition and is composed of different promoter elements. The process of transcription begins with the RNA polymerase (RNAP) binding to DNA in the promoter region, which is in the immediate vicinity of the transcription start site (TSS). A typical promoter sequence is thought to comprise some sequence motifs positioned at specific sites relative to the TSS. For example, a prokaryotic promoter is observed to have two hexameric motifs centered at or near −10 (Pribnow box) and −35 positions relative to the TSS. Furthermore, there can be an AT rich UP (“upstream”) element upstream of the −35 region. Procaryotic promoters are recognized by sigma factors as transcription factors. The structure of eukaryotic promoters is generally more complex and they have several different sequence motifs, such as TATA box, INR box, BRE, CCAAT-box and GC-box (Bucher P., J. Mol. Biol. 1990 Apr. 20; 212 (4): 563-78.). Eukaryotic cells posses three RNAPs, RNA polymerase I, II, and III, respectively. RNAP I generates ribosomal RNA (rRNA), RNAP II generates messenger RNA (mRNA) and small nuclear RNA (snRNA), and RNAP III generates transfer RNA (tRNA), snRNA and 5S-RNA.

The term “regulatory sequence” as used herein refers to a nucleic acid or amino acid sequence, which can direct the transcription and/or translation and/or modification of a nucleic acid sequence of interest. Regulatory sequences can comprise sequences acting in cis or acting in trans. Exemplary regulatory sequences comprise promoters, enhancers, terminators, operators, transcription factors, transcription factor binding sites, introns and the like.

The term “terminator”, as used herein, refers to DNA sequences located downstream, i.e. in 3′ direction, of a coding sequence and can include a polyadenylation signal and other sequences, i.e. further sequences encoding regulatory signals that are capable of affecting mRNA processing and/or gene expression. The polyadenylation signal is usually characterized in that it adds poly-A-nucleotides at the 3′-end of an mRNA precursor.

The terms “transient” or “transient introduction” as used herein refer to the transient introduction of at least one nucleic acid and/or amino acid sequence according to the present disclosure, preferably incorporated into a delivery vector and/or into a recombinant construct, with or without the help of a delivery vector, into a target structure, for example, a plant cell or cellular system, wherein the at least one nucleic acid or nucleotide sequence is introduced under suitable reaction conditions so that no integration of the at least one nucleic acid sequence into the endogenous nucleic acid material of a target structure, the genome as a whole, occurs, so that the at least one nucleic acid sequence will not be integrated into the endogenous DNA of the target cell. As a consequence, in the case of transient introduction, the introduced genetic construct will not be inherited to a progeny of the target structure, for example a prokaryotic, an animal or a plant cell. The at least one nucleic acid and/or amino acid sequence or the products resulting from transcription, translation, processing, post-translational modifications or complex building thereof are only present temporarily, i.e., in a transient way, in constitutive or inducible form, and thus can only be active in the target cell for exerting their effect for a limited time. Therefore, the at least one sequence introduced via transient introduction will not be heritable to the progeny of a cell. The effect mediated by at least one sequence or effector introduced in a transient way can, however, potentially be inherited to the progeny of the target cell. A “stable” introduction therefore implies the integration of a nucleic acid or nucleotide sequence into the genome of a target cell or cellular system of interest, wherein the genome comprises the nuclease genome as well as the genome comprised by further organelles.

The term “variant(s)” as used herein in the context of amino acid or nucleic acid sequences is intended to mean substantially similar sequences. For nucleic acid sequences, a variant comprises a deletion and/or addition of one or more nucleotides at one or more internal sites within the native polynucleotide and/or a substitution of one or more nucleotides at one or more sites in the native polynucleotide. As used herein, a “native” polynucleotide or polypeptide comprises a naturally occurring nucleotide sequence or amino acid sequence, respectively. For nucleic acid sequences, conservative variants include those sequences that, because of the degeneracy of the genetic code, encode the same amino acid sequence as a reference sequence of the present disclosure. A variant of a given nucleic acid sequence will thus also include synthetically derived nucleic acid sequences, such as those generated, for example, by using site-directed mutagenesis but which still encode the same protein as the reference sequence. Generally, variants of a particular polynucleotide of the disclosure will have at least about 60%, 65%, 70%, 75%, 80%, 85%, 90%, 91%, 92%, 93%, 94%, 95%, 96%, 97%, 98%, 99% or more sequence identity to that particular nucleic acid sequence has determined by sequence alignment programs and parameters described further below under this section. Notably, two sequences being a parent and a variant will be compared over their whole length of contiguous sequences.

Whenever the present disclosure relates to the percentage of identity of nucleic acid or amino acid sequences to each other these values define those values as obtained by using the EMBOSS Water Pairwise Sequence Alignments (nucleotide) programme (www.ebi.ac.uk/Tools/psa/emboss_water/nucleotide.html) nucleic acids or the EMBOSS Water Pairwise Sequence Alignments (protein) programme (www.ebi.ac.uk/Tools/psa/emboss_water/) for amino acid sequences. Alignments or sequence comparisons as used herein refer to an alignment over the whole length of two sequences compared to each other. Those tools provided by the European Molecular Biology Laboratory (EMBL) European Bioinformatics Institute (EBI) for local sequence alignments use a modified Smith-Waterman algorithm (see www.ebi.ac.uk/Tools/psa/ and Smith, T. F. & Waterman, M. S. “Identification of common molecular subsequences” Journal of Molecular Biology, 1981 147 (1): 195-197). When conducting an alignment, the default parameters defined by the EMBL-EBI are used. Those parameters are (i) for amino acid sequences: Matrix=BLOSUM62, gap open penalty=10 and gap extend penalty=0.5 or (ii) for nucleic acid sequences: Matrix=DNAfull, gap open penalty=10 and gap extend penalty=0.5. The skilled person is well aware of the fact that, for example, a sequence encoding a protein can be “codon-optimized” if the respective sequence is to be used in another organism in comparison to the original organism a molecule originates from.

Whenever the present invention discloses vector plasmid sequences as disclosed in the attached sequence listing, the skilled person can easily visualize and analyze the relevant sequences by using software for visualizing sequences like DNA plasmid sequences, e.g., SnapGene Viewer (www.snapgene.com/products/snapgene_viewer/) and further to identify restriction enzyme sites, primers and individual sub-elements of a plasmid sequence, including promoter sequences, nuclear localization sequences, terminator sequences, selection marker encoding sequences and coding sequences, etc.

### DETAILED DESCRIPTION

The skilled person will understand that the herein described aspects and embodiments of the present invention are not limited to the specific context in which they are disclosed but may rather be combined with other aspects and embodiments disclosed throughout the present specification independently from their context.

The present invention provides several individual aspects to be modulated to establish and improve the efficiency of CRISPR/Cpf1 system in plants or plant cells; in particular in monocots, e.g., corn (e.g. Zea mays).

In a first aspect, there is provided a plant delivery system, wherein the delivery system may comprise (a) at least one Cpf1 enzyme or an active fragment thereof, or a nucleic acid sequence encoding the same; and (b) at least one Cpf1 guide RNA system, or a nucleic acid sequence encoding the same, the at least one Cpf1 guide RNA system comprising at least one Cpf1 guide RNA specific for a genomic target sequence of interest in a plant or part of a plant; wherein the at least one Cpf1 guide RNA, or the nucleic acid sequence encoding the same, is (i) flanked by a Hammerhead ribozyme sequence at the 5′ end and by a plant-derived Hepatitis Delta Virus (HDV)-like ribozyme sequence at the 3′-end; and/or is (ii) embedded within a non-coding region, preferably a 3′ untranslated region (UTR), of a sequence encoding a frame sequence.

In one embodiment according to the various aspects of the present disclosure, the plant delivery system may comprise a first nucleotide molecule comprising a nucleic acid sequence encoding the at least one Cpf1 enzyme or an active fragment thereof, and a second nucleotide molecule comprising a nucleic acid sequence encoding the at least one Cpf1 guide RNA system, preferably wherein the first and the second molecule are provided on separate constructs, or wherein the first and the second molecule are provided on a single transcript construct. Therefore, as further detailed below, the constructs of the present invention will have a modular character. Separate constructs may have certain advantages by individually regulating the expression of the at least one Cpf1 enzyme or an active fragment thereof, and an individually provided properly regulated at least one Cpf1 guide RNA system. Single transcript units simultaneously providing the Cpf1 nuclease and the Cpf1 guide RNA system—both components under the control of individual regulatory sequences—may be preferred depending on the plant target cell or cellular system of interest. In one embodiment, the invention thus provides a single transcript CRISPR/Cpf1 system for efficient gene editing. Specifically, the invention provides the generation of a single mRNA transcript comprising a Cpf1 open reading frame at the 5′ end followed by gRNA and a terminator sequence.

In one embodiment according to the various aspects of the present disclosure, the first construct comprising the first nucleotide molecule and the second construct comprising the second nucleotide molecule may each comprise at least one promoter functional in a plant or part of a plant, preferably wherein the at least one promoter driving expression of the first and the second nucleotide molecule is different in the first and the second construct, respectively. In another embodiment, the promoters may be the same. Choosing different promoters, as shown herein, might provide for an optimum performance of these regulatory sequences to provide the CRISPR components such expressed in a functional manner in an amount sufficient to allow successful genome editing (GE) outcomes.

A CRISPR system in its natural environment describes a molecular complex comprising at least one small and individual non-coding RNA in combination with a Cas nuclease or another CRISPR nuclease like a Cpf1 nuclease (Zetsche et al., 2015, supra) which can produce a specific DNA double-stranded break. Presently, CRISPR systems are categorized into 2 classes comprising five types of CRISPR systems, the type II system, for instance, using Cas9 as effector and the type V system using Cpf1 as effector molecule (Makarova et al., Nature Rev. Microbiol., 2015). In artificial CRISPR systems, a synthetic non-coding RNA and a CRISPR nuclease and/or optionally a modified CRISPR nuclease, modified to act as nickase or lacking any nuclease function, can be used in combination with at least one synthetic or artificial guide RNA or gRNA combining the function of a crRNA and/or a tracrRNA (Makarova et al., 2015, supra). The immune response mediated by CRISPR/Cas in natural systems requires CRISPR-RNA (crRNA), wherein the maturation of this guiding RNA, which controls the specific activation of the CRISPR nuclease, varies significantly between the various CRISPR systems which have been characterized so far. Firstly, the invading DNA, also known as a spacer, is integrated between two adjacent repeat regions at the proximal end of the CRISPR locus. Type II CRISPR systems, for example, can code for a Cas9 nuclease as key enzyme for the interference step, which system contains both a crRNA and also a trans-activating RNA (tracrRNA) as the guide motif. These hybridize and form double-stranded (ds) RNA regions which are recognized by RNAseIII and can be cleaved in order to form mature crRNAs. These then in turn associate with the Cas molecule in order to direct the nuclease specifically to the target nucleic acid region. Recombinant gRNA molecules can comprise both the variable DNA recognition region and also the Cas interaction region and thus can be specifically designed, independently of the specific target nucleic acid and the desired Cas nuclease. As a further safety mechanism, PAMs (protospacer adjacent motifs) must be present in the target nucleic acid region; these are DNA sequences which follow on directly from the Cas9/RNA complex-recognized DNA. The PAM sequence for the Cas9 from Streptococcus pyogenes has been described to be “NGG” or “NAG” (Standard IUPAC nucleotide code) (Jinek et al, “A programmable dual-RNA-guided DNA endonuclease in adaptive bacterial immunity”, Science 2012, 337:816-821). The PAM sequence for Cas9 from Staphylococcus aureus is “NNGRRT” or “NNGRR(N)”. Further variant CRISPR/Cas9 systems are known. Thus, a Neisseria meningitidis Cas9 cleaves at the PAM sequence NNNNGATT. A Streptococcus thermophilus Cas9 cleaves at the PAM sequence NNAGAAW. Recently, a further PAM motif NNNNRYAC has been described for a CRISPR system of Campylobacter (WO 2016/021973 A1). For Cpf1 nucleases it has been described that the Cpf1-crRNA complex, without a traceRNA, efficiently recognize and cleave target DNA proceeded by a short T-rich PAM in contrast to the commonly G-rich PAMs recognized by Cas9 systems (Zetsche et al., supra). Furthermore, by using modified CRISPR polypeptides, specific single-stranded breaks can be obtained. The combined use of Cas nickases with various recombinant gRNAs can also induce highly specific DNA double-stranded breaks by means of double DNA nicking. By using two gRNAs, moreover, the specificity of the DNA binding and thus the DNA cleavage can be optimized. Further CRISPR effectors like CasX and CasY effectors originally described for bacteria, are meanwhile available and represent further effectors, which can be used for genome engineering purposes (Burstein et al., “New CRISPR-Cas systems from uncultivated microbes”, Nature, 2017, 542, 237-241).

Synthetic CRISPR systems consisting of two components, a guide RNA (gRNA) also called single guide RNA (sgRNA) and a non-specific CRISPR-associated endonuclease can be used to generate knock-out plant cells by co-expressing a gRNA specific to the gene to be targeted and capable of association with the cognate CRISPR endonuclease. Despite the huge research efforts in the field of CRISPR for providing suitable genome editing (GE) tools, no plant-optimized system for highly efficient and reliable GE even for difficult to access target sites is available relying on a Cpf1 effector nuclease.

A “plant delivery system” as used herein thus means a vector construct, or at least two or a plurality of vector constructs suitable to introduce all effector components of a Cpf1-based CRISPR system optimized for GE in a plant cell or cellular system. A plant delivery system as provided herein thus provides a plant-optimized Cpf1 effector, preferably a LbCpf1 effector originating from Lachnospiraceae bacterium, preferably Lachnospiraceae bacterium ND2006, comprising an optimum sequence and individually tested regulatory sequences, including promoters and terminators, as well as a Cpf1 guide RNA system, or a nucleic acid sequence encoding the same, the at least one Cpf1 guide RNA system comprising at least one Cpf1 guide RNA, i.e., a guide RNA specific for the cognate Cpf1 effector, which Cpf1 guide RNA system can also comprise more than one gRNA to be suitable for multiplex targeting. Furthermore, the Cpf1 guide RNA system has an specific architecture either relying on a plant-derived Hepatitis Delta Virus (HDV)-like ribozyme and/or using an embedding technique, i.e., a construct design, wherein the at least one Cpf1 crRNA is placed within a non-coding region, preferably a 3′ untranslated region (UTR), of a sequence encoding a frame sequence to guarantee optimum stability, transcription and thus efficient GE rates.

In one aspect, it was found that Cpf1 has certain features in comparison to Cas9 that make it advantageous for certain use cases, e.g., the editing in AT-rich genomes/regions, or single nucleotide polymorphism (SNP)-specific editing. All characterized genome editing nucleases before Cpf1 required at least one G in their PAMs. Cpf1′s PAM is TTTN, so it broadens the range of genome editing experiments. This is particularly useful in AT-rich genomes, or regions, e.g. scaffold/matrix attachment regions. It may also be useful if a particular SNP is present that results in an AT-rich site, or for applications (e.g. chemically modified guides) where shorter RNA species are useful. Cpf1 only requires a crRNA to process crRNA arrays without needing tracrRNA. Cpf1-crRNA can cut target DNA without needing any other RNA types. Furthermore, the staggered cut introduced by Cpf1 may be advantageous for certain HDR-free knock-in strategies. Furthermore, an increased efficiency of HDR-based knock-ins may be achieved using Cpf1. Finally, Cpf1 cleaves its target DNA relatively far away from the PAM at the end of the protospacer, unlike Cas9.The indels caused by Cpf1 will therefore be located far from the target site. Cpf1 can continue cutting at the target site as a result, which may increase the chance that new DNA can be inserted at that site.

Still, many of this aspects have only been confirmed in an animal/mammalian system for selected Cpf1 species, wherein data for successful optimizations of a Cpf1 of interest together with specific crRNA tools used in combination with said optimized Cpf1 to achieve efficient GE by synergistically optimizing a Cpf1 and the cognate crRNA guide RNA system are not available for relevant crop plants, including Zea mays.

In one aspect of the present invention, there is thus provided an LbCpf1 enzyme or a catalytically active fragment thereof, or the nucleic acid sequence encoding the same, which sequence originates from Lachnospiraceae bacterium (Lb) and which has been optimized for the expression in corn. In one embodiment, the plant-optimized LbCpf1 sequence (comprising a 5′ sequence encoding an enhancer and a SV40 NLS and a 3′ sequence encoding a nucleoplasmin NLS) is SEQ ID NO: 13 or 15, or the core sequence is selected from SEQ ID NO: 14 or 16, or a sequence having at least 85%, 86%, 87%, 88%, 89%, 90%, 91%, 92%, 93%, 94%, 95%, 96%, 97%, 98%, or 99% identity over the whole length of the respective sequence of SEQ ID NOs: 13 to 16. A suitable expression construct for SEQ ID NO: 13 is provided with SEQ ID NO: 37. Notably, the skilled person is well aware of the fact that certain regulatory elements and markers within SEQ ID NO: 37 may be replaced, or that the sequence encoding SEQ ID NO: 16 may be further truncated or modified within the scope of the present invention.

Notably, the nucleic acid sequence encoding LbCpf1 of SEQ ID NO: 13 has been specifically corn codon-optimized and further carries a 5′/N-terminal portion derived from eGFP (enhanced green fluorescent protein), a mass enhancer and a SV40 NLS as well as a 3′ located nucleoplasmin NLS.

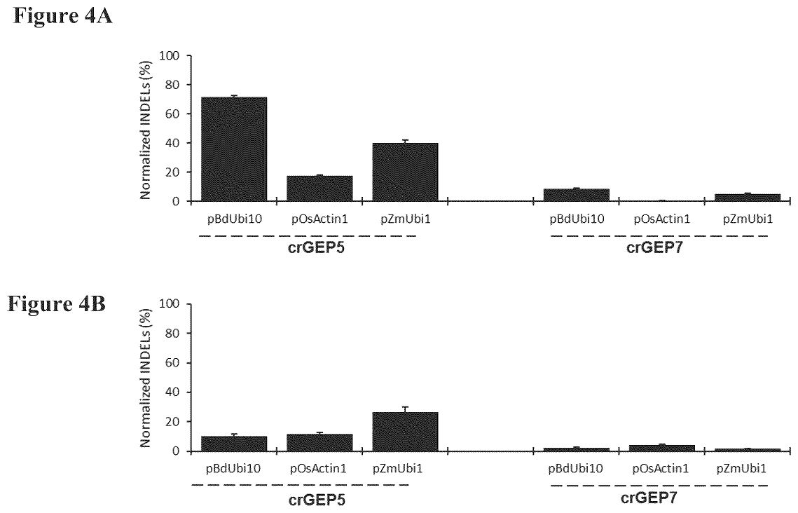

In one embodiment, the codon-optimized LbCpf1 construct sequence may comprise two NLS sequences on both its 5′ and 3′-ends and is flanked by a BdUbi10 promoter sequence, or a ZmUbi1 promoter sequence and a Nos terminator (NosT) sequence as further disclosed herein separately in the functional construct (SEQ ID NO: 37). This novel codon optimized sequence has shown high indel activities at multiple target sites when delivered together with a guide RNA delivery construct according to the prior art and according to the present invention. This effect can be attributed to the specific structure of the LbCpf1 sequence of the present invention and the optimized translation context. Furthermore, as detailed in Example 7 and FIG. 4 it could be systematically shown that the choice of the promoter for the LbCpf1 construct for use in plants can play a significant role.

In a further embodiment, the codon-optimized LbCpf1 construct sequence may comprise an additional (Zm)Ubi1 intron sequence of SEQ ID NO: 5 or 10, or a sequence having at least 90%, 91%, 92%, 93%, 94%, 95%, 96%, 97%, 98%, or 99% identity when compared over the whole length of the respective sequence of any one of SEQ ID NO: 5 or 10, to enhance expression.

In yet a further embodiment, the functional LbCpf1 construct may comprise a different promoter and/or terminator sequence in comparison to SEQ ID NO: 37 as further disclosed herein, or as known to the skilled person.

In one embodiment of the present invention, the construct for expression of a plant optimized LbCpf1 may be a sequence of SEQ ID NO: 37, or a sequence comprising the LbCpf1 sequence of SEQ ID NO: 13 or 14, or a sequence having at least 85%, 86%, 87%, 88%, 89%, 90%, 91%, 92%, 93%, 94%, 95%, 96%, 97%, 98%, or 99% identity over the whole length of the respective sequence of SEQ ID NO: 13 or 14.

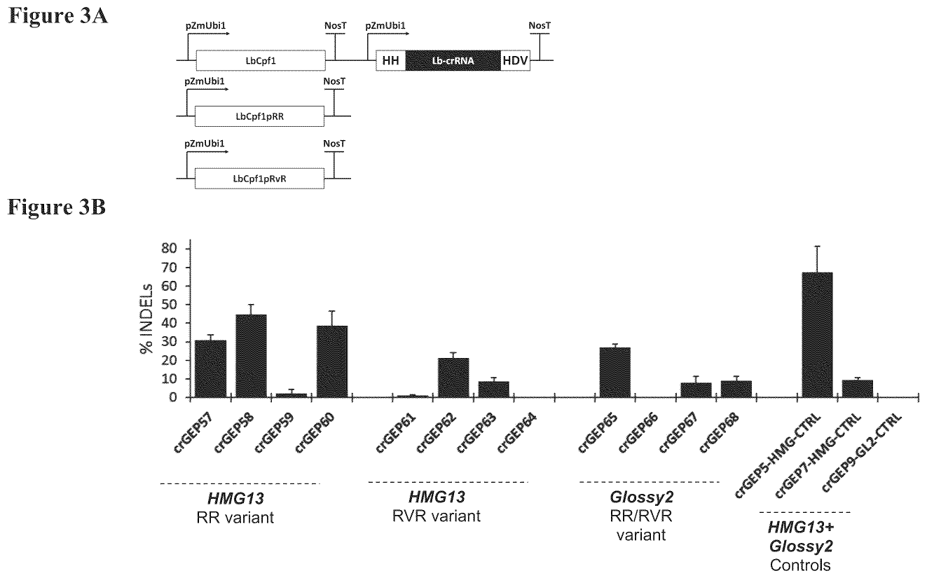

The LbCpf1 nuclease requires a TTTV PAM sequence motif for target site recognition which potentially limits its usage in GC rich genomic regions. Thus, a further aspect of the present invention relates to the combination of codon-optimized LbCpf1 with several relaxed PAM sequence variants of LbCpf1 in order to broaden LbCpf1 recognition of target sites. In particular, two versions of the PAM motif, namely the G532R/K595R (RR) version and the G532R/K538V/Y542R (RVR) version, which have been shown to be active in mammalian cells but so far not in plant cells (Gao et al. (2017) Nat Biotechnol, 35 (8): 789-792), were used in the present invention to broaden target specificity of LbCpf1 in plant cells. These modifications result in alternative PAM recognition sequences, namely TYCV and TATV. The present invention provides evidence that the use of these alternative PAM variants increases the range of target site recognition across diverse sequence loci in plant genomes (see FIG. 3B).

In one embodiment of the present invention, the construct for expression of a plant optimized LbCpf1 may be a sequence of SEQ ID NOs: 35, 36, or a sequence comprising the LbCpf1 sequence of SEQ ID NOs: 38 or 39, or a sequence having at least 85%, 86%, 87%, 88%, 89%, 90%, 91%, 92%, 93%, 94%, 95%, 96%, 97%, 98%, or 99% identity over the whole length of the respective sequence of SEQ ID NOs: 38 or 39, or the respective protein sequence encoded.

Furthermore, this invention thus relates to the combined use of a plant-optimized LbCpf1 enzyme, or an active fragment thereof, or of an alternative RR and RVR LbCpf1 with a specific Cpf1 guide RNA system comprising a ribozyme delivery system comprising plant-derived HDV sequences, and/or embedded within a non-coding region, preferably a 3′ untranslated region (UTR), of a sequence encoding a frame sequence. This approach produces high rates of indel generation activity at various genomic loci.

In preferred embodiments of the various aspects of the present invention, the nucleic acid sequence encoding the Cpf1 enzyme or an active fragment thereof may be codon-optimized for expression in a plant or part of a plant.

In certain embodiments of the various aspects of the present invention, the nucleic acid sequence encoding the Cpf1 enzyme or an active fragment thereof of a plant delivery system of the present invention may be selected from SEQ ID NOs: 13, 14, 38, 39, 72, 73, 74, 75, 76, 157, or 158, or a sequence having at least 85%, 86%, 87%, 88%, 89%, 90%, 91%, 92%, 93%, 94%, 95%, 96%, 97%, 98%, or 99% identity over the whole length of the respective sequence of SEQ ID NOs: 13, 14, 38, 39, 72, 73, 74, 75, 76, 157, or 158.

In one embodiment of the various aspects of the present invention, the at least one construct of a plant delivery system comprises a promoter, wherein the at least one promoter may be independently selected from a (p)BdUbi10 promoter (SEQ ID NO: 1), a (p)ZmUbi1 promoter (SEQ ID NO: 2), a (p)OsActin promoter (SEQ ID NO: 3), and a single or double 35S promoter (SEQ ID NO: 4), optionally including an ZmUbi1 intron, an BdUbi10 intron and/or an Adh1 intron, (SEQ ID NOs: 5 to 10, or 67), or any combination thereof, or a sequence having at least 90%, 91%, 92%, 93%, 94%, 95%, 96%, 97%, 98%, or 99% identity when compared over the whole length of the respective sequence of any one of SEQ ID NOs: 1 to 10, or 67. Bd means originating from Brachypodium distachyon, Zm means originating from Zea mays, Adh1 means originating from alcohol dehydrogenase-1, and Os means originating from Oryza sativa.

As further disclosed herein, the choice of the promoter and the choice of different promoters for individual constructs may be crucial to obtain optimized expression of the respective effector sequences encoded by the constructs, or the plant delivery system (cf. Example 7).

Therefore, the present invention also provides for promoter optimization for Cpf1 nuclease and ribozyme-mediated gRNA expression. In particular, the present invention relates to the fine-tuning of the expression of a Cpf1 nuclease and a ribozyme-mediated gRNA delivery system. The invention relates to the use of different promoter sequences at different genomic loci in order to improve CRISPR/Cpf1 expression. In certain embodiments, the invention pertains to the use of highly constitutive promoters such as pZmUbi1, pBdUbi10, pOsActin and p2x35S+Adh1 intron as disclosed herein to drive high levels of synchronous expression of both Cpf1 nuclease and gRNA in plant cells.

Other suitable promoters may be selected from pNOS, pEF1-alphae, ubiquitin promoters from monocotyledonous and dicotyledonous plants, or a tubulin promoter. The sequences of said promoters are known to the skilled person and can be individually tested for their efficiency in the constructs of the present invention. As it is known to the skilled person, certain target cells will respond differently to a given promoter or terminator or any other regulatory sequence disclosed herein. Methods for replacing a regulatory sequence, including a promoter or terminator, are disclosed herein and rely on standard techniques of molecular cloning so that a suitable construct with a different regulatory sequence can be designed for a target cell, preferably a plant target cell of interest.

In certain embodiments, an inducible promoter, including a heat shock (e.g. HSP70 promoter like pZmERD2) or drought inducible promoter (e.g. Rab17 promoter), or a wound (e.g. Wun1 promoter) and/or pathogen inducible promoter may be used according to the present invention. Inducible promoters may have the advantage of a targeted switching on or off. In other embodiments, strong constitutively active promoter may be preferred to obtain a high level expression of a construct of interest.

In one embodiment, the first construct of a plant delivery system may comprise a first nucleotide molecule and the second construct of a plant delivery system may comprise a second nucleotide molecule comprising at least one terminator functional in a plant or part of a plant.

In one embodiment, the at least one terminator may be independently selected from a nopaline synthase terminator (NosT) (SEQ ID NO: 11), a 35S terminator (SEQ ID NO: 12), a Rbcs9 terminator, a Arabidopsis HSP terminator, Octopine Synthase terminator, an Ef1b terminator, a Tapal terminator, a Tapox3 terminator, a bvpa1 terminator, or a ZmEf terminator, or any combination thereof. Further terminators to be tested in accordance with the disclosure of the present invention for their suitability in the regulating termination in one of the individual constructs disclosed herein are available to the skilled person.

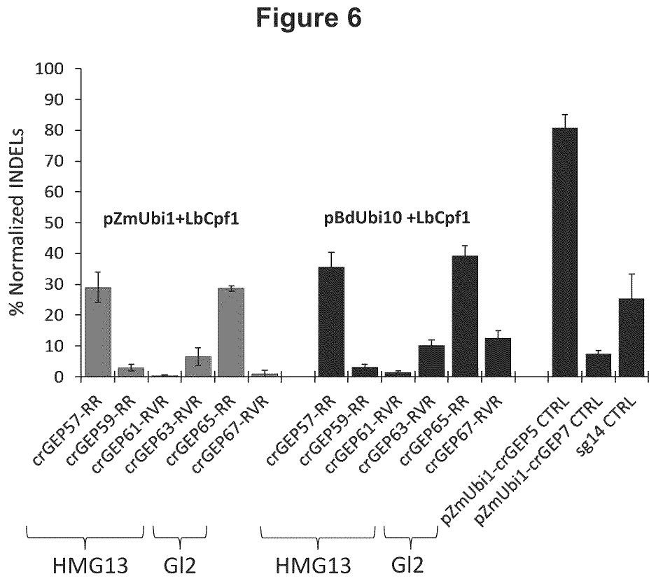

In certain embodiments, the scaffold RNA sequence, or a sequence encoding the same, of an LbCpf1 construct, or of a plant delivery system of the present invention may be selected from SEQ ID NO: 29 or 30, or a sequence having at least 95%, 96%, 97%, 98%, or 99% identity over the whole length of SEQ ID NO: 29 or 30. As it is known to the skilled person, scaffold sequences will inherently vary for individual Cpf1 effectors. Other scaffold sequences for other Cpf1 effectors in addition to Lachnospiraceae bacterium ND2006 (LbCpf1; pY016), including Francisella tularensis subsp. novicida U112 (FnCpf1; pY004), Lachnospiraceae bacterium MC2017 (Lb3Cpf1; pY005), Butyrivibrio proteoclasticus (BpCpf1; pY006), Peregrinibacteria bacterium GW2011_GWA_33_10 (PeCpf1; pY007), Parcubacteria bacterium GWC2011_GWC2_44_17 (PbCpf1; pY008), Smithella sp. SC_K08D17 (SsCpf1; pY009), Acidaminococcus sp. BV3L6 (AsCpf1; pY010), Lachnospiraceae bacterium MA2020 (Lb2Cpf1; pY011), Candidatus Methanoplasma termitum (CMtCpf1; pY012), Eubacterium eligens (EeCpf1; pY013), Moraxella bovoculi 237 (MbCpf1; pY014), Leptospira inadai (LiCpf1; pY015), Lachnospiraceae bacterium ND2006 (LbCpf1; pY016), Porphyromonas crevioricanis (PcCpf1; pY017), Prevotella disiens (PdCpf1; pY018), or Porphyromonas macacae (PmCpf1; pY09), are known to the skilled person (cf. Zetsche et al., 2015, supra, FIG. 6B).

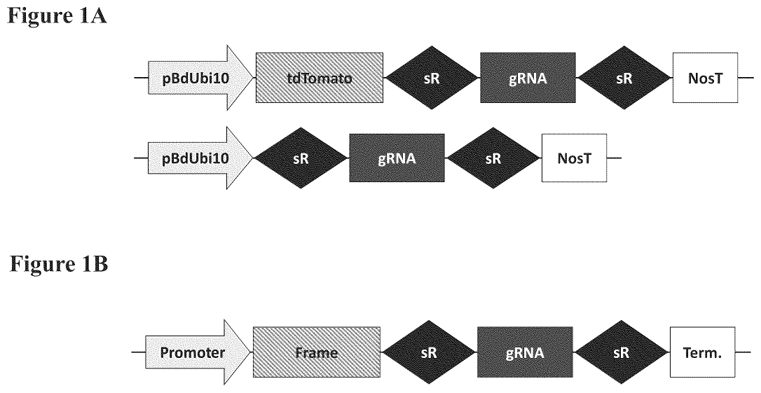

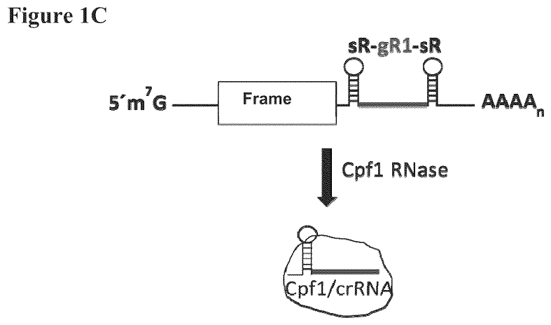

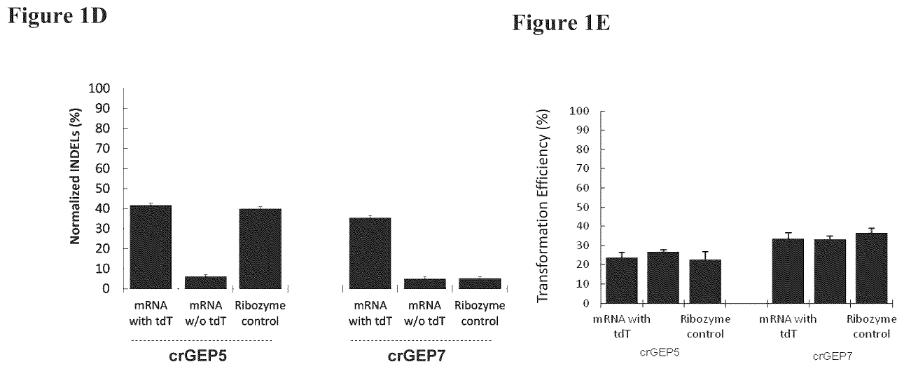

In one embodiment, a single scaffold RNA sequence, or a sequence encoding the same, may be used, for example, located in the 5′ direction of a sequence encoding a gRNA or a crRNA, preferably in embodiments relying on the provision of embedded gRNA(s)/crRNA(s) of the present invention. In other embodiments, more than one scaffold RNA sequence, or a sequence encoding the same, may be used, e.g., for a multiplexing approach simultaneously targeting more than one target site and thus using more than one gRNA/crRNA. In this setting, each gRNA/crRNA encoding sequence will be preceded by a scaffold RNA sequence. In certain embodiments, the sequence encoding a gRNA/crRNA in a construct of the present invention may comprise at least one RNA scaffold sequence located in the 5′ direction, and/or at least one additional RNA scaffold sequence located in the 3′ direction. A “scaffold RNA sequence” in the context of at least one Cpf1 guide RNA system of the present invention thus implies a recognition sequence for a Cpf1 enzyme, or a catalytically active fragment thereof, which allows the activation of the inherent RNA processing function of a Cpf1 molecule further detailed below to excise at least one gRNA/crRNA from an embedded mRNA construct carrying at least one cognate scaffold RNA sequence of the Cpf1 enzyme, or the catalytically active fragment thereof (see FIG. 1C).

In one embodiment of the various aspects of the present invention, the at least one Cpf1 enzyme or an active fragment thereof, or a nucleic acid sequence encoding the same, is selected from any one of SEQ ID NOs: 13 to 16, or a sequence having at least 60%, 65%, 70%, 75%, 80%, 85%, 90%, 91%, 92%, 93%, 94%, 95%, 96%, 97%, 98%, 99% or more sequence identity over the whole length of the respective nucleic acid sequence of SEQ ID NOs: 13 or 14, or a sequence having at least 90%, 91%, 92%, 93%, 94%, 95%, 96%, 97%, 98%, or 99% identity over the whole length of the respective amino acid sequence of SEQ ID NOs: 15 or 16, respectively.

In further embodiments, another CRISPR effector nuclease, or the sequence encoding the same may be used. Sequences for further CRISPR effector nucleases can be obtained from publicly available databases (as permanent sequence links) Francisella tularensis subsp. novicida U112 (FnCpf1; pY004)) benchling.com/s/0xgyNBMK/edit, Lachnospiraceae bacterium MC2017 (Lb3Cpf1; pY005) benchling.com/s/Oo2fP2pu/edit, Butyrivibrio proteoclasticus (BpCpf1; PY006) benchling.com/s/b9izZiQ3/edit, Peregrinibacteria bacterium GW2011_GWA_33_10 (PeCpf1; pY007) benchling.com/s/JcCPhiwB/edit, Parcubacteria bacterium GWC2011_GWC2_44_17 (PbCpf1; pY008) benchling.com/s/yAlizdZH/edit, Smithella sp. SC_K08D17 (SsCpf1; pY009) benchling.com/s/bUG7ykgA/edit, Acidaminococcus sp. BV3L6 (AsCpf1; pY010) benchling.com/s/wXO8WZJ7/edit, Lachnospiraceae bacterium MA2020 (Lb2Cpf1; pY011) benchling.com/s/R9HC1JbY/edit, Candidatus Methanoplasma termitum (CMtCpf1; pY012) benchling.com/s/ZC673QLc/edit, Eubacterium eligens (EeCpf1; pY013) benchling.com/s/0DPsrCES/edit, Moraxella bovoculi 237 (MbCpf1; pY014) benchling.com/s/bCMe5bll/edit, Leptospira inadai (LiCpf1; pY015) benchling.com/s/pqFOk5Rn/edit, Lachnospiraceae bacterium ND2006 (LbCpf1; pY016) benchling.com/s/HVlyGqQs/edit, Porphyromonas crevioricanis (PcCpf1; pY017) benchling.com/s/wDDn4cBS/edit, Prevotella disiens (PdCpf1; pY018) benchling.com/s/B8eGa0Ky/edit, Porphyromonas macacae (PmCpf1; pY09) benchling.com/s/NDZsqgFs/edit.

In a further embodiment of the various aspects disclosed herein, the at least one Cpf1 enzyme, or an active fragment thereof, or a nucleic acid sequence encoding the same, comprises at least one mutation in comparison to a wild-type sequence (SEQ ID NO: 16) resulting in an altered PAM recognition, preferably wherein the at least mutation is selected from G532R/K595R (cf. SEQ ID NO: 40), or G532R/K538V/Y542R (cf. SEQ ID NO: 41), or another Cpf1 sequence carrying the respective mutation in comparison to the wild-type sequence of SEQ ID NO: 16. As detailed above, PAM recognition by any naturally occurring or recombinant CRISPR system due to the interplay of a CRISPR effector and a tracr/crRNA or an artificial gRNA is crucial for the site-specificity of GE event effected. Therefore, altering PAM recognition properties of a CRISPR system is of high relevance to increase the targeting range of a recombinant CRISPR system.

Further mutations within various CRISPR effector nucleases may be selected from (data presented as “accession number”, “organism name”, “specific mutation”) WP_013282991 Butyrivibrio proteoclasticus BpCpf1 R527 E535 N540 K590, WP_044910712 Lachnospiraceae bacterium MC2017 Lb3Cpf1 N520 E528 K533 K582, KKR91555 Candidatus Falkowbacteria bacterium GW2011_GWA2_41_14 E633 K639 Y643 G705, KKP36646 Candidatus Peregrinibacteria bacterium GW2011_GWA2_33_10 PeCpf1 G623 K629 N633 K703, KKQ36153 candidate division WS6 bacterium GW2011_GWA2_37_6 G568 Q574 K578 R619, EKE28449 uncultured bacterium (gcode 4) T552 K558 R562 R615, KKQ38174 Candidatus Roizmanbacteria bacterium GW2011_GWA2_37_7 N592 K598 N602 K660, KKT48220 Parcubacteria group bacterium GW2011_GWC2_44_17 PbCpf1 K624 K630 F634 R689, WP_005398606 Helcococcus kunzii D554 K560 N564 N614, WP_028830240 Proteocatella sphenisci K483 K489 N493 K551, WP_015504779 Candidatus Methanomethylophilus alvus D515 K521 N525 K577, CUP14506 Lachnospira pectinoschiza S548 K554 N558 K614, CUM80100 Eubacterium rectale D529 K535 N539 K594, WP_012739647 Eubacterium eligens EeCpf1 N535 K541 N545 K601, AIZ56868 Candidatus Methanoplasma termitum CMtCpf1 N528 K534 Y538 R591, WP_037975888 Synergistes jonesii K539 K545 N549 K602, WP_021736722 Acidaminococcus sp. BV3L6 AsCpf1 S542 K548 N552 K607, WP_031492824 Succinivibrio dextrinosolvens E564 K570 C574 K629, WP_018359861 Porphyromonas macacae PmCpf1 S559 K565 N569 K623, WP_050786240 Prevotella disiens T588 N600 Y604 K674, WP_027407524 Anaerovibrio sp. RM50 A525 N531 N535 K594, KDN25524 Moraxella bovoculi 237 MbCpf1 N576 K582 N586 K637, AJI61006 Francisella tularensis subsp. novicida U112 FnCpf1 N607 K613 N617 K671, KUJ74576 Thiomicrospira sp. XS5 S575 K581 N585 K658, WP_051666128 Lachnospiraceae bacterium ND2006 (*) LbCpf1 G550 K556 Y560 K613, WP_027109509 Lachnospiraceae bacterium NC2008 G511 K517 C521 K574, WP_027216152 Butyrivibrio fibrisolvens D510 N516 Y520 N573, WP_028248456 Pseudobutyrivibrio ruminis N511 K517 N521 K574, WP_049895985 Oribacterium sp. NK2B42 D528 K534 N538 K591, WP_035798880 Butyrivibrio sp. NC3005 N512 K518 N522 K575, WP_044919442 Lachnospiraceae bacterium MA2020 Lb2Cpf1 N512 K518 N522 K575, WP_044910713 Lachnospiraceae bacterium MC2017 C537 K543 Y547 K599, WP_020988726 Leptospira inadai LiCpf1 K580 R586 N590 R644, WP_016301126 Lachnospiraceae bacterium COE1 D545 K551 N555 R608, KIE18657 Smithella sp. SC_K08D17 SsCpf1 G561 K567 N571 K625, WP_014085038 Flavobacterium branchiophilum N588 K594 Y598 K649, WP_045971446 Flavobacterium sp. 316 N586 K592 Y596 K647, KXB38146 Bacteroidales bacterium KA00251 A550 K556 N560 K613, WP_036890108 Porphyromonas crevioricanis PcCpf1 S575 K581 N585 K641, WP_044110123 Prevotella brevis D541 K547 N551 K603, WP_009217842 Bacteroidetes oral taxon 274 D564 K570 N574 K628, WP_006283774 Prevotella bryantii G566 K572 N576 K629, or WP_024988992 Prevotella albensis G561 K567 C571 K624 (cf. Gao et al., Nat. Biotech. doi: 10.1038/nbt.3900).

In one embodiment of the present invention, the at least one Cpf1 enzyme, or an active fragment thereof, or a nucleic acid sequence encoding the same, may comprise at least one mutation in comparison to a wild-type sequence (SEQ ID NO: 16) resulting in an altered PAM recognition, wherein the altered PAM recognition is a recognition of a TYCV, or of a TATV PAM sequence. The present invention provides evidence that the use of these alternative PAM variants increases the range of target site recognition across diverse sequence loci in plant genomes (see FIG. 3 and SEQ ID NOs: 35, 36 and 38-41).

In one embodiment according to the various aspects of the present invention, the at least one Cpf1 enzyme or an active fragment thereof, or a nucleic acid sequence encoding the same, and/or the at least one Cpf1 guide RNA system, or a nucleic acid sequence encoding the same, may be provided as at least one vector construct, or they may be provided as at least one linear construct.

In a further embodiment according to the various aspects of the present invention, the at least one Cpf1 guide RNA system may comprises at least two guide RNAs, wherein the at least two guide RNAs are separated by a nucleotide sequence comprising direct repeats. Therefore, in one aspect, the present invention relates to the delivery of multiple Cpf1 gRNAs, e.g. using a ribozyme delivery system as disclosed herein. This aspect of the present invention combines the advantages of the ribozyme delivery system with the finding that Cpf1 is able to process its own gRNAs from an array format (Zetschke et al. Nat Biotechnol (2017) 35 (1): 31-34) which was not previously shown to work in plant cells. Specifically, the invention relates to the delivery of at least two crRNAs in array format spaced by mature direct repeat sequences all of which are flanked by the HH and plant-derived HDR-like ribozyme sequences.

In a further embodiment according to the various aspects of the present invention, the at least one Cpf1 guide RNA system, preferably the guide RNA system of a plant delivery system, may comprise at least two Cpf1 guide RNAs, wherein the at least two Cpf1 guide RNAs are separated by a nucleotide sequence comprising direct repeats. This architecture may be specifically suitable for multiplexing approaches targeting more than one genomic target sequence of interest to be edited/modified. “Direct repeats” are a type of genetic sequence that consists of two or more repeats of a specific sequence. In other words, the direct repeats are nucleotide sequences present in multiple copies. Generally, a direct repeat occurs when a sequence is repeated with the same pattern downstream. There is no inversion and no reverse complement associated with a direct repeat. It may or may not have intervening nucleotides. In the constructs of the present invention, direct repeats may be used as spacers to link gRNA/crRNA encoding sequences in a construct of interest. In one embodiment of the present invention, the at least two gRNA/crRNA encoding sequences which are linked via direct repeats further comprise scaffold RNA sequences at their 5′ ends which function as recognition sites for a Cpf1 enzyme. In a particular embodiment of the present invention, a LbCpf1 scaffold RNA sequence, or a sequence encoding the same, is set forth in SEQ ID NO: 29 or 30. This sequence may comprise variations in the non-repeat positions. The sequence may be different depending on the Cpf1 effector enzyme used. The sequence of SEQ ID NO: 29 or 30 has been successfully used for multiplexing experiments using either a ribozyme construct or an embedded mRNA construct of the present invention. In multiplexing experiments direct repeat sequences are used as spacer sequences for separating either individual crRNA encoding sequences (comprising at least one scaffold RNA sequence) from each other, or to separate a HH ribozyme encoding sequence from a crRNA encoding sequence (comprising at least one scaffold RNA sequence), or for separating a crRNA encoding sequence (comprising at least one scaffold RNA sequence) from any other element of a vector construct, e.g., a terminator or another regulatory sequence, or a nuclear localization signal, or from another coding sequence.

In one embodiment according to the various aspects of the present invention, at least one construct of a plant delivery system of the present invention will comprise at least one nuclear localization sequence (NLS). In embodiment, where at least two separate constructs are used, each construct will comprise at least one NLS. Suitable NLS sequences may be selected from a SV40 NLS (SEQ ID NO: 42), or a nucleoplasmin NLS (SEQ ID NO: 43). For example, a NLS may be placed 5′, and another NLS may be placed 3′ flanking the coding region of a Cpf1 gene, or a construct encoding at least one gRNA or crRNA of the present invention. The skilled person is aware of further NLS sequences which may be used in accordance with the teaching of the present invention.

The various modular plant delivery systems of the present invention can be particularly suitable for transient expression approaches in a plant cell or cellular system. Therefore, the elements of at least one vector construct will not be stably integrated into the genome of a cell of interest. This can be a huge advantage from a regulatory point of view, but additionally for practical reasons, as the transient expression allows much shorter turnaround cycles in product development to obtain successfully edited homozygous cells and plants being vector and backbone free.

In certain aspects, the present invention provides plant-derived Hepatitis Delta Virus (HDV)-like ribozyme sequences strongly improving the efficiency of CRISPR-based systems by providing a component for the delivery of a guide RNA system, e.g., a Cas or a Cpf1 guide RNA system, comprising at least one gRNA or crRNA.

Besides the improvement of Cpf1 nuclease delivery/activity, further aspects of the present invention relate to improved strategies of Cpf1 guide RNA (gRNA) delivery.

Cpf1 gRNA delivery strategies have shown very high indel activity at multiple target sites in corn cells (see e.g. Tang et al. (2017) Nature Plants 3, 17018). In this approach, the sequence of the target gRNA is cloned in between two autocatalytic ribozyme sequences, i.e. a Hammerhead ribozyme (HH) sequence on the 5′-end of the construct and a Hepatitis Delta virus (HDV) ribozyme sequence at the 3′-end of the construct. A prototypic HDV ribozyme sequence, or a sequence encoding the same, is shown in SEQ ID NO: 27 or 28, respectively. More than 10 genomic loci have been targeted using this strategy with high indel frequencies/activity. One major drawback of this technology, however, is the use of human pathogen-derived HDV ribozyme sequences that might hamper the development of agricultural and/or food products due to regulatory concerns.

It was surprisingly and in clear contrast to the teaching of the prior art found that a plant-derived HDV-like ribozyme sequence has activity of in vitro or in vivo when using a plant-derived HDV-like ribozyme in a recombinant way. The data presented herein indicate that plant-derived HDV-like ribozyme sequences could potentially replace the original HDV sequences in gRNA ribozyme delivery systems and beyond thereby avoiding the use of human pathogen-derived sequences and time consuming and expensive regulatory issues in product development in any CRISPR system relying on various effector nucleases to properly transport, transcribe and process gRNAs or, for Cpf1 systems, crRNAs.

The present invention thus provides, in one aspect, a ribozyme construct comprising at least one plant-derived Hepatitis Delta Virus (HDV)-like ribozyme sequence, or the sequence encoding the same. The plant-derived Hepatitis Delta Virus (HDV)-like ribozyme sequence can be used together with a plant delivery system of the present invention, or it can be used in a eukaryotic cell, going beyond the use as cargo for any gRNA or crRNA, particularly for applications where the use of a hepatitis virus derived ribozyme system might complicate regulatory procedures.

A “ribozyme” as used herein is an RNA molecule that—in its natural environment—catalyses the cleavage and/or ligation of another RNA molecules. This capacity of ribozymes has, for example, also huge applications for therapy for the inactivation of deleterious genes and the repair of mutated genes involved in many disease states. Hepatitis delta virus (HDV) is a small, ˜1,700-nucleotide (nt), single-stranded RNA virus first isolated from human hepatocytes infected with hepatitis B virus.1 HDV harbors two structurally related self-cleaving ribozymes in its genome, one in the genomic and one in the complementary, antigenomic strand. Like other small self-cleaving ribozymes, these RNAs catalyze a transesterification reaction, promoting a nucleophilic attack by a 2′ hydroxyl on the adjacent phosphate and yield both a 2′-3′ cyclic phosphate and a liberated 5′ hydroxyl. The HDV antigenomic ribozyme was identified by in vitro transcription of cloned HDV genome (Sharmeen et al, 1988, J. Virol., 62:2674-9). The genomic RNA is a template for the synthesis of the concatemers of antigenomic RNA, which is then self-cleaved by its cis-ribozyme followed by host-aided ligation to generate circular, monomer length molecules. The circular “antigenomic” RNAs then serve as the templates for the genomic RNA synthesis followed by the similar self-cleaving and ligation processes.

The structures of the HDV ribozymes consist of five paired (P) regions that form two coaxial stacks (P1 stacks on P1.1 and P4, while P2 stacks on P3), which are linked by single-stranded joining (J) strands J1/2 and J4/2 (cf. Webb and Lupták, 2011, RNA biology, 8:5, 719-727). The genomic ribozyme was shown to self-cleave. The cleavage site for genomic ribozyme is between positions 685 and 686, whereas in the antigenomic RNA it is between positions 900 and 901.4 Both ribozymes require divalent metal ions, such as Mg²⁺, Mn²⁺ or Ca²⁺, for efficient catalysis and exhibit a drastically lower activity in monovalent ions. The cleavage site of the antigenomic ribozyme is 33 nts downstream of the polyadenylation site of the mRNA that encodes HDV's only protein, the delta antigen (Webb and Lupták, 2011, supra).

HDV ribozymes can be converted into trans-active forms by bisection of the J1/2 and/or L4 regions to make those molecules available for applications in molecular biology. Although splitting the ribozymes in the L4 loop results in more extensive base pairing, which increases binding specificity and allows incorporation of modified nucleotides in the active site, the J1/2-bisected molecule preserves the ribozyme core and recognizes a target strand exclusive of any catalytic components. The design of J1/2-split HDV molecule has led to ribozymes that could cleave target RNAs in vitro and in vivo (Kawakami et al., 1996, FEBS Lett., 394:132-6). Still, HDV represents a ribozyme derived from a human pathogen so that there is a great need in identifying and optimizing ribozyme sequences originating from a non-pathogenic organism.

Based on excessive genome sequencing and structure-related searches, potential candidates for ribozymes suitable as recombinant molecular tools have been identified. Still, no ribozyme originating from a plant and having a high rate constant and specific activity in vitro and thus being suitable for recombinant trans-activating applications has been identified and optimized. Particularly, no plant-derived ribozyme has been recombinantly used for genome editing purposes in combination with a plant-optimized CRISPR system to synergistically increase the successful outcome of a GE event in a plant genome even at difficult to access target sites by constructing a complex vector relying on a plant-derived HDV like structure system for optimum performance in a plant cell.

In one embodiment of the various aspects of the present invention, there is provided a plant delivery system, wherein the Hammerhead ribozyme sequence, or a sequence encoding the same, of the Cpf1 guide RNA system may be selected from SEQ ID NO: 17 or 18, and/or wherein the plant-derived Hepatitis Delta Virus (HDV)-like ribozyme sequence, or a sequence encoding the same, is selected from any one of SEQ ID NOs: 19 to 26, or a sequence having at least 95%, 96%, 97%, 98%, or 99% identity over the whole length of any one of SEQ ID NOs: 17 to 26.

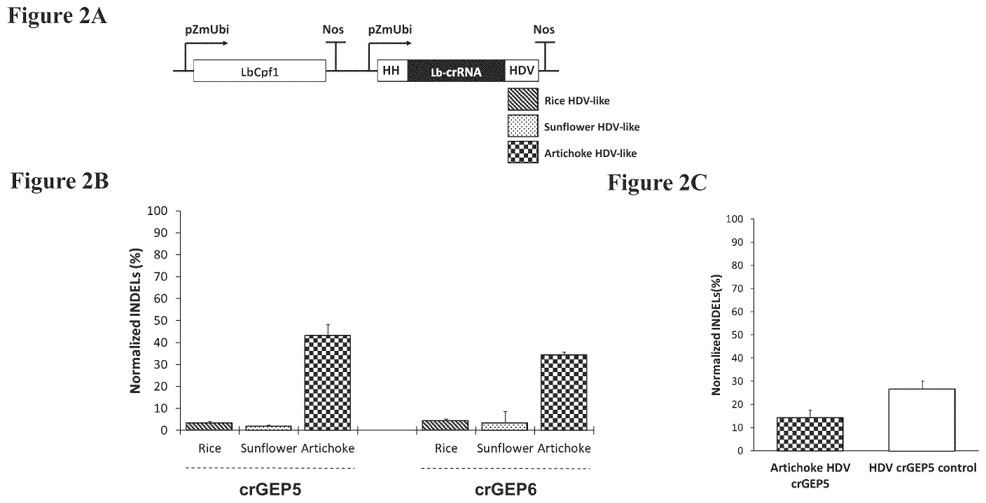

As demonstrated in Example 4 and FIG. 2, the plant-derived HDV like ribozymes of the present invention demonstrate high indel activities and are suitable to replace hepatitis delta virus derived sequences for constructing recombinant ribozyme systems. This make the plant-derived HDV like sequences of the present invention an attractive target for a variety of applications in molecular biology suitable for use in a variety of different cellular systems, comprising prokaryotic and eukaryotic cells or systems.

Furthermore, the plant-derived HDV like ribozymes can be advantageously used in a plant delivery system as disclosed herein. Suitably designed vector systems for providing a ribozyme flanked crRNA for a plant delivery system are provided in SEQ ID NOs: 45 to 48). SEQ ID NO: 44 further provides a ribozyme strategy vector comprising a conventional HDV ribozyme sequence, which was used as control herein. Notably, As it is known to the skilled person, certain variations of the vector backbones can be effected not influencing the coding sequence of a Cpf1 guide RNA system, or a nucleic acid sequence encoding the same, of the present invention. Such variations are encompassed by the sequences provided herein. As illustrated in Example 9 below, the plant-ribozyme based systems of the present invention are even suitable for multiplexing, i.e., the targeted modification of more than one genomic target site, preferably for a CRISPR-based site-directed GE approach, more preferably for Cpf1-based GE in a plant. FIGS. 2A, 3A and 5A show architectures for LbCpf1 genome editing constructs relying on a ribozyme Cpf1 guide RNA system of the present invention. Notably, the ribozyme system may be provided on a single transcript construct together with a CRISPR effector nuclease of interest. As further disclosed herein, it may be advantageous to provide a CRISPR effector nuclease and a guide RNA system, including a Cpf1 guide RNA system, on separate constructs under the control of separate regulatory elements to optimize the expression and thus the availability of the effector nuclease and the cognate gRNA. Providing separate constructs may allow a better fine-tuning of regulation of the plant delivery system. The various ribozyme constructs of the present invention, whether provided as single transcript unit, or as separate expression constructs together with further constructs, for example, encoding a CRISPR effector nuclease, including a Cas9 or a Cpf1 effector, have the advantage that they do not represent T-DNA vector, i.e., vectors for Agrobacterium mediated transformation comprising a right boarder and a left boarder sequence subject to regulatory hurdles.

In one embodiment of the present invention, to be used alone or preferably in combination in a plant delivery system of the present invention, there is provided a system, wherein the at least one Cpf1 guide RNA, or the nucleotide sequence encoding the same, which is (i) flanked by a Hammerhead ribozyme sequence at the 5′ end and a plant-derived Hepatitis Delta Virus (HDV)-like ribozyme sequence at the 3′ end, may further comprise a scaffold

RNA sequence, or a sequence encoding the same, at the 5′ end; and/or which is (ii) embedded within the non-coding region, preferably the 3′ untranslated region (UTR), of the sequence encoding a frame sequence, may further comprise a scaffold RNA sequence, or a sequence encoding the same, at the 5′ and 3′-end.

In one embodiment, the Hammerhead ribozyme sequence and/or the plant-derived Hepatitis Delta Virus (HDV)-like ribozyme sequence may be individually replaced against another HH or plant-derived HDV sequence.

Thus, a further aspect of the present invention relates to a guide RNA system, preferably a Cpf1 gRNA system, wherein the construct is flanked by autocatalytic ribozyme sequences in which the HDV ribozyme at the 3′-end of the construct is replaced with a plant-derived HDV-like ribozyme sequence. Three plant-derived HDV-like ribozyme sequences from Rice, Sunflower and Artichoke were identified and tested for their suitability as a HDV replacement. Surprisingly, it was found out that plant-derived HDV-like ribozymes, in particular Artichoke HDV-like ribozymes, as further detailed herein can replace HDV ribozyme sequences at the 3′-end of the construct to obtain indel activity similar to the ones observed with HDV ribozyme sequences (see FIG. 2) or even better than the prior art sequences. This was neither known nor expected from the known prior art so far not showing any activity of plant-derived HDV-like sequences.

In a further aspect, to be used alone or in combination in a plant delivery system of the present invention, there is provided a at least one Cpf1 guide RNA system, or a nucleic acid sequence encoding the same, the at least one Cpf1 guide RNA system comprising at least one Cpf1 guide RNA specific for a genomic target sequence of interest in a plant or part of a plant; wherein the at least one Cpf1 guide RNA, or the nucleic acid sequence encoding the same, may be embedded within a non-coding region, preferably a 3′ untranslated region (UTR), of a sequence encoding a frame sequence.

To further improve gRNA delivery in a ribozyme-independent manner, for applications, where this might not represent a suitable strategy due to poor GE rates, an mRNA based expression system for gRNA/crRNA was established. Recently, it was shown that delivery of gRNA using an mRNA based system in mammalian cells could be used to improve gRNA delivery to the cells (Zhong et al. (2017) Nat Chem Biol. 13 (8): 839-841). This report demonstrated indel activity from expression of multiple gRNAs embedded in an mRNA construct expressed from a Pol II promoter. Importantly, however, this system was so far not shown to work in plant cells.

According to a further aspect, the present invention therefore relates to at least one LbCpf1 gRNA/crRNA embedded in the 3′ untranslated region of an mRNA construct and its use for improved delivery of gRNA at one or more genomic loci in plant cells. In particular, the present invention relates to embedding a CRISPR gRNA scaffold and target sequence within the 3′ untranslated region of a reporter gene mRNA sequence in one construct, since it was shown that Cpf1 nuclease activity can process gRNA from mRNA constructs (Fonfara et al., 2016 Nature 532 7600; 517-21). The inventors surprisingly found out that the use of the mRNA delivery system could significantly improve the accessibility of genomic target sequences which are only poorly modified by using the ribozyme system (see FIG. 1, crGEP7 FIG. 1E). The mRNA based guide RNA delivery system therefore provides a novel approach to efficiently modify genomic target sequences in a plant cell which otherwise would be difficult to access, or not targetable at all. Notably, this aspect can be used alone for a variety of different genome editing purposes using a CRISPR effector nuclease, nickase or nuclease-dead variant of interest, as it serves the central purpose of embedding, transporting and providing at least one functional guide RNA suitable for any kind of CRISPR-based GE assay. The mRNA embedding as enclosed herein can be advantageously combined in a plant delivery system of the present invention.

The above aspect can be advantageously combined with the various aspects and embodiments relating to at least one Cpf1 enzyme or an active fragment thereof, or a nucleic acid sequence encoding the same, or relating to a plant delivery system of the present invention, and/or relating to at least one Cpf1 guide RNA system, to obtain a significantly enhanced efficiency rate of CRISPR/Cpf1 based genome editing (GE) in a plant, even when targeting difficult to access target sites, preferably in a plant genome. The gRNA embedding aspects as disclosed herein can, however, be used to deliver at least one or a multitude of gRNA(s)/cRNA(s) for optimizing gRNA/crRNA availability and in turn GE efficiency in any eukaryotic or prokaryotic genome using any CRISPR/Cpf1 based effector nuclease due to the inherent activity of Cpf1 to process precursor CRISPR RNA. I.e., the above aspect relies on the dual use of the Cpf1 capability to process its own crRNA and in turn to recognize and cleave a genomic target sequence of interest (cf. Fonfara et al., 2016, supra).

In one embodiment of the present invention, the sequence encoding a frame sequence may be selected from a translatable or non-translatable sequence being selected from a marker gene, including an antibiotic marker or a fluorescent marker, a gene encoding a structural protein, a gene encoding an RNA species, an internal ribosomal entry site (IRES) encoding sequence, or at least part of the aforementioned sequences. The term “frame sequence” according to the present disclosure may thus be construed broadly to comprise any naturally occurring or artificial sequence that is transcribable when introduced into a target cell or cellular system of interest. Marker genes, including fluorescent marker encoding genes, for example, tdTomato (e.g., comprised by SEQ ID NO: 33 or 31), mNeon Green (e.g., comprised by SEQ ID NO: 37), GFP or eGFP and variants thereof, luciferase and variants thereof, mOrange and variants thereof, mCherry or variants thereof, or any other commercially available marker gene, can have certain advantages as markers, as the activation of the embedded at least one gRNA/crRNA and/or a multitude of gRNA(s)/crRNA(s) can be monitored by quantifying the fluorescence in a target cell or cellular system of interest. In principal, without wishing to be bound by theory, any coding or non-coding RNA could serve as embedding tool for at least one gRNA/crRNA as long as the respective sequence is long enough to guarantee that it can be properly transcribed by RNA polymerase II and/or recognized by a Cpf1 enzyme of interest.

In one preferred embodiment, the at least one gRNA/crRNA may be embedded within a non-coding region, preferably a 3′ untranslated region (UTR), or a 5′ UTR, of the sequence encoding a frame sequence, the frame sequence per se being a coding or non-coding region.

In another embodiment, the at least one gRNA/crRNA may be embedded within a coding region, in case that the frame sequence represents a sequence encoding a RNA or protein.

In one embodiment, the sequence encoding a frame sequence may be thus selected from a gene, or part of a gene, encoding an RNA, for example an RNA taking a defined secondary or tertiary structure upon transcription.

In one embodiment, the sequence encoding a frame sequence may be selected from any one of SEQ ID NOs: 31, 32, 13, 14, 38, 39, 72, 73, 74, 75, 76, 157, or 158, or a sequence having at least 85%, 86%, 87%, 88%, 89%, 90%, 91%, 92%, 93%, 94%, 95%, 96%, 97%, 98%, or 99% identity over the whole length of the respective sequence of SEQ ID NOs: 31, 32, 13, 14, 38, 39, 72, 73, 74, 75, 76, 157, or 158.

In one particular embodiment, the sequence encoding a frame sequence may be selected from any one of SEQ ID NOs: 13 or 14, or a sequence having at least 85%, 86%, 87%, 88%, 89%, 90%, 91%, 92%, 93%, 94%, 95%, 96%, 97%, 98%, or 99% identity over the whole length of the respective sequence of SEQ ID NOs: 13 or 14, or a sequence portion of any one of SEQ ID NOs: 13 or 14 encoding an active fragment of an LbCpf1 enzyme of the present invention. In certain embodiments, the frame sequence may be selected from a sequence encoding a Cpf1 enzyme or an active fragment thereof. These embodiments using the effector nuclease itself as frame sequence can be particularly suitable to reduce the complexity of the systems and in turn to increase the success rate. The Cpf1 enzyme or the active fragment thereof will thus serve a dual function in (i) activating its own at least one gRNA; (ii) interacting with the excised at least one gRNA to bind to a target sequence of interest to be cleaved and/or modified.

In one embodiment, the at least one Cpf1 guide RNA, or the nucleic acid sequence encoding the same, embedded within a coding or a non-coding region, preferably a 3′ untranslated region (UTR), of a sequence encoding a frame sequence, may comprise at least one scaffold RNA sequence as detailed above in the context of a plant delivery system of the present invention. The scaffold sequence will allow the recognition of the provided, or the transcribed, mRNA construct by at least one Cpf1 enzyme, or a catalytically active fragment thereof, so that the at least one Cpf1 enzyme, or the catalytically active fragment thereof can process its gRNA/crRNA properly. In embodiments, where a multiplexing is envisaged, more than one gRNA/crRNA individually flanked by at least one scaffold RNA sequence may be present. An exemplary mRNA embedded construct comprising two scaffold regions is shown in FIG. 1A. Using a plant delivery construct relying on a construct as shown in FIG. 1A and further relying on a Cpf1 construct disclosed herein, on a separate construct, it could be demonstrated for the first time that a Cpf1 variant cleaves, processes and releases functional gRNA/crRNA in a plant cell from an embedded Cpf1 guide RNA system of the present invention which resulted in an unexpected high indel rate and thus GE efficiency (cf. Example 4 an FIG. 1D). Therefore, specifically combining a Cpf1 construct under the control of a suitable promoter and at least one Cpf1 guide RNA system, wherein the at least one Cpf1 guide RNA, or the nucleic acid sequence encoding the same, is embedded within a coding or a non-coding region, preferably a 3′ untranslated region (UTR), of a sequence encoding a frame sequence in the form of a plant delivery system of the present invention targets crGEP5 and 7 (Table 1 and FIG. 1D) with equivalent or even improved activity compared to a conventional ribozyme-delivered guide RNA control from the prior art (Tang et al., 2017), even when targeting difficult to access sites like crGEP7. The non-mRNA embedded guides clearly show highly diminished activities at those two sites.

In one embodiment, at least one Cpf1 guide RNA system, or a nucleic acid sequence encoding the same, is provided, wherein the at least one Cpf1 guide RNA, or the nucleic acid sequence encoding the same, is embedded within a non-coding region, preferably a 3′ untranslated region (UTR), of a sequence encoding a frame sequence, wherein the frame sequence is a sequence encoding a CRISPR nuclease enzyme, preferably a nucleic acid sequence encoding a Cpf1 enzyme or an active fragment thereof as disclosed herein. This arrangement may have the outstanding advantage of providing a CRISPR enzyme and the cognate at least one gRNA/crRNA on a single transcript for approaches, where the transformation or transfection of a single entity is favorable. Stability of the gRNA component is ensured by the fact that the gRNA/crRNA will only be activated as soon as the Cpf1 enzyme or the active fragment thereof will be available in a cell in functional form.

In a particular embodiment, a plant optimized promoter as disclosed above may be favorably used with the at least one Cpf1 guide RNA system, or a nucleic acid sequence encoding the same, embedded within a coding or within a non-coding region, preferably a 3′ untranslated region (UTR), of a sequence encoding a frame sequence. This may allow optimized transcription of the construct, particularly in a plant system, where a polymerase II promoter may fail.

In one embodiment, the use of two construct requires to deliver them separately by two individual vectors like Agrobacterium cultures, a process that does not guarantee that you will be able to deliver both T-DNAs into the same cell making the construct encoding a Cpf1 enzyme or an active fragment thereof will be provided on two separate constructs. The two construct approach has been highly efficient as evidenced by sometimes 80-90% normalized INDEL efficiency. Another advantage is that, for example, T-DNA in a one construct Agrobacterium system is delivered in very low dosage into a cell (1-5 copies) while in the approach using separate construct it is possible to deliver more than 10-100 copies per cell, which leads to higher likelihood of activity at the target site. Also advantageous is the fact that in the system of the present invention INDEL (insertion/deletion) activity can be detected from transient expression of the delivered plasmids with no integration. With T-DNA based vectors, for example, the likelihood of T-DNA integration is extremely high. Furthermore, a two construct approach also allows the fine-tuning of the expression of the two components (Cpf1 and cognate gRNA/crRNA) to get the best possible outcome from the system at various target sites, as both Cpf1 representing a protein and the cognate RNA portion inherently have different turnover times and stabilities. This applies for any Cpf1 guide RNA system, or a nucleic acid sequence encoding the same, of the present invention.

In one embodiment of the various aspects provided herein, the part of a plant may be selected from the group consisting of a plant cell, a plant tissue and a plant organ. leaves, stems, roots, emerged radicles, flowers, flower parts, petals, fruits, pollen, pollen tubes, anther filaments, ovules, embryo sacs, egg cells, ovaries, zygotes, embryos, zygotic embryos, somatic embryos, apical meristems, vascular bundles, pericycles, seeds, roots, gametophytes, spores and cuttings. In one specific embodiment, the plant or part of a plant may originate from a genus selected from the group consisting of Hordeum, Sorghum, Saccharum, Zea, Setaria, Oryza, Triticum, Secale, Triticale, Malus, Brachypodium, Aegilops, Daucus, Beta, Eucalyptus, Nicotiana, Solanum, Coffea, Vitis, Erythrante, Genlisea, Cucumis, Marus, Arabidopsis, Crucihimalaya, Cardamine, Lepidium, Capsella, Olmarabidopsis, Arabis, Brassica, Eruca, Raphanus, Citrus, Jatropha, Populus, Medicago, Cicer, Cajanus, Phaseolus, Glycine, Gossypium, Astragalus, Lotus, Torenia, Allium, or Helianthus. More preferably, the plant or part of a plant may originate from a species selected from the group consisting of Hordeum vulgare, Hordeum bulbusom, Sorghum bicolor, Saccharum officinarium, Zea spp., including Zea mays, Setaria italica, Oryza minuta, Oryza sativa, Oryza australiensis, Oryza alta, Triticum aestivum, Triticum durum, Secale cereale, Triticale, Malus domestica, Brachypodium distachyon, Hordeum marinum, Aegilops tauschii, Daucus glochidiatus, Beta spp., including Beta vulgaris, Daucus pusillus, Daucus muricatus, Daucus carota, Eucalyptus grandis, Nicotiana sylvestris, Nicotiana tomentosiformis, Nicotiana tabacum, Nicotiana benthamiana, Solanum lycopersicum, Solanum tuberosum, Coffea canephora, Vitis vinifera, Erythrante guttata, Genlisea aurea, Cucumis sativus, Marus notabilis, Arabidopsis arenosa, Arabidopsis lyrata, Arabidopsis thaliana, Crucihimalaya himalaica, Crucihimalaya wallichii, Cardamine nexuosa, Lepidium virginicum, Capsella bursa pastoris, Olmarabidopsis pumila, Arabis hirsute, Brassica napus, Brassica oleracea, Brassica rapa, Raphaus sativus, Brassica juncacea, Brassica nigra, Eruca vesicaria subsp. sativa, Citrus sinensis, Jatropha curcas, Populus trichocarpa, Medicago truncatula, Cicer yamashitae, Cicer bijugum, Cicer arietinum, Cicer reticulatum, Cicer judaicum, Cajanus cajanifolius, Cajanus scarabaeoides, Phaseolus vulgaris, Glycine max, Gossypium sp., Astragalus sinicus, Lotus japonicas, Torenia fournieri, Allium cepa, Allium fistulosum, Allium sativum, Helianthus annuus, Helianthus tuberosus and/or Allium tuberosum. Particularly preferred are Beta vulgaris, Zea mays, Triticum aestivum, Hordeum vulgare, Secale cereale, Helianthus annuus, Solanum tuberosum, Sorghum bicolor, Brassica rapa, Brassica napus, Brassica juncacea, Brassica oleracea, Raphanus sativus, Oryza sativa, Glycine max, and/or Gossypium sp.

In one specific aspect of the present invention, there is provided a host cell, wherein the host cell may comprise any of the plant delivery system as disclosed herein. The plant delivery system may be introduced into the host cell by any technique as disclosed herein, or as known to the skilled person in the field of plant molecular biology.

In one aspect, there is provided a plant, or a plant cell, tissue, organ or material, or a derivative or progeny thereof, which may comprise at least one of the plant delivery systems as disclosed herein.

In one embodiment of the various aspects disclosed herein, the genomic target sequence of interest to be modified as disclosed herein may be a difficult to access target site. A “difficult to access target site” as used herein implies a target site, which is hard to be modified by genetic engineering and genome editing techniques. Such a “difficult to access target site” may occur, for example, in condensed chromatin, highly GC rich areas and areas with high methylation coverage in a complex eukaryotic genome. Using the example of target crGEP7 as disclosed herein which showed low INDEL activity using a common ribozyme system (under 10%), it was possible to obtain >40% activity using a plant delivery system comprising an embedded mRNA system of the present invention. It is speculated that the processing of the gRNA within the mRNA context of the various constructs disclosed herein results in more efficient processing and allows for better binding to the CRISPR Cpf1 nuclease versus at least a conventional ribozyme system, where the nuclease does not have to process the gRNA out of the larger RNA.

In a further aspect of the present invention, there is provided a method for modifying a genomic target sequence of interest in a plant or part of a plant, wherein the method may comprise the steps of: (a) providing at least one Cpf1 enzyme or an active fragment thereof, or a nucleic acid sequence encoding the same; preferably, wherein the at least one nucleic acid sequence encoding the Cpf1 enzyme or an active fragment thereof is codon-optimized for the expression in the plant or part of the plant; and (b) providing at least one Cpf1 guide RNA system, or a nucleic acid sequence encoding the same, the at least one Cpf1 guide RNA system comprising at least one Cpf1 guide RNA specific for a genomic target sequence of interest in the plant or part of the plant; wherein the at least one Cpf1 guide RNA, or the nucleic acid sequence encoding the same, is (i) flanked by a Hammerhead ribozyme sequence at the 5′ and a plant-derived Hepatitis Delta Virus (HDV)-like ribozyme sequence at the 3′-end; and/or is (ii) embedded within a coding or within a non-coding region, preferably a 3′ untranslated region (UTR), of a sequence encoding a frame sequence (c) optionally: providing at least one repair template nucleic acid sequence, wherein the at least one repair template nucleic acid sequence is preferably flanked by one or more homology sequence(s) complementary to one or both adjacent region(s) of the genomic sequence of interest in the plant or part of the plant; (d) introducing the at least one Cpf1 enzyme or an active fragment thereof, or a nucleic acid sequence encoding the same from step (a); and introducing the at least one Cpf1 guide RNA system, or a nucleic acid sequence encoding the same from step (b) and optionally: introducing the at least one repair template nucleic acid sequence from step (c) into the plant or part of the plant; and (e) obtaining a plant or part of a plant, or a progeny thereof, comprising a modification in the genomic target sequence of interest.

In eukaryotic cells, genome integrity is ensured by robust and partially redundant mechanisms for repairing DNA DSBs caused by environmental stresses and errors of cellular DNA processing machinery. In most eukaryotic cells and at most stages of the respective cell cycle, the non-homologous end-joining (NHEJ) DNA repair pathway is the highly dominant form of repair. A second pathway uses homologous recombination (HR) of similar DNA sequences to repair DSBs. This pathway can usually be used in the S and G2 stages of the cell cycle by templating from the duplicated homologous region of a paired chromosome to precisely repair the DSB. However, an artificially-provided repair template (RT) with homology to the target can also be used to repair the DSB, in a process known as homology-directed repair (HDR) or gene targeting. By this strategy it is possible to introduce very precise, targeted changes in the genomes of eukaryotic cells.

A “homology sequence”, if present, may be part of the at least one RT of interest according to the various embodiments of the present invention, to be introduced to modify the genetic material of a cell or cellular system according to the present disclosure at a genomic target sequence or site of interest. Therefore, the at least one homology sequence is physically associated with the at least one RT within one molecule. As such, the homology sequence may be part of the at least one RT to be introduced and it may be positioned within the 5′ and/or 3′ position of the at least one RT of interest, optionally including at least one spacer nucleotide, or within the at least one RT sequence of interest to be introduced. As such, the homology sequence(s) serve as templates to mediate homology-directed repair by having complementarity to at least one region, the upstream and/or the downstream region, adjacent to the genomic target sequence or the predetermined location (said terms being used interchangeably herein) within the genetic material of a cell or cellular system to be modified. In certain embodiments, the RT may be further associated with another DNA and/or RNA sequence as mediated by complementary base pairing. In an alternative embodiment the RT may be associated with other sequence, for example, sequences of a vector, e.g., a plasmid vector, which vector can be used to amplify the RT prior to transformation. Furthermore, the RT may also be physically associated with at least part of an amino acid component, preferably a site-specific nuclease, more preferably a CRISPR nuclease, and most preferably a Cpf1 enzyme or an active fragment thereof. This configuration and association allows the availability of the RT in close physical proximity to the site of a DSB, i.e., exactly at the position a targeted GE event is to be effected to allow even higher efficiency rates. For example, the at least one RT may also be associated with at least one gRNA interacting with the at least one RT and further interacting with at least one portion of a CRISPR nuclease, e.g., a Cpf1 nuclease, as site-specific nuclease.

The one or more homology region(s) may each have a certain degree of complementarity to the respective region flanking the at least one predetermined location upstream and/or downstream of the double-strand break induced by the at least one site-specific nuclease, i.e., the upstream and downstream adjacent region, respectively. Preferably, the one or more homology region(s) will hybridize to the upstream and/or downstream adjacent region under conditions of high stringency. The longer the at least one homology region, the lower the degree of complementarity may be. The complementarity is usually calculated over the whole length of the respective region of homology. In case only one homology region is present, this single homology region will usually have a higher degree of complementarity to allow hybridization. Complementarity under stringent hybridization conditions will be at least 85%, at least 90%, at least 91%, at least 92%, at least 93%, at least 94%, at least 95%, at least 96%, and preferably at least 97%, at least 98%, at least 99%, or even 100%. At least in the region directly flanking a DSB induced (about 5 to 10 bp upstream and downstream of a DSB), complementarities of at least 98%, at least 99%, and preferably 100% should be present. Notably, as further disclosed herein below, the degree of complementarity can also be lower than 85%. This will largely depend on the target genetic material and the complexity of the genome it is derived from, the length of the nucleic acid sequence of interest to be introduced, the length and nature of the further homology arm or flanking region, the relative position and orientation of the flanking region in relation to the site at least one DSB is induced, and the like.

The term “adjacent” or “adjacent to” as used herein in the context of a predetermined location or a genomic target sequence of interest, and the one or more homology region(s) may comprise an upstream and a downstream adjacent region, or both. Therefore, the adjacent region is determined based on the genetic material of a cellular system to be modified, said material comprising the predetermined location.

There may be an upstream and/or downstream adjacent region near the predetermined location/the genomic target sequence of interest. For site-specific nucleases (SSNs), including CRISPR nucleases like Cpf1, Cas9, CasX or CasY, and variants thereof, leaving overhangs after double-strand break (DSB) induction, the predetermined location means the region between the cut in the 5′ end on one strand and the 3′-end on the other strand. The adjacent regions in the case of sticky end SSNs thus may be calculated using the two different DNA strands as reference. The term “adjacent to a predetermined location” thus may imply the upstream and/or downstream nucleotide positions in a genetic material to be modified, wherein the adjacent region is defined based on the genetic material of a cellular system before inducing a DSB or modification.

If present, the upstream adjacent region defines the region directly upstream of the 5′-end of the cutting site of a CRISPR nuclease of interest, preferably a Cpf1 nuclease, with reference to a predetermined location before initiating a double-strand break, e.g., during targeted genome engineering. Correspondingly, a downstream adjacent region defines the region directly downstream of the 3′-end of the cutting site of a SSN of interest with reference to a predetermined location before initiating a double-strand break, e.g., during targeted genome engineering. The 5′-end and the 3′-end can be the same, depending on the site-specific nuclease of interest.

In certain embodiments, it may also be favorable to design at least one homology region in a distance away from the DSB to be induced, i.e., not directly flanking the predetermined location/the DSB site. In this scenario, the genomic sequence between the predetermined location and the homology sequence (the homology arm) would be “deleted” after homologous recombination had occurred, which may be preferred for certain strategies as this allows the targeted deletion of sequences near the DSB. Different kinds of RT configuration and design are thus contemplated according to the present invention for those embodiments relying on a RT. RTs may be used to introduce site-specific mutations, or RTs may be used for the site-specific integration of nucleic acid sequences of interest, or RTs may be used to assist a targeted deletion.

A “homology sequence(s)” introduced and the corresponding “adjacent region(s)” can each have varying and different length from about 15 bp to about 15.000 bp, i.e., an upstream homology region can have a different length in comparison to a downstream homology region. Only one homology region may be present. There is no real upper limit for the length of the homology region(s), which length is rather dictated by practical and technical issues. According to certain embodiments, depending on the nature of the RT and the targeted modification to be introduced, asymmetric homology regions may be preferred, i.e., homology regions, wherein the upstream and downstream flanking regions have varying length. In certain embodiments, only one upstream and downstream flanking region may be present.

In one embodiment of the above method of the present invention, the at least one Cpf1 enzyme or an active fragment thereof, or a nucleic acid sequence encoding the same, and the at least one Cpf1 guide RNA system, or a nucleic acid sequence encoding the same, and optionally the at least one repair template nucleic acid sequence, may be provided on separate constructs, wherein the at least two separate constructs are introduced simultaneously, or subsequently. The use of separate constructs, whether introduced simultaneously, or subsequently may have the advantage of a better regulation of the expression and thus the availability of the respective effector RNA (gRNA/crRNA), DNA (RT) and/or Cpf1 enzyme such encoded.

In another embodiment, the at least one Cpf1 enzyme or an active fragment thereof, or a nucleic acid sequence encoding the same, and the at least one Cpf1 guide RNA system, or a nucleic acid sequence encoding the same, may be provided on a single transcript construct. This setting may be particularly suitable, when Agrobacterium based transformation is intended.

In yet another embodiment, the at least one Cpf1 enzyme or an active fragment thereof, and the at least one Cpf1 guide RNA system, and optionally at least one RT, may be provided as an ex vivo synthesized and pre-assembled complex. This setting may be particularly suitable to transfect a cell, e.g., a cell recalcitrant to transformation in one shot with a pre-assembled and thus fully functional complex without the need of the cellular machinery to express at least one construct.

In still another embodiment, the at least one Cpf1 enzyme or an active fragment thereof, or a nucleic acid sequence encoding the same, and the at least one Cpf1 guide RNA system, or a nucleic acid sequence encoding the same, may be provided on a multiplexing construct. This approach is particularly suitable in case more than one genomic target sequence of interest is intended to be modified simultaneously using more than one gRNA/crRNA (cf. Example 9).

According to all of the above embodiments, the RT may be transformed as an individual construct, or it may be transfected as a separate construct, or it may be synthesized and assembled with at least one of the other components of a plant delivery system of the present invention ex vivo before introducing a complex into a cell of interest.

In one specific embodiment of the above method, the molecules of step (a), (b) and optionally of step (c) may thus be provided as a plant delivery system as disclosed herein, wherein the plant delivery system may comprise a first nucleotide molecule comprising a nucleic acid sequence encoding the at least one Cpf1 enzyme or an active fragment thereof, and a second nucleotide molecule comprising a nucleic acid sequence encoding the at least one Cpf1 guide RNA system, wherein the first and the second molecule are provided on separate constructs, or wherein the first and the second molecule are provided on a single transcript construct.

In one embodiment of the methods of the present invention, (i) the at least one nucleic acid sequence encoding the Cpf1 enzyme or an active fragment thereof, and the nucleic acid sequence encoding at least one Cpf1 guide RNA system; or (ii) the first construct comprising the first nucleotide molecule and the second construct comprising the second nucleotide molecule, may each comprise at least one promoter functional in a plant or part of a plant.

In one embodiment of the above methods, the at least one promoter may be independently selected from a (p)BdUbi10 promoter (SEQ ID NO: 1, a (p)ZmUbi1 promoter (SEQ ID NO: 2), a (p)OsActin promoter (SEQ ID NO: 3), and a single or double 35S promoter (SEQ ID NO: 4), optionally including an ZmUbi1 intron, an BdUbi10 intron and/or an Adh1 intron, (SEQ ID NOs: 5 to 10, or 67), or any combination thereof, or a sequence having at least 90%, 91%, 92%, 93%, 94%, 95%, 96%, 97%, 98%, or 99% identity when compared over the whole length of the respective sequence of any one of SEQ ID NOs: 1 to 10, or 67.

As further disclosed herein, the choice of the promoter and the choice of different promoters for individual constructs may be crucial to obtain optimized expression of the respective effector sequences encoded by the constructs, or the plant delivery system (cf. Example 7).

In yet a further embodiment of the above methods, (i) the at least one nucleic acid sequence encoding the Cpf1 enzyme or an active fragment thereof, and the nucleic acid sequence encoding at least one Cpf1 guide RNA system; or (ii) the first construct comprising the first nucleotide molecule and the second construct comprising the second nucleotide molecule; may comprise at least one terminator functional in a plant or part of a plant, preferably wherein the at least one terminator is independently selected from a nopaline synthase terminator (NosT) (SEQ ID NO: 11), or a 35S terminator (SEQ ID NO: 12) or any combination thereof, or a sequence having at least 90%, 91%, 92%, 93%, 94%, 95%, 96%, 97%, 98%, or 99% identity when compared over the whole length of the respective sequence of any one of SEQ ID NOs: 11 or 12. Further terminators to be tested in accordance with the disclosure of the present invention for their suitability in the regulating termination in one of the individual constructs disclosed herein are available to the skilled person.

In one embodiment of the methods of the present invention, the nucleic acid sequence encoding the Cpf1 enzyme or an active fragment thereof is codon-optimized for expression in a plant or part of a plant. Specific codon-optimization may significantly enhance the performance of a CRISPR-based nuclease naturally originating from non-plant organisms so that the CRISPR effector, preferably a Cpf1 effector, can be functionally transcribed and translated in a plant cell of interest.

In another embodiment of the methods of the present invention, the nucleic acid sequence encoding the Cpf1 enzyme or an active fragment thereof may be selected from SEQ ID NOs: 13 or 14, or a sequence having at least 85%, 86%, 87%, 88%, 89%, 90%, 91%, 92%, 93%, 94%, 95%, 96%, 97%, 98%, or 99% identity over the whole length of the respective sequence of SEQ ID NOs: 13 or 14.

In yet another embodiment of the methods of the present invention, the Hammerhead ribozyme sequence, or a sequence encoding the same, is selected from SEQ ID NO: 17 or 18, and/or wherein the plant-derived Hepatitis Delta Virus (HDV)-like ribozyme sequence, or a sequence encoding the same, is selected from any one of SEQ ID NOs: 19 to 26, or a sequence having at least 95%, 96%, 97%, 98%, or 99% identity over the whole length of any one of SEQ ID NOs: 17 to 26.

In another embodiment of the methods of the present invention, the at least one Cpf1 guide RNA, or the nucleotide sequence encoding the same, which is (i) flanked by the Hammerhead ribozyme sequence at the 5′ and the plant-derived Hepatitis Delta Virus (HDV)-like ribozyme sequence at the 3′ end, further comprises a scaffold RNA sequence, or a sequence encoding the same, at the 5′ end; and/or which is (ii) embedded within the coding or within the non-coding region, preferably the 3′ untranslated region (UTR), of the sequence encoding a frame sequence, further comprises a scaffold RNA sequence, or a sequence encoding the same, at the 5′ and 3′ end.

As detailed above for the embodiments directed to a plant delivery system, the at least one Cpf1 guide RNA may comprise at least one scaffold RNA sequence being specific for a cognate Cpf1 enzyme or an active fragment thereof. In embodiments of the methods disclosed herein relying on a ribozyme system, one scaffold RNA sequence, or the sequence encoding the same, may be used, wherein in embodiments relying on an embedded mRNA approach two scaffold RNA sequences, or the sequence encoding the same, may be used. As detailed above, more than one scaffold RNA sequence may be used, particularly, where a multiplex targeting in one experiment is envisaged. Usually, the at least one scaffold RNA sequence will be located directly adjacent to the 5′ and/or 3′-end of a gRNA/crRNA of interest.

In one embodiment of the methods of the present invention, the scaffold RNA sequence, or a sequence encoding the same, may be selected from SEQ ID NO: 29 or 30, or a sequence having at least 95%, 96%, 97%, 98%, or 99% identity over the whole length of a SEQ ID NO: 29 or 30, or it may be selected from any further scaffold RNA sequence, or a DNA sequence encoding the same, as disclosed herein.

In one further embodiment of the methods of the present invention, the at least one Cpf1 enzyme or an active fragment thereof, or a nucleic acid sequence encoding the same, may be selected from any one of SEQ ID NOs: 13 to 16, or a sequence having at least 60%, 65%, 70%, 75%, 80%, 85%, 90%, 91%, 92%, 93%, 94%, 95%, 96%, 97%, 98%, 99% or more sequence identity over the whole length of the respective nucleic acid sequence of SEQ ID NOs: 13 or 14, or a sequence having at least 90%, 91%, 92%, 93%, 94%, 95%, 96%, 97%, 98%, or 99% identity over the whole length of the respective amino acid sequence of SEQ ID NOs: 15 or 16, respectively.

In one embodiment of the methods of the present invention, the at least one Cpf1 enzyme, or an active fragment thereof, or a nucleic acid sequence encoding the same, may comprise at least one mutation in comparison to a wild-type sequence (SEQ ID NO: 16) resulting in an altered PAM recognition, preferably wherein the at least one mutation is selected from G532R/K595R, or G532R/K538V/Y542R in comparison to the sequence of SEQ ID NO: 16.

In yet a further embodiment of the methods of the present invention, the at least one Cpf1 enzyme, or an active fragment thereof, or a nucleic acid sequence encoding the same, may comprise at least one mutation in comparison to a wild-type sequence (SEQ ID NO: 16) resulting in an altered PAM recognition, wherein the altered PAM recognition is a recognition of a TYCV, or of a TATV PAM sequence.

In certain embodiments, suitable Cpf1 mutant variants according to the present invention may be selected from any one of SEQ ID NOs: 38 to 41, or a sequence having at least 60%, 65%, 70%, 75%, 80%, 85%, 90%, 91%, 92%, 93%, 94%, 95%, 96%, 97%, 98%, 99% or more sequence identity over the whole length of the respective nucleic acid sequence of SEQ ID NOs: 38 or 39, or a sequence having at least 90%, 91%, 92%, 93%, 94%, 95%, 96%, 97%, 98%, or 99% identity over the whole length of the respective amino acid sequence of SEQ ID NOs: 40 and 41, respectively.

In another embodiment of the methods of the present invention, the at least one Cpf1 enzyme or an active fragment thereof, or a nucleic acid sequence encoding the same, and/or the at least one Cpf1 guide RNA system, or a nucleic acid sequence encoding the same, may be provided as at least one vector construct, or are provided as at least one linear construct.

In one embodiment of the methods of the present invention, the at least one Cpf1 guide RNA system comprises at least two guide RNAs, wherein the at least two guide RNAs may be separated by a nucleotide sequence comprising direct repeats, as disclosed herein above.

In one embodiment of the methods of the present invention, the sequence encoding a frame sequence is selected from a translatable or non-translatable sequence being selected from a marker gene, including an antibiotic marker or a fluorescent marker, a gene encoding a structural protein, a gene encoding an RNA species, an IRES encoding sequence. Further coding and non-coding sequences may serve the purpose of embedding and thus enhancing the efficiency and availability of at least one gRNA/crRNA as disclosed above for the at least one Cpf1 guide RNA system of the present invention, wherein the at least one gRNA/crRNA is embedded into a coding or non-coding region, preferably a 3′ untranslated region (UTR), of a sequence encoding a frame sequence.

In still another embodiment of the methods of the present invention, the sequence encoding a frame sequence may be selected from any one of SEQ ID NOs: 13, 14, 38, 39, 31, 32, 72, 73, 74, 75, 76, 157, or 158, or a sequence having at least 85%, 86%, 87%, 88%, 89%, 90%, 91%, 92%, 93%, 94%, 95%, 96%, 97%, 98%, or 99% identity over the whole length of the respective sequence of SEQ ID NOs: 13, 14, 38, 39, 31, 32, 72, 73, 74, 75, 76, 157, or 158.

In one embodiment of the methods of the present invention, the part of a plant may be selected from the group consisting of a plant cell, a plant tissue and a plant organ, preferably wherein the plant or a part of a plant may be selected from a genus selected from the group consisting of Hordeum, Sorghum, Saccharum, Zea, Setaria, Oryza, Triticum, Secale, Triticale, Malus, Brachypodium, Aegilops, Daucus, Beta, Eucalyptus, Nicotiana, Solanum, Coffea, Vitis, Erythrante, Genlisea, Cucumis, Marus, Arabidopsis, Crucihimalaya, Cardamine, Lepidium, Capsella, Olmarabidopsis, Arabis, Brassica, Eruca, Raphanus, Citrus, Jatropha, Populus, Medicago, Cicer, Cajanus, Phaseolus, Glycine, Gossypium, Astragalus, Lotus, Torenia, Allium, or Helianthus. More preferably, the plant or a part of a plant may be selected from a species selected from the group consisting of Hordeum vulgare, Hordeum bulbusom, Sorghum bicolor, Saccharum officinarium, Zea spp., including Zea mays, Setaria italica, Oryza minuta, Oryza sativa, Oryza australiensis, Oryza alta, Triticum aestivum, Triticum durum, Secale cereale, Triticale, Malus domestica, Brachypodium distachyon, Hordeum marinum, Aegilops tauschii, Daucus glochidiatus, Beta spp., including Beta vulgaris, Daucus pusillus, Daucus muricatus, Daucus carota, Eucalyptus grandis, Nicotiana sylvestris, Nicotiana tomentosiformis, Nicotiana tabacum, Nicotiana benthamiana, Solanum lycopersicum, Solanum tuberosum, Coffea canephora, Vitis vinifera, Erythrante guttata, Genlisea aurea, Cucumis sativus, Marus notabilis, Arabidopsis arenosa, Arabidopsis lyrata, Arabidopsis thaliana, Crucihimalaya himalaica, Crucihimalaya wallichii, Cardamine nexuosa, Lepidium virginicum, Capsella bursa pastoris, Olmarabidopsis pumila, Arabis hirsute, Brassica napus, Brassica oleracea, Brassica rapa, Raphanus sativus, Brassica juncacea, Brassica nigra, Eruca vesicaria subsp. sativa, Citrus sinensis, Jatropha curcas, Populus trichocarpa, Medicago truncatula, Cicer yamashitae, Cicer bijugum, Cicer arietinum, Cicer reticulatum, Cicer judaicum, Cajanus cajanifolius, Cajanus scarabaeoides, Phaseolus vulgaris, Glycine max, Gossypium sp., Astragalus sinicus, Lotus japonicas, Torenia fournieri, Allium cepa, Allium fistulosum, Allium sativum, Helianthus annuus, Helianthus tuberosus and/or Allium tuberosum. Particularly preferred are Beta vulgaris, Zea mays, Triticum aestivum, Hordeum vulgare, Secale cereale, Helianthus annuus, Solanum tuberosum, Sorghum bicolor, Brassica rapa, Brassica napus, Brassica juncacea, Brassica oleracea, Raphanus sativus, Oryza sativa, Glycine max, and/or Gossypium sp.

In one specific embodiment of the methods of the present invention, the plant or part of the plant may be a monocotyledonous plant, preferably Zea mays.

In one aspect, there is provided a plant or a part of a plant, or a progeny thereof, which may be obtained, or which may be obtainable by any of the methods of the present invention.

In another aspect, there is provided a use of a plant delivery system of the present invention; or a use of a Cpf1 enzyme or an active fragment thereof, or of a nucleic acid sequence encoding the same of the present invention; and/or a use of at least one guide RNA system, or the nucleic acid sequence encoding the same of the present invention, in a method of modifying a genomic target sequence of interest in a plant or part of a plant.

In one embodiment of the above use, the genomic target sequence of interest is a difficult to access target site, i.e., a target site hard to modify by any genome editing methods due to the specific environment of the genomic target site of interest at a specific locus of a complex eukaryotic genome.

In another aspect, there is provided method for modifying a genomic target sequence of interest in a eukaryotic cell of interest, wherein the method may comprise the steps of: (a) providing at least one Cpf1 enzyme or an active fragment thereof, or a nucleic acid sequence encoding the same; preferably, wherein the at least one nucleic acid sequence encoding the Cpf1 enzyme or an active fragment thereof is codon-optimized for the expression in the eukaryotic cell of interest; and (b) providing at least one Cpf1 guide RNA system, or a nucleic acid sequence encoding the same, the at least one Cpf1 guide RNA system comprising at least one Cpf1 guide RNA specific for a genomic target sequence of interest in the eukaryotic cell of interest; wherein the at least one Cpf1 guide RNA, or the nucleic acid sequence encoding the same, is (i) flanked by a Hammerhead ribozyme sequence at the 5′ and a plant-derived Hepatitis Delta Virus (HDV)-like ribozyme sequence at the 3′-end; and/or is (ii) embedded within a coding or non-coding region, preferably a 3′ untranslated region (UTR), of a sequence encoding a frame sequence (c) optionally: providing at least one repair template nucleic acid sequence, wherein the at least one repair template nucleic acid sequence is preferably flanked by one or more homology sequence(s) complementary to one or both adjacent region(s) of the genomic sequence of interest in the eukaryotic cell of interest; (d) introducing the at least one Cpf1 enzyme or an active fragment thereof, or a nucleic acid sequence encoding the same from step (a); and introducing the at least one Cpf1 guide RNA system, or a nucleic acid sequence encoding the same from step (b) and optionally: introducing the at least one repair template nucleic acid sequence from step (c) into the eukaryotic cell of interest; and (e) obtaining a eukaryotic cell of interest, or a progeny thereof, comprising a modification in the genomic target sequence of interest.

The specific Cpf1 guide RNA systems of the present invention may be suitably used in any eukaryotic cell or cellular system of interest. Preferably, the sequence encoding a Cpf1 enzyme or an active fragment thereof will then be codon-optimized for the specific eukaryotic target cell of interest. Furthermore, suitable regulatory sequences may be chosen for the constructs to be used, wherein the regulatory sequences, including inter alia promoters and terminators, should be chosen to be functional in a eukaryotic target cell of interest. The skilled person is able to define suitable promoters, preferably strong promoters, either with inducible or constitutive expression, depending on a cellular system of interest. An example for a very strong constitutive promoter in the plant system, e.g., Zea mays, is BdUbi10. A weaker promoter would be the BdEF1 for example. Inducible plant promoters are the tetracycline-, the dexamethasone-, and salicylic acid inducible promoters. Other promoters suitable according to the present invention are a CaMV (Cauliflower mosaic virus) 35S or a double 35S promoter. Other constitutive eukaryotic promoters are CMV (Cytomegalovirus), EF1a, TEF1, SV40, PGK1 (human or mouse), Ubc (ubiquitin 1), human beta-actin, GDS, GAL1 or 2 (for a yeast system), CAG (comprising a CMV enhancer, chicken beta actin promoter, and rabbit beta-globin splice acceptor), H1, or U6. A variety of inducible promoters is known to the skilled person. Suitable terminators can likewise be determined by the skilled person and include, inter alia, a SV40 terminator, or a HGH terminator.

In particular at least one Cpf1 guide RNA system, or a nucleic acid sequence encoding the same, wherein the at least one Cpf1 guide RNA, or the nucleic acid sequence encoding the same, is (i) flanked by a Hammerhead ribozyme sequence at the 5′-end and by a plant-derived Hepatitis Delta Virus (HDV)-like ribozyme sequence at the 3′-end may be particularly suitable for any GE method in an animal cell, or a fungal cell, etc., as there is a great need in replacing virus-derived sequences by functional sequences from other non-human pathogenic organisms. The plant-derived HDV-like sequences of the present invention can thus substitute for the original HDV sequences in any ribozyme system, when used in a pre-transcribed form, or when provided on a construct optionally comprising codon-optimization and/or regulatory sequences functional in a eukaryotic target cell of interest.

### Delivery and Analytical Methods

According to the present disclosure, any suitable delivery method to introduce at least one plant delivery system, or a component thereof, or any other biomolecule into a cell or cellular system can be applied, depending on the cell or cellular system of interest. The term “introduction” as used herein thus implies a functional transport of a biomolecule or genetic construct (DNA, RNA, single-or double-stranded, protein, comprising natural and/or synthetic components, or a mixture thereof) into at least one cell or cellular system, which allows the transcription and/or translation and/or the catalytic activity and/or binding activity, including the binding of a nucleic acid molecule to another nucleic acid molecule, including DNA or RNA, or the binding of a protein to a target structure within the at least one cell or cellular system, and/or the catalytic activity of an enzyme such introduced, optionally after transcription and/or translation. Where pertinent, a functional integration of a genetic construct may take place in a certain cellular compartment of the at least one cell, including the nucleus, the cytosol, the mitochondrium, the chloroplast, the vacuole, the membrane, the cell wall and the like. Consequently, the term “functional integration” implies that a molecular complex of interest is introduced into the at least one cell or cellular system by any means of transformation, transfection or transduction by biological means, including Agrobacterium transformation, or physical means, including particle bombardment, as well as the subsequent step, wherein the molecular complex can exert its effect within or onto the at least one cell or cellular in which it was introduced regardless of whether the construct or complex is introduced in a stable or in a transient way.

According to the various embodiments, at least one plant delivery system according to the present invention may thus be provided in the form of at least one vector, e.g., a plasmid vector, as at least one linear molecule, or as at least one complex pre-assembled ex vivo.

Depending on the nature of the genetic construct or biomolecule to be introduced, said effect naturally can vary and including, alone or in combination, inter alia, the transcription of a DNA encoded by the genetic construct to a ribonucleic acid, the translation of an RNA to an amino acid sequence, the activity of an RNA molecule within a cell, comprising the activity of a guide RNA, a crRNA, a tracrRNA, or an miRNA or an siRNA for use in RNA interference, and/or a binding activity, including the binding of a nucleic acid molecule to another nucleic acid molecule, including DNA or RNA, or the binding of a protein to a target structure within the at least one cell, or including the integration of a sequence delivered via a vector or a genetic construct, either transiently or in a stable way. Said effect can also comprise the catalytic activity of an amino acid sequence representing an enzyme or a catalytically active portion thereof within the at least one cell and the like. Said effect achieved after functional integration of the molecular complex according to the present disclosure can depend on the presence of regulatory sequences or localization sequences which are comprised by the genetic construct of interest as it is known to the person skilled in the art.

A variety of suitable transient and stable delivery techniques suitable according to the methods of the present invention for introducing genetic material, biomolecules, including any kind of single-stranded and double-stranded DNA and/or RNA, or amino acids, synthetic or chemical substances, into a eukaryotic cell, preferably a plant cell, or into a cellular system comprising genetic material of interest, are known to the skilled person, and comprise inter alia choosing direct delivery techniques ranging from polyethylene glycol (PEG) treatment of protoplasts (Potrykus et al. 1985), procedures like electroporation (D'Halluin et al., 1992), microinjection (Neuhaus et al., 1987), silicon carbide fiber whisker technology (Kaeppler et al., 1992), viral vector mediated approaches (Gelvin, Nature Biotechnology 23, “Viral-mediated plant transformation gets a boost”, 684-685 (2005)) and particle bombardment (see e.g. Sood et al., 2011, Biologia Plantarum, 55, 1-15). Transient transfection of mammalian cells with PEI is disclosed in Longo et al., Methods Enzymol., 2013, 529:227-240. Protocols for transformation of mammalian cells are disclosed in Methods in Molecular Biology, Nucleic Acids or Proteins, ed. John M. Walker, Springer Protocols.

For plant cells to be modified, despite transformation methods based on biological approaches, like Agrobacterium transformation or viral vector mediated plant transformation, and methods based on physical delivery methods, like particle bombardment or microinjection, have evolved as prominent techniques for introducing genetic material into a plant cell or tissue of interest. Helenius et al. (“Gene delivery into intact plants using the Helios™ Gene Gun”, Plant Molecular Biology Reporter, 2000, 18 (3): 287-288) discloses a particle bombardment as physical method for introducing material into a plant cell.

Currently, there thus exists a variety of plant transformation or transfection methods to introduce genetic material in the form of a genetic construct into a plant cell or cellular system of interest, comprising biological and physical means known to the skilled person on the field of plant biotechnology which are applicable to the various introduction techniques of biomolecules or complexes thereof according to the present invention. Notably, said delivery methods for transformation and transfection can be applied to introduce the tools of the present invention simultaneously. A common biological means is transformation with Agrobacterium spp. which has been used for decades for a variety of different plant materials. Viral vector mediated plant transformation represents a further strategy for introducing genetic material into a cell of interest. Physical means finding application in plant biology are particle bombardment, also named biolistic transfection or microparticle-mediated gene transfer, which refers to a physical delivery method for transferring a coated microparticle or nanoparticle comprising a nucleic acid or a genetic construct of interest into a target cell or tissue. Physical introduction means are suitable to introduce nucleic acids, i.e., RNA and/or DNA, and proteins. Likewise, specific transformation or transfection methods exist for specifically introducing a nucleic acid or an amino acid construct of interest into a plant cell, including electroporation, microinjection, nanoparticles, and cell-penetrating peptides (CPPs). Furthermore, chemical-based transfection methods exist to introduce genetic constructs and/or nucleic acids and/or proteins, comprising inter alia transfection with calcium phosphate, transfection using liposomes, e.g., cationic liposomes, or transfection with cationic polymers, including DEAD-dextran (“DEAD” disclosed as SEQ ID NO: 164) or polyethylenimine, or combinations thereof. Said delivery methods and delivery vehicles or cargos thus inherently differ from delivery tools as used for other eukaryotic cells, including animal and mammalian cells and every delivery method may have to be specifically fine-tuned and optimized for a construct of interest for introducing and/or modifying the genetic material of at least one cellular system, plant cell, tissue, organ, or whole plant; and/or can be introduced into a specific compartment of a target cell of interest in a fully functional and active way.

The above delivery techniques, alone or in combination, can be used for in vivo (in planta) or in vitro approaches. According to the various embodiments of the present invention, different delivery techniques may be combined with each other, simultaneously or subsequently, for example, using a chemical transfection for the at least synthetic transcription factor, or the sequence encoding the same, one site-specific nuclease, or a mRNA or DNA encoding the same, and optionally further molecules, for example, a gRNA, whereas this is combined with the transient provision of the (partial) inactivation(s) using an Agrobacterium based technique.

A plant delivery system, or a sub-component thereof, of the present invention may thus be introduced together with, before, or subsequently to the transformation and/or transfection of relevant tools for inducing a targeted genomic edit.

Likewise, methods for analyzing a successful transformation or transfection event according to the present invention are known to the person skilled in the art and comprise, but are not limited to polymerase chain reaction (PCR), including inter alia real time quantitative PCR, multiplex PCR, RT-PCR, nested PCR, analytical PCR and the like, microscopy, including bright and dark field microscopy, dispersion staining, phase contrast, fluorescence, confocal, differential interference contrast, deconvolution, electron microscopy, UV microscopy, IR microscopy, scanning probe microscopy, the analysis of plant or plant cell metabolites, RNA analysis, proteome analysis, functional assays for determining a functional integration, e.g. of a marker gene or a transgene of interest, or of a knock-out, Southern-Blot analysis, sequencing, including next generation sequencing, including deep sequencing or multiplex sequencing and the like, and combinations thereof.

In yet another embodiment of the above aspect according to the present invention, the introduction of a construct of interest is conducted using physical and/or biological means selected from the group consisting of a device suitable for particle bombardment, including a gene gun, including a hand-held gene gun (e.g. Helios® Gene Gun System, BIO-RAD) or a stationary gene gun, transformation, including transformation using Agrobacterium spp. or using a viral vector, microinjection, electroporation, whisker technology, including silicon carbide whisker technology, and transfection, or a combination thereof.

The practice of the disclosed methods employs, unless otherwise indicated, conventional techniques in molecular biology, biochemistry, genetics, computational chemistry, cell culture, recombinant DNA and related fields as are within the skill of the art. These techniques are fully explained in the literature. See, for example, Sambrook et al. MOLECULAR CLONING: A LABORATORY MANUAL, Second edition, Cold Spring Harbor Laboratory Press, 1989; Ausubel et al., CURRENT PROTOCOLS IN MOLECULAR BIOLOGY, John Wiley & Sons, New York, 1987 and periodic updates; and the series METHODS IN ENZYMOLOGY, Academic Press, San Diego.

The present invention is further described with reference to the following non-limiting examples.

### EXAMPLES

### Example 1: Target Sequences

A variety of different gene editing plasmids (GEPs) was designed and used for the purpose of the present analysis. As shown below, Table 1 provides the internal name (crGEP) of the different gene editing plasmids (left column), information about the targeted gene (middle column), as well as the respective target site of selected crRNAs or sgRNAs (right column). The target sites represent the actual binding sites in a genomic target region or gene of interest.

TABLE 1

| crGEP | Gene information | Target sequence in gene |
|---|---|---|
| crGEP5 | HMG13 intron 3 near sgGEP8 | CTCGTCACGATTCCCCTCTCCTGG |
| | | (SEQ ID NO: 49) |
| crGEP6 | HMG13 intron 3 near sgGEP9 | CCCACCTGAAAAGTTCGACCAGGA |
| | | (SEQ ID NO: 50) |
| crGEP7 | HMG13 exon 7 near gRNA-18 | TGTGTGGTCACACTTGCCAGCCAG |
| | | (SEQ ID NO: 51) |
| crGEP9 | Glossy 2 (GI 2) intron 1 | GTGGTCGGATTTCTGGCATCGCTG |
| | near exon 1 | (SEQ ID NO: 52) |
| crGEP43 | ZM-GLYK autoinhibitor | GTCTATGTCGATGACCAGCAGAT |
| | | (SEQ ID NO: 53) |
| crGEP57 | HMG13 (for RR LbCpf1 PAM | CCTCTCCTGGTCGAACTTTTCAGG |
| | Variant) near crGEP5 | (SEQ ID NO: 54) |
| crGEP58 | HMG13 (for RR LbCpf1 PAM | ACCAGGAGAGGGGAATCGTGACGA |
| | Variant) near crGEP5 | (SEQ ID NO: 55) |
| crGEP59 | HMG13 (for RR LbCpf1 PAM | TTATAGCACGACAAAAGTAAAAAT |
| | Variant) near crGEP7 | (SEQ ID NO: 56) |
| crGEP60 | HMG13 (for RR LbCpf1 PAM | ATTGTCGTCATCATCGGCTAACAT |
| | Variant) near crGEP7 | (SEQ ID NO: 57) |
| crGEP61 | HMG13 (for RVR LbCpf1 PAM | TACTTTGACTTTTCCCTTAATGAC |
| | Variant) near crGEP5 | (SEQ ID NO: 58) |
| crGEP62 | HMG13 (for RVR LbCpf1 PAM | GGGCCGGTCATAAAGCAGCTCTCA |
| | Variant) near crGEP5 | (SEQ ID NO: 59) |
| crGEP63 | HMG13 (for RVR LbCpf1 PAM | ACGGATAGCGCTCCTCGTTGGCGC |
| | Variant) near crGEP7 | (SEQ ID NO: 60) |
| crGEP64 | HMG13 (for RVR LbCpf1 PAM | ACAATGTTAGCCGATGATGACGAC |
| | Variant) near crGEP7 | (SEQ ID NO: 61) |
| crGEP65 | Glossy2 (for RR LbCpf1 PAM | GGTAACCGTCCTCCGTACGTCGTC |
| | Variant) near sgGEP14 | (SEQ ID NO: 62) |
| crGEP66 | Glossy2 (for RR LbCpf1 PAM | CCTCTCTACGACGACGTACGGAGG |
| | Variant) near sgGEP14 | (SEQ ID NO: 63) |
| crGEP67 | Glossy2 (for RVR LbCpf1 | GTTACGGGCAGTGCAGTTGAGCAA |
| | PAM Variant) near sgGEP14 | (SEQ ID NO: 64) |
| crGEP68 | Glossy2 (for RVR LbCpf1 | CTGACTGTCCAGTGGCCACCTAGA |
| | PAM Variant) near sgGEP14 | (SEQ ID NO: 65) |
| crGEP52 | Wheat TDF (For RR LbCpf1 | CAGCATGGCATGGAGGGTGACGAT |
| | PAM variant) | (SEQ ID NO: 77) |
| crGEP53 | Wheat TDF (For RR LbCpf1 | AGCATGGCATGGAGGGTGACGATG |
| | PAM variant) | (SEQ ID NO: 78) |
| crGEP54 | Wheat TDF (For RR LbCpf1 | CGCAGGAGGAGGAGGAGCTCATCG |
| | PAM variant) | (SEQ ID NO: 79) |
| crGEP55 | Wheat TDF (For RR LbCpf1 | CGCACCGCTTCAGCCCTGCAGCAC |
| | PAM variant) | (SEQ ID NO: 80) |
| crGEP56 | Wheat TDF (For RR LbCpf1 | GCACCGCTTCAGCCCTGCAGCACG |
| | PAM variant) | (SEQ ID NO: 81) |
| crGEP26 | Sugar beet ALS | GCTGCTAAACAATCAACATTT (SEQ |
| | | ID NO: 82) |
| crGEP27 | Sugar beet ALS | TAAACAATCAACATTTAGGTA (SEQ |
| | | ID NO: 83) |
| crGEP28 | Sugar beet ALS | ATTTAGGTATGGTTGTCCAA (SEQ |
| | | ID NO: 84) |
| crGEP29 | Sugar beet ALS | TTTAGCAGCATTATCTTAAC (SEQ |
| | | ID NO: 85) |
| crGEP30 | Sugar beet ALS | TATAGAACCTATCTTCCCAT (SEQ |
| | | ID NO: 86) |

### Example 2: Plant Protoplast Transfection

Whenever a plant protoplast transfection was used for the purpose of the experiments disclosed herein, protocols known in the art were used relying on the following steps:

The buffers and solutions used were an enzyme solution, an enzyme wash solution, an enzyme wash buffer (EWB), an MMG (glycol-mannitol magensium) buffer, e.g., containing 0.1 to 0.5 M mannitol, 15 mM to 20 mM magnesium chloride and 4 mM MES (pH10 to 40% PEG (polyethylene glycol) calcium, a stop buffer, and a W5 buffer (e.g., comprising 154 mM NaCl, 125 mM CaCl₂, 5 mM KCl, 2 mM MES (pH 5.7), with or without glucose).

First 20 μg (or unless otherwise specified) plasmid DNA were added to 2 ml tubes place at 4° C. Next, leaves were harvested from greenhouse of first and/or second fully expanded true leaves from 10-14 day old etiolated seedlings, and they were placed in a bag with wet paper towels. The leaves were cut into fine strips and weigh out 4.5 g. They were placed into a deep petri dish with 30 ml of enzyme solution and then into vacuum at 714 mbar for 30 minutes. Digestion was continued for 2.5 more hours on a rocker (40 rpm (between 1 and 2) at 28° C. in an incubator. During the last 5 minutes, the rocker was set to 80 rpm. Equal amounts (30 ml) of EWB were added and the mixture was mixed by gentle swirling. In a hood, a 40 Um cell strainer was pre-wetted with ˜2 ml of EWB in a 50 ml conical tube. The tube was holded at angle and the protoplast solution was gently put through the filter so that the cells could run down the side of the tube. The resulting material was split into two tubes with 30 ml per tube.

Next, the cells were pelleted at 70 g for 5 minutes at RT (room temperature) and as much supernatant as possible was removed. The material was resuspended in 20 ml EWB, by letting it fall down the side of the tube and it was mixed by gentle rocking. Next, the material was centrifuged at 70 g for 5 minutes at RT. Supernatant was removed followed by a resuspension in 5 ml of EWB. It may be rocked gently to break up clumps. Cells were allowed to settle for 30 minutes. Then, cells were counted by adding 10 μl of one tube to a hemacytometer. The cells within the small squares on both sides were counted. Calculation was performed by: number of protoplasts×10\hat{ }4×5 ml, for example 158 cells×10\hat{ }4×5 ml =7925000.

The pellet was resuspended in 7.925 ml MMG for 10\hat{ }6 cells per ml. Supernatant from settled cells was removed and the pellet was resuspended in a calculated amount of MMG. Cells should not be left in MMG for longer than 15 minutes. Next, 200 μl of resuspended protoplasts were added to each tube with DNA. 220 μl of 40% PEG-CaCl₂ buffer were added and it was mixed by tapping. Incubation time: for 5-10 minutes. The transfection was stopped with 880 μl of stop buffer and the mixture was mixed by gently inverting. Next, a centrifugation at 70 g for 5 minutes at RT was done and the supernatant was removed. Cells were resuspended in 1 ml of W5 buffer. 1 ml of W5 buffer was added to 6-well plate and the 1 ml of cells was added to the plate for a total of 2 ml. The cells were placed in dark cabinet for 24 hours.

Analysis was performed by taking pictures on the inverted scope, (Brightfield and either Green or Red). Place all 2 ml into a 2 ml tube and flow 1,000 cells. Spin at 70 g for 5 minutes. Finally, the supernatant was removed and the pellet was frozen.

Suitable variations of the above protocol for different target cells, plants or cellular systems are known to the skilled person. Protocols, including transient expression assays for monocot and dicot plant cells, can be obtained from Sheen, J. 2002, A transient expression assay using Arabidopsis mesophyll protoplasts, genetics.mgh.harvard.edu/sheenweb/; Yanagisawa et al., 2003, Nature 425: 521-525; Asai et al., 2002, Nature 415:977-983; Sheen, 2001, Plant Physiol. 127: 1466-1475; Hwang & Sheen, 2001, Nature 413:383-389; Kovtun et al., 2000, PNAS 97: 2940-2945; Abel & Theologis, 1994, Plant J. 5: 421-427; Masson & Paszkowski, 1992, Plant J. 2: 829-833; Damm et al., 1989, MGG 217: 6-12; or Negrutiu et al., 1987, Plant Mol Biol 8:363-373.

### Example 3: Next Generation Sequencing (NGS) Protocol

Whenever NGS was used for the experiments, the following protocol was followed: Library preparation: Libraries were prepared by two PCR steps to amplify target region and add sequencing adaptors. Barcodes were designed with the primers and added during 1^{st} PCR step for sample differentiating. Adaptors were added during 2^{nd} PCR for sequencing. Next generation sequencing (NGS): Amplicons were sequenced with Illumina Miseq 150 PEplatform. Protoplast populations were sequenced with 100,000× coverage, immature embryo and callus, agrobacteria transformed leaf samples and bombardment wheat leaf samples were sequenced with 250,000×, 300,000× and 50,000× coverage respectively. Data were analyzed using FastQC+Jemultiplexer+Trimmomatic for reads QC and demultiplexing, CRISPResso for Indel identification at the targets, and in house bash customer script for editing events calling. The whole analysis pipeline was used automatically using an in-house bash customer script.

### Example 4: mRNA Embedded Cpf1 Guide RNA Delivery is a Viable Option to Replace Ribozyme Based Delivery Systems in Plant Cells

This experiment was performed to compare gRNA induced indel activity from mRNA-embedded gRNA with the indel activity of gRNAs delivered by the ribozyme system. The data clearly show equivalent or even improved indel activity for mRNA embedded gRNA compared to gRNAs delivered by the ribozyme system for target gRNAs crGEP5 (easily to access target site, cf. Table 1 above) and crGEP7 (difficult to access target site, cf. Table 1 above). In contrast, the non-mRNA embedded guides clearly show highly diminished activities at those two target sites (FIG. 1C). The data provide evidence that an mRNA delivery system when properly configured is a potent tool in order to improve modification of genomic target sequences which are hardly accessible by conventional CRISPR/Cpf1 delivery systems.

The experiment was performed by co-delivery of the LbCpf1 plasmid (SEQ ID NO: 37) and the mRNA/non-mRNA embedded guide RNAs into protoplasts of corn leaf cells. Exemplary sequences of the concept vectors are shown in SEQ ID Nos: 33 and 34, respectively. A conventional ribozyme control vector is shown in SEQ ID NO: 44. Different fluorescent markers and crRNA repeats (cf. Table 1 above) were cloned into the basic vector to test the general applicability of the technique and to evaluate the efficiency for different target sites in different settings. After 24 hrs, cells were counted in a flow cytometer using reporter gene fluorescence. After DNA isolation from these cells, the target sites were amplified and the obtained PCR products were sequenced using NGS technology. The indel frequency observed for the different samples were then normalized against the protoplast transformation efficiency seen from the flow cytometer (data not shown).

To test whether mRNA embedded gRNAs and/or gRNAs delivered in the ribozyme system result in InDel activity at target genomic loci in plants of the species Beta vulgaris, the following experiment was performed.

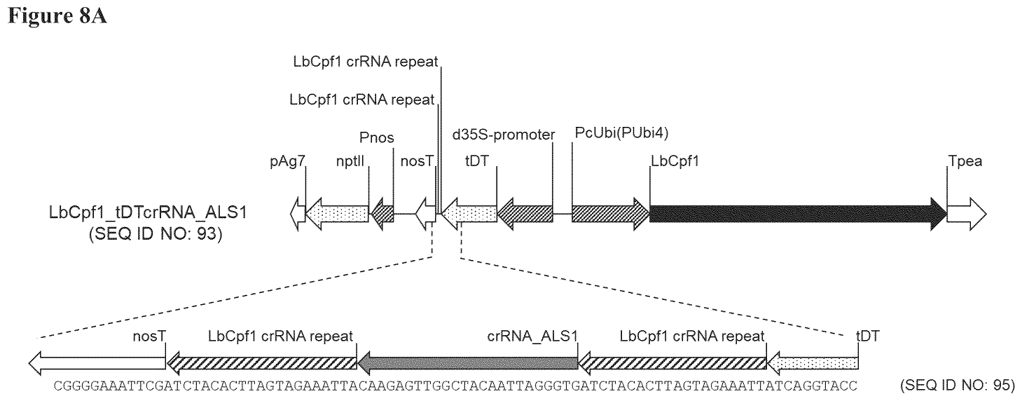

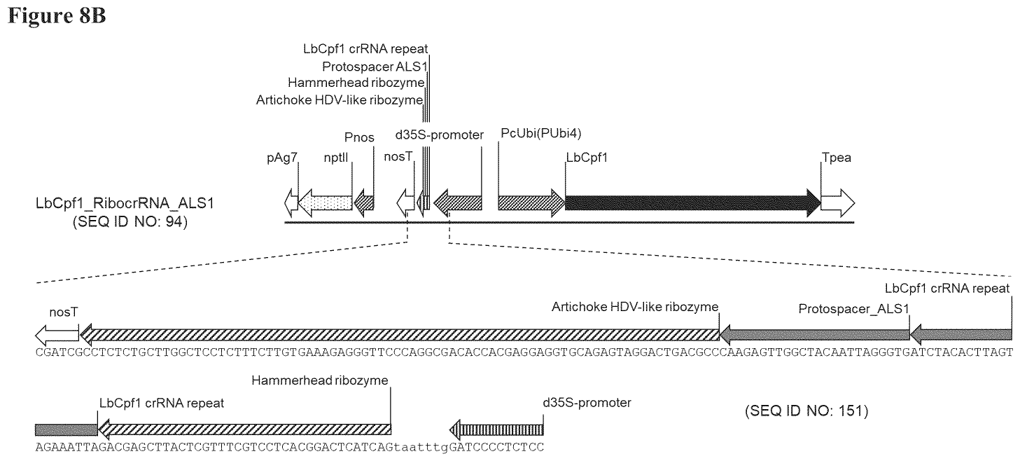

Binary vectors were created, which harbor (i) an nptII resistance cassette for in planta selection, (ii) a PcUbi4::Cpf1 expression cassette and (iii) a crRNA, which targets one of five different target regions within the target gene BvALS (Table 2). For each of the five protospacer two different crRNA versions were tested. In a first version (tDTcrRNA_ALS1-ALS5) the crRNA is embedded in a Pol II-driven RNA transcript as described before in Zhong et al., 2017 (Zhong, G., et al. (2017). “Cpf1 proteins excise CRISPR RNAs from mRNA transcripts in mammalian cells.” Nat Chem Biol 13 (8): 839-841). This transcript includes an mRNA encoding the fluorescent protein tDT (FIG. 8A).

The second crRNA version (RibocrRNA_ALS1-ALS5) produces the crRNA from a Pol II promoter using the ribozyme-based strategy described in He et al., 2017 (He, Y., et al. (2017). “Self-cleaving ribozymes enable the production of guide RNAs from unlimited choices of promoters for CRISPR/Cas9 mediated genome editing.” J Genet Genomics 44 (9): 469-472). Thereby, the crRNA production unit is composed of three parts: the 5′-end encoding the Hammerhead (HH) ribozyme, the middle part encoding the crRNA, and the 3′-end encoding the Artichoke HDV-like ribozyme (FIG. 8B). The primary transcripts will undergo self-cleavage catalyzed by the intrinsic nuclease activities of the ribozymes to release the mature and desired crRNA.

TABLE 2
Selected PAM plus protospacer within the
target gene BvALS and nomenclature
of the corresponding crRNAs.

| crRNA | PAM | Protosparer | |
|---|---|---|---|
| crRNA_ALS #1 | TTCT | CACCCTAATTGTAGCCAACTCTTG | SEQ ID |
| | | | NO: 146 |
| crRNA_ALS #2 | TTTA | GCAGCATTATCTTAACTGGGAGAT | SEQ ID |
| | | | NO: 147 |
| crRNA ALS #3 | TTTA | GGTATGGTTGTCCAATGGGAAGAT | SEQ ID |
| | | | NO: 148 |
| crRNA ALS #4 | TTTC | CAAGGTATGTATGTGCCCGGTTAG | SEQ ID |
| | | | NO: 149 |
| crRNA_ALS #5 | TTTG | GAAGGGTTTCCAAGGTATGTATGT | SEQ ID |
| | | | NO: 150 |

The binary vectors were transformed into Agrobacteria strain AGL1-1. AGL-1 harbouring the genome editing constructs were cultivated in medium A, supplemented with 100 mg/l Carbenicillin, 100 mg/l Rifampicin, 50 mg/l Spectinomycin and 25 mg/l Streptomycin for cultivation. Afterwards, Agrobacterium was suspended in medium B at OD600=0.8 and used for vacuum infiltration of 5×5 mm large leaf explants from three weeks old in vitro shoots of the genotype S706. Co-Cultivation was done on Medium C at 21° C. in the dark. Three days after infiltration explants were transferred to Medium D containing 500 mg/l Timentin. Sampling of the infiltrated leaf explants for subsequent DNA extraction and NGS analysis was done 10 days after infiltration. For each infiltrated construct two times 15 leaf explants were pooled for DNA extraction and handled as two biological replicates.

For detailed medium composition see table 3.

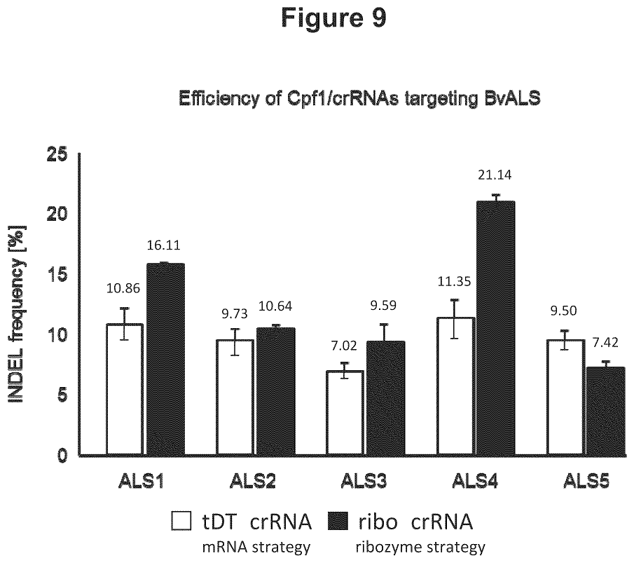

Vacuum infiltrated sugar beet leaf explants were analyzed via NGS to determine the LbCpf1-mediated INDEL frequency within the target gene BvALS (FIG. 9). Over all, InDel frequencies between 7% and 21% were observed. With one exemption, direct comparison of the two crRNA versions (tDTcrRNA vs. RibocrRNA) revealed higher InDel frequencies of the crRNAs, which are produced via the ribozyme strategy.

TABLE 3
Medium composition.

| Medium | Composition |
|---|---|
| Medium A | 5 g/l tryptone + 2.5 g/l yeast extract + 1 g/l NaCl + |
| | 5 g/l mannitol + 0.1 g/l MgSO₄x7H2O + 0.25 g/l |
| | KH₂PO₄ + 1 g/l glutamic acid, pH 7.0, pH 7.0 |
| Medium B | 440 mg/l CaCl₂x2H₂O + 170 mg/l KH₂PO₄ + 1.9 g/l |
| | KNO₃ + 180.7 mg/l MgSO₄ + 1.65 g/l NH₄NO₃ + 2 mg/l |
| | BAP + 40 μg/ml Acetosyringone + 20 g/l sucrose + |
| | 2 g/l glucose, pH 6.0 |
| Medium C | 440 mg/l CaCl₂x2H₂O + 170 mg/l KH₂PO₄ + 1.9 g/l |
| | KNO₃ + 180.7 mg/l MgSO₄ + 1.65 g/l NH₄NO₃ + 2 mg/l |
| | BAP + 40 μg/ml Acetosyringone + 20 g/l sucrose + |
| | 2 g/l glucose + 10 g/l agar, pH 6.0 |
| Medium D | MS salts + 15 g/l sucrose + 2 mg/l BAP + |
| | 8 g/l agar, pH 6. |
| Medium E | MS salts + 30 g/l sucrose + 0.25 mg/l benzyladenine |
| | (BAP) + 10 g/l agar, pH 6.0 |
| Medium F | MS salts + 30 g/l sucrose + 1 mg/l GA3 + 1 mg/l |
| | Thidiazuron (TDZ) + 10 g/l agar, pH 6.0 |
| Medium G | MS salts + 30 g/l sucrose + 1 mg/l GA3 + 1 mg/l |
| | TDZ + 500 mg/l Timentin + 10 g/l agar, pH 6.0 |
| Medium H | MS salts + 30 g/l sucrose + 1 mg/l GA3 + 1 mg/l |
| | TDZ + 500 mg/l Timentin + 100 mg/l paromomycin + |
| | 10 g/l agar, pH 6.0 |
| Medium I | MS salts + 30 g/l sucrose + 0.25 mg/l benzyladenine |
| | (BAP) + 100 mg/l kanamycin + 10 g/l agar, pH 6.0 |
| Medium J | MS salts + 30 g/l sucrose + 6.25 mg/l NAA + 10 |
| | g/l agar, pH 6.0 |

### Example 5: HDV-like Plant-Derived Ribozymes Show High Indel Activity and are Suitable as HDV Replacements

This experiment was performed in order to test the activity of specific plant-derived autocatalytic ribozymes and their suitability to replace HDV ribozymes. Therefore, plant-derived HDV-like sequences from Rice, Sunflower and Artichoke were identified, cloned and specifically tested for their ability to replace the original HDV ribozyme sequence of Hepatitis-delta virus. Specifically, the constructs with plant-derived HDV ribozyme sequences were tested for their indel activity at two previously validated targets sites (cf. Table 1 above) using the crGEP5 and crGEP6 gRNAs flanked by the HH ribozyme at the 3′-end (see SEQ ID NOs: 17 and 45, 46 and 48) and the respective plant-derived HDV-like ribozyme at the 5′-end (SEQ ID NOs: 19, 21 and 25 and 45, 46 and 48). Further, a specific strategy vector was constructed comprising a HH and a conventional HDV ribozyme sequence, a LbCpf1 RNA scaffold sequence, and suitable regulatory elements and a beta-lactamase encoding gene (SEQ ID NO: 44). Sequences of interest, in particular suitable gRNAs/crRNAs can be easily cloned into this vector. Plasmid cloning vectors suitable to insert a coding sequence of interest were constructed for the HDV-like ribozymes characterized, i.e., for rice-, sunflower-and artichoke-derived HDV sequences (see SEQ ID NOs: 45, 46 and 48).

As can be seen from the results shown in FIG. 2B, the HDV-like sequence derived from Artichoke showed significant indel activity at both target sites (crGEP5 and crGEP6, see also Table 1), while the Rice and Sunflower derived sequences showed poor indel activity. Furthermore, the activity of the Artichoke derived sequence was compared with the activity of the HDV ribozyme sequence. The data indicate that the Artichoke derived sequence results in up to 50-60% indel activity (2-5% indel activity for Rice and Sunflower sequences) compared to the original HDV sequence (FIG. 2C).

This surprising observation is in clear contrast to the teaching of the prior art which could not present evidence for the activity of a plant-derived HDV-like ribozyme sequence in vitro or in vivo when using a plant-derived HDV-like ribozyme in a recombinant way. The data of the present experiment therefore indicate that plant-derived HDV-like ribozyme sequences could potentially replace the original HDV sequences in gRNA ribozyme delivery systems thereby avoiding the use human pathogen-derived sequences and time consuming and expensive deregulation processes in product development in biotechnology.

The above described experiment was performed in the same manner as outlined in Examples 2 and 3 above. Constructs were introduced into protoplasts, and indel activity from the action of the RR or RVR versions was determined by NGS.

The sunflower HDV-like ribozyme sequence is highly homologous to the HDV-like ribozyme sequence derived from artichoke. Due to the accidental usage of a truncated sunflower HDV-like sequence (SEQ ID NO: 22) which showed low indel activity, it was further tested whether the correct full-length sunflower HDV-like ribozyme sequence (SEQ ID NO: 24) results in increased indel activity comparable to the indel activity observed for the artichoke HDV-like ribozyme sequence. Therefore, the indel activity at two target sites crGEP7 and crGEP43 was investigated using delivery by either the HDV ribozyme sequence (SEQ ID NO: 28) or the full-length sunflower HDV-like ribozyme sequence (SEQ ID NO: 24) in the expression plasmids. These constructs were co-delivered into protoplasts along with the LbCpf1 nuclease and samples were collected after 24 hrs for analysis after flow cytometry was carried out for transformation efficiency. The NGS analysis of target sites gave % raw indels which were multiplied by each samples' % protoplast transformation efficiency to give % indel formation, normalized to the protoplast transformation efficiency.

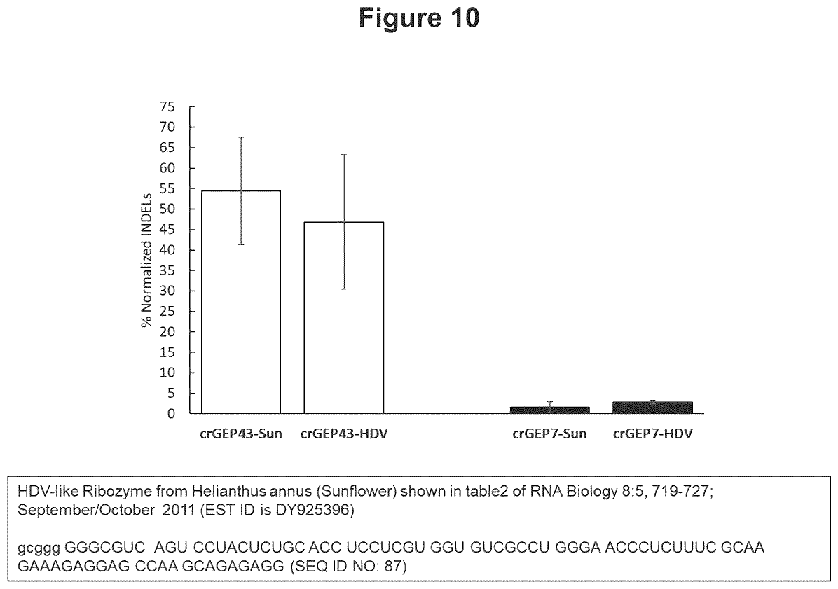

The results are summarized in FIG. 10 and indicate that the % indel activity of full-length sunflower HDV-like ribozyme sequence is equivalent or even greater than the indel activity of the original HDV ribozyme sequence confirming previous data obtained for the artichoke HDV-like ribozyme sequence. The herein presented data therefore strengthens the hypothesis that plant-derived HDV-like ribozyme sequences could potentially replace the original HDV sequences in a gRNA ribozyme delivery systems, making the use of human pathogen-derived sequences superfluous.

### Example 6: LbCpf1 PAM Variants RR and RVR Show Activity at Multiple Target Sites, including Difficult to Access Genomic Loci

The experiment was performed to test different PAM variants (i.e. a RR and RVR versions previously described for the mammalian cell system (Gao et al. (2017) Nat Biotechnol, 35 (8): 789-792)). Those PAM variants (SEQ ID NOs: 35, 36, 38 and 39) were specifically codon-optimized in contrast to the variants available. Specifically, the PAM variants were tested against targets in the neighborhood of the control target sites of crGEP5, crGEP7 and sgGEP14, and crGEP9 in the HMG13 and Glossy2 genes (cf. Table 1 above). Based on this data the inventors were able to identify multiple target sites at which the RR PAM motif shows more than 50% indel activity compared to the wildtype control PAM sequences of LbCpf1 (SEQ ID NO: 16). Based on this data the authors further concluded that previously difficult-to-cut-target sites like glossy2 (due to their GC-rich sequences), are now accessible by using modified PAM sequences like the RR PAM motif. This motif resulted in 30% indel activity rates compared to 0.36% of the original PAM sequence (control) (see FIG. 3B). The data indicate that the expanded PAM repertoire can be a promising alternative to target genomic sequences which would be otherwise not accessible by using the wildtype Cpf1 PAM sequences (SEQ ID NO: 16). The experiment was performed in the same manner as described in Examples 2 and 3 above.

The above results were also confirmed by post-published data which show that Cpf1 PAM variants RR and RVR are able to recognize difficult to access genomic loci (Zhong Z. et al. (2018), Plant genome editing using FnCpf1 and LbCpf1 nucleases at redefined and altered PAM sites, Molecular Plant 11, 7:999-1002; and Li S. et al. (2018), Expanding the scope of CRISPR/Cpf1-mediated genome editing in rice, Molecular Plant 11, 7:995-998).

### Example 7: Promoter Swaps on Cpf1 and crRNA Modules to Identify Best Expression Strategy

This experiment was performed in order to validate different promoter sequences and their ability to induce expression and indel activity of nuclease and/or ribozyme constructs (see FIGS. 4A and 4B). As can be seen from the respective data, highest expression and/or activity of the Cpf1 nuclease construct was observed for the expression under the control of the promoter pBdUbi10 (SEQ ID NO: 1), whereas highest activity for the gRNA/ribozyme construct was observed for the expression under the control of the pZmUbi1 promoter sequence (SEQ ID NO: 2).

The effect of different promoter sequences on LbCpf1 nuclease expression was tested by validating their indel activity at the two target sites crGEP5 and crGEP7 (cf. Table 1 above). The results in FIG. 4A indicate that replacement of the promoter pZmUbi1 for the LbCpf1 construct (cf. SEQ ID NO: 37 with the promoter sequence pBdUbi10 significantly improves nuclease expression and indel activity compared to pZmUbi1, whereas the use of pOsActin1 (SEQ ID NO: 3) does not improve the expression and/or indel activity of the nuclease construct. The data therefore clearly indicate that the promoter pBdUbi10 is a promising alternative to replace the promoter pZmUbi1 in the respective construct in order to increase LbCpf1 expression and indel activity at the tested target sites.

Similar experiments were performed for gRNA/ribozyme constructs by replacing the ZmUbi1 promoter in pGEP296 with either the BdUbi10 promoter or the OsActin promoter (FIG. 4B). Here, the data indicate that best expression and indel activity was obtained for the promoter sequence pZmUbi1.

In summary, the data provide evidence that the expression and indel activity of the CRISPR/Cpf1 system can be fine-tuned by the use of specific promoter sequences for the different expression constructs needed to provide a functional CRISPR system.

### Example 8: Head to Head Comparison of pUbi1 vs pBdUbi10 Activity Driving Either RR or RVR Version of LbCpf1 Across Target Sites in HMG13 or GI2

This experiment was performed to test the promoter strength to drive expression of RR (cf. SEQ ID NOs: 35 and 38) or RVR (SEQ ID NOs: 36 and 39) version of LbCpf1. The promoters pZmUbi1 versus pBdUbi10 were tested for their capability to drive nuclease expression in a targeted way by also testing activity at the same targets for both constructs. The data indicate that the BdUbi10 promoter clearly outperformed the ZmUbi2 promoter at each target, further bolstering the results that the pBdUbi10 promoter is superior in driving nuclease expression in the plant system (cf. FIG. 6, Table 1).

### Example 9: Multiplexing-Ribozyme-based gRNA Arrays Efficiently Disrupt Multiple Target Sites

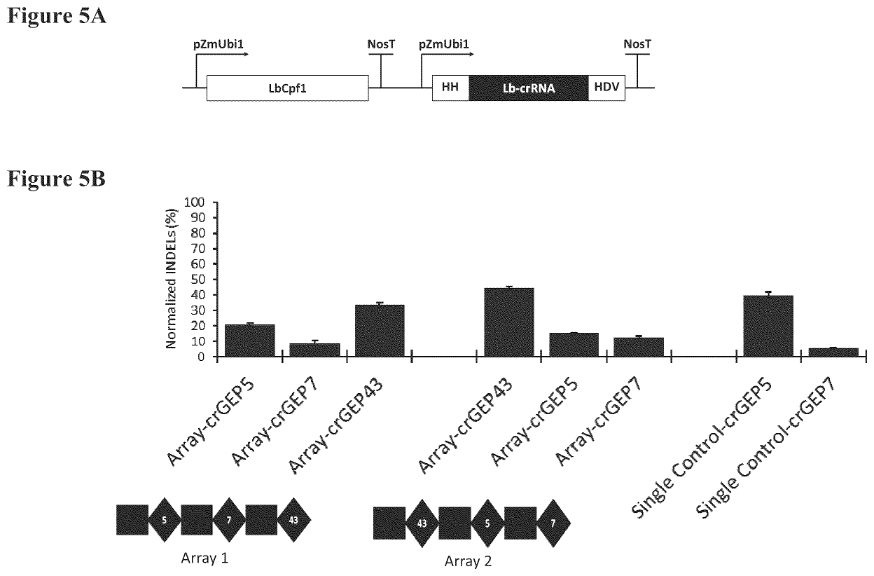

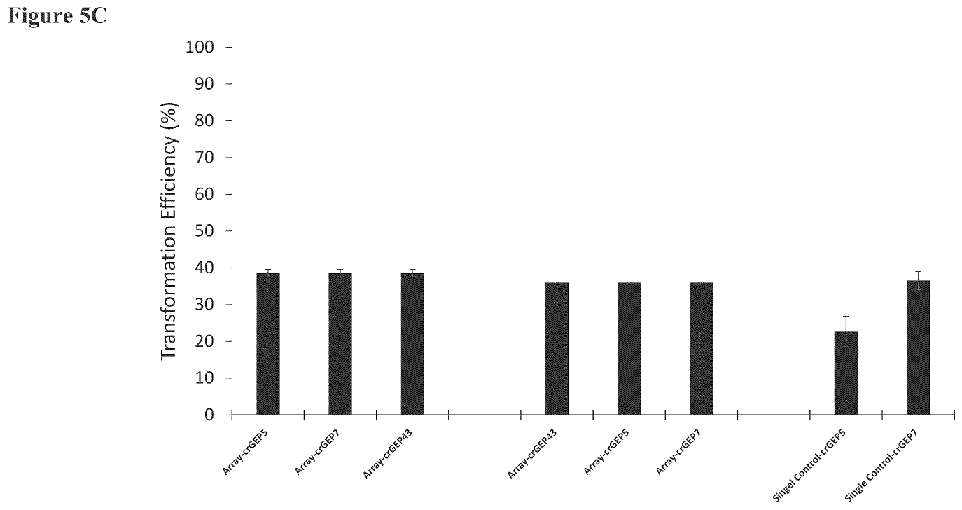

This experiment was performed in order to test the ability of the gRNA/ribozyme system to disrupt more than one target simultaneously by the expression of three gRNAs, namely crGEP5, crGEP7 and crGEP43 from an array format (cf. Table 1). Based on the data shown in FIG. 5B, the authors concluded that the indel activity for each gRNA remains unchanged irrespective of its position in the array (see indel activity for array 1 compared to the indel activity of array 2). Although the activity of gRNAs tested in the arrays were slightly lower and/or comparable to the activity of the respective gRNA when expressed in isolation, the data nevertheless indicate that gRNA arrays can be used to simultaneously target multiple genomic sequences in plant cells. The activity from the arrays was lower or comparable than control single guides and this experiment thus provided a proof of concept for multiplex targeting using the Ribozyme strategy.

### Example 10: mRNA-based Guide Arrays and Artichoke Ribozyme-based Arrays do Show Activities at Individual Sites Comparable or Better than Control Single Guides

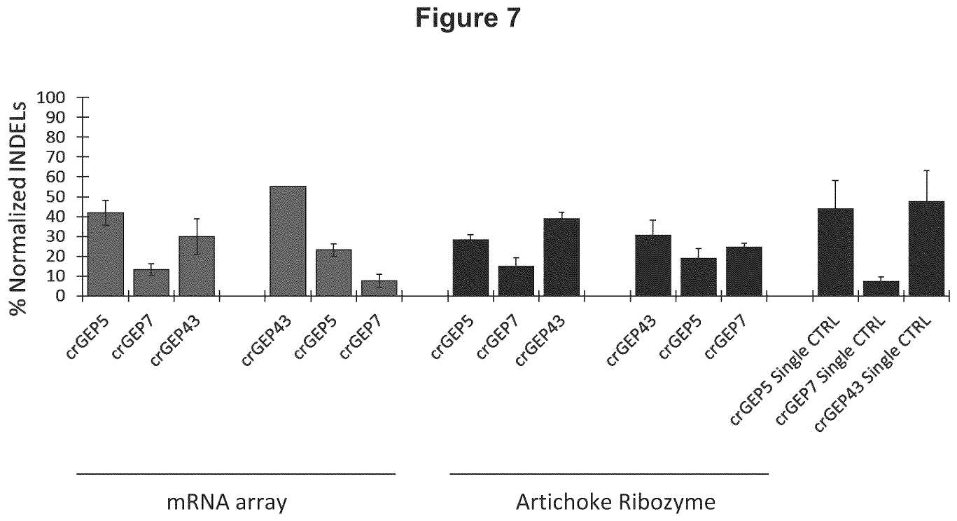

In this experiment two arrays with three targets (Table 1 and FIG. 7) in different order across the two systems analyzed herein, the mRNA embedded delivery system carrying the corresponding LbCpf1 crRNA as detailed in Table 1 and the Artichoke Ribozyme based array carrying the corresponding LbCpf1 crRNA as detailed in Table 1, were extensively compared. Results are shown in FIG. 7. The controls are the single targets in the HDV ribozyme version. Clearly both systems work well. In the artichoke array the target crGEP43 is best performing irrespective of its position while in the mRNA array the first crRNA in the in the array performs the best. Both systems seem to show activity at least comparable to, or even better than single guide RNAs.

The array systems were introduced into base vectors pGEP424 for mRNA array or pGEP376 for Artichoke based array. The array sequences sequence of crGEPs 5/7/43 and crGEPs 43/5/7 correspond to the same sequence as described above. The vector sequences used in the mRNA arrays (i.e. for vectors pGEP471 and pGEP472) and the vector sequences used for the artichoke arrays (i.e. for vectors pGEP473 and pGEP474) are set forth in SEQ ID NO: 68 to 71.

### Example 11: An Optimized Cpf1 Nuclease System for Corn and Wheat Cells

To proof functionality of the Cpf1 system in corn, corn ears were harvested at 9-10 days post pollination and immature embryos (IE) were extracted from individual kernels. The embryos were placed on culture medium containing 2,4 D and cultured 2 days in preparation for transformation by particle bombardment. On the day of transformation the IE were transferred to another medium with high osmotic potential at least 4 hrs prior to particle bombardment to further prepare them. Following the 4 hrs pre-treatment, the IE were bombarded 2× with 0.6 μM gold particles coated with the nuclease plasmid and crRNA plasmid. The day after bombardment, embryos were transferred off of the osmotic medium and then cultured for 3 days. During this process, the fluorescent marker was expressed and seen as distinct fluorescent spots on the surface of the treated IE. Excess IE explants are bombarded and then a subset of the explants were selected based on having the highest number of fluorescent cells. Approximately half of the bombarded explants are added together as a pooled sample for DNA extraction and then submitted for amplicon deep sequencing via the NGS process. After bombardment of these explants the nuclease and crRNAs are expressed in the cells where DNA was delivered and provided the activity needed for genome editing (SDN-1 approach). This activity was detected using amplicon deep sequencing despite the dominant presence of wild type cells without editing.

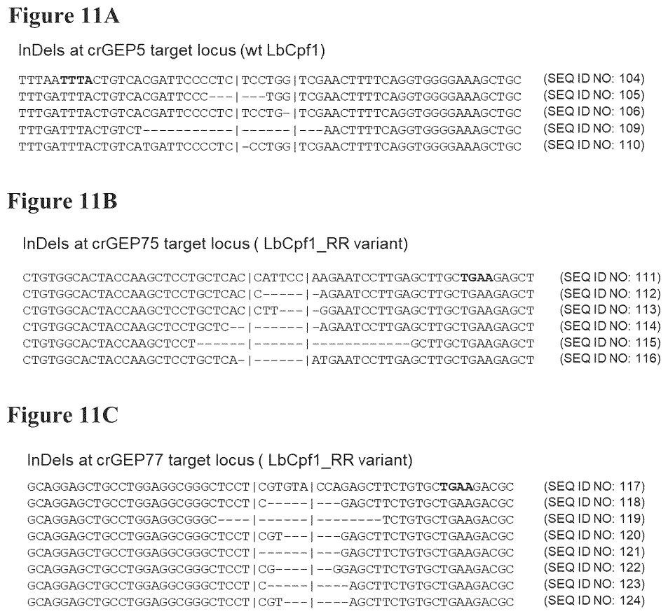

FIG. 11 shows a number of high efficiency targets based on protoplast data. In IE tissue, the highest values that have been observed for transient activity are between 0.2 to 0.3%. In this experiment the inventors had two samples, one with crGEP75 and one with crGEP77, that were approaching 0.1% activity at their respective target sites. The sequence data shows the highest numbers of sequence reads and are demonstrating that the deletions are in the expected region of the proto-spacer.

Furthermore, it was tested whether a wheat protoplast system can be used to identify best guides for a given target locus. Therefore, five guides for Cpf1 (RR variant) were designed at the TaTDF locus in the same vicinity of an efficient Cas9 guide location. Each of these guides were cloned into the ribozyme delivery base vector and co-delivered with the RRLbCp1 nuclease into wheat protoplasts by PEG transformation. 24 hrs later, samples were collected and after flow cytometry were analysed for transformation efficiency. NGS analysis for target sites gave % raw InDels which were multiplied by each samples % protoplast transformation efficiency to give the normalized InDel efficiency. From the NGS data it was evident that guide crGEP56 was the most efficiently cut target in all three genomes of wheat (see FIG. 12).

Furthermore, five additional Cpf1 genes (SEQ ID NO: 72-76) encoding the same protein have been designed and generated and are currently tested in monocots for their gene editing efficiency. The five variants of LbCpf1 that have been synthesized are sequences generated as codon-optimized variants from three vendors or a published version from Tang et al 2017, Nature Plants 3:17018.

### Example 12: The Cpf1 Nuclease can be Used to Generate Heritable Edits in Corn Plants

To test whether the Cpf1 nuclease can be used to generate heritable edits in corn plants high type II immature embryos were co-bombarded with 2 constructs and 15 ng per shot with the chemical compound Trichostatin A (TSA), which was included to stimulate cell division and/or gene editing events. The constructs encoded the genes needed to obtain SDN-1 events at the HMG13 gene locus in corn. The first construct comprises a ZmUbi1 promoter controlling the expression of LbCpf1 gene and a separate fluorescent marker gene under control of the 2XCaMV35S promoter. The second plasmid encodes the crRNA gene under control of a ZmUbi1 promoter. The constructs were bombarded to either transiently or stably express the LbCpf1 gene and the crRNA in order to obtain SDN-1 edits early in the cell culture process that could later be captured in regenerated plants from cultures. Regenerated T₀ plants were moved to phyta-trays and placed in the growth chamber for continued growth and recovery. Until sufficient amount of plants of the T₀ population had grown, plants were sampled to detect SDN-1 events by capillary electrophoresis (CE) assay. The CE assay was developed to identify insertions and deletions (InDels) in amplicon pools generated by PCR amplification of a selected Cpf1 target locus. Pooled leaf tip samples were taken from each individual plant to facilitate detection of SDN-1 events even if the pants were chimeric for the editing event. Positive T₀ plants were moved to soil in flats within approximately 3 weeks of sampling to ensure the recovery of viable plants to the greenhouse. Once the plants were established in soil, a second round of analysis was initiated consisting of repeating the capillary electrophoresis with a new leaf sample and later as the plant was developing, additional samples for PCR and sequence analysis were collected. By this approach, plants which were chimeric for the SDN-1 edit were easier to detect.

Following molecular confirmation of SDN-1 in plants and their sufficient growth in flats, plants were transferred to pots for growth to fertility and seed recovery. SDN-1 and wild type A188 plants were used in reciprocal crosses to insure good pollination and to better insure the recovery of the SDN-1 event in the next generation (Hill X A188, A188 X Hill). Approximately 15-20 days after pollination, one ear was harvested and surface sterilized for immature embryo extraction. Immature embryos were extracted from kernels and placed embryo axis side down on a basic MS based embryo culture medium and cultured for 3 days in the dark which initiated radicle and plumule development. Next, they were transferred to the light for normal plant development and once the plants were of sufficient size they were sampled for detection of the edit to confirm transmission to the T₁ generation.

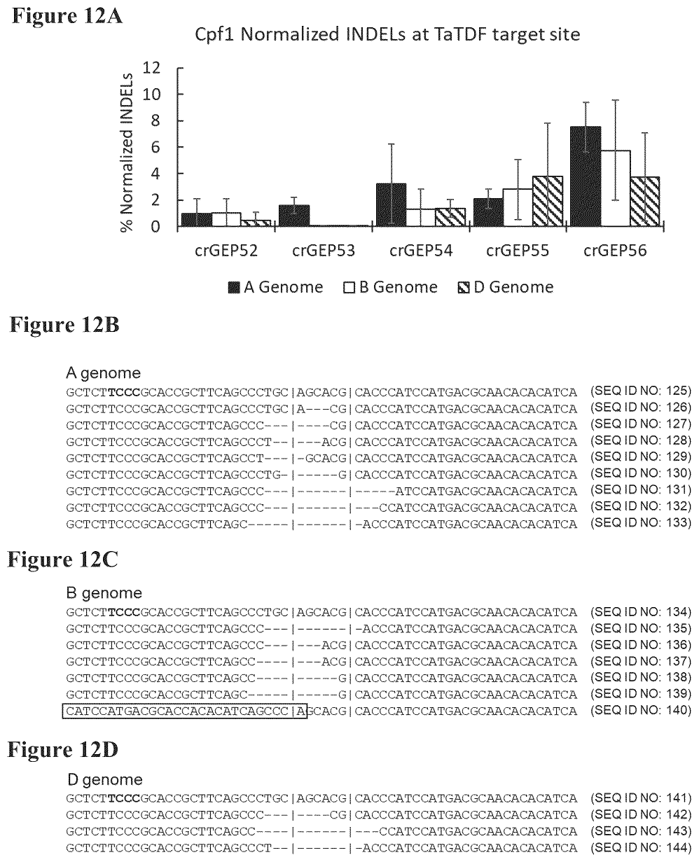

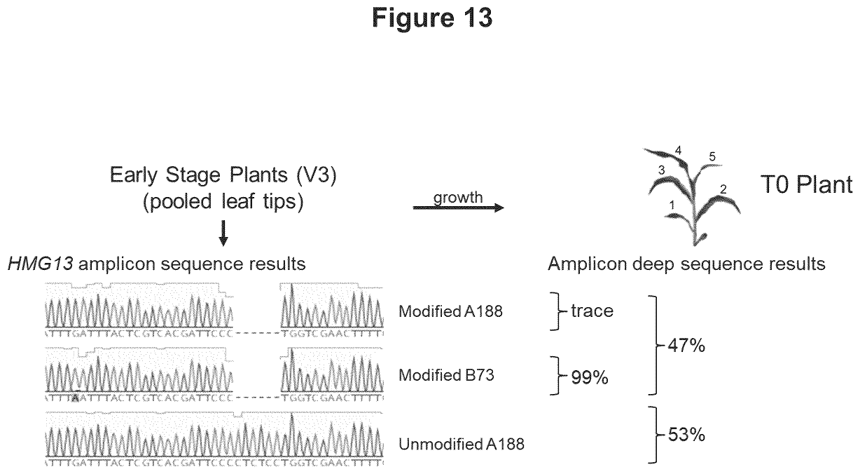

The obtained data confirmed SDN-1 edits and inheritance of two InDel events generated by Cpf1 in corn embryos which regenerated into whole plants (FIG. 13 and Table 4). Approximately 185 high type II immature embryos were co-bombarded with the constructs described for SDN-1 at the HMG13 target site. The Cpf1 gene construct also carried a constitutive red fluorescent marker gene (tdTomato). Embryos were cultured to induce type II callus and calli were analyzed for the expression of the red fluorescent marker gene. Approximately 1100 T₀ plants were regenerated and transferred to phyta-trays for additional growth and recovery. Pooled leaf tip samples were taken from each plant or small plant cluster for DNA extraction in multi-well plates. Genomic DNA was extracted and submitted for the capillary electrophoresis assay. One event had 5 regenerated shoots and each of the 5 demonstrated the presence of the same 6 bp deletion in the amplicon (FIG. 13). These plants were introduced into the plant recovery process and 3/5 were able to be recovered to the greenhouse. Following transplant to soil and establishment of the plants by demonstration of new growth, new pooled leaf tip samples were taken for the CE assay and used to confirm that the deletion was still detectable in the growing T₀ plants at the V4 to V7 stage. Individual leaf samples were taken following additional plant growth and transplant to pots. PCR was used to generate amplicons from each individual leaf sample and then individual amplicons were sequenced by first capturing them in a plasmid and then cloning them for sequencing. The inventors were able to show that all of the amplicons shared the same InDel sequence and it appeared that the plants were not chimeric for the genome edit. These same DNA samples were submitted to an NGS process based on amplicon deep sequencing. The results shown in FIG. 13 demonstrate that approximately 53% of the samples was unmodified at the target site and 47% was modified with the 6 bp deletion. Although there was a trace amount of this deletion detected in the A188 genome, most was in the B73 genome as was evident by the presence of a G to A single nucleotide polymorphism (SNP).

Next, plants were grown to fertility and then pollinated. In the F₁ generation, 160 plants were recovered from extracted 180 kernels. Individual leaf samples were taken from these plants and submitted for the CE assay. The assay was positive for 63 of 160 of these samples thereby confirming transmission of the InDel to the next generation.

In a second experiment, five Hi-II T₀ plants were found to contain KO mutations in the crGEP5 target which were then transferred to soil. PCR and sequence analysis of DNA showed a bi-allelic deletion profile with a 5 bp deletion in the B73 chromosome and a 32 bp deletion in the A188 chromosome. T₁ transmission was confirmed by PCR and sequence analysis according to the pattern shown in the following table:

TABLE 4
Cpf1 causes heritable edits in corn plants.

| | | Number of | Sequence-confirmed |
|---|---|---|---|
| T0 genotype | Crosses | T1 plants | T1 genotypes |
| 5 bp/32 bp bi- | KO × A188 | 36 | 31/33 heterozygous KO |
| allelic KO | | | |
| 5 bp/32 bp bi- | KO × A188 | 36 | 23/23 heterozygous KO |
| allelic KO | | | |
| 5 bp/32 bp bi- | KO × KO | 36 | 33 homozygous KO |
| allelic KO | (self) | | |
| 5 bp/32 bp bi- | KO × A188 | 36 | 32/32 heterozygous KO |
| allelic KO | | | |
| wt Control | A188 × A188 | 18 | 18/18 A188 wt |
| | (control) | | |

### Example 13: Demonstration of Efficient Cpf1-mediated Editing in T0 Sugar Beet Plants

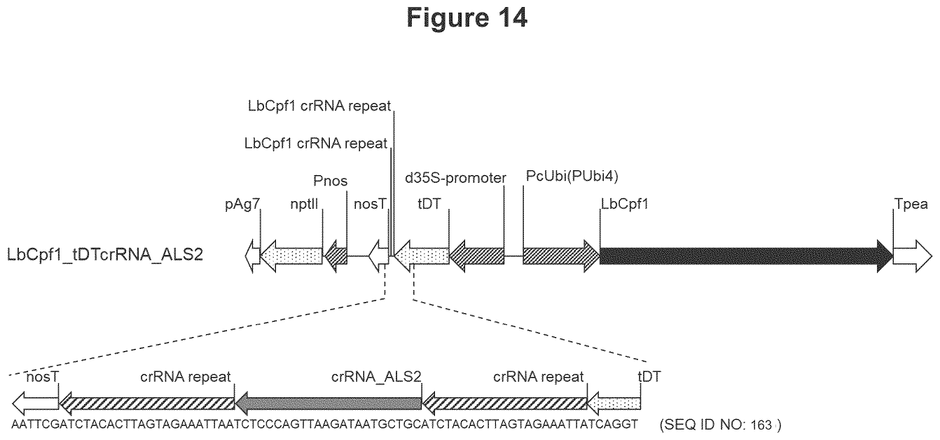

To test whether the herein described Cpf1 systems can be used to generate edits in T0 sugar beet plants, the following experiments were performed. For stable transformation of sugar beet plants the binary vector construct LbCpf1_tDTcrRNA_ALS #2 was used (FIG. 14). This construct includes (i) an nptII resistance cassette for in planta selection (ii) the PcUbi4::Cpf1 expression cassette and (iii) a crRNA, which is directed against target region BvALS #2 within the target gene BvALS. The crRNA is embedded in a Pol II-driven RNA transcript as described before in Zhong et al., 2017. This transcript includes an mRNA encoding the fluorescent protein tDT.

The stable callus transformation method described below is based on the method published by Kishchenko et al, 2005 (Kishchenko, E. M., et al. (2005). “Production of transgenetic sugarbeet (Beta vulgaris L.) plants resistant to phosphinothricin.” Cell Biology International 29 (1): 15-19).

Micropropagated shoots of the genotype S706 were used as starting material. Shoots were multiplied in medium E. To induce friable callus, leaf explants were incubated in medium D at 28° C. for 7-8 weeks. Friable calli were harvested in medium F and kept for 1 week in the dark at 24° C. Agrobacterium AGL-1 harbouring the binary vector of interest was grown in medium A supplemented with the appropriate antibiotics at 28° C. for 24 h. Calli were inoculated with Agrobacterium suspension prepared in medium B at an OD600 of 0.6-0.8. The co-culture of the callus tissue and the Agrobacterium was done in medium C at 21° C. for three days in the dark. Calli were sub-cultured to medium G and incubated in the dark at 24° C. for one week. For the selection of transgenic cells, calli were transferred to medium H and incubated at 24° C. in the light (16 h) for three weeks. Transgenic calli were selected and sub-cultured for several times in the same medium and conditions. Regenerating shoots were isolated and propagated in medium I. Selected shoots were rooted in medium J and transferred to the green house for seed production.

Callus transformation of sugar beet using the construct LbCpf1_tDTcrRNA_ALS#2 resulted in 115 regenerated shoots, which were analyzed for presence of Cpf1 and SDN-1 genome editing. The results of this analysis are summarized in Table 5. 22.6% (26/115) of the regenerated shoots have been positively tested for the presence of LbCpf1 using qPCR. Those shoots are termed “transgenic”. 73.1% (19/26) of the transgenic shoots have been positively tested for the presence of SDN-1 editing events (INDELS) via sequencing and/or capillary electrophoresis of the amplified target region. This ratio of 73.1% therefore corresponds to the LbCpf1 editing efficiency. In addition to the 19 stably transformed edited shoots, we obtained three shoots, which do not have LbCpf1 stably integrated but harbour SDN-1 events in the desired target location within BvALS. Those shoots represent transient editing events (Table 5).

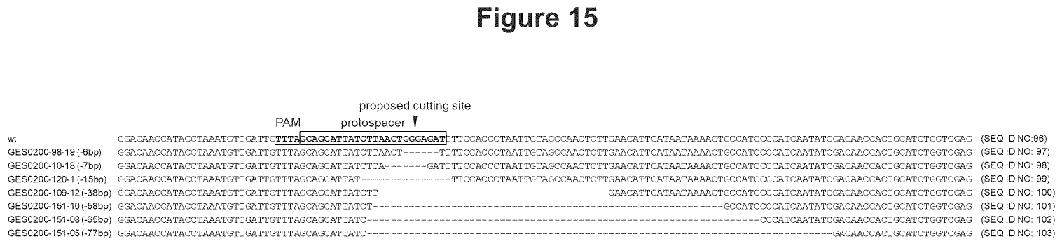

A selection of edited shoots was analyzed in detail by performing sequencing of the amplified BvALS target region. This analysis revealed the presence of varying deletions (−6 bp to −77 bp) in the different analyzed shoots (FIG. 15). No insertions or substitutions were identified.

TABLE 5
Callus transformation of sugar beet using the construct
LbCpf1_tDTcrRNA_ALS#2 resulted in 115
regenerated shoots, which were analyzed for the
presence of Cpf1 and SDN-1 genome editing.

| | |
|---|---|
| total number of shoots analyzed | 115 |
| transgenic | 26 |
| edited (transgenic) | 19 |
| non-edited (transgenic) | 7 |
| Cpf1 efficiency (transgenic) | 73.08% |
| Edited non-transgenic | 3 |

### Example 14: Guide RNA Abundance can be Modulated by Repeated Arrays of the Same crRNA Sequence

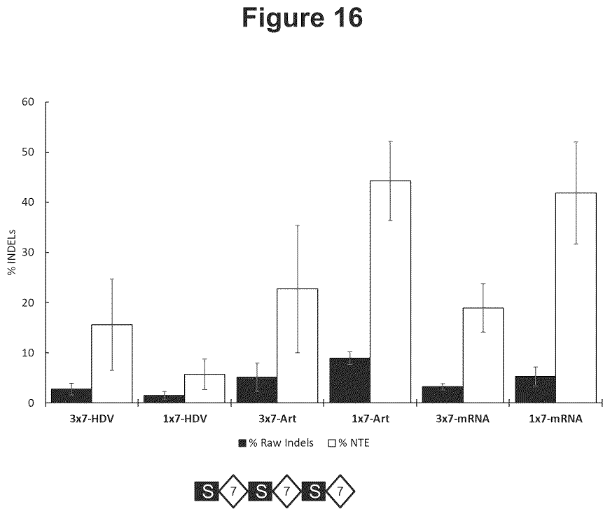

The intention was to test whether an increased dosage of target crRNA could lead to increased InDel events at a given target site. Target crGEP7 in gene HMG13 always gives low InDel % using a HDV ribozyme system. To test whether an array of 3 guide RNAs could increase activity at target site locus, a 3× scaffold(s) and target 7 guide module was cloned into constructs which are base vectors for HDV, Artichoke ribozyme and mRNA delivery systems (FIG. 16). The control was the 1× crGEP7 vector for each delivery system. Each construct was co-transformed into protoplasts with the LbCpf1 nuclease vector and samples collected at 24 hrs for analysis after flow cytometry was carried out for transformation efficiency. NGS analysis of target site crGEP7 gave us 7% raw InDels which were multiplied by each samples % protoplast transformation efficiency to give the 5 NTE. FIG. 16 shows that the 3× guide delivery system seems to be beneficial for HDV delivered target sites but not for the other delivery methods.

### Example 15: Modifying the Relative Dosage of the Cpf1 Protein and crRNA Modulates the Editing Efficiency in Plant Cells

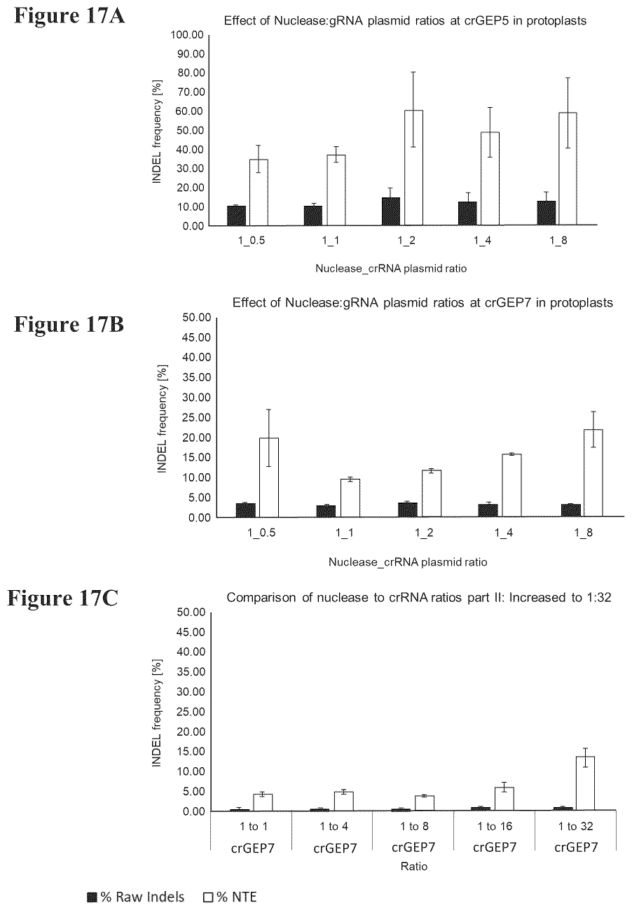

Maize A188 protoplast were transfected with different molar ratios of 2 plasmids to examine the effect on cutting efficiency. One of these plasmids has a plant gene cassette including the Cpf1 nuclease gene plus a fluorescent marker gene and the second plasmid has a similar cassette with one of two crRNA in Rm-HMG13, crGEP5 or crGEP7. Typically, these plasmid are transfected at a 1:1 molar ratio while holding the overall DNA quantity for transfection constant at 20 μg per transfection. In order to hold the DNA concentration constant in these experiments while increasing the crRNA plasmid amount, the nuclease gene plasmid concentration, was dropped as the crRNA plasmid concentration increased incrementally. Following transfection and a standard culture time for protoplasts, the cells exhibiting a fluorescent phenotype were counted using a flow cytometer to measure the transformation frequency. The recovered cells were pelleted and frozen in preparation for genomic DNA extraction. DNA was extracted and submitted for the NGS process, better described as amplicon deep sequencing, to measure the presence of insertions and deletions (InDels) at the expected target site. Amplicons with InDels were counted using NGS and reported as a percentage of total amplicon reads (short bars in FIG. 17). These values were finally reported as a function of the protoplast transformation frequency (taller bars in FIG. 17).

The experiments involved two genomic target sites, crGEP5 (FIG. 17) and crGEP7 (FIGS. 17 B and C), that had previously been characterized as having high and low activity, respectively. The previous characterization experiments also used maize A188 protoplasts and a 1:1 molar ratio of nuclease to crRNA plasmids. The variation in target site activity was of interest in these tests to determine if there would be different responses. The higher active site, target crGEP5 did not show a significant difference in activity with increasing crRNA plasmid concentrations (FIG. 17A). There might be an increase of approximately 30% when increasing the ratio from 1:1 to 1:2 but no further increase from 1:4 and 1:8 ratios. Interestingly, there was no significant difference in InDel frequency when half the crRNA quantity was used (FIG. 17A, 1_0.05). In contrast, the low efficiency target crGEP7 showed incremental increases with each increase in the crRNA concentration (FIG. 17 B) ending at the 1:8 ratio which had about twice the activity of the 1:1 ratio. FIG. 17 C represents an experiment which was designed to extend the quantity of the crRNA even higher compared to that shown in FIG. 17 B.

### Example 16: Codon Optimized Variants of LbCpf1 Show Enhanced Activity at Target Sites in Corn Leaf Protoplasts or Non-dividing Cells

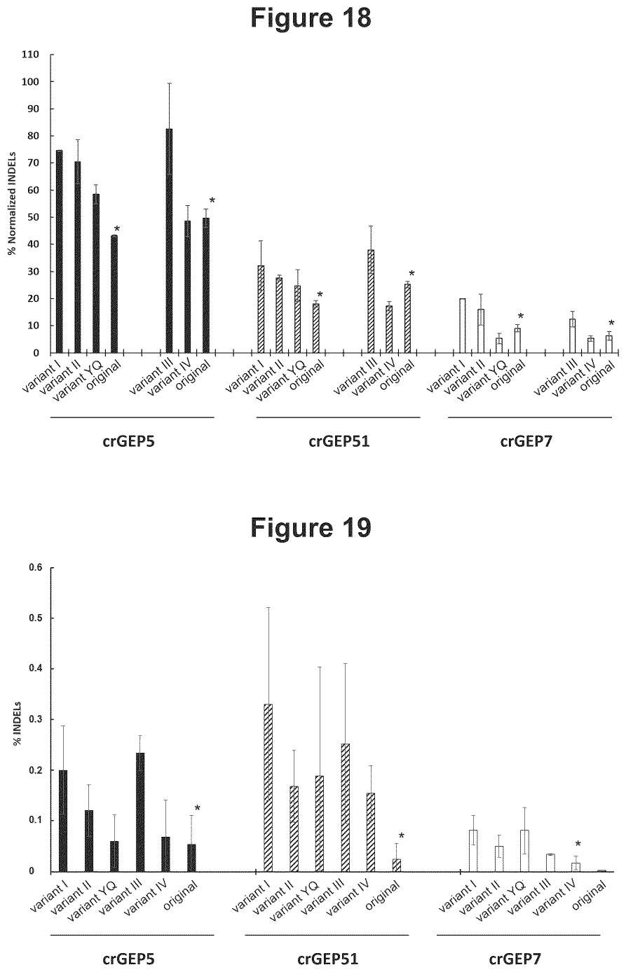

Five Codon optimized variants of LbCpf1 were made and tested across three target sites in a protoplast assay. Four designed versions ordered at commercial sequence providers (variants I (SEQ ID NO: 72), II (SEQ ID NO: 73), III (SEQ ID NO: 75) and IV (SEQ ID NO: 76)) and one published version from Yiping Qi's group (Tang et al., 2017 Nature Plants 3:17018) (variant YQ (SEQ ID NO: 74)). A total of 15 μg of Nuclease plasmid and 8 μg of target guide plasmid were transformed into corn leaf protoplasts by PEG method and reporter gene expression from nuclease vector was quantified by flow cytometry for transformation efficiency. Protoplast samples were collected at 24 hrs and INDEL activity from the LbCpf1 variants was compared to the original version of LbCpf1 (see FIG. 18).

In Each experiment the results show that that multiple variants show activity equal to or even better than the original LbCpf1 version. Based on these results two variants I and III showed consistent enhanced activity against all three targets over the other variants.

### Example 17: Codon Optimized Variants of LbCpf1 Show Enhanced Activity at Target Sites in Corn Immature Embryos (IE) or Dividing Cells

The five Codon optimized variants of LbCpf1 of example 16 were tested also across three target sites in corn IEs. A total of 100 ng of Nuclease plasmid and 150 ng of target guide plasmid were bombarded into corn IE (9 days after pollination) and reporter gene expression from nuclease vector was observed by microscopy for transformation efficiency. Samples were collected at 48 hrs and INDEL activity from the LbCpf1 variants was compared to the original version of LbCpf1 (see FIG. 19).

In Each experiment the results show that that multiple variants show activity equal to or better than the original LbCpf1 version. Like in experiment 16 the results showed that the two variants I and III consistently enhance the activity against all three targets over the other variants.

### Example 18: Intron-sequence Containing Cpf1 Variants Show Improved INDEL Activity Compared to Intron-less Versions

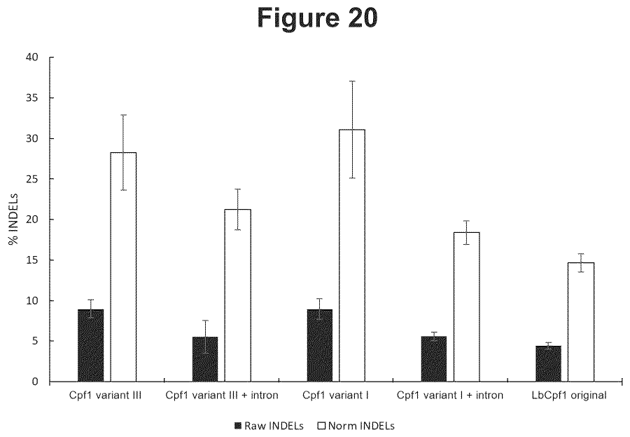

To prevent sequence rearrangement in Agrobacterium T-DNA vectors due to expression of the Cpf1 (lacking intron to prevent bacterial expression) we constructed Intron containing Cpf1 variants of the two best versions (variants I and III; SEQ ID NOs: 157 and 158) as described in the experiments 16 and 17 and compared them to the LbCpf1 original version (without intron) in protoplast assays. A total of 15 μg of Nuclease plasmid and 8 μg of Target guide plasmid were transformed into corn leaf protoplasts by PEG method and reporter gene expression from nuclease vector was quantified by flow cytometry for transformation efficiency. Protoplast samples were collected at 24 hrs and INDEL activity from the LbCpf1 variants was compared to the original version (see FIG. 20).

It is evident that the intron-containing Cpf1 variants show lesser activity than the control intron-less versions of themselves but still show higher activity over the original version of LbCpf1 (intron-free). These results indicate that these intron containing variants still outperform the original Cpf1 and can be used to make stable Agrobacterium T-DNA vectors.

1-52. (canceled)

53. A plant delivery system, the delivery system comprising
(a) at least one Cpf1 enzyme or an active fragment thereof, or a nucleic acid sequence encoding the same; and
(b) at least one Cpf1 guide RNA system, or a nucleic acid sequence encoding the same, the at least one Cpf1 guide RNA system comprising at least one Cpf1 guide RNA specific for a target site in the genome of a plant or part of a plant; wherein the at least one Cpf1 guide RNA, or the nucleic acid sequence encoding the same, is embedded within a 3′ untranslated region (UTR) of a fluorescent marker encoding gene encoding for a fluorescent protein that facilitates monitoring of activation of the embedded at least one Cpf1 guide RNA by quantifying a fluorescence in a target cell or a cellular system of interest, and further comprises a scaffold RNA sequence, or a sequence encoding the same, at the 5′ and 3′ end,
wherein the at least one Cpf1 enzyme, or active fragment thereof, or nucleic acid sequence encoding the same, comprises at least one mutation in comparison to a wild-type sequence resulting in an altered PAM recognition, wherein the at least mutation is selected from G532R/K595R, or G532R/K538V/Y542R in comparison to the sequence of SEQ ID NO: 16.

54. The plant delivery system of claim 53, wherein the plant delivery system comprises a first nucleotide molecule comprising a nucleic acid sequence encoding the at least one Cpf1 enzyme or an active fragment thereof, and a second nucleotide molecule comprising a nucleic acid sequence encoding the at least one Cpf1 guide RNA system, preferably wherein the first and the second molecule are provided on separate constructs, or wherein the first and the second molecule are provided on a single transcript construct.

55. The plant delivery system according to claim 54, wherein the first nucleotide molecule and the second nucleotide molecule are delivered in a ratio selected from the group consisting of: 1.0 to 0.5; 1.0 to 01.0; 1.0 to 1.5; 1.0 to 2.0; 1.0 to 2.5; 1.0 to 3.0; 1.0 to 3.5; 1.0 to 4.0; 1.0 to 4.5; 1.0 to 5.0; 1.0 to 5.5; 1.0 to 6.0; 1.0 to 6.5; 1.0 to 7.0; 1.0 to 7.5; and 1.0 to 8.0.

56. The plant delivery system of claim 55, wherein the first construct comprising the first nucleotide molecule and the second construct comprising the second nucleotide molecule each comprise at least one promoter functional in a plant or part of a plant, preferably wherein the at least one promoter driving expression of the first and the second nucleotide molecule is different in the first and the second construct, respectively.

57. The plant delivery system of claim 53, wherein the nucleic acid sequence encoding the Cpf1 enzyme or an active fragment thereof is selected from the group consisting of SEQ ID NOs: 13 and 14, or a sequence having at least 85% identity over the whole length of the respective sequence of SEQ ID NOs: 13 or 14; or
wherein the at least one Cpf1 enzyme or an active fragment thereof, or a nucleic acid sequence encoding the same, is selected from the group consisting of SEQ ID NOs: 38, 39, 72, 73, 74, 75, 76, 157, and 158, or a sequence having at least 80% or more sequence identity over the whole length of the respective nucleic acid sequence of SEQ ID NOs: 38, 39, 72, 73, 74, 75, 76, 157, or 158; or
wherein the at least one Cpf1 enzyme or an active fragment thereof, or a nucleic acid sequence encoding the same, is selected from the group consisting of SEQ ID NOs: 15, 16, 40, 41, 152, 153, 154, 155, and 156, or a sequence having at least 90% identity over the whole length of the respective amino acid sequence of SEQ ID NOs: 15, 16, 40, 41, 152, 153, 154, 155, or 156, respectively.

58. The plant delivery system of claim 53, wherein the scaffold RNA sequence, or a sequence encoding the same, is selected from the group consisting of SEQ ID NO: 29 and 30, or a sequence having at least 95% identity over the whole length of SEQ ID NO: 29 or 30.

59. The plant delivery system of claim 53, wherein the at least one Cpf1 guide RNA system comprises at least two guide RNAs, wherein the at least two guide RNAs are separated by a nucleotide sequence comprising direct repeats.

60. The plant delivery system of claim 53, wherein the translatable or non-translatable sequence is selected from the group consisting of SEQ ID NOs: 31, 32, 13, 14, 38, 39, 72, 73, 74, 75, 76, 157, and 158, or a sequence having at least 85% identity over the whole length of the respective sequence of SEQ ID NOs: 31, 32, 13, 14, 38, 39, 72, 73, 74, 75, 76, 157, or 158.

61. A plant, or a plant cell, tissue, organ or material, or a derivative or progeny thereof, comprising a plant delivery system of claim 53.

62. A method for modifying a target site in the genome of a plant or part of a plant, wherein the method comprises the steps of:
(a) providing at least one Cpf1 enzyme or an active fragment thereof, or a nucleic acid sequence encoding the same; preferably, wherein the at least one nucleic acid sequence encoding the Cpf1 enzyme or an active fragment thereof is codon-optimized for the expression in the plant or part of the plant, wherein the at least one Cpf1 enzyme, or active fragment thereof, or nucleic acid sequence encoding the same, comprises at least one mutation in comparison to a wild-type sequence resulting in an altered PAM recognition, wherein the at least mutation is selected from G532R/K595R or G532R/K538V/Y542R in comparison to the sequence of SEQ ID NO: 16; and
(b) providing at least one Cpf1 guide RNA system, or a nucleic acid sequence encoding the same, the at least one Cpf1 guide RNA system comprising at least one Cpf1 guide RNA specific for a genomic target sequence of interest in the plant or part of the plant;
wherein the at least one Cpf1 guide RNA, or the nucleic acid sequence encoding the same, is embedded within a 3′ untranslated region (UTR) of a fluorescent marker encoding gene encoding for a fluorescent protein that facilitates monitoring of activation of the embedded at least one Cpf1 guide RNA by quantifying a fluorescence in a target cell or a cellular system of interest,
and further comprises a scaffold RNA sequence, or a sequence encoding the same, at the 5′ and 3′ end;
(c) optionally: providing at least one repair template nucleic acid sequence, wherein the at least one repair template nucleic acid sequence is preferably flanked by one or more homology sequence(s) complementary to one or both adjacent region(s) of the genomic sequence of interest in the plant or part of the plant;
(d) introducing the at least one Cpf1 enzyme or an active fragment thereof, or a nucleic acid sequence encoding the same from step (a); and introducing the at least one Cpf1 guide RNA system, or a nucleic acid sequence encoding the same from step (b) and optionally: introducing the at least one repair template nucleic acid sequence from step (c) into the plant or part of the plant, wherein the introducing is done by transformation
(e) thereby obtaining the plant or the part of the plant, comprising a modification in the genomic target sequence of interest.

63. Use of a plant delivery system as defined in claim 53; or use of a Cpf1 enzyme or an active fragment thereof, or of a nucleic acid sequence encoding the same as defined in claim 53; and/or a use of at least one guide RNA system, or the nucleic acid sequence encoding the same as defined in claim 53; in a method of modifying a target site in the genome of a plant or part of a plant.
